(19) United States
(12) Patent Application Publication
(10) Pub. No.: US 2021/0163594 A1
(43) Pub. Date: Jun. 3, 2021

(54) BINDING MOLECULES

(71) Applicant: INVENRA INC., Madison, WI (US)

(72) Inventors: Lucas BAILEY, Suzhou (CN); Bryan GLASER, Fitchburg, WI (US); Qufei LI, Suzhou (CN); Roland GREEN, Madison, WI (US); Dileep Kumar PULUKKUNAT, Middleton, WI (US); Malgorzata Agnieszka NOCULA-LUGOWSKA, Brighton, MA (US); Daniel Justin GERHARDT, Madison, WI (US); Matthew Joseph BISSEN, Madison, WI (US)

(21) Appl. No.: 17/048,508

(22) PCT Filed: Apr. 17, 2019
(86) PCT No.: PCT/US2019/027991

§ 371 Date: Oct. 16, 2020

Related U.S. Application Data
(60) Provisional application No. 62/659,079, filed on Apr. 17, 2018.

(51) Int. Cl. C07K 16/28

(52) U.S. Cl. CPC C07K 16/2809; A61K 2039/505

(57) ABSTRACT

Binding molecules, constructs, pharmaceutical compositions comprising the constructs, and methods of use thereof are presented.

### 1. CROSS-REFERENCE TO RELATED APPLICATIONS

This application claims the benefit of U.S. Provisional Application No. 62/659,079, filed on Apr. 17, 2018, which application is hereby incorporated in its entirety by reference.

### 2. SEQUENCE LISTING

The instant application contains a Sequence Listing which has been submitted via EFS-Web and is hereby incorporated herein by reference in its entirety. Said ASCII copy, created on Month XX, 2019, is named XXXXXUS_sequencelisting.txt, and is X,XXX,XXX bytes in size.

### 3. BACKGROUND

Antibodies are an invaluable tool in the medical field. In particular, the importance of monoclonal antibodies, including their roles in scientific research and medical diagnostics, have been widely recognized for several decades. However, the full potential of antibodies, especially their successful use as therapeutic agents, has only more recently been demonstrated, as demonstrated by the successful therapies adalimumab (Humira), rituximab (Rituxan), infliximab (Remicade), bevacizumab (Avastin), trastuzumab (Herceptin), pembrolizumab (Keytruda), and ipilimumab (Yervoy). Following these clinical successes, interest in antibody therapies will likely only continue to increase. Therefore, a need for efficient generation and manufacturing of antibodies exists in the field, both in the research drug development and downstream clinical settings.

An area of active research in the antibody therapeutic field is the design and use of multispecific antibodies, i.e. a single antibody engineered to recognize multiple targets. These antibodies offer the promise of greater therapeutic control. For example, a need exists to improve target specificity to reduce the off-target effects associated with many antibody therapies, particularly in the case of antibody-based immunotherapies. In addition, multispecific antibodies offer new therapeutic strategies, such as synergistic targeting of multiple cell receptors, especially in an immunotherapy context. One such immunotherapy is the use of bispecific antibodies to recruit T cells to target and kill specific tumor cell populations through bispecific engagement of a T cell marker and a tumor cell marker. For example, the targeting of B cell lymphoma using CD3×CD19 bispecific antibodies is described in U.S. Pub. No. 2006/0193852.

Despite the promise of multispecific antibodies, their production and use has been plagued by numerous constraints that have limited their practical implementation. In general, all multispecific antibody platforms must solve the problem of ensuring high fidelity pairing between cognate heavy and light chain pairs. However, a multitude of issues exist across the various platforms. For example, antibody chain engineering can result in poor stability of assembled antibodies, poor expression and folding of the antibody chains, and/or generation of immunogenic peptides. Other approaches suffer from impractical manufacturing processes, such as complicated in vitro assembly reactions or purification methods. In addition, several platforms suffer from the inability to easily and efficiently plug in different antibody binding domains. These various problems associated with multispecific antibody manufacturing limit the applicability of many platforms, especially their use in high-throughput screens necessary for many therapeutic drug pipelines, such as in screening for improved antigen binding specificity or affinity.

There is, therefore, a need for an antibody platform capable of high-level expression and efficient purification. In particular, there is a need for a multispecific antibody platform that improves the manufacturing capabilities of multispecific antibodies with direct applicability in both research and therapeutic settings. There is also a need for improved multispecific antibodies that specifically bind to distinct cell populations, including tumor cell populations, with improvements including increased affinity or avidity, reduced off-target binding, and/or reduced unintended immune activation.

### 4. SUMMARY

The present disclosure provides various binding molecules, pharmaceutical compositions, and methods of treatment.

In a first aspect the disclosure provides a binding molecule comprising, a first, second, third, fourth, and fifth polypeptide chain, wherein: the first polypeptide chain comprises a domain A, a domain B, a domain D, and a domain E, wherein the domains are arranged, from N-terminus to C-terminus, in a A-B-D-E orientation, wherein domain A comprises a variable region domain amino acid sequence, domain B comprises a CH3 domain amino acid sequence, domain D comprises a constant region domain amino acid sequence, and domain E comprises a constant region domain amino acid sequence; the second polypeptide chain comprises a domain F and a domain G, wherein the domains are arranged, from N-terminus to C-terminus, in a F-G orientation, and wherein domain F has a variable region domain amino acid sequence and domain G comprises a CH3 domain amino acid sequence; the third polypeptide chain comprises a domain H, a domain I, a domain J, and a domain K, wherein the domains are arranged, from N-terminus to C-terminus, in a H-I-J-K orientation, and wherein domain H has a variable region domain amino acid sequence, domain I has a constant region amino acid sequence, and domains J and K have a constant region domain amino acid sequence; the fourth polypeptide chain comprises a domain L and a domain M, wherein the domains are arranged, from N-terminus to C-terminus, in a L-M orientation, and wherein domain L has a variable region domain amino acid sequence, and domain M comprises a constant region amino acid sequence, or portion thereof; the fifth polypeptide chain comprises a domain P and a domain Q, wherein the domains are arranged, from N-terminus to C-terminus, in a P-Q orientation, and wherein domain P has a variable region domain amino acid sequence and domain Q has a CH3 domain acid sequence; the first polypeptide chain or the third polypeptide chain further comprises a domain N and a domain O, wherein domain N has a variable region domain amino acid sequence, wherein domain O has a CH3 domain amino acid sequence, wherein domains N and O are arranged, from N-terminus to C-terminus, in a N—O orientation, and wherein the C-terminus of domain O is attached, directly or indirectly, to the N-terminus of domain A of the first polypeptide chain or to the N-terminus of domain H of the third polypeptide chain; domains A and F form a first antigen binding site (ABS), domains H and L form a second ABS, and domains N and P form a third ABS; domains B and G form a first domain pair of associated constant region domains (“first domain pair”), domains I and M form a second domain pair of associated constant region domains (“second domain pair”), and domains Q and O form a third domain pair of associated constant region domains (“third domain pair”); at least one of the first and third domain pairs is an IgA-CH3 domain pair and the second domain pair is a CH1/CL domain pair; and the first and the second polypeptides are associated through an interaction between the A and the F domains and an interaction between the B and the G domains, the third and the fourth polypeptides are associated through an interaction between the H and the L domains and an interaction between the I and the M domains, the first and the third polypeptides are associated through an interaction between the D and the J domains and an interaction between the E and the K domains, and either the first or third polypeptide chain is associated with the fifth polypeptide chain through an interaction between the N and the P domains and an interaction between the O and the Q domains to form the binding molecule.

In some embodiments, the first polypeptide chain further comprises domain N and domain O. In some embodiments, the third polypeptide chain further comprises domain N and a domain O.

In some embodiments, the domain A comprises a VL amino acid sequence, and optionally the domain A is VL; and the domain F comprises a VH amino acid sequence, optionally the domain F is VH. In some embodiments the domain I is CL and domain M is CH1. In some embodiments the domain H comprises a VL amino acid sequence, optionally the domain H is VL; and the domain L comprises a VH amino acid sequence, optionally the domain L is VH. In some embodiments, the domain N comprises a VL amino acid sequence, optionally wherein domain N is VL; and the domain P comprises a VH amino acid sequence, optionally wherein domain P is VH. In some embodiments, the domains B and G are IgA-CH3 domains and the first domain pair is an IgA-CH3/IgA-CH3 domain pair. In some embodiments the domains O and Q are IgA-CH3 domains and the third domain pair is an IgA-CH3/IgA-CH3 domain pair. In some embodiments the domain B and domain G each comprise an orthogonal amino acid substitution that provides a non-endogenous cysteine, the non-endogenous cysteines capable of forming a disulfide bridge, and domain O and Q comprise a first and second CH3 linker sequence, respectively; or the domain B and domain G comprise a first and second CH3 linker, respectively, and domain O and Q each comprise an orthogonal amino acid substitution that provides a non-endogenous cysteine, the non-endogenous cysteines capable of forming a disulfide bridge; wherein the first and second CH3 linker sequence each comprise a cysteine capable of forming a disulfide bridge with the cysteine in the other CH3 linker sequence.

In some embodiments, the orthogonal amino acid substitutions providing non-endogenous cysteines are a P355C substitution in one IgA-CH3 domain and a H350C substitution in the other IgA-CH3 domain.

In some embodiments, one of the first and second CH3 linkers is AGC and the other CH3 linker is AGKGSC. In some embodiments, either: domain B and domain G each comprise an orthogonal amino acid substitution that provides a non-endogenous cysteine, wherein domain B comprises an H350C mutation and domain G comprises a P355C mutation, and wherein domain O comprises a first CH3 linker which is AGC and domain Q comprises a second CH3 linker which is AGKGSC; domain B and domain G each comprise an orthogonal amino acid substitution that provides a non-endogenous cysteine, wherein domain B comprises an H350C mutation and domain G comprises a P355C mutation, and wherein domain O comprises a first CH3 linker which is AGKGSC and domain Q comprises a second CH3 linker which is AGC; or domain B comprises a first CH3 linker which is AGC, domain G comprises a second CH3 linker which is AGKGSC, domain O comprises an H350C mutation and domain Q comprises a P355C mutation.

In some embodiments, the domains O and Q are IgG CH3 domains, optionally the IgG1-CH3 domains. In some embodiments, the domain O and domain Q each comprise an orthogonal charge pair mutation. In some embodiments, the orthogonal charge pair mutations comprise a T366K mutation in one of domains O and Q and a L351D mutation in the other domain. In some embodiments, the domain O comprises the T366K mutation and domain Q comprises the L351D mutation. In some embodiments, the domain O and domain Q each comprise an amino acid modification that provides a non-endogenous cysteine, the non-endogenous cysteines capable of forming a disulfide bridge. In some embodiments, the amino acid modifications are 447C modifications.

In some embodiments, either: domain B comprises a first CH3 linker which is AGC, domain G comprises a second CH3 linker which is AGKGSC, domain O comprises a T366K mutation and 447C modification, and domain Q comprises a L351D mutation and 447C modification; or domain B comprises a H350C mutation, domain G comprises a P355C mutation, domain O comprises a T366K mutation and 447C modification, and domain Q comprises a L351D mutation and 447C modification.

In some embodiments, the domains B and G are IgG-CH3 domains, optionally IgG1-CH3 domains, and the first domain pair is an IgG-CH3/IgG-CH3 domain pair; and domains O and Q are IgA-CH3 domains and the third domain pair is an IgA-CH3/IgA-CH3 domain pair.

In some embodiments, the domain B and domain G each comprise an amino acid modification that provides a non-endogenous cysteine, the non-endogenous cysteines capable of forming a disulfide bridge.

In some embodiments, the amino acid modifications are a 349C mutation in one of domains B and G and a 354C mutation in the other domain.

In some embodiments, the domain B comprises a P343V mutation.

In some embodiments, the amino acid modifications are 447C modifications.

In some embodiments, the domain B and domain G each comprise an orthogonal charge pair mutation. In some embodiments, the orthogonal charge pair mutations comprise a T366K mutation in one of domains B and G and a L351D mutation in the other domain. In some embodiments, the domain B comprises the T366K mutation and domain G comprises the L351D mutation.

In some embodiments, the domain O and domain Q each comprise an amino acid modification that provides a non-endogenous cysteine, the non-endogenous cysteines capable of forming a disulfide bridge, wherein the amino acid modifications of domains O and Q providing the non-endogenous cysteines are different from the amino acid modifications of domains B and G providing the non-endogenous cysteines.

In some embodiments, one of domains O and Q comprise an H350C mutation and the other domain comprises a P355C mutation, optionally wherein domain O comprises the H350C mutation and domain Q comprises the P355C mutation.

In some embodiments, the domain O comprises a first CH3 linker and domain Q comprises a second CH3 linker, the first and second CH3 linkers each comprising the non-endogenous cysteines capable of forming the disulfide bridge.

In some embodiments, the domain B comprises a T366K mutation and 447C amino acid modification; the domain G comprises an L351D mutation and 447C amino acid modification; the first CH3 linker is the amino acid sequence AGC; and the second CH3 linker is the amino acid sequence AGKGSC. In some embodiments, the domain O comprises a first CH3 linker which is the amino acid sequence AGC and domain Q comprises a second CH3 linker which is the amino acid sequence AGKGSC. In some embodiments, the domain A comprises a VH amino acid sequence, optionally the domain A is VH; and the domain F comprises a VL amino acid sequence, optionally the domain F is VL. In some embodiments, the domain H comprises a VH amino acid sequence, optionally the domain H is VH; and the domain L comprises a VL amino acid sequence, optionally the domain L is VL. In some embodiments, the domain N comprises a VH amino acid sequence, optionally wherein domain N is VH. In some embodiments, the domain I is CH1 and domain M is CL.

In a second aspect, the disclosure provides a binding molecule comprising a first, second, third, and fourth polypeptide chain, wherein: the first polypeptide chain comprises a domain A, a domain B, a domain D, and a domain E, wherein the domains are arranged, from N-terminus to C-terminus, in a A-B-D-E orientation, wherein domain A comprises a VL amino acid sequence, domain B comprises an IgA-CH3 domain sequence, domain D comprises a constant region domain amino acid sequence, and domain E comprises a constant region domain amino acid sequence; the second polypeptide chain comprises a domain F and a domain G, wherein the domains are arranged, from N-terminus to C-terminus, in a F-G orientation, and wherein domain F has a VH amino acid sequence and domain G comprises an IgA-CH3 domain sequence; the third polypeptide chain comprises a domain H, a domain I, a domain J, and a domain K, wherein the domains are arranged, from N-terminus to C-terminus, in a H-I-J-K orientation, and wherein domain H has a variable region domain amino acid sequence, domain I has a constant region amino acid sequence, and domains J and K have a constant region domain amino acid sequence; the fourth polypeptide chain comprises a domain L and a domain M, wherein the domains are arranged, from N-terminus to C-terminus, in a L-M orientation, and wherein domain L has a variable region domain amino acid sequence, and domain M comprises a constant region amino acid sequence, or portion thereof; domains A and F form a first antigen binding site (ABS) and domains H and L form a second ABS; the first and the second polypeptides are associated through an interaction between the A and the F domains and an interaction between the B and the G domains, the third and the fourth polypeptides are associated through an interaction between the H and the L domains and an interaction between the I and the M domains, and the first and the third polypeptides are associated through an interaction between the D and the J domains and an interaction between the E and the K domains to form the binding molecule.

In some embodiments, the domains B and G are IgA-CH3 domains.

In some embodiments, the domains E and K comprise an IgG-CH3 amino acid sequence.

In some embodiments, the domains E and K are IgG-CH3 amino acid domains.

In some embodiments, the domains E and K comprise an IgG1-CH3 amino acid sequence.

In some embodiments, the domains E and K are IgG1-CH3 domains.

In some embodiments, the domains E and K further comprise knob-hole mutations.

In some embodiments, the knob-hole mutations comprise a T366W mutation in one domain selected from domains E and K and a 366S, 368A, and 407V mutations in the other domain selected from domains E and K.

In some embodiments, the domain E comprises the T366W mutation and domain K comprises the 366S, 368A, and 407V mutations.

In some embodiments, the domains A and H comprise a VL amino acid sequence, and wherein domains F and L comprise a VH amino acid sequence.

In some embodiments, one of domains I and M is a CH1 domain and the other of domains I and M is a CL domain, and wherein domain I and domain M form a pair of associated CH1/CL domains (“CH1/CL domain pair”).

In some embodiments, the CH1 domain is an IgG CH1 domain and wherein the C domain is an IgG CL domain.

In some embodiments, the domain I is a CL domain and domain M is a CH1 domain.

In some embodiments, the domain B and domain D are attached via a first CH3 linker and wherein domain G comprises, at its C-terminus, a second CH3 linker, wherein the first CH3 linker and the second CH3 linker each comprise a cysteine capable of forming a disulfide bridge with the cysteine of the other CH3 linker.

In some embodiments, the domain B and domain G each comprise an engineered mutation, wherein the engineered mutation of domain B and the engineered mutation of domain G form a disulfide bond.

In some embodiments, either (i) the engineered mutation of domain B is a H350C mutation and the engineered mutation of domain G is a P355C mutation, or (ii) the engineered mutation of domain B is a P355C mutation and the engineered mutation of domain G is a H350C mutation.

In some embodiments, the engineered mutation of domain B is a H350C mutation and the engineered mutation of domain G is a P355C mutation.

In some embodiments, the binding molecule is a bivalent molecule.

In some embodiments, the first ABS and second ABS bind to the same epitope of the same antigen.

In some embodiments, the first ABS and second ABS bind to different epitopes of the same antigen.

In some embodiments, the first ABS binds to a first antigen and the second ABS binds to a second antigen which is different from the first antigen.

In some embodiments, the domain D and domain J each comprise a CH2 amino acid sequence. In some embodiments, the CH2 amino acid sequence is an IgG CH2 amino acid sequence. In some embodiments, the domains D and J are IgG CH2 domains. In some embodiments, the domain D and domain J comprise one or more engineered mutations at positions L234, L235, and P329 of the CH2 domain. In some embodiments, the domain E and domain K each comprise a CH3 amino acid sequence. In some embodiments, the CH3 amino acid sequences of domains E and K are IgG CH3 amino acid sequences. In some embodiments, the domain E and domain K are IgG CH3 domains. In some embodiments, the CH1/CL domain pair comprises a first orthogonal modification which comprises an L128C mutation in the CH1 sequence and an F118C mutation in the CL sequence, and wherein the CH1/CL pair comprises a second orthogonal modification which is a charged-pair modification selected from Table 7. In some embodiments, the domain A comprises a VL amino acid sequence and wherein domain F comprises a VH amino acid sequence. In some embodiments, the domain H comprises a VH amino acid sequence and domain L comprises a VL amino acid sequence. In some embodiments, the domain H comprises a VL amino acid sequence and domain L comprises a VH amino acid sequence. In some embodiments, the domain N comprises a VL amino acid sequence and domain P comprises a VH amino acid sequence. In some embodiments, the first CH3 linker is not identical to the second CH3 linker, optionally the first CH3 linker is of a different length than the second CH3 linker.

In some embodiments, (a) the first CH3 linker sequence is the amino acid sequence AGC and the second CH3 linker sequence is the amino acid sequence AGKGSC, (b) the first CH3 linker sequence is the amino acid sequence AGKGC and the second CH3 linker sequence is the amino acid sequence AGC, (c) the first CH3 linker sequence is the amino acid sequence AGKGSC and the second CH3 linker sequence is the amino acid sequence AGC, (d) the first CH3 linker sequence is the amino acid sequence AGKC and the second CH3 linker sequence is the amino acid sequence AGC, (e) the first CH3 linker sequence is the amino acid sequence GEC and the second CH3 linker sequence is the amino acid sequence GEC, (f) the first CH3 linker sequence is the amino acid sequence AGC and the second CH3 linker sequence is the amino acid sequence AGKGSC, (g) the first CH3 linker sequence is the amino acid sequence AGKGC and the second CH3 linker sequence is the amino acid sequence AGC, (h) the first CH3 linker sequence is the amino acid sequence AGKGSC and the second CH3 linker sequence is the amino acid sequence AGC, (i) the first CH3 linker sequence is the amino acid sequence AGKC and the second CH3 linker sequence is the amino acid sequence AGC, (j) the first CH3 linker sequence is the amino acid sequence AGC and the second CH3 linker sequence is the amino acid sequence AGC, (k) the first CH3 linker sequence is the amino acid sequence AGC and the second CH3 linker sequence is the amino acid sequence AGKGC, (l) the first CH3 linker sequence is the amino acid sequence AGC and the second CH3 linker sequence is the amino acid sequence AGKC, (m) the first CH3 linker sequence is the amino acid sequence AGC and the second CH3 linker sequence is the amino acid sequence GEC, (n) the first CH3 linker sequence is the amino acid sequence AGC and the second CH3 linker sequence is the amino acid sequence PGKC, (o) the first CH3 linker sequence is the amino acid sequence AGKGC and the second CH3 linker sequence is the amino acid sequence AGKGC, (p) the first CH3 linker sequence is the amino acid sequence AGKGC and the second CH3 linker sequence is the amino acid sequence AGKGSC, (q) the first CH3 linker sequence is the amino acid sequence AGKGC and the second CH3 linker sequence is the amino acid sequence AGKC, (r) the first CH3 linker sequence is the amino acid sequence AGKGC and the second CH3 linker sequence is the amino acid sequence GEC, (s) the first CH3 linker sequence is the amino acid sequence AGKGC and the second CH3 linker sequence is the amino acid sequence PGKC, (t) the first CH3 linker sequence is the amino acid sequence AGKGSC and the second CH3 linker sequence is the amino acid sequence AGKGC, (u) the first CH3 linker sequence is the amino acid sequence AGKGSC and the second CH3 linker sequence is the amino acid sequence AGKGSC, (v) the first CH3 linker sequence is the amino acid sequence AGKGSC and the second CH3 linker sequence is the amino acid sequence AGKC, (w) the first CH3 linker sequence is the amino acid sequence AGKGSC and the second CH3 linker sequence is the amino acid sequence GEC, (x) the first CH3 linker sequence is the amino acid sequence AGKGSC and the second CH3 linker sequence is the amino acid sequence PGKC, (y) the first CH3 linker sequence is the amino acid sequence AGKC and the second CH3 linker sequence is the amino acid sequence AGKGC, (z) the first CH3 linker sequence is the amino acid sequence AGKC and the second CH3 linker sequence is the amino acid sequence AGKGSC, (aa) the first CH3 linker sequence is the amino acid sequence AGKC and the second CH3 linker sequence is the amino acid sequence AGKC, (bb) the first CH3 linker sequence is the amino acid sequence AGKC and the second CH3 linker sequence is the amino acid sequence GEC, (cc) the first CH3 linker sequence is the amino acid sequence AGKC and the second CH3 linker sequence is the amino acid sequence PGKC, (dd) the first CH3 linker sequence is the amino acid sequence GEC and the second CH3 linker sequence is the amino acid sequence AGC, (ee) the first CH3 linker sequence is the amino acid sequence GEC and the second CH3 linker sequence is the amino acid sequence AGKGC, (ff) the first CH3 linker sequence is the amino acid sequence GEC and the second CH3 linker sequence is the amino acid sequence AGKGSC, (gg) the first CH3 linker sequence is the amino acid sequence GEC and the second CH3 linker sequence is the amino acid sequence AGKC, (hh) the first CH3 linker sequence is the amino acid sequence GEC and the second CH3 linker sequence is the amino acid sequence GEC, (ii) the first CH3 linker sequence is the amino acid sequence GEC and the second CH3 linker sequence is the amino acid sequence PGKC, (jj) the first CH3 linker sequence is the amino acid sequence PGKC and the second CH3 linker sequence is the amino acid sequence AGC, (kk) the first CH3 linker sequence is the amino acid sequence PGKC and the second CH3 linker sequence is the amino acid sequence AGKGC, (11) the first CH3 linker sequence is the amino acid sequence PGKC and the second CH3 linker sequence is the amino acid sequence AGKGSC, (mm) the first CH3 linker sequence is the amino acid sequence PGKC and the second CH3 linker sequence is the amino acid sequence AGKC, (nn) the first CH3 linker sequence is the amino acid sequence PGKC and the second CH3 linker sequence is the amino acid sequence GEC, (oo) and the first CH3 linker sequence is the amino acid sequence PGKC and the second CH3 linker sequence is the amino acid sequence PGKC.

In some embodiments, the first CH3 linker sequence is the amino acid sequence AGC and the second CH3 linker sequence is the amino acid sequence AGKGSC, the first CH3 linker sequence is the amino acid sequence AGKGC and the second CH3 linker sequence is the amino acid sequence AGC, the first CH3 linker sequence is the amino acid sequence AGKGSC and the second CH3 linker sequence is the amino acid sequence AGC, and the first CH3 linker sequence is the amino acid sequence AGKC and the second CH3 linker sequence is the amino acid sequence AGC.

In some embodiments, the first CH3 linker sequence is the amino acid sequence AGC and the second CH3 linker sequence is the amino acid sequence AGKGSC.

In a third aspect, the disclosure provides a binding molecule comprising a first, second, third, and fourth polypeptide chain, wherein: a) the first polypeptide chain comprises a domain A, a domain B, a domain D, and a domain E, wherein the domains are arranged, from N-terminus to C-terminus, in a A-B-D-E orientation, wherein domain A has a variable region domain amino acid sequence, and wherein domain B, domain D, and domain E have a constant region domain amino acid sequence; (b) the second polypeptide chain comprises a domain F and a domain G, wherein the domains are arranged, from N-terminus to C-terminus, in a F-G orientation, and wherein domain F has a variable region domain amino acid sequence and domain G has a constant region domain amino acid sequence; (c) the third polypeptide chain comprises a domain H, a domain I, a domain J, and a domain K, wherein the domains are arranged, from N-terminus to C-terminus, in a H-I-J-K orientation, and wherein domain H has a variable region domain amino acid sequence, domain I has a constant region amino acid sequence, and domains J and K have a constant region domain amino acid sequence; (d) the fourth polypeptide chain comprises a domain L and a domain M, wherein the domains are arranged, from N-terminus to C-terminus, in a L-M orientation, and wherein domain L has a variable region domain amino acid sequence, and domain M comprises a constant region amino acid sequence, or portion thereof; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the binding molecule; (h) domains D and J comprise CH2; (i) domains E and K comprise CH3; (j) domains D and J comprise one or more engineered mutations at positions L234, L235, and P329 of the CH2 domain.

In some embodiments, the domains A and H comprise VL, domains B and G comprise CH3, domain I comprises CL or CH1, and domain M comprises CH1 or CL, optionally the domain I comprises CL and domain M comprises CH1.

In some embodiments, the binding molecule has one or more engineered mutations at positions L234, L235, and P329 of the CH2 domain are L234A, L235A, and P329K of the CH2 domain.

In some embodiments, the binding molecule has one or more engineered mutations at positions L234, L235, and P329 of the CH2 domain are L234A, L235A, and P329A of the CH2 domain.

In some embodiments, the binding molecule has one or more engineered mutations at positions L234, L235, and P329 of the CH2 domain are L234A, L235A, and P329G of the CH2 domain.

In some embodiments, the binding molecule has one or more engineered mutations at positions L234, L235, and P329 of the CH2 domain are L234G, L235G, and P329A of the CH2 domain.

In some embodiments, the binding molecule has one or more engineered mutations at positions L234, L235, and P329 of the CH2 domain are L234G, L235G, and P329G of the CH2 domain.

In some embodiments, the binding molecule has one or more engineered mutations at positions L234, L235, and P329 of the CH2 domain are L234G, L235G, and P329K of the CH2 domain.

In a fourth aspect, the disclosure provides a CD3 binding molecule, the binding molecule comprising, a first antigen binding site specific for a CD3 antigen, wherein the first antigen binding site comprises: a CDR1, a CDR2, and a CDR3 amino acid sequences of a specific light chain variable region (VL) from a specific CD3 antigen binding site, wherein the CDR1, CDR2, and CDR3 VL sequences are selected from Table 8; and a CDR1, a CDR2, and a CDR3 amino acid sequences of a specific heavy chain variable region (VH) from the specific CD3 antigen binding site, wherein the CDR1, CDR2, and CDR3 VH sequences are selected from Table 8.

In a fifth aspect, the disclosure provides a humanized CD3 binding molecule, comprising: a VH amino acid sequence from an SP34-89 antibody; and a VL amino acid sequence from an SP34-89 antibody; wherein the VH amino acid sequence comprises a VH mutation selected from N30S, G65D, and S68T.

In some embodiments, the VL amino acid sequence is a wild-type SP34-89 VL sequence.

In some embodiments, the VL amino acid sequence comprises a W57G mutation.

In some embodiments, the VH mutation is N30S. In some embodiments, the VH mutation is G65D. In some embodiments the VH mutation is S68T.

In a sixth aspect, the disclosure provides a binding molecule comprising, a first, second, third, and fourth polypeptide chain, wherein: (a) the first polypeptide chain comprises a domain A, a domain B, a domain D, and a domain E, wherein the domains are arranged, from N-terminus to C-terminus, in a A-B-D-E orientation, wherein domain A has a variable region domain amino acid sequence, and wherein domain B, domain D, and domain E have a constant region domain amino acid sequence; (b) the second polypeptide chain comprises a domain F and a domain G, wherein the domains are arranged, from N-terminus to C-terminus, in a F-G orientation, and wherein domain F has a variable region domain amino acid sequence and domain G has a constant region domain amino acid sequence; (c) the third polypeptide chain comprises a domain H, a domain I, a domain J, and a domain K, wherein the domains are arranged, from N-terminus to C-terminus, in a H-I-J-K orientation, and wherein domain H has a variable region domain amino acid sequence, domain I has a constant region amino acid sequence, and domains J and K have a constant region domain amino acid sequence; (d) the fourth polypeptide chain comprises a domain L and a domain M, wherein the domains are arranged, from N-terminus to C-terminus, in a L-M orientation, and wherein domain L has a variable region domain amino acid sequence, and domain M comprises a constant region amino acid sequence, or portion thereof; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the binding molecule; (h) at least one of domains B and G or I and M form a CH1/CL domain pair; wherein the CH1/CL domain pair comprises a first orthogonal modification which comprises an L128C mutation in the CH1 sequence and an F118C mutation in the CL sequence, and wherein the CH1/CL pair comprises a second orthogonal modification which is a charged-pair modification selected from Table 7.

In some embodiments, the binding molecule is multispecific.

In some embodiments, the domain A is a VL domain and domain F is a VH domain.

In some embodiments, the domain B and domain G are each immunoglobulin CH3 domains.

In some embodiments, the domain I is a CL domain and domain M is a CH1 domain.

In some embodiments, the second orthogonal modification comprises a G166D mutation in the CH1 sequence and a N138K mutation in the CL sequence.

In some embodiments, the second orthogonal modification comprises a G166K mutation in the CH1 sequence and a N138D mutation in the CL sequence.

In some embodiments, the sequence that forms the junction between the A domain and the B domain is selected from IKRTPRP, IKRTTFRP, IKRTPREP, and IKRTVREP.

In some embodiments, the sequence that forms the junction between the A domain and the B domain is selected from IKRTPRP and IKRTTFRP.

In some embodiments, at least one CH3 amino acid sequence has a C-terminal tripeptide insertion connecting the CH3 amino acid sequence to a hinge amino acid sequence, wherein the tripeptide insertion is selected from the group consisting of PGK, KSC, and GEC.

In some embodiments, the sequences are human sequences.

In some embodiments, at least one CH3 amino acid sequence has one or more isoallotype mutations. In some embodiments, the isoallotype mutations are D356E and L358M. In some embodiments, the CL amino acid sequence is a Ckappa sequence.

In a seventh aspect, the disclosure provides an isolated polynucleotide or set of polynucleotides encoding one or more polypeptide chains of a binding molecule as provided herein.

In an eighth aspect, the disclosure provides a vector or set of vectors comprising the isolated polynucleotide or set of polynucleotides of a binding molecule as provided herein.

In a ninth aspect, the disclosure provides a pharmaceutical composition, comprising, a binding molecule as provided herein; and a pharmaceutically acceptable carrier.

In a tenth aspect, the disclosure provides a method of treatment, comprising administering to a subject in need of treatment a pharmaceutical composition, comprising, a binding molecule as provided herein; and a pharmaceutically acceptable carrier.

### 5. BRIEF DESCRIPTION OF THE DRAWINGS

FIG. 1 shows an alignment of the CH3-CH3 IgG1 dimer pair with CH1-CL. The quaternary structures align with an RMSD of ˜1.6 A2.

FIG. 2 presents schematic architectures, with respective naming conventions, for various binding molecules (also called antibody constructs) described herein.

FIG. 3 presents a higher resolution schematic of polypeptide chains and their domains, with respective naming conventions, for the bivalent 1×1 antibody constructs described herein.

FIG. 4 shows the architecture of an exemplary bivalent, monospecific, construct.

FIG. 5 shows data from a biolayer interferometry (BLI) experiment, described in Example 1, in which a bivalent monospecific binding molecule having the architecture illustrated in FIG. 4 [polypeptide 1: VL-CH3(Knob)-CH2-CH3/polypeptide 2: VH-CH3(Hole)] was assayed. The antigen binding site was specific for TNFα. The BLI response from binding molecule immobilization and TNFα binding to the immobilized construct demonstrates robust, specific, bivalent binding to the antigen. The data are consistent with a molecule having a high percentage of intended pairing of polypeptide 1 and polypeptide 2.

FIG. 6 illustrates features of an exemplary bivalent 1×1 bispecific binding molecule, “BC1”.

FIG. 7A shows size exclusion chromatography (SEC) analysis of “BC1”, demonstrating that a single-step CH1 affinity purification step (CaptureSelect™ CH1 affinity resin) yields a single, monodisperse peak via gel filtration in which >98% is unaggregated bivalent protein. FIG. 7B shows comparative literature data of SEC analysis of a CrossMab bivalent antibody construct [data from Schaefer et al. (Proc Natl Acad Sci USA. 2011 Jul. 5; 108(27):11187-92)].

FIG. 8A is a cation exchange chromatography elution profile of “BC1” following one-step purification using the CaptureSelect™ CH1 affinity resin, showing a single tight peak. FIG. 8B is a cation exchange chromatography elution profile of “BC1” following purification using standard Protein A purification.

FIG. 9 shows nonreducing SDS-PAGE gels of “BC1” at various stages of purification.

FIGS. 10A and 10B compare SDS-PAGE gels of “BC1” after single-step CH1-affinity purification under both non-reducing and reducing conditions (FIG. 10A) with SDS-PAGE gels of a CrossMab bispecific antibody under non-reducing and reducing conditions as published in the referenced literature (FIG. 10B).

FIGS. 11A and 11B show mass spec analysis of “BC1”, demonstrating two distinct heavy chains (FIG. 11A) and two distinct light chains (FIG. 11B) under reducing conditions.

FIG. 12 presents a mass spectrometry analysis of purified “BC1” under non-reducing conditions, confirming the absence of incomplete pairing after purification.

FIG. 13 presents accelerated stability testing data demonstrating stability of “BC1” over 8 weeks at 40° C., compared to two IgG control antibodies.

FIG. 14 illustrates features of an exemplary bivalent 1×1 bispecific binding molecule, “BC6”, further described in Example 3.

FIG. 15A presents size exclusion chromatography (SEC) analysis of “BC6” following one-step purification using the CaptureSelect™ CH1 affinity resin, demonstrating that the single step CH1 affinity purification yields a single monodisperse peak and the absence of non-covalent aggregates. FIG. 15B shows a SDS-PAGE gel of “BC6” under non-reducing conditions.

FIG. 16 illustrates features of an exemplary bivalent bispecific binding molecule, “BC28”, further described in Example 4.

FIG. 17 shows SDS-PAGE analysis under non-reducing conditions following single-step CH1 affinity purification of “BC28”, “BC29”, “BC30”, “BC31”, and “BC32”.

FIG. 18 shows SEC analysis of “BC28” and “BC30”, each following one-step purification using the CaptureSelect™ CH1 affinity resin.

FIG. 19 illustrates features of an exemplary bivalent bispecific binding molecule, “BC44”, further described in Example 5.

FIGS. 20A and 20B show size exclusion chromatography data of two bivalent binding molecules, “BC15” and “BC16”, respectively, under accelerated stability testing conditions. “BC15” and “BC16” have different variable region-CH3 junctions.

FIG. 21 presents a schematic of polypeptide chains and their domains, with respective naming conventions, for the trivalent 2×1 antibody constructs described herein.

FIG. 22 illustrates features of an exemplary trivalent 2×1 bispecific binding molecule, “BC1-2×1”, further described in Example 7.

FIG. 23 shows non-reducing SDS-PAGE of “BC1” and “BC1-2×1” protein expressed using the ThermoFisher Expi293 transient transfection system, at various stages of purification.

FIG. 24 compares the avidity of the bivalent 1×1 construct “BC1” to the avidity of the trivalent 2×1 construct “BC1-2×1” using an Octet (Pall ForteBio) biolayer interferometry analysis.

FIG. 25 illustrates salient features of a trivalent 2×1 construct, “TB111.”

FIG. 26 presents a schematic of polypeptide chains and their domains, with respective naming conventions, for the trivalent 1×2 antibody constructs described herein.

FIG. 27 illustrates features of an exemplary trivalent 1×2 construct “CTLA4-4×Nivo×CTLA4-4”, further described in Example 10.

FIG. 28 is a SDS-PAGE gel in which the lanes showing the trivalent 1×2 construct “CTLA4-4×Nivo×CTLA4-4” construct under non-reducing (“-DTT”) and reducing (“+DTT”) conditions have been boxed.

FIG. 29 shows a comparison of antigen binding between two antibodies: bivalent 1×1 construct “CTLA4-4×OX40-8” and the trivalent 1×2 construct “CTLA4-4×Nivo×CTLA4-4.” “CTLA4-4×OX40-8” binds to CTLA4 monovalently, while “CTLA4-4×Nivo×CTLA4-4” binds to CTLA4 bivalently.

FIG. 30 illustrates features of an exemplary trivalent 1×2 trispecific construct, “BC28-1×1×1a”, further described in Example 11.

FIG. 31 shows size exclusion chromatography of “BC28-1×1×1a” following transient expression and single step CH1 affinity resin purification, demonstrating a single well-defined peak.

FIG. 32 shows SDS-PAGE results with bivalent and trivalent constructs, each after transient expression and one-step purification using the CaptureSelect™ CH1 affinity resin, under non-reducing and reducing conditions, as further described in Example 12.

FIGS. 33A-33C show Octet binding analyses to 3 antigens: PD1, Antigen “A”, and CTLA4. As further described in Example 13, FIG. 33A shows binding of “BC1” to PD1 and Antigen “A”; FIG. 33B shows binding of a bivalent bispecific construct “CTLA4-4×OX40-8” to CTLA4, Antigen “A”, and PD1; FIG. 33C shows binding of trivalent trispecific “BC28-1×1×1a” to PD1, Antigen “A”, and CTLA4.

FIG. 34 presents a schematic of polypeptide chains and their domains, with respective naming conventions, for certain tetravalent 2×2 constructs described herein.

FIG. 35 illustrates certain salient features of the exemplary tetravalent 2×2 construct, “BC22-2×2” further described in Example 14.

FIG. 36 is a non-reducing SDS-PAGE gel comparing the 2×2 tetravalent “BC22-2×2” construct to a 1×2 trivalent construct “BC12-1×2” and a 2×1 trivalent construct “BC21-2×1” at different stages of purification.

FIG. 37 provides architecture for an exemplary tetravalent 2×2 construct.

FIG. 38 presents a schematic of polypeptide chains and their domains, with respective naming conventions, for certain tetravalent constructs described herein.

FIG. 39 provides exemplary architecture of a bispecific tetravalent construct.

FIG. 40 provides exemplary architecture for a trispecific tetravalent construct utilizing a common light chain strategy.

FIG. 41 shows bispecific antigen engagement by the tetravalent construct schematized in FIG. 39, demonstrating that this construct was capable of simultaneous engagement. The biolayer interferometry (BLI) response from B-Body immobilization and TNFα binding to the immobilized construct are consistent with a molecule with a high percentage of intended chain pairing.

FIG. 42 provides flow cytometry analysis of B-Body binding to cell-surface antigen. Cross-hatched signal indicates cells without antigen; dotted signal indicates transiently transfected cells with surface antigen.

FIG. 43 provides exemplary architecture of a trivalent construct.

FIG. 44 provides exemplary architecture of a trivalent construct.

FIG. 45 shows SDS-PAGE results with bivalent and trivalent constructs, each after transient expression and one-step purification using the CaptureSelect™ CH1 affinity resin, under non-reducing and reducing conditions, as further described in Example 17.

FIG. 46 shows differences in the thermal transitions for “BC24jv”, “BC26jv”, and “BC28jv” measured to assess pairing stability of junctional variants.

FIG. 47 demonstrates Octet (Pall ForteBio) biolayer interferometry analysis of a two-fold serial dilution (200-12.5 nM) used to determine binding affinity to CD3 for a non-mutagenized SP34-89 monovalent B-Body.

FIG. 48 shows SDS-PAGE analysis of bispecific antibodies comprising standard knob-hole orthogonal mutations introduced into CH3 domains found in their native positions within the Fc portion of the bispecific antibody that have been purified using a single-step CH1 affinity purification step (CaptureSelect™ CH1 affinity resin).

FIG. 49 depicts a three-dimensional model of a human IgA CH3 dimer. The white spheres denote residues that differ from a CH3 domain from human IgG.

FIG. 50 depicts an exemplary structure of a trivalent binding molecule.

FIG. 51 depicts an exemplary structure of a binding molecule comprising one or more CH1/CL orthogonal modifications.

FIG. 52 depicts an exemplary structure of a binding molecule comprising one or more CH1/CL orthogonal modifications.

FIG. 53 depicts an exemplary structure of a binding molecule comprising one or more CH1/CL orthogonal modifications.

FIG. 54 depicts an exemplary structure of a binding molecule comprising one or more CH1/CL orthogonal modifications.

FIG. 55 depicts an exemplary structure of a binding molecule comprising one or more CH1/CL orthogonal modifications.

FIG. 56 depicts an exemplary structure of a binding molecule comprising one or more CH1/CL orthogonal modifications.

FIG. 57 depicts an exemplary structure of a binding molecule comprising one or more CH1/CL orthogonal modifications.

FIG. 58 depicts an exemplary structure of a binding molecule comprising one or more CH1/CL orthogonal modifications.

FIG. 59 depicts an exemplary structure of a binding molecule comprising one or more CH1/CL orthogonal modifications.

FIG. 60 depicts an exemplary structure of a binding molecule comprising one or more CH1/CL orthogonal modifications.

FIG. 61 depicts Octet analysis of an exemplary binding molecule comprising a modification that reduces effector function.

FIG. 62 depicts results from an ADCC assay of various exemplary binding molecules comprising modifications that reduce effector function.

FIG. 63 depicts results from a C1q binding assay of various exemplary binding molecules comprising modifications that reduce effector function.

FIG. 64 depicts a schematic of the architecture of binding molecule MR-15.

FIG. 65 depicts SDS-PAGE analysis of binding molecule MR-15.

FIG. 66 depicts mass spectrogram results from an analysis of MR-15.

FIG. 67 depicts SDS-PAGE analysis of Variant 5.

FIG. 68 depicts SDS-PAGE analysis of various configurations of Variant 6.

FIG. 69 depicts results from an Octet assay assessing binding properties of a bispecific binding molecule comprising an IgA-CH3 domain swap.

FIG. 70 depicts SDS-PAGE analysis of BC1 and bispecific binding molecules comprising an IgA-CH3 domain swap and various CH3 linker sequences.

FIG. 71 depicts presents a schematic of polypeptide chains and their domains, with respective naming conventions, described herein.

FIG. 72 shows SDS-PAGE analysis of the BA variants in Table 16.

FIG. 73 depicts architectures of the various trivalent molecules (“T26,” “T27,” “T28,” “T33,” “T34,” “T35,” “T36”, “T37,” and “T38”).

FIGS. 74A and 74B shows SDS-PAGE results of Example 1. FIG. 74A shows SDS-PAGE results with the trivalent molecules T26, T27, T28, T33, T34, T35, and T37. FIG. 74B shows SDS-PAGE results with the trivalent molecules T27, T28, T33, T34, T35, and T36.

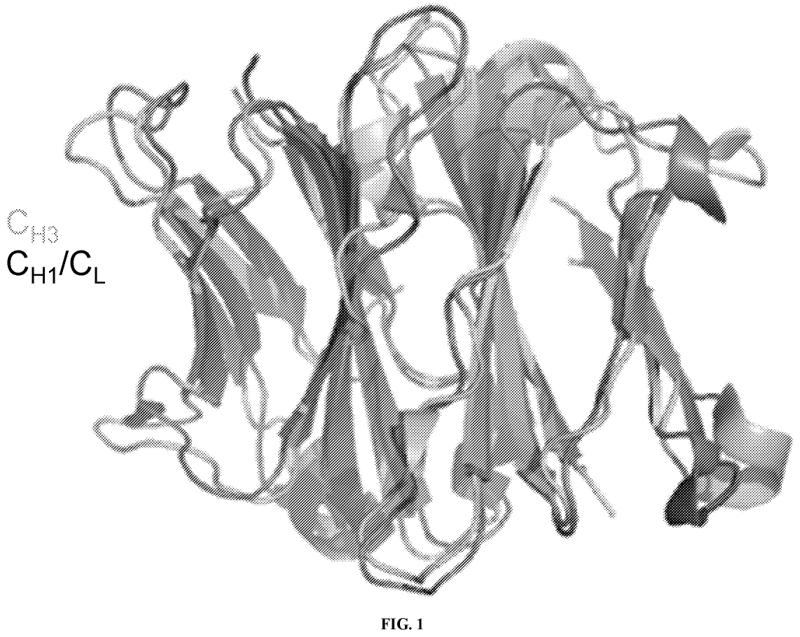

The figures depict various embodiments of the present invention for purposes of illustration only. One skilled in the art will readily recognize from the following discussion that alternative embodiments of the structures and methods illustrated herein may be employed without departing from the principles of the invention described herein.

### 6. DETAILED DESCRIPTION

### 6.1. Definitions

Unless defined otherwise, all technical and scientific terms used herein have the meaning commonly understood by a person skilled in the art to which this invention belongs. As used herein, the following terms have the meanings ascribed to them below.

As used herein, the singular forms “a,” “an,” and “the” include the plural referents unless the context clearly indicates otherwise. The terms “include,” “such as,” and the like are intended to convey inclusion without limitation, unless otherwise specifically indicated.

By “antigen binding site” is meant a region of a binding molecule that specifically recognizes or binds to a given antigen or epitope.

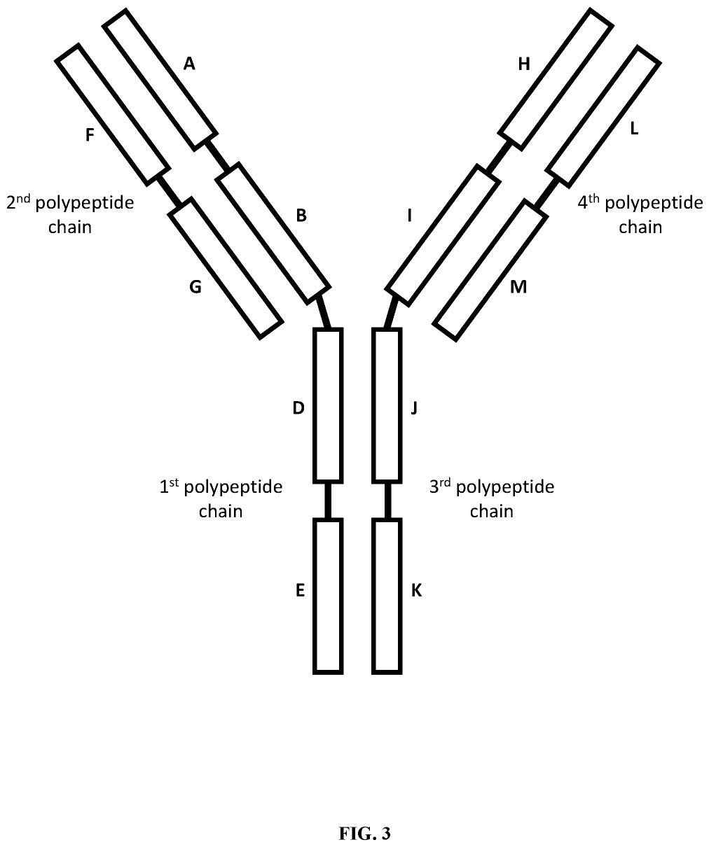

“B-Body,” as used herein and with reference to FIG. 3, refers to binding molecules comprising the features of a first and a second polypeptide chain, wherein: (a) the first polypeptide chain comprises a domain A, a domain B, a domain D, and a domain E, wherein the domains are arranged, from N-terminus to C-terminus, in a A-B-D-E orientation, and wherein domain A has a VL amino acid sequence, domain B has a CH3 amino acid sequence, domain D has a CH2 amino acid sequence, and domain E has a constant region domain amino acid sequence; (b) the second polypeptide chain comprises a domain F and a domain G, wherein the domains are arranged, from N-terminus to C-terminus, in a F-G orientation, and wherein domain F has a VH amino acid sequence and domain G has a CH3 amino acid sequence; and (c) the first and the second polypeptides are associated through an interaction between the A and the F domains and an interaction between the B and the G domains to form the binding molecule. B-bodies are described in more detail in International Patent Application No. PCT/US2017/057268, herein incorporated by reference in its entirety.

As used herein, the terms “treat” or “treatment” refer to both therapeutic treatment and prophylactic or preventative measures, wherein the object is to prevent or slow down (lessen) an undesired physiological change or disorder, such as the progression of multiple sclerosis, arthritis, or cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, and so on.

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. A therapeutically effective amount can be a “prophylactically effective amount” as prophylaxis can be considered therapy.

### 6.2. Other Interpretational Conventions

Unless otherwise specified, all references to sequences herein are to amino acid sequences.

Unless otherwise specified, antibody constant region residue numbering is according to the Eu index as described at //www.imgt.org/IMGTScientificChart/Numbering/Hu_IGHGnber.html#refs// (accessed Aug. 22, 2017) and in Edelman et al., Proc. Natl. Acad. USA, 63:78-85 (1969), which are hereby incorporated by reference in their entireties, and identifies the residue according to its location in an endogenous constant region sequence regardless of the residue's physical location within a chain of the binding molecules described herein. By “endogenous sequence” or “native sequence” is meant any sequence, including both nucleic acid and amino acid sequences, which originates from an organism, tissue, or cell and has not been artificially modified or mutated.

Polypeptide chain numbers (e.g., a “first” polypeptide chains, a “second” polypeptide chain. etc. or polypeptide “chain 1,” “chain 2,” etc.) are used herein as a unique identifier for specific polypeptide chains that form a binding molecule and is not intended to connote order or quantity of the different polypeptide chains within the binding molecule.

In this disclosure, “comprises,” “comprising,” “containing,” “having,” “includes,” “including,” and linguistic variants thereof have the meaning ascribed to them in U.S. Patent law, permitting the presence of additional components beyond those explicitly recited.

Ranges provided herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless specifically stated or apparent from context, as used herein the term “or” is understood to be inclusive. Unless specifically stated or apparent from context, as used herein, the terms “a”, “an”, and “the” are understood to be singular or plural.

Unless specifically stated or otherwise apparent from context, as used herein the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

### 6.3. C111 and CL Regions

CH1 amino acid sequences, as described herein, are antibody heavy chain constant domain sequences. In some embodiments, CH1 sequences are sequences of the second domain of an antibody heavy chain, with reference from the N-terminus to C-terminus. In certain embodiments, the CH1 sequences are endogenous sequences. In a variety of embodiments, the CH1 sequences are mammalian sequences, including, but not limited to mouse, rat, hamster, rabbit, camel, donkey, goat, and human sequences. In a preferred embodiment, the CH1 sequences are human sequences. In certain embodiments, the CH1 sequences are from an IgA1, IgA2, IgD, IgE, IgG1, IgG2, IgG3, IgG4, or IgM isotype. In a preferred embodiment, the CH1 sequences are from an IgG1 isotype. In preferred embodiments, the CH1 sequence is UniProt accession number P01857 amino acids 1-98.

The CL amino acid sequences useful in the binding molecules described herein are antibody light chain constant domain sequences. In certain embodiments, the CL sequences are endogenous sequences. In a variety of embodiments, the CL sequences are mammalian sequences, including, but not limited to mouse, rat, hamster, rabbit, camel, donkey, goat, and human sequences. In a preferred embodiment, CL sequences are human sequences.

In certain embodiments, the CL amino acid sequences are lambda (κ) light chain constant domain sequences. In particular embodiments, the CL amino acid sequences are human lambda light chain constant domain sequences. In preferred embodiments, the lambda (λ) light chain sequence is UniProt accession number P01834.

In certain embodiments, the CL amino acid sequences are kappa (κ) light chain constant domain sequences. In a preferred embodiment, the CL amino acid sequences are human kappa (κ) light chain constant domain sequences. In a preferred embodiment, the kappa light chain sequence is UniProt accession number P01834.

In certain embodiments, the CH1 sequence and the CL sequences are both endogenous sequences. In certain embodiments, the CH1 sequence and the CL sequences separately comprise respectively orthogonal modifications in endogenous CH1 and CL sequences, as discussed in greater detail in Sections 6.3.1.1 and 6.3.1.2. It is to be understood that orthogonal mutations in the CH1 sequence do not eliminate the specific binding interaction between the CH1 binding reagent and the CH1 domain. However, in some embodiments, the orthogonal mutations may reduce, though not eliminate, the specific binding interaction. CH1 and CL sequences can also be portions thereof, either of an endogenous or modified sequence, such that a domain having the CH1 sequence, or portion thereof, can associate with a domain having the CH1 sequence, or portion thereof. Furthermore, the binding molecule having a portion of the CH1 sequences described herein can be bound by the CH1 binding reagent.

Without wishing to be bound by theory, the CH1 domain is also unique in that it's folding is typically the rate limiting step in the secretion of IgG (Feige et al. Mol Cell. 2009 Jun. 12; 34(5):569-79; herein incorporated by reference in its entirety). Thus, purifying the binding molecules based on the rate limiting component of CH1 comprising polypeptide chains can provide a means to purify complete complexes from incomplete chains, e.g., purifying complexes having a limiting CH1 domain from complexes only having one or more non-CH1 comprising chains.

While the CH1 limiting expression may be a benefit in some aspects, as discussed, there is the potential for CH1 to limit overall expression of the complete binding molecules. Thus, in certain embodiments, the expression of the polypeptide chain comprising the CH1 sequence(s) is adjusted to improve the efficiency of the binding molecules forming complete complexes. In an illustrative example, the ratio of a plasmid vector constructed to express the polypeptide chain comprising the CH1 sequence(s) can be increased relative to the plasmid vectors constructed to express the other polypeptide chains. In another illustrative example, the polypeptide chain comprising the CH1 sequence(s) when compared to the polypeptide chain comprising the CL sequence(s) can be the smaller of the two polypeptide chains. In another specific embodiment, the expression of the polypeptide chain comprising the CH1 sequence(s) can be adjusted by controlling which polypeptide chain has the CH1 sequence(s). For example, engineering the binding molecule such that the CH1 domain is present in a two-domain polypeptide chain (e.g., the 4th polypeptide chain described herein), instead of the CH1 sequence's native position in a four-domain polypeptide chain (e.g., the 3rd polypeptide chain described herein), can be used to control the expression of the polypeptide chain comprising the CH1 sequence(s). However, in other aspects, a relative expression level of CH1 containing chains that is too high compared to the other chains can result in incomplete complexes the have the CH1 chain, but not each of the other chains. Thus, in certain embodiments, the expression of the polypeptide chain comprising the CH1 sequence(s) is adjusted to both reduce the formation incomplete complexes without the CH1 containing chain, and to reduce the formation incomplete complexes with the CH1 containing chain but without the other chains present in a complete complex.

### 6.3.1.1. CH1 and CL Orthogonal Modifications

In certain embodiments, the CH1 sequence and the CL sequences separately comprise respectively orthogonal modifications in endogenous CH1 and CL sequences.

“Orthogonal modifications” or synonymously “orthogonal mutations” as described herein are one or more engineered mutations in an amino acid sequence of an antibody domain that alter the affinity of binding of a first domain having orthogonal modification for a second domain having a complementary orthogonal modification, as compared to binding of the first and second domains in the absence of the orthogonal modifications. In some embodiments, the orthogonal modifications decrease the affinity of binding of the first domain having the orthogonal modification for the second domain having the complementary orthogonal modification, as compared to binding of the first and second domains in the absence of the orthogonal modifications. In preferred embodiments, the orthogonal modifications increase the affinity of binding of the first domain having the orthogonal modification for the second domain having the complementary orthogonal modification, as compared to binding of the first and second domains in the absence of the orthogonal modifications. In certain preferred embodiments, the orthogonal modifications decrease the affinity of a domain having the orthogonal modifications for a domain lacking the complementary orthogonal modifications.

In certain embodiments, orthogonal modifications are mutations in an endogenous antibody domain sequence. In a variety of embodiments, orthogonal modifications are modifications of the N-terminus or C-terminus of an endogenous antibody domain sequence including, but not limited to, amino acid additions or deletions. In particular embodiments, orthogonal modifications include, but are not limited to, engineered disulfide bridges, knob-in-hole mutations, and charge-pair mutations, as described in greater detail below. In particular embodiments, orthogonal modifications include a combination of orthogonal modifications selected from, but not limited to, engineered disulfide bridges, knob-in-hole mutations, and charge-pair mutations. In particular embodiments, the orthogonal modifications can be combined with amino acid substitutions that reduce immunogenicity, such as isoallotype mutations, as described in greater detail in Section 6.4.2.1.

In certain embodiments, the CH1 sequence and the CL sequence of the CH1/CL pair separately comprise respectively orthogonal modifications in endogenous CH1 and CL sequences. In other embodiments, one sequence of the CH1/CL pair comprises at least one modification while the other sequence of the CH1/CL pair does not comprise a modification in the respectively orthogonal amino acid position.

A CH1/CL orthogonal modification may affect the CH1/CL domain pairing via an interaction between a modified residue in the CH1 domain and a corresponding modified or unmodified residue in the CL domain.

It is to be understood that orthogonal mutations in the CH1 sequence do not eliminate the specific binding interaction between the CH1 binding reagent and the CH1 domain. However, in some embodiments, the orthogonal mutations may reduce, though not eliminate, the specific binding interaction. CH1 and CL sequences can also be portions thereof, either of an endogenous or modified sequence, such that a domain having the CH1 sequence, or portion thereof, can associate with a domain having the CH1 sequence, or portion thereof. Furthermore, the binding molecule having a portion of the CH1 sequences described herein can be bound by the CH1 binding reagent.

Exemplary CH1/CL Orthogonal Modifications: Engineered Disulfide Bridges

Some embodiments of a CH1/CL orthogonal modification comprise an engineered disulfide bridge between engineered cysteines in CH1 and CL. Such engineered disulfide bridges may stabilize an interaction between the polypeptide comprising the modified CH1 and the polypeptide comprising the corresponding modified CL.

An orthogonal CH1/CL modification comprising an engineered disulfide bridge can comprise, by way of example only, a CH1 domain having an engineered cysteine at position 128, 129, 138, 141, 168, or 171, as numbered by the EU index. Such an orthogonal CH1/CL modification comprising an engineered disulfide bridge may further comprise, by way of example only, a CL domain having an engineered cysteine at position 116, 118, 119, 164, 162, or 210, as numbered by the EU index.

For example, a CH1/CL orthogonal modification may be selected from engineered cysteines at position 138 of the CH1 sequence and position 116 of the CL sequence, at position 128 of the CH1 sequence and position 119 of the CL sequence, or at position 129 of the CH1 sequence and position 210 of the CL sequence, as numbered and discussed in more detail in U.S. Pat. Nos. 8,053,562 and 9,527,927, each incorporated herein by reference in its entirety. In some embodiments, the CH1/CL orthogonal modification comprises an engineered cysteine at position 141 of the CH1 sequence and position 118 of the CL sequence, as numbered by the EU index.

In some embodiments, the CH1/CL orthogonal modification comprises an engineered cysteine at position 168 of the CH1 sequence and position 164 of the CL sequence, as numbered by the EU index. In some embodiments, the CH1/CL orthogonal modification comprises an engineered cysteine at position 128 of the CH1 sequence and position 118 of the CL sequence, as numbered by the EU index. In some embodiments, the CH1/CL orthogonal modification comprises an engineered cysteine at position 171 of the CH1 sequence and position 162 of the CL sequence, as numbered by the EU index. In some embodiments, the CL sequence is a CL-lambda sequence. In preferred embodiments, the CL sequence is a CL-kappa sequence. In some embodiments, the engineered cysteines are at position 128 of the CH1 sequence and position 118 of the CL Kappa sequence, as numbered by the EU index.

Table 6 below provides exemplary CH1/CL orthogonal modifications comprising an engineered disulfide bridge between CH1 and CL, numbered according to the EU index.

TABLE 6
exemplary CH1/CL engineered disulfide bridges

| CH1 mutation | CL mutation |
|---|---|
| A141C | F118C |
| H168C | T164C |
| L128C | F118C |
| P171C | S162C |

In a series of preferred embodiments, the mutations that provide non-endogenous (engineered) cysteine amino acids are a F118C mutation in the CL sequence with a corresponding A141C in the CH1 sequence, or a F118C mutation in the CL sequence with a corresponding L128C in the CH1 sequence, a T164C mutation in the CL sequence with a corresponding H168C mutation in the CH1 sequence, or a S162C mutation in the CL sequence with a corresponding P171C mutation in the CH1 sequence, as numbered by the Eu index.

CH1/CL Orthogonal Modifications: Charged-Pair Mutations

In a variety of embodiments, the orthogonal modifications in the CL sequence and the CH1 sequence are charge-pair mutations. As used herein, charge-pair mutations are amino acid substitutions that affect the charge of a residue in a domain's surface such that the domain will preferentially associate with a second domain having complementary charge-pair mutations relative to association with domains without the complementary charge-pair mutations. In certain embodiments, charge-pair mutations improve orthogonal association between specific domains. Charge-pair mutations are described in greater detail in U.S. Pat. Nos. 8,592,562, 9,248,182, and 9,358,286, each of which is incorporated by reference herein for all they teach. In certain embodiments, charge-pair mutations improve stability between specific domains. In specific embodiments the charge-pair mutations are a F118S, F118A or F118V mutation in the CL sequence with a corresponding A141L in the CH1 sequence, or a T129R mutation in the CL sequence with a corresponding K147D in the CH1 sequence, as numbered by the Eu index and described in greater detail in Bonisch et al. (Protein Engineering, Design & Selection, 2017, pp. 1-12), herein incorporated by reference for all that it teaches.

In some cases, the CH1/CL charge-pair mutations are a N138K mutation in the CL sequence with a corresponding G166D in the CH1 sequence, or a N138D mutation in the CL sequence with a corresponding G166K in the CH1 sequence, as numbered by the Eu index. In some embodiments, the charge-pair mutations are a P127E mutation in CH1 sequence with a corresponding E123K mutation in the corresponding Cl sequence. In some embodiments, the charge-pair mutations are a P127K mutation in CH1 sequence with a corresponding E123 (not mutated) in the corresponding CL sequence.

Table 7 below provides exemplary CH1/CL orthogonal charged-pair modifications.

TABLE 7
exemplary CH1/CL orthogonal charged-pair modifications

| CH1 mutation | CL mutation |
|---|---|
| G166D | N138K |
| G166D | N138D |
| G166K | N138K |
| G166K | N138D |
| P127E | E123K |
| P127E | No mutation (E123) |
| P127K | E123K |
| P127K | No mutation (E123) |

### 6.3.1.2. Combinations of CH1/CL Orthogonal Modifications

In certain embodiments, the CH1 and CL domains of a single CH1/CL pair separately contain two or more respectively orthogonal modifications in endogenous CH1 and CL sequences. For instance, the CH1 and CL sequence may contain a first orthogonal modification and a second orthogonal modification in the endogenous CH1 and CL sequences. The two or more respectively orthogonal modifications in endogenous CH1 and CL sequences can be selected from any of the CH1/CL orthogonal modifications described herein.

In some embodiments, the first orthogonal modification is an orthogonal charge-pair mutation, and the second orthogonal modification is an orthogonal engineered disulfide bridge. In some embodiments, the first orthogonal modification is an orthogonal charge-pair mutation as described in Table 7, and the additional orthogonal modification comprise an engineered disulfide bridge selected from engineered cysteines at position 138 of the CH1 sequence and position 116 of the CL sequence, at position 128 of the CH1 sequence and position 119 of the CL sequence, or at position 129 of the CH1 sequence and position 210 of the CL sequence, as numbered and discussed in more detail in U.S. Pat. Nos. 8,053,562 and 9,527,927, each incorporated herein by reference in its entirety. In some embodiments, the first orthogonal modification is an orthogonal charge-pair mutation as described in Table 7, and the additional orthogonal modification comprise an engineered disulfide bridge as described in Table 6. In some embodiments, the first orthogonal modification comprises an L128C mutation in the CH1 sequence and an F118C mutation in the CL sequence, and the second orthogonal modification comprises a modification of residue 166 in the same CH1 sequence and a modification of residue 138 in the same CL sequence as described in Section 6.322. In some embodiments, the first orthogonal modification comprises an L128C mutation in the CH1 sequence and an F118C mutation in the CL sequence, and the second orthogonal modification comprises a G166D mutation in the CH1 sequence and a N138K mutation in the CL sequence. In some embodiments, the first orthogonal modification comprises an L128C mutation in the CH1 sequence and an F118C mutation in the CL sequence, and the second orthogonal modification comprises a G166K mutation in the CH1 sequence and a N138D mutation in the CL sequence.

### 6.4. Binding Molecules

Further aspects of the binding molecules useful for the invention are provided.

With reference to FIG. 3, in a series of embodiments, the binding molecules comprise a first and a second polypeptide chain, wherein: (a) the first polypeptide chain comprises a domain A, a domain B, a domain D, and a domain E, wherein the domains are arranged, from N-terminus to C-terminus, in a A-B-D-E orientation, wherein domain A has a variable region domain amino acid sequence, and wherein domain B, domain D, and domain E have a constant region domain amino acid sequence; (b) the second polypeptide chain comprises a domain F and a domain G, wherein the domains are arranged, from N-terminus to C-terminus, in a F-G orientation, and wherein domain F has a variable region domain amino acid sequence and domain G has a constant region domain amino acid sequence; (c) the third polypeptide chain comprises a domain H, a domain I, a domain J, and a domain K, wherein the domains are arranged, from N-terminus to C-terminus, in a H-I-J-K orientation, and wherein domain H has a variable region domain amino acid sequence, domain I has a constant region amino acid sequence, and domains J and K have a constant region domain amino acid sequence; (d) the fourth polypeptide chain comprises a domain L and a domain M, wherein the domains are arranged, from N-terminus to C-terminus, in a L-M orientation, and wherein domain L has a variable region domain amino acid sequence, and domain M comprises a constant region amino acid sequence, or portion thereof; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the binding molecule.

In a series of embodiments, (a) the first polypeptide chain comprises a domain A, a domain B, a domain D, and a domain E, wherein the domains are arranged, from N-terminus to C-terminus, in a A-B-D-E orientation, wherein domain A has a variable region domain amino acid sequence, and wherein domain B, domain D, and domain E have a constant region domain amino acid sequence; (b) the second polypeptide chain comprises a domain F and a domain G, wherein the domains are arranged, from N-terminus to C-terminus, in a F-G orientation, and wherein domain F has a variable region domain amino acid sequence and domain G has a constant region domain amino acid sequence; (c) the third polypeptide chain comprises a domain H, a domain I, a domain J, and a domain K, wherein the domains are arranged, from N-terminus to C-terminus, in a H-I-J-K orientation, and wherein the third polypeptide chain comprises the CH1 domain and domain I is the CH1 domain, or portion thereof, domain H has a variable region domain amino acid sequence, and domains J and K have a constant region domain amino acid sequence; (d) the fourth polypeptide chain comprises a domain L and a domain M, wherein the domains are arranged, from N-terminus to C-terminus, in a L-M orientation, and wherein domain L has a variable region domain amino acid sequence, and wherein domain M has a CL amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the binding molecule.

In some embodiments, the binding molecule comprises a native antibody architecture, wherein domains A and H comprise VH amino acid sequences, domains F and L comprise VL amino acid sequences, domains B and I comprise CH1, domains G and M comprise CL, domains D and J comprise CH2, and domains E and K comprise CH3.

In preferred embodiments, the binding molecule is a B-Body™. B-Body™ binding molecules are described in International patent Application No. PCT/US2017/057268. In some embodiments, the binding molecule is structured as described in [0126], wherein domains A and H comprise VL, domains B and G comprise CH3, domain I comprises CL or CH1, domain M comprises CH1 or CL, domains D and J comprise CH2, and domains E and K comprise CH3. In some embodiments, domain I comprises CL and domain M comprises CH1. In some embodiments, domain I is CH1 and domain M is CL.

In some embodiments, the binding molecule is a CrossMab™. CrossMab™ antibodies are described in U.S. Pat. Nos. 8,242,247; 9,266,967; and 8,227,577, U.S. Patent Application Pub. No. 20120237506, U.S. Patent Application Pub. No. US20090162359, WO2016016299, WO2015052230. In some embodiments, the binding molecule is a bivalent, bispecific antibody, comprising: a) the light chain and heavy chain of an antibody specifically binding to a first antigen; and b) the light chain and heavy chain of an antibody specifically binding to a second antigen, wherein constant domains CL and CH1 from the antibody specifically binding to a second antigen are replaced by each other. In some embodiments, the binding molecule is structured as described in paragraph [0126], wherein A is VH, B is CH1, D is CH2, E is CH3, F is VL, G is CL, H is VL or VH, I is CL, J is CH2, K is CH3, L is VH or VL, and M is CH1.

In some embodiments, the binding molecule is an antibody having a general architecture described in U.S. Pat. No. 8,871,912 and WO2016087650. In some embodiments, the binding molecule is a domain-exchanged antibody comprising a light chain (LC) composed of VL-CH3, and a heavy chain (HC) comprising VH-CH3-CH2-CH3, wherein the VL-CH3 of the LC dimerizes with the VH-CH3 of the HC thereby forming a domain-exchanged LC/HC dimer comprising a CH3LC/CH3HC domain pair. In some embodiments, the binding molecule is structured as described in paragraph [0126], wherein A is VH, B is CH3, D is CH2, E is CH3, F is VL, G is CH3, H is VH, I is CH1, J is CH2, K is CH3, L is VL, and M is CL.

In some embodiments, the binding molecule is as described in WO2017011342. In some embodiments, the binding molecule is structured as described in paragraph [0126], wherein A is VH or VL, B is CH2 from IgM or IgE, D is CH2, E is CH3, F is VL or VH, G is CH2 from IgM or IgE, H is VH, I is CH1, J is CH2, K is CH3, L is VL, and M is CL.

In some embodiments, the binding molecule is as described in WO2006093794. In some embodiments, the binding molecule is structured as described in paragraph [0126], wherein A is VH, B is CH1, D is CH2, E is CH3, F is VL, G is CL, H is VL, I is CL or CH1, J is CH2, K is CH3, L is VH, and M is CH1 or CL.

### 6.4.1. Domain A (Variable Region)

In the binding molecules, domain A has a variable region domain amino acid sequence. Variable region domain amino acid sequences, as described herein, are variable region domain amino acid sequences of an antibody including VL and VH antibody domain sequences. VL and VH sequences are described in greater detail in Sections 6.4.1.1 and 6.4.1.4, respectively. In a preferred embodiment, domain A has a VL antibody domain sequence and domain F has a VH antibody domain sequence. In some embodiments, domain A has a VH antibody domain sequence and domain F has a VL antibody domain sequence.

### 6.4.1.1. VL Regions

The VL amino acid sequences useful in the binding molecules described herein are antibody light chain variable domain sequences. In a typical arrangement in both natural antibodies and the antibody constructs described herein, a specific VL amino acid sequence associates with a specific VH amino acid sequence to form an antigen-binding site. In various embodiments, the VL amino acid sequences are mammalian sequences, including human sequences, synthesized sequences, or combinations of human, non-human mammalian, mammalian, and/or synthesized sequences, as described in further detail in Sections 6.4.1.2 and 6.4.1.3.

In various embodiments, VL amino acid sequences are mutated sequences of naturally occurring sequences. In certain embodiments, the VL amino acid sequences are lambda (κ) light chain variable domain sequences. In certain embodiments, the VL amino acid sequences are kappa (κ) light chain variable domain sequences. In a preferred embodiment, the VL amino acid sequences are kappa (κ) light chain variable domain sequences.

In the binding molecules described herein, the C-terminus of domain A is connected to the N-terminus of domain B. In certain embodiments, domain A has a VL amino acid sequence that is mutated at its C-terminus at the junction between domain A and domain B, as described in greater detail in Section 6.4.19.1 and in Example 6.

### 6.4.1.2. Complementarity Determining Regions

VH and VL amino acid sequences may comprise highly variable sequences termed “complementarity determining regions” (CDRs), typically three CDRs (CDR1, CD2, and CDR3). In a variety of embodiments, the CDRs are mammalian sequences, including, but not limited to, mouse, rat, hamster, rabbit, camel, donkey, goat, and human sequences. In a preferred embodiment, the CDRs are human sequences. In various embodiments, the CDRs are naturally occurring sequences. In various embodiments, the CDRs are naturally occurring sequences that have been mutated to alter the binding affinity of the antigen-binding site for a particular antigen or epitope. In certain embodiments, the naturally occurring CDRs have been mutated in an in vivo host through affinity maturation and somatic hypermutation. In certain embodiments, the CDRs have been mutated in vitro through methods including, but not limited to, PCR-mutagenesis and chemical mutagenesis. In various embodiments, the CDRs are synthesized sequences including, but not limited to, CDRs obtained from random sequence CDR libraries and rationally designed CDR libraries.

### 6.4.1.3. Framework Regions and CDR Grafting

VH and VL amino acid sequences may comprise “framework region” (FR) sequences. FRs are generally conserved sequence regions that act as a scaffold for interspersed CDRs (see Section 6.4.1.2.), typically in a FR1-CDR1-FR2-CDR2-FR3-CDR3-FR4 arrangement (from N-terminus to C-terminus). In a variety of embodiments, the FRs are mammalian sequences, including, but not limited to mouse, rat, hamster, rabbit, camel, donkey, goat, and human sequences. In a preferred embodiment, the FRs are human sequences. In various embodiments, the FRs are naturally occurring sequences. In various embodiments, the FRs are synthesized sequences including, but not limited, rationally designed sequences.

In a variety of embodiments, the FRs and the CDRs are both from the same naturally occurring variable domain sequence. In a variety of embodiments, the FRs and the CDRs are from different variable domain sequences, wherein the CDRs are grafted onto the FR scaffold with the CDRs providing specificity for a particular antigen. In certain embodiments, the grafted CDRs are all derived from the same naturally occurring variable domain sequence. In certain embodiments, the grafted CDRs are derived from different variable domain sequences. In certain embodiments, the grafted CDRs are synthesized sequences including, but not limited to, CDRs obtained from random sequence CDR libraries and rationally designed CDR libraries. In certain embodiments, the grafted CDRs and the FRs are from the same species. In certain embodiments, the grafted CDRs and the FRs are from different species. In a preferred grafted CDR embodiment, an antibody is “humanized”, wherein the grafted CDRs are non-human mammalian sequences including, but not limited to, mouse, rat, hamster, rabbit, camel, donkey, and goat sequences, and the FRs are human sequences. Humanized antibodies are discussed in more detail in U.S. Pat. No. 6,407,213, the entirety of which is hereby incorporated by reference for all it teaches. In various embodiments, portions or specific sequences of FRs from one species are used to replace portions or specific sequences of another species' FRs.

### 6.4.1.4. VH Regions

The VH amino acid sequences in the binding molecules described herein are antibody heavy chain variable domain sequences. In a typical antibody arrangement in both nature and in the binding molecules described herein, a specific VH amino acid sequence associates with a specific VL amino acid sequence to form an antigen-binding site. In various embodiments, VH amino acid sequences are mammalian sequences, including human sequences, synthesized sequences, or combinations of non-human mammalian, mammalian, and/or synthesized sequences, as described in further detail in Sections 6.4.1.2 and 6.4.1.3. In various embodiments, VH amino acid sequences are mutated sequences of naturally occurring sequences.

### 6.4.2. Domain B (Constant Region)

In the binding molecules, Domain B has a constant region domain sequence.

Constant region domain amino acid sequences, as described herein, are sequences of a constant region domain of an antibody.

In a variety of embodiments, the constant region sequences are mammalian sequences, including, but not limited to, mouse, rat, hamster, rabbit, camel, donkey, goat, and human sequences. In a preferred embodiment, the constant region sequences are human sequences. In certain embodiments, the constant region sequences are from an antibody light chain. In particular embodiments, the constant region sequences are from a lambda or kappa light chain. In certain embodiments, the constant region sequences are from an antibody heavy chain. In particular embodiments, the constant region sequences are an antibody heavy chain sequence that is an IgA1, IgA2, IgD, IgE, IgG1, IgG2, IgG3, IgG4, or IgM isotype. In a specific embodiment, the constant region sequences are from an IgG isotype. In a preferred embodiment, the constant region sequences are from an IgG1 isotype.

In preferred specific embodiments, the constant region sequence is a CH3 sequence. CH3 sequences are described in greater detail in Section 6.4.2.1. In other preferred embodiments, the constant region sequence is an orthologous CH2 sequence. Orthologous CH2 sequences are described in greater detail in Section 6.4.2.2.

In some embodiments, domain B has a CH1 sequence. CH1 sequences are described herein. In some embodiments, domain B has a CH2 sequence from IgE. In some embodiments, domain B has a CH2 sequence from IgM.

In particular embodiments, for example wherein the valency of the binding molecule is three or greater than three, the constant region sequence is a CH1 or Cl sequence. CH1 and Cl sequences are described herein. In some embodiments, the constant region sequence is a Cl sequence. In some embodiments, the CH1 or Cl sequence comprises one or more CH1 or Cl orthogonal modifications described herein.

In particular embodiments, the constant region sequence has been mutated to include one or more orthogonal mutations. In a preferred embodiment, domain B has a constant region sequence that is a CH3 sequence comprising knob-hole (synonymously, “knob-in-hole,” “KIH”) orthogonal mutations, as described in greater detail in Section 6.4.14.2, and either a S354C or a Y349C mutation that forms an engineered disulfide bridge with a CH3 domain containing an orthogonal mutation, as described in in greater detail in Section 6.4.14.1. In some preferred embodiments, the knob-hole orthogonal mutation is a T366W mutation.

### 6.4.2.1. CH3 Regions

CH3 amino acid sequences, as described herein, are sequences of the C-terminal domain of an antibody heavy chain.

In a variety of embodiments, the CH3 sequences are mammalian sequences, including, but not limited to, mouse, rat, hamster, rabbit, camel, donkey, goat, and human sequences. In a preferred embodiment, the CH3 sequences are human sequences. In certain embodiments, the CH3 sequences are from an IgA1, IgA2, IgD, IgE, IgM, IgG1, IgG2, IgG3, IgG4 isotype or CH4 sequences from an IgE or IgM isotype. In a specific embodiment, the CH3 sequences are from an IgG isotype. In a preferred embodiment, the CH3 sequences are from an IgG1 isotype. In some embodiments, the CH3 sequence is from an IgA isotype.

In certain embodiments, the CH3 sequences are endogenous sequences. In particular embodiments, the CH3 sequence is UniProt accession number P01857 amino acids 224-330. In various embodiments, a CH3 sequence is a segment of an endogenous CH3 sequence. In particular embodiments, a CH3 sequence has an endogenous CH3 sequence that lacks the N-terminal amino acids G224 and Q225. In particular embodiments, a CH3 sequence has an endogenous CH3 sequence that lacks the C-terminal amino acids P328, G329, and K330. In particular embodiments, a CH3 sequence has an endogenous CH3 sequence that lacks both the N-terminal amino acids G224 and Q225 and the C-terminal amino acids P328, G329, and K330. In preferred embodiments, a binding molecule has multiple domains that have CH3 sequences, wherein a CH3 sequence can refer to both a full endogenous CH3 sequence as well as a CH3 sequence that lacks N-terminal amino acids, C-terminal amino acids, or both.

In certain embodiments, the CH3 sequences are endogenous sequences that have one or more mutations. In particular embodiments, the mutations are one or more orthogonal mutations that are introduced into an endogenous CH3 sequence to guide specific pairing of specific CH3 sequences, as described in more detail in Sections 6.4.14.1-6.4.14.3.

In certain embodiments, the CH3 sequences are engineered to reduce immunogenicity of the antibody by replacing specific amino acids of one allotype with those of another allotype and referred to herein as isoallotype mutations, as described in more detail in Stickler et al. (Genes Immun. 2011 April; 12(3): 213-221), which is herein incorporated by reference for all that it teaches. In particular embodiments, specific amino acids of the G1m1 allotype are replaced. In a preferred embodiment, isoallotype mutations D356E and L358M are made in the CH3 sequence.

In a preferred embodiment, domain B has a human IgG1 CH3 amino acid sequence with the following mutational changes: P343V; Y349C; and a tripeptide insertion, 445P, 446G, 447K. In other preferred embodiments, domain B has a human IgG1 CH3 sequence with the following mutational changes: T366K; and a tripeptide insertion, 445K, 446S, 447C. In still other preferred embodiments, domain B has a human IgG1 CH3 sequence with the following mutational changes: Y349C and a tripeptide insertion, 445P, 446G, 447K.

In certain embodiments, domain B has a human IgG1 CH3 sequence with a 447C mutation incorporated into an otherwise endogenous CH3 sequence.

In the binding molecules described herein, the N-terminus of domain B is connected to the C-terminus of domain A. In certain embodiments, domain B has a CH3 amino acid sequence that is mutated at its N-terminus at the junction between domain A and domain B, as described in greater detail in Section 6.4.19.1 and Example 6.

In the binding molecules, the C-terminus of domain B is connected to the N-terminus of domain D. In certain embodiments, domain B has a CH3 amino acid sequence that is extended at the C-terminus at the junction between domain B and domain D, as described in greater detail in Section 6.4.19.3.

In some embodiments, domain B comprises a human IgA CH3 sequence. An exemplary human IgA CH3 sequence is

(SEQ ID NO: 184)
TFRPEVHLLPPPSEELALNELVTLTCLARGFSPKDVLVRWLQGSQELPREK
YLTWASRQEPSQGTTTFAVTSILRVAAEDWKKGDTFSCMVGHEALPLAFTQ
KTIDRL.

In some embodiments, the IgA-CH3 sequence comprises a CH3 linker sequence described herein.

### 6.4.2.2. Orthologous CH2 Regions

CH2 amino acid sequences, as described herein, are sequences of the third domain of an antibody heavy chain, with reference from the N-terminus to C-terminus. CH2 amino acid sequences, in general, are discussed in more detail in Section 6.4.3. In a series of embodiments, a binding molecule has more than one paired set of CH2 domains that have CH2 sequences, wherein a first set has CH2 amino acid sequences from a first isotype and one or more orthologous sets of CH2 amino acid sequences from another isotype. The orthologous CH2 amino acid sequences, as described herein, are able to interact with CH2 amino acid sequences from a shared isotype, but not significantly interact with the CH2 amino acid sequences from another isotype present in the binding molecule. In particular embodiments, all sets of CH2 amino acid sequences are from the same species. In preferred embodiments, all sets of CH2 amino acid sequences are human CH2 amino acid sequences. In other embodiments, the sets of CH2 amino acid sequences are from different species. In particular embodiments, the first set of CH2 amino acid sequences is from the same isotype as the other non-CH2 domains in the binding molecule. In a specific embodiment, the first set has CH2 amino acid sequences from an IgG isotype and the one or more orthologous sets have CH2 amino acid sequences from an IgM or IgE isotype. In certain embodiments, one or more of the sets of CH2 amino acid sequences are endogenous CH2 sequences. In other embodiments, one or more of the sets of CH2 amino acid sequences are endogenous CH2 sequences that have one or more mutations. In particular embodiments, the one or more mutations are orthogonal knob-hole mutations, orthogonal charge-pair mutations, or orthogonal hydrophobic mutations. Orthologous CH2 amino acid sequences useful for the binding molecules are described in more detail in international PCT applications WO2017/011342 and WO2017/106462, herein incorporated by reference in their entirety.

### 6.4.3. Domain D (Constant Region)

In the binding molecules described herein, domain D has a constant region amino acid sequence. Constant region amino acid sequences are described in more detail herein, for example in Sections 6.3 and 6.4.2.

In a preferred series of embodiments, domain D has a CH2 amino acid sequence. CH2 amino acid sequences, as described herein, are CH2 amino acid sequences of the third domain of a native antibody heavy chain, with reference from the N-terminus to C-terminus. In a variety of embodiments, the CH2 sequences are mammalian sequences, including but not limited to mouse, rat, hamster, rabbit, camel, donkey, goat, and human sequences. In a preferred embodiment, the CH2 sequences are human sequences. In certain embodiments, the CH2 sequences are from an IgA1, IgA2, IgD, IgE, IgG1, IgG2, IgG3, IgG4, or IgM isotype. In a preferred embodiment, the CH2 sequences are from an IgG1 isotype.

In certain embodiments, the CH2 sequences are endogenous sequences. In particular embodiments, the sequence is UniProt accession number P01857 amino acids 111-223. In a preferred embodiment, the CH2 sequences have an N-terminal hinge region peptide that connects the N-terminal variable domain-constant domain segment to the CH2 domain, as discussed in more detail in Section 6.4.19.3. In some embodiments, the CH2 sequence comprises one or more mutations that reduce effector function, as discussed in more detail in Section 6.10.4.

In the binding molecules, the N-terminus of domain D is connected to the C-terminus of domain B. In certain embodiments, domain B has a CH3 amino acid sequence that is extended at the C-terminus at the junction between domain D and domain B, as described in greater detail in Section 6.4.19.3.

### 6.4.4. Domain E (Constant Region)

In the binding molecules, domain E has a constant region domain amino acid sequence. Constant region amino acid sequences are described in more detail in Section 6.4.2.

In certain embodiments, the constant region sequence is a CH3 sequence. CH3 sequences are described in greater detail in Section 6.4.2.1. In particular embodiments, the constant region sequence has been mutated to include one or more orthogonal mutations. In a preferred embodiment, domain E has a constant region sequence that is a CH3 sequence comprising knob-hole (synonymously, “knob-in-hole,” “KIH”) orthogonal mutations, as described in greater detail in Section 6.4.14.2, and either a S354C or a Y349C mutation that forms an engineered disulfide bridge with a CH3 domain containing an orthogonal mutation, as described in in greater detail in Section 6.4.14.1. In some preferred embodiments, the knob-hole orthogonal mutation is a T366W mutation.

In certain embodiments, the constant region domain sequence is a CH1 sequence. In particular embodiments, the CH1 amino acid sequence of domain E is the only CH1 amino acid sequence in the binding molecule. In certain embodiments, the N-terminus of the CH1 domain is connected to the C-terminus of a CH2 domain, as described in greater detail in Section 6.4.19.5. In certain embodiments, the constant region sequence is a CL sequence. In certain embodiments, the N-terminus of the CL domain is connected to the C-terminus of a CH2 domain, as described in greater detail in 6.4.19.5. CH1 and CL sequences are described in further detail in Section 6.3.

### 6.4.5. Domain F (Variable Region)

In the binding molecules, domain F has a variable region domain amino acid sequence. Variable region domain amino acid sequences, as discussed in greater detail in Section 6.4.1, are variable region domain amino acid sequences of an antibody including VL and VH antibody domain sequences. VL and VH sequences are described in greater detail in Sections 6.4.1.1 and 6.4.1.4, respectively. In a preferred embodiment, domain F has a VH antibody domain sequence. In some embodiments, domain F has a VL antibody domain sequence.

### 6.4.6. Domain G

In the binding molecules, domain G has a constant region amino acid sequence. Constant region amino acid sequences are described in more detail in Sections 6.3 and 6.4.2.

In preferred embodiments, domain G has a CH3 amino acid sequence. CH3 sequences are described in greater detail in Section 6.4.2.1.

In certain preferred embodiments, domain G has a human IgG1 CH3 sequence with the following mutational changes: S354C; and a tripeptide insertion, 445P, 446G, 447K. In some preferred embodiments, domain G has a human IgG1 CH3 sequence with the following mutational changes: S354C; and 445P, 446G, 447K tripeptide insertion. In some preferred embodiments, domain G has a human IgG1 CH3 sequence with the following changes: L351D, and a tripeptide insertion of 445G, 446E, 447C.

In some embodiments, domain G has a human IgA CH3 sequence. An exemplary human IgA CH3 sequence is described in Section 6.4.2.1.

In some embodiments, domain G has a CL sequence. In some embodiments, domain G has a CH2 sequence from IgE. In some embodiments, domain G has a CH2 sequence from IgM.

In particular embodiments, for example wherein the valency of the binding molecule is three or greater than three, the constant region sequence is a CH1 or Cl sequence. In some embodiments wherein domain B is a Cl sequence, domain G is a CH1 sequence. CH1 and Cl sequences are described herein. In some embodiments, the CH1 or Cl sequence comprises one or more CH1 or Cl orthogonal modifications described herein.

In some embodiments of the binding molecules, the C-terminus of domain G is connected to the N-terminus of domain D. In certain embodiments, domain G has a CH3 amino acid sequence that is extended at the C-terminus at the junction between domain G and domain D, as described in greater detail in Section 6.4.19.3.

### 6.4.7. Domain H (Variable Region)

In the binding molecules, domain H has a variable region domain amino acid sequence. Variable region domain amino acid sequences, as discussed in greater detail in Section 6.4.1, are variable region domain amino acid sequences of an antibody including VL and VH antibody domain sequences. VL and VH sequences are described in greater detail in Sections 6.4.1.1. and 6.4.1.4, respectively. In a preferred embodiment, domain H has a VL antibody domain sequence. In some embodiments, domain H has a VH antibody domain sequence.

### 6.4.8. Domain I (Constant Region)

In the binding molecules, domain I has a constant region domain amino acid sequence. Constant region domain amino acid sequences are described in greater detail in Sections 6.3 and 6.4.2. In a series of preferred embodiments of the binding molecules, domain I has a CL amino acid sequence. In another series of embodiments, domain I has a CH1 amino acid sequence. CH1 and CL amino acid sequences are described in further detail in Section 6.3.

### 6.4.9. Domain J (CH2)

In the binding molecules, domain J has a CH2 amino acid sequence. CH2 amino acid sequences are described in greater detail in Section 6.4.3. In a preferred embodiment, the CH2 amino acid sequence has an N-terminal hinge region that connects domain J to domain I, as described in more detail in Section 6.4.19.4. In some embodiments, the CH2 sequence comprises one or more mutations that reduce effector function, as discussed in more detail in Section 6.10.4.

In the binding molecules, the C-terminus of domain J is connected to the N-terminus of domain K. In particular embodiments, domain J is connected to the N-terminus of domain K that has a CH1 amino acid sequence or CL amino acid sequence, as described in further detail in Section 6.4.19.5.

### 6.4.10. Domain K (Constant Region)

In the binding molecules, domain K has a constant region domain amino acid sequence. Constant region domain amino acid sequences are described in greater detail in Section 6.4.2. In certain embodiments, the constant region sequence is a CH3 sequence. CH3 sequences are described in greater detail in Section 6.4.2.1. In a preferred embodiment, domain K has a constant region sequence that is a CH3 sequence comprising knob-hole orthogonal mutations, as described in greater detail in Section 6.4.14.2; isoallotype mutations, as described in more detail above in Section 6.4.2.1.; and either a S354C or a Y349C mutation that forms an engineered disulfide bridge with a CH3 domain containing an orthogonal mutation, as described in in greater detail in Section 6.4.14.1. In some preferred embodiments, the knob-hole orthogonal mutations combined with isoallotype mutations are the following mutational changes: D356E, L358M, T366S, L368A, and Y407V.

In certain embodiments, the constant region domain sequence is a CH1 sequence. In particular embodiments, the CH1 amino acid sequence of domain K is the only CH1 amino acid sequence in the binding molecule. In certain embodiments, the N-terminus of the CH1 domain is connected to the C-terminus of a CH2 domain, as described in greater detail in Section 6.4.19.5. In certain embodiments, the constant region sequence is a CL sequence. In certain embodiments, the N-terminus of the CL domain is connected to the C-terminus of a CH2 domain, as described in greater detail in Section 6.4.19.5. CH1 and CL sequences are described in further detail in Section 6.3.

### 6.4.11. Domain L (Variable Region)

In the binding molecules, domain L has a variable region domain amino acid sequence. Variable region domain amino acid sequences, as discussed in greater detail in Section 6.4.1, are variable region domain amino acid sequences of an antibody including VL and VH antibody domain sequences. VL and VH sequences are described in greater detail in Sections 6.4.1.1. and 6.4.1.4, respectively. In a preferred embodiment, domain L has a VH antibody domain sequence. In some embodiments, domain L has a VL antibody domain sequence.

### 6.4.12. Domain M (Constant Region)

In the binding molecules, domain M has a constant region domain amino acid sequence. Constant region domain amino acid sequences are described in greater detail in Section 6.4.2. In a series of preferred embodiments of the binding molecules, domain I has a CH1 amino acid sequence and domain M has a CL amino acid sequence. In another series of preferred embodiments, domain I has a CL amino acid sequence and domain M has a CH1 amino acid sequence. CH1 and CL amino acid sequences are described in further detail in Section 6.3.

### 6.4.13. Pairing of Domains A & F

In the binding molecules, a domain A VL or VH amino acid sequence and a cognate domain F VH or VL amino acid sequence are associated and form an antigen binding site (ABS). The A:F antigen binding site (ABS) is capable of specifically binding an epitope of an antigen. Antigen binding by an ABS is described in greater detail in Section 6.4.13.1.

In a variety of multivalent embodiments, the ABS formed by domains A and F (A:F) is identical in sequence to one or more other ABSs within the binding molecule and therefore has the same recognition specificity as the one or more other sequence-identical ABSs within the binding molecule.

In a variety of multivalent embodiments, the A:F ABS is non-identical in sequence to one or more other ABSs within the binding molecule. In certain embodiments, the A:F ABS has a recognition specificity different from that of one or more other sequence-non-identical ABSs in the binding molecule. In particular embodiments, the A:F ABS recognizes a different antigen from that recognized by at least one other sequence-non-identical ABS in the binding molecule. In particular embodiments, the A:F ABS recognizes a different epitope of an antigen that is also recognized by at least one other sequence-non-identical ABS in the binding molecule. In these embodiments, the ABS formed by domains A and F recognizes an epitope of antigen, wherein one or more other ABSs within the binding molecule recognizes the same antigen but not the same epitope.

### 6.4.13.1. Binding of Antigen by ABS

An ABS, and the binding molecule comprising such ABS, is said to “recognize” the epitope (or more generally, the antigen) to which the ABS specifically binds, and the epitope (or more generally, the antigen) is said to be the “recognition specificity” or “binding specificity” of the ABS.

The ABS is said to bind to its specific antigen or epitope with a particular affinity. As described herein, “affinity” refers to the strength of interaction of non-covalent intermolecular forces between one molecule and another. The affinity, i.e. the strength of the interaction, can be expressed as a dissociation equilibrium constant (K_{D}), wherein a lower K_{D} value refers to a stronger interaction between molecules. K_{D} values of antibody constructs are measured by methods well known in the art including, but not limited to, bio-layer interferometry (e.g. Octet/FORTEBIO®), surface plasmon resonance (SPR) technology (e.g. Biacore), and cell binding assays. For purposes herein, affinities are dissociation equilibrium constants measured by bio-layer interferometry using Octet/FORTEBIO®.

“Specific binding,” as used herein, refers to an affinity between an ABS and its cognate antigen or epitope in which the K_{D} value is below 10⁻⁶M, 10⁻⁷M, 10⁻⁸M, 10⁻⁹M, or 10⁻¹⁰M.

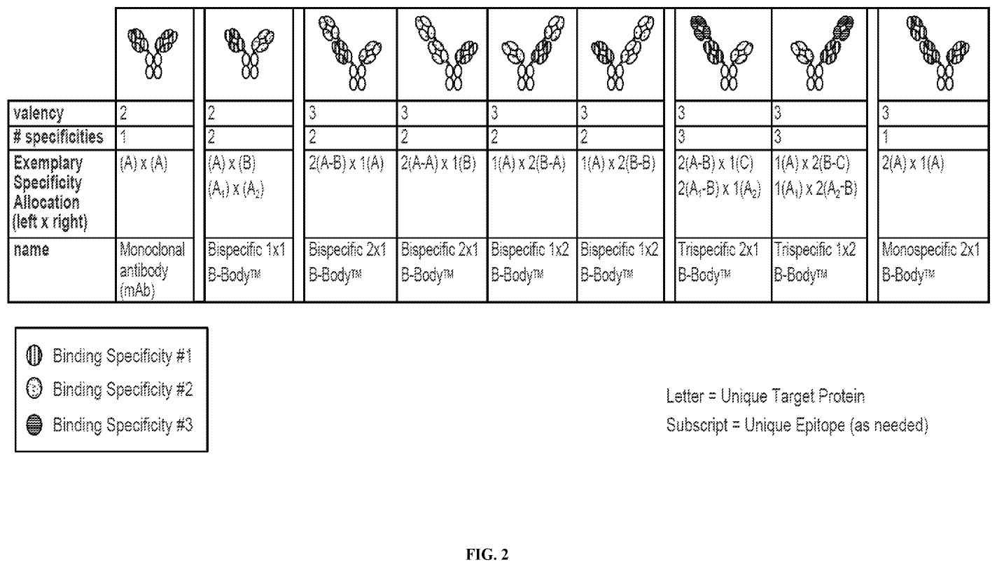

The number of ABSs in a binding molecule as described herein defines the “valency” of the binding molecule, as schematized in FIG. 2. A binding molecule having a single ABS is “monovalent”. A binding molecule having a plurality of ABSs is said to be “multivalent”. A multivalent binding molecule having two ABSs is “bivalent.” A multivalent binding molecule having three ABSs is “trivalent.” A multivalent binding molecule having four ABSs is “tetravalent.”

In various multivalent embodiments, all of the plurality of ABSs have the same recognition specificity. As schematized in FIG. 2, such a binding molecule is a “monospecific” “multivalent” binding construct. In other multivalent embodiments, at least two of the plurality of ABSs have different recognition specificities. Such binding molecules are multivalent and “multispecific”. In multivalent embodiments in which the ABSs collectively have two recognition specificities, the binding molecule is “bispecific.” In multivalent embodiments in which the ABSs collectively have three recognition specificities, the binding molecule is “trispecific.”

In multivalent embodiments in which the ABSs collectively have a plurality of recognition specificities for different epitopes present on the same antigen, the binding molecule is “multiparatopic.” Multivalent embodiments in which the ABSs collectively recognize two epitopes on the same antigen are “biparatopic.”

In various multivalent embodiments, multivalency of the binding molecule improves the avidity of the binding molecule for a specific target. As described herein, “avidity” refers to the overall strength of interaction between two or more molecules, e.g. a multivalent binding molecule for a specific target, wherein the avidity is the cumulative strength of interaction provided by the affinities of multiple ABSs. Avidity can be measured by the same methods as those used to determine affinity, as described above. In certain embodiments, the avidity of a binding molecule for a specific target is such that the interaction is a specific binding interaction, wherein the avidity between two molecules has a K_{D} value below 10⁻⁶M, 10⁻⁷M, 10⁻⁸M, 10⁻⁹M, or 10⁻¹⁰M. In certain embodiments, the avidity of a binding molecule for a specific target has a K_{D} value such that the interaction is a specific binding interaction, wherein the one or more affinities of individual ABSs do not have has a K_{D} value that qualifies as specifically binding their respective antigens or epitopes on their own. In certain embodiments, the avidity is the cumulative strength of interaction provided by the affinities of multiple ABSs for separate antigens on a shared specific target or complex, such as separate antigens found on an individual cell. In certain embodiments, the avidity is the cumulative strength of interaction provided by the affinities of multiple ABSs for separate epitopes on a shared individual antigen.

### 6.4.14. Pairing of Domains B & G

In the binding molecules described herein, a domain B constant region amino acid sequence and a domain G constant region amino acid sequence are associated. Constant region domain amino acid sequences are described in greater detail in Section 6.4.2.

In a series of preferred embodiments, domain B and domain G have CH3 amino acid sequences. CH3 sequences are described in greater detail in Section 6.4.2.1. In various embodiments, the amino acid sequences of the B and the G domains are identical. In certain of these embodiments, the sequence is an endogenous CH3 sequence. The sequence may be a CH3 sequence from human IgG1. The sequence may be a sequence from human IgA.

In a variety of embodiments, the amino acid sequences of the B and the G domains are different, and separately comprise respectively orthogonal modifications in an endogenous CH3 sequence, wherein the B domain interacts with the G domain, and wherein neither the B domain nor the G domain significantly interacts with a CH3 domain lacking the orthogonal modification.

Orthogonal modifications” or synonymously “orthogonal mutations” as described herein are one or more engineered mutations in an amino acid sequence of an antibody domain that alter the affinity of binding of a first domain having orthogonal modification for a second domain having a complementary orthogonal modification, as compared to binding of the first and second domains in the absence of the orthogonal modifications. In some embodiments, the orthogonal modifications decrease the affinity of binding of the first domain having the orthogonal modification for the second domain having the complementary orthogonal modification, as compared to binding of the first and second domains in the absence of the orthogonal modifications. In preferred embodiments, the orthogonal modifications increase the affinity of binding of the first domain having the orthogonal modification for the second domain having the complementary orthogonal modification, as compared to binding of the first and second domains in the absence of the orthogonal modifications. In certain preferred embodiments, the orthogonal modifications decrease the affinity of a domain having the orthogonal modifications for a domain lacking the complementary orthogonal modifications.

In certain embodiments, orthogonal modifications are mutations in an endogenous antibody domain sequence. In a variety of embodiments, orthogonal modifications are modifications of the N-terminus or C-terminus of an endogenous antibody domain sequence including, but not limited to, amino acid additions or deletions. In particular embodiments, orthogonal modifications include, but are not limited to, engineered disulfide bridges, knob-in-hole mutations, and charge-pair mutations, as described in greater detail in Sections 6.3.1.1, 6.3.1.2, 6.4.14.1-6.4.14.3. In particular embodiments, orthogonal modifications include a combination of orthogonal modifications selected from, but not limited to, engineered disulfide bridges, knob-in-hole mutations, and charge-pair mutations. In particular embodiments, the orthogonal modifications can be combined with amino acid substitutions that reduce immunogenicity, such as isoallotype mutations, as described in greater detail in Section 6.4.2.1.

### 6.4.14.1. Orthogonal Engineered Disulfide Bridges in CH3

In a variety of embodiments, the orthogonal modifications comprise mutations that generate engineered disulfide bridges between a first and a second domain. As described herein, “engineered disulfide bridges” are mutations that provide non-endogenous cysteine amino acids in two or more domains such that a non-native disulfide bond forms when the two or more domains associate. Engineered disulfide bridges are described in greater detail in Merchant et al. (Nature Biotech (1998) 16:677-681), the entirety of which is hereby incorporated by reference for all it teaches. In certain embodiments, engineered disulfide bridges improve orthogonal association between specific domains. In a particular embodiment, the mutations that generate engineered disulfide bridges are a K392C mutation in one of a first or second CH3 domains, and a D399C in the other CH3 domain. In a preferred embodiment, the mutations that generate engineered disulfide bridges are a S354C mutation in one of a first or second CH3 domains, and a Y349C in the other CH3 domain. In another preferred embodiment, the mutations that generate engineered disulfide bridges are a 447C mutation in both the first and second CH3 domains that are provided by extension of the C-terminus of a CH3 domain incorporating a KSC tripeptide sequence.

In some embodiments, the orthogonal engineered disulfide bridge is between a first IgA-CH3 domain and a second IgA-CH3 domain. In some embodiments, the mutations that generate such engineered disulfide bridge is a H350C mutation in one of the first or second IgA-CH3 domains and a P355C mutation in the other IgA-CH3 domain.

For clarity, the residue designated “H350” in the IgA-CH3 domain sequence is the underlined “H” residue in the following endogenous IgA-CH3 amino acid sequence:

TFRPEVHLLPPPSEELALNELVTLTCLARGFSPKDVLVRWLQGSQELPREK
YLTWASRQEPSQGTTTFAVTSILRVAAEDWKKGDTFSCMVGHEALPLAFTQ
KTIDRL.

By way of example, an IgA-CH3 amino acid domain sequence with a “H350C” mutation in an otherwise endogenous IgA-CH3 domain has the following sequence:

TFRPEVCLLPPPSEELALNELVTLTCLARGFSPKDVLVRWLQGSQELPREK
YLTWASRQEPSQGTTTFAVTSILRVAAEDWKKGDTFSCMVGHEALPLAFTQ
KTIDRL.

For clarity, the residue designated “P355” in the IgA-CH3 domain sequence is the underlined “P” residue in the following endogenous IgA-CH3 amino acid sequence:

TFRPEVHLLPPPSEELALNELVTLTCLARGFSPKDVLVRWLQGSQELPREK
YLTWASRQEPSQGTTTFAVTSILRVAAEDWKKGDTFSCMVGHEALPLAFTQ
KTIDRL.

By way of example, an IgA-CH3 amino acid domain sequence with a “P355C” mutation in an otherwise endogenous IgA-CH3 domain has the following sequence:

TFRPEVHLLPPCSEELALNELVTLTCLARGFSPKDVLVRWLQGSQELPREK
YLTWASRQEPSQGTTTFAVTSILRVAAEDWKKGDTFSCMVGHEALPLAFTQ
KTIDRL.

### 6.4.14.2. Orthogonal Knob-Hole Mutations

In a variety of embodiments, orthogonal modifications comprise knob-hole (synonymously, knob-in-hole) mutations. As described herein, knob-hole mutations are mutations that change the steric features of a first domain's surface such that the first domain will preferentially associate with a second domain having complementary steric mutations relative to association with domains without the complementary steric mutations. Knob-hole mutations are described in greater detail in U.S. Pat. Nos. 5,821,333 and 8,216,805, each of which is incorporated herein in its entirety. In various embodiments, knob-hole mutations are combined with engineered disulfide bridges, as described in greater detail in Merchant et al. (Nature Biotech (1998) 16:677-681)), incorporated herein by reference in its entirety. In various embodiments, knob-hole mutations, isoallotype mutations, and engineered disulfide mutations are combined.

In certain embodiments, the knob-in-hole mutations are a T366Y mutation in a first domain, and a Y407T mutation in a second domain. In certain embodiments, the knob-in-hole mutations are a F405A in a first domain, and a T394W in a second domain. In certain embodiments, the knob-in-hole mutations are a T366Y mutation and a F405A in a first domain, and a T394W and a Y407T in a second domain. In certain embodiments, the knob-in-hole mutations are a T366W mutation in a first domain, and a Y407A in a second domain. In certain embodiments, the combined knob-in-hole mutations and engineered disulfide mutations are a S354C and T366W mutations in a first domain, and a Y349C, T366S, L368A, and aY407V mutation in a second domain. In a preferred embodiment, the combined knob-in-hole mutations, isoallotype mutations, and engineered disulfide mutations are a S354C and T366W mutations in a first domain, and a Y349C, D356E, L358M, T366S, L368A, and a Y407V mutation in a second domain.

### 6.4.14.3. Orthogonal Charge-Pair Mutations

In a variety of embodiments, orthogonal modifications are charge-pair mutations. As used herein, charge-pair mutations are mutations that affect the charge of an amino acid in a domain's surface such that the domain will preferentially associate with a second domain having complementary charge-pair mutations relative to association with domains without the complementary charge-pair mutations. In certain embodiments, charge-pair mutations improve orthogonal association between specific domains. Charge-pair mutations are described in greater detail in U.S. Pat. Nos. 8,592,562, 9,248,182, and 9,358,286, each of which is incorporated by reference herein for all they teach. In certain embodiments, charge-pair mutations improve stability between specific domains. In a preferred embodiment, the charge-pair mutations are a T366K mutation in a first domain, and a L351D mutation in the other domain.

### 6.4.14.4. IgA-CH3 Isotype Domain Substitution

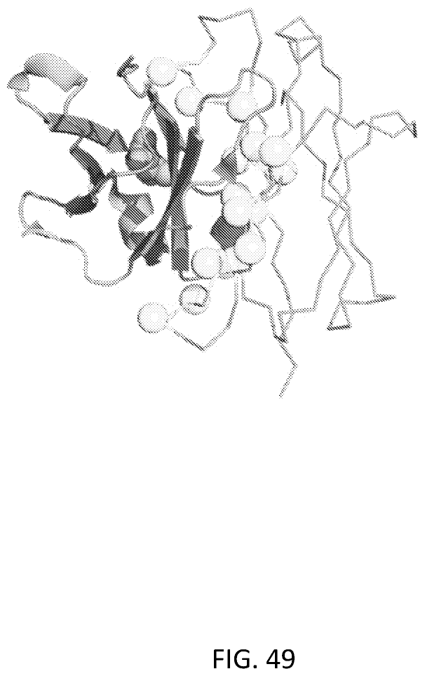

FIG. 49 depicts a rendition of a human IgA CH3 dimer. Non-identical residues in reference to human IgG CH3 are depicted as white spheres. In some embodiments, it is desirable to reduce an undesired association of a first and second domain, which may contain CH3 sequences, with a third and fourth domain, which may also contain CH3 sequences. In such cases, use of CH3 sequences from human IgA (IgA-CH3) in the first and/or second domain may improve antibody assembly and stability by reducing such undesired associations. In some embodiments of a binding molecule wherein the third and fourth domain comprise IgG-CH3 sequences, the first and/or second domain comprises IgA-CH3 sequences.

In some embodiments, at least one of the first or second domain comprise a CH3 linker sequence as described in Section 6.4.19.3. In some embodiments, both the first and second domain comprise a CH3 linker sequence as described in Section 6.4.19.3. In some embodiments, the first comprises a first CH3 linker sequence and the second domain comprises a second CH3 linker sequence. In some embodiments, the first CH3 linker sequence associates with the second CH3 linker sequence by formation of a disulfide bridge between cysteine residues of the first and second CH3 linker sequences. In some embodiments, the first CH3 linker and the second CH3 linker are identical. In some embodiments, the first CH3 linker and second CH3 linker are non-identical. In some embodiments, the first CH3 linker and second CH3 linker differ in length by 1-6 amino acids. In some embodiments, the first CH3 linker and second CH3 linker differ in length by 1-3 amino acids.

In particular embodiments, it is desirable to reduce an undesired association of domains B or G, which may contain CH3 sequences, with domains E and K, which may also contain CH3 sequences. In such cases, use of CH3 sequences from human IgA (IgA-CH3) in domains B and/or G may improve antibody assembly and stability by reducing such undesired associations. In some embodiments of a binding molecule wherein domains E and K comprise IgG-CH3 sequences, domains B and G comprises IgA-CH3 sequences.

In particular embodiments, at least one of domains B and G comprise a CH3 linker sequence as described in Section 6.4.19.3. In some embodiments, both domains B and G comprise a CH3 linker sequence as described in Section 6.4.19.3. In some embodiments, domain B comprises a first CH3 linker sequence and domain G comprises a second CH3 linker sequence. In some embodiments, the first CH3 linker sequence associates with the second CH3 linker sequence by formation of a disulfide bridge between cysteine residues of the first and second CH3 linker sequences. In some embodiments, the first CH3 linker and the second CH3 linker are identical. In some embodiments, the first CH3 linker and second CH3 linker are non-identical. In some embodiments, the first CH3 linker and second CH3 linker differ in length by 1-6 amino acids. In some embodiments, the first CH3 linker and second CH3 linker differ in length by 1-3 amino acids. In some embodiments, the first CH3 linker and second CH3 linker are 1-10, 2-8, or 3-6 amino acids in length. In some embodiments, the first CH3 linker is 3 amino acids in length and the second CH3 linker is 5 or 6 amino acids in length.

In some embodiments, the first CH3 linker and second CH3 linker each comprise an amino acid cysteine substitution in the endogenous IgA-CH3 sequence. In some embodiments, the first CH3 linker and second CH3 linker each consist of an amino acid cysteine substitution in the endogenous IgA-CH3 sequence. In some embodiments, the first CH3 linker is a H350C substitution and the second CH3 linker is a P355C substitution. In some embodiments, the first CH3 linker is a P355C substitution and the second CH3 linker is a H350C substitution.

For clarity, the residue designated “H350” in the IgA-CH3 domain sequence is the underlined “H” residue in the following endogenous IgA-CH3 sequence:

TFRPEVHLLPPPSEELALNELVTLTCLARGFSPKDVLVRWLQGSQELPR
EKYLTWASRQEPSQGTTTFAVTSILRVAAEDWKKGDTFSCMVGHEALPL
AFTQKTIDRL.

By way of example, an IgA-CH3 amino acid domain sequence with a “H350C” mutation in an otherwise endogenous IgA-CH3 domain has the following sequence:

TFRPEVCLLPPPSEELALNELVTLTCLARGFSPKDVLVRWLQGSQELPR
EKYLTWASRQEPSQGTTTFAVTSILRVAAEDWKKGDTFSCMVGHEALPL
AFTQKTIDRL.

For clarity, the residue designated “P355” in the IgA-CH3 domain sequence is the underlined “P” residue in the following endogenous IgA-CH3 sequence:

TFRPEVHLLPPPSEELALNELVTLTCLARGFSPKDVLVRWLQGSQELPR
EKYLTWASRQEPSQGTTTFAVTSILRVAAEDWKKGDTFSCMVGHEALPL
AFTQKTIDRL.

By way of example, an IgA-CH3 amino acid domain sequence with a “P355C” mutation in an otherwise endogenous IgA-CH3 domain has the following sequence:

TFRPEVHLLPPCSEELALNELVTLTCLARGFSPKDVLVRWLQGSQELPR
EKYLTWASRQEPSQGTTTFAVTSILRVAAEDWKKGDTFSCMVGHEALPL
AFTQKTIDRL.

In some embodiments, the first CH3 linker and the second CH3 linker are provided in Table 13, as described in Section 6.15.27.

In some embodiments, the first CH3 linker and the second CH3 linker are provided in Table 16, as described in Section 6.15.28.

In preferred embodiments, the first CH3 linker is AGC and the second CH3 linker is AGKGSC. In some embodiments, the first CH3 linker is AGKGC and the second CH3 linker is AGC. In some embodiments, the first CH3 linker is AGKGSC and the second CH3 linker is AGC. In some embodiments, the first CH3 linker is AGKC and the second CH3 linker is AGC. In some embodiments, the first CH3 linker is a H350C amino acid substitution and the second CH3 linker is a P355C amino acid substitution. In some embodiments, the first CH3 linker is a P355C amino acid substitution and the second CH3 linker is a H350C amino acid substitution.

In some embodiments, wherein the first and second domains comprise IgA-CH3 sequences and the third and fourth domains comprise IgA-CH3 sequences, unwanted associations between the first or second domains with either the third or fourth domains are reduced when the first and second domains comprise a first and second CH3 linker, respectively, and the third and fourth domains comprise a third and fourth CH3 linker, respectively. In some embodiments, the first and second CH3 linkers on the first and second domains preferentially pair with each other and do not preferentially pair with the third or fourth CH3 linkers on the third and fourth domains. In some embodiments, the third and fourth CH3 linkers on the third and fourth domains preferentially pair with each other and do not preferentially pair with the first or second CH3 linkers on the first and second domains. In some embodiments, the first and second CH3 linkers are selected from Table 13, and the third and fourth CH3 linkers each comprise an amino acid cysteine substitution in the endogenous IgA-CH3 sequence. In some embodiments, the third CH3 linker and fourth CH3 linker each consist of an amino acid cysteine substitution in the endogenous IgA-CH3 sequence. Exemplary cysteine substitutions in endogenous IgA-CH3 sequences are described herein.

### 6.4.15. Pairing of Domains E & K

In various embodiments, the E domain has a CH3 amino acid sequence.

In various embodiments, the K domain has a CH3 amino acid sequence.

In a variety of embodiments, the amino acid sequences of the E and K domains are identical, wherein the sequence is an endogenous CH3 sequence. CH3 sequences are described in Section 6.4.2.1. In some embodiments, the CH3 sequences of domains E and K are IgG-CH3 sequences.

In a variety of embodiments, the sequences of the E and K domains are different. In a variety of embodiments, the different sequences separately comprise respectively orthogonal modifications in an endogenous CH3 sequence, wherein the E domain interacts with the K domain, and wherein neither the E domain nor the K domain significantly interacts with a CH3 domain lacking the orthogonal modification. In certain embodiments, the orthogonal modifications include, but are not limited to, engineered disulfide bridges, knob-in-hole mutations, and charge-pair mutations, as described in greater detail in Sections 6.4.14.1-6.4.14.3. In particular embodiments, orthogonal modifications include a combination of orthogonal modifications selected from, but not limited to, engineered disulfide bridges, knob-in-hole mutations, and charge-pair mutations. In particular embodiments, the orthogonal modifications can be combined with amino acid substitutions that reduce immunogenicity, such as isoallotype mutations.

### 6.4.16. Pairing of Domains I & M and Domains H & L

In a variety of embodiments, domain I has a CL sequence and domain M has a CH1 sequence. In a variety of embodiments, domain H has a VL sequence and domain L has a VH sequence. In a preferred embodiment, domain H has a VL amino acid sequence, domain I has a CL amino acid sequence, domain L has a VH amino acid sequence, and domain M has a CH1 amino acid sequence. In another preferred embodiment, domain H has a VL amino acid sequence, domain I has a CL amino acid sequence, domain L has a VH amino acid sequence, domain M has a CH1 amino acid sequence, and domain K has a CH3 amino acid sequence.

In a variety of embodiments, the amino acid sequences of the I domain and the M domain separately comprise respectively orthogonal modifications in an endogenous sequence, wherein the I domain interacts with the M domain, and wherein neither the I domain nor the M domain significantly interacts with a domain lacking the orthogonal modification. In a series of embodiments, the orthogonal mutations in the I domain are in a CL sequence and the orthogonal mutations in the M domain are in CH1 sequence. Orthogonal mutations are in CH1 and CL sequences are described in more detail in Sections 6.3.1.1 and 6.3.1.2.

In a variety of embodiments, the amino acid sequences of the H domain and the L domain separately comprise respectively orthogonal modifications in an endogenous sequence, wherein the H domain interacts with the L domain, and wherein neither the H domain nor the L domain significantly interacts with a domain lacking the orthogonal modification. In a series of embodiments, the orthogonal mutations in the H domain are in a VL sequence and the orthogonal mutations in the L domain are in VH sequence. In specific embodiments, the orthogonal mutations are charge-pair mutations at the VH/VL interface. In preferred embodiments, the charge-pair mutations at the VH/VL interface are a Q39E in VH with a corresponding Q38K in VL, or a Q39K in VH with a corresponding Q38E in VL, as described in greater detail in Igawa et al. (Protein Eng. Des. Sel., 2010, vol. 23, 667-677), herein incorporated by reference for all it teaches.

In certain embodiments, the interaction between the A domain and the F domain form a first antigen binding site specific for a first antigen, and the interaction between the H domain and the L domain form a second antigen binding site specific for a second antigen. In certain embodiments, the interaction between the A domain and the F domain form a first antigen binding site specific for a first antigen, and the interaction between the H domain and the L domain form a second antigen binding site specific for the first antigen.

### 6.4.17. Trivalent Binding Molecules

In another series of embodiments, the binding molecules have three antigen binding sites and are therefore termed “trivalent.”

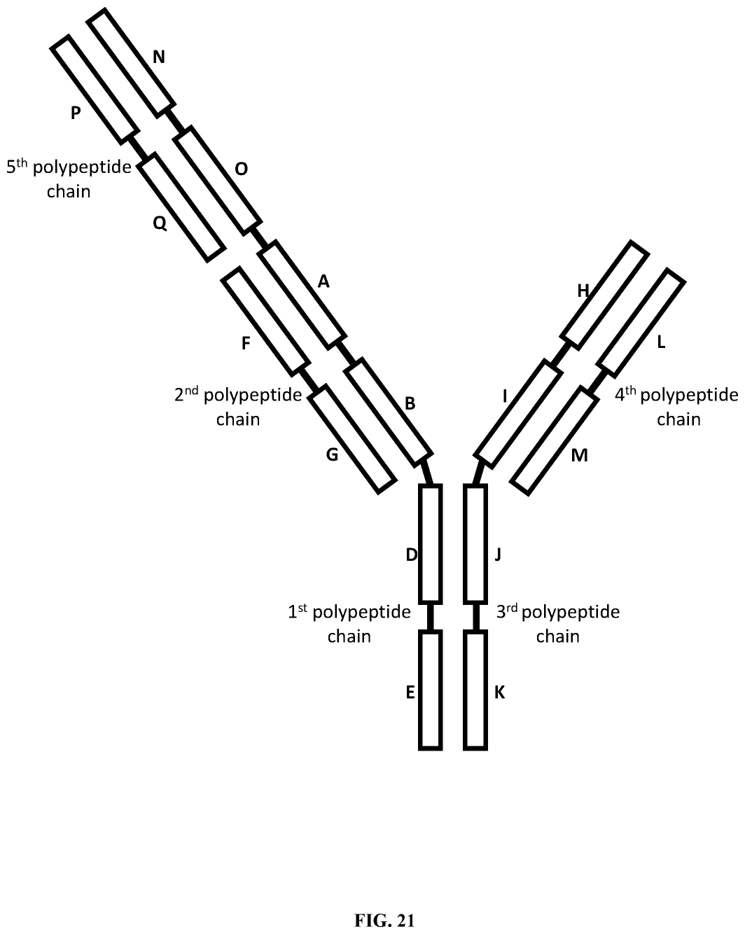

With reference to FIG. 21, in various trivalent embodiments the binding molecules further comprise a fifth polypeptide chain, wherein (a) the first polypeptide chain further comprises a domain N and a domain O, wherein the domains are arranged, from N-terminus to C-terminus, in a N-O-A-B-D-E orientation, and wherein domain N has a VL amino acid sequence, domain O has a constant region amino acid sequence; (b) the binding molecule further comprises a fifth polypeptide chain, comprising: a domain P and a domain Q, wherein the domains are arranged, from N-terminus to C-terminus, in a P-Q orientation, and wherein domain P has a VH amino acid sequence and domain Q has a constant region amino acid sequence; and (c) the first and the fifth polypeptides are associated through an interaction between the N and the P domains and an interaction between the O and the Q domains to form the binding molecule. As schematized in FIG. 2, these trivalent embodiments are termed “2×1” trivalent constructs.

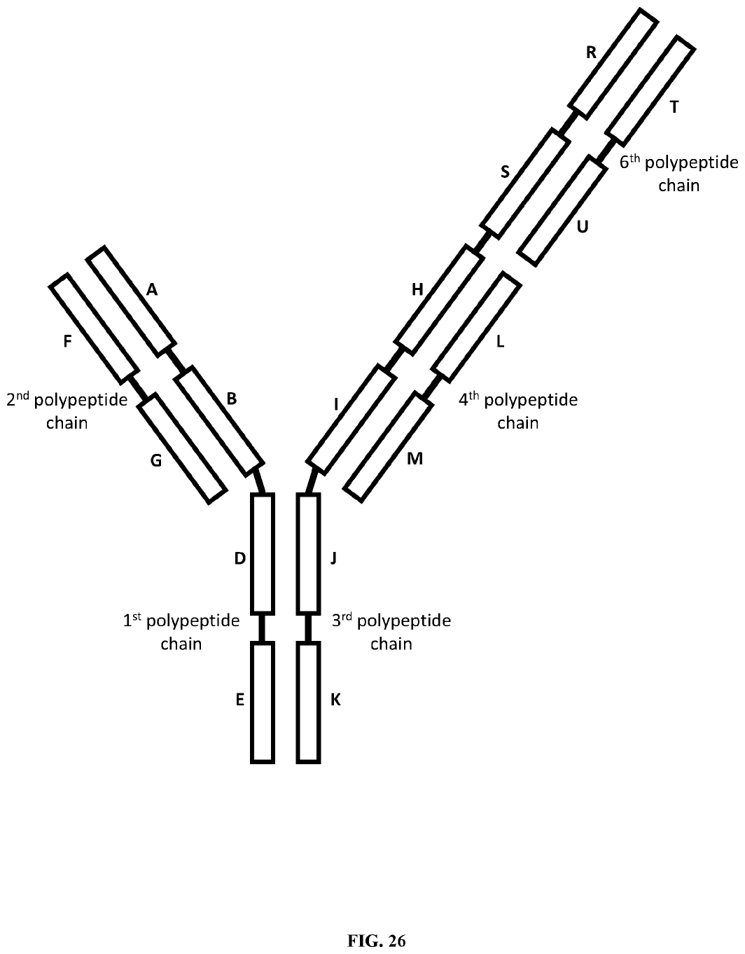

With reference to FIG. 26, in a further series of trivalent embodiments, the binding molecules further comprise a sixth polypeptide chain, wherein (a) the third polypeptide chain further comprises a domain R and a domain S, wherein the domains are arranged, from N-terminus to C-terminus, in a R-S-H-I-J-K orientation, and wherein domain R has a VL amino acid sequence and domain S has a constant domain amino acid sequence; (b) the binding molecule further comprises a sixth polypeptide chain, comprising: a domain T and a domain U, wherein the domains are arranged, from N-terminus to C-terminus, in a T-U orientation, and wherein domain T has a VH amino acid sequence and domain U has a constant domain amino acid sequence; and (c) the third and the sixth polypeptides are associated through an interaction between the R and the T domains and an interaction between the S and the U domains to form the binding molecule. As schematized in FIG. 2, these trivalent embodiments are termed “1×2” trivalent constructs.

In a variety of embodiments, the domain O is connected to domain A through a peptide linker. In a variety of embodiments, the domain S is connected to domain H through a peptide linker. In a preferred embodiment, the peptide linker connecting either domain O to domain A or connecting domain S to domain H is a 6 amino acid GSGSGS peptide sequence, as described in more detail in Section 6.4.19.6.

### 6.4.17.1. Trivalent 2×1 Bispecific Constructs [2(A-A)×1(B)]

With reference to FIG. 21, in a variety of embodiments the amino acid sequences of domain N and domain A are identical, the amino acid sequences of domain H is different from domains N and A, the amino acid sequences of domain O and domain B are identical, the amino acid sequences of domain I is different from domains O and B, the amino acid sequences of domain P and domain F are identical, the amino acid sequences of domain L is different from domains P and F, the amino acid sequences of domain Q and domain G are identical, the amino acid sequences of domain M is different from domains Q and G; and the interaction between the A domain and the F domain form a first antigen binding site specific for a first antigen, the interaction between the H domain and the L domain form a second antigen binding site specific for a second antigen, and the domain N and domain P form a third antigen binding site specific for the first antigen.

### 6.4.17.2. Trivalent 2×1 Bispecific Constructs [2(A-B)×1(A)]

With reference to FIG. 21, in a variety of embodiments the amino acid sequences of domain N and domain H are identical, the amino acid sequences of domain A is different from domains N and H, the amino acid sequences of domain O and domain I are identical, the amino acid sequences of domain B is different from domains O and I, the amino acid sequences of domain P and domain L are identical, the amino acid sequences of domain F is different from domains P and L, the amino acid sequences of domain Q and domain M are identical, the amino acid sequences of domain G is different from domains Q and M; and the interaction between the A domain and the F domain form a first antigen binding site specific for a first antigen, the interaction between the H domain and the L domain form a second antigen binding site specific for a second antigen, and the domain N and domain P form a third antigen binding site specific for the second antigen.

### 6.4.17.3. Trivalent 2×1 Trispecific Constructs [2(A-B)×1(C)]

With reference to FIG. 21, in a variety of embodiments, the amino acid sequences of domain N, domain A, and domain H are different, the amino acid sequences of domain O, domain B, and domain I are different, the amino acid sequences of domain P, domain F, and domain L are different, and the amino acid sequences of domain Q, domain G, and domain M are different; and the interaction between the A domain and the F domain form a first antigen binding site specific for a first antigen, the interaction between the H domain and the L domain form a second antigen binding site specific for a second antigen, and the domain N and domain P form a third antigen binding site specific for a third antigen.

In certain embodiments, domain O has a constant region sequence that is a CL from a kappa light chain and domain Q has a constant region sequence that is a CH1 from an IgG1 isotype, as discussed in more detail in Section 6.3. In a preferred embodiment, domain O and domain Q have CH3 sequences such that they specifically associate with each other, as discussed in more detail in Section 6.4.14.

### 6.4.17.4. Trivalent 1×2 Bispecific Constructs [1(A)×2(B-A)]

With reference to FIG. 26, in a variety of embodiments, the amino acid sequences of domain R and domain A are identical, the amino acid sequences of domain H is different from domain R and A, the amino acid sequences of domain S and domain B are identical, the amino acid sequences of domain I is different from domain S and B, the amino acid sequences of domain T and domain F are identical, the amino acid sequences of domain L is different from domain T and F, the amino acid sequences of domain U and domain G are identical, the amino acid sequences of domain M is different from domain U and G and the interaction between the A domain and the F domain form a first antigen binding site specific for a first antigen, the interaction between the H domain and the L domain form a second antigen binding site specific for a second antigen, and the domain R and domain T form a third antigen binding site specific for the first antigen.

### 6.4.17.5. Trivalent 1×2 Bispecific Constructs [1(A)×2(B-B)]

In a variety of embodiments, the binding molecule further comprises a second CH1 domain, or portion thereof. With reference to FIG. 26, in specific embodiments, the amino acid sequences of domain R and domain H are identical, the amino acid sequences of domain A is different from domain R and H, the amino acid sequences of domain S and domain I are identical, the amino acid sequences of domain B is different from domain S and I, the amino acid sequences of domain T and domain L are identical, the amino acid sequences of domain F is different from domain T and L, the amino acid sequences of domain U and domain M are identical, the amino acid sequences of domain G is different from domain U and M and the interaction between the A domain and the F domain form a first antigen binding site specific for a first antigen, the interaction between the H domain and the L domain form a second antigen binding site specific for a second antigen, and the domain R and domain T form a third antigen binding site specific for the second antigen.

In particular embodiments, the amino acid sequences of domain S and domain I are CH1 sequences. In particular embodiments, the amino acid sequences of domain U and domain M are CH1 sequences.

### 6.4.17.6. Trivalent 1×2 Trispecific Constructs [1(A)×2(B-C)]

With reference to FIG. 26, in a variety of embodiments, the amino acid sequences of domain R, domain A, and domain H are different, the amino acid sequences of domain S, domain B, and domain I are different, the amino acid sequences of domain T, domain F, and domain L are different, and the amino acid sequences of domain U, domain G, and domain M are different; and the interaction between the A domain and the F domain form a first antigen binding site specific for a first antigen, the interaction between the H domain and the L domain form a second antigen binding site specific for a second antigen, and the domain R and domain T form a third antigen binding site specific for a third antigen.

In particular embodiments, domain S has a constant region sequence that is a CL from a kappa light chain and domain U has a constant region sequence that is a CH1 from an IgG1 isotype, as discussed in more detail in Section 6.3. In a preferred embodiment, domain S and domain U have CH3 sequences such that they specifically associate with each other, as discussed in more detail in Section 6.4.14.

In certain embodiments, the binding molecule further comprises a second CH1 domain, or portion thereof. In particular embodiments, the amino acid sequences of domain S and domain I are CH1 sequences. In particular embodiments, the amino acid sequences of domain U and domain M are CH1 sequences.

### 6.4.18. Tetravalent 2×2 Binding Molecules

In a variety of embodiments, the binding molecules have 4 antigen binding sites and are therefore termed “tetravalent.”

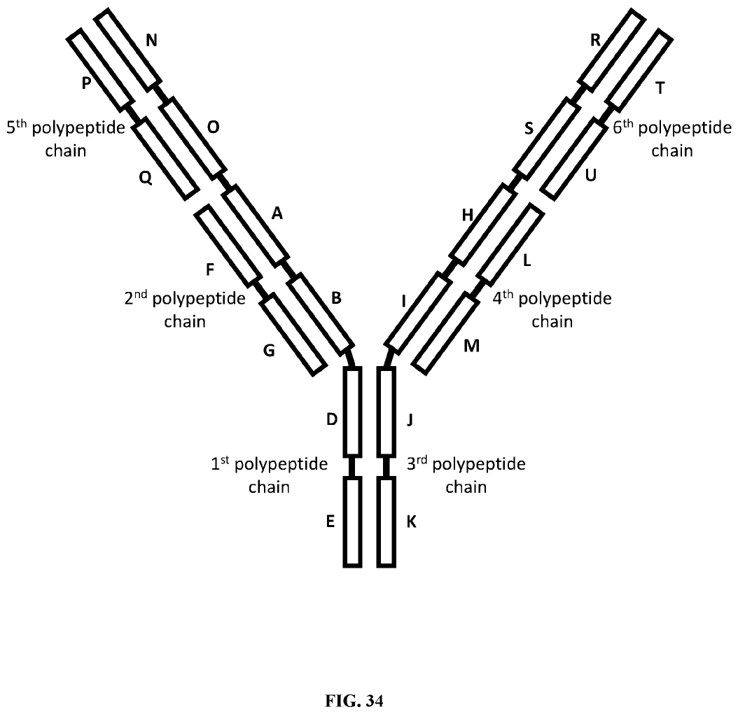

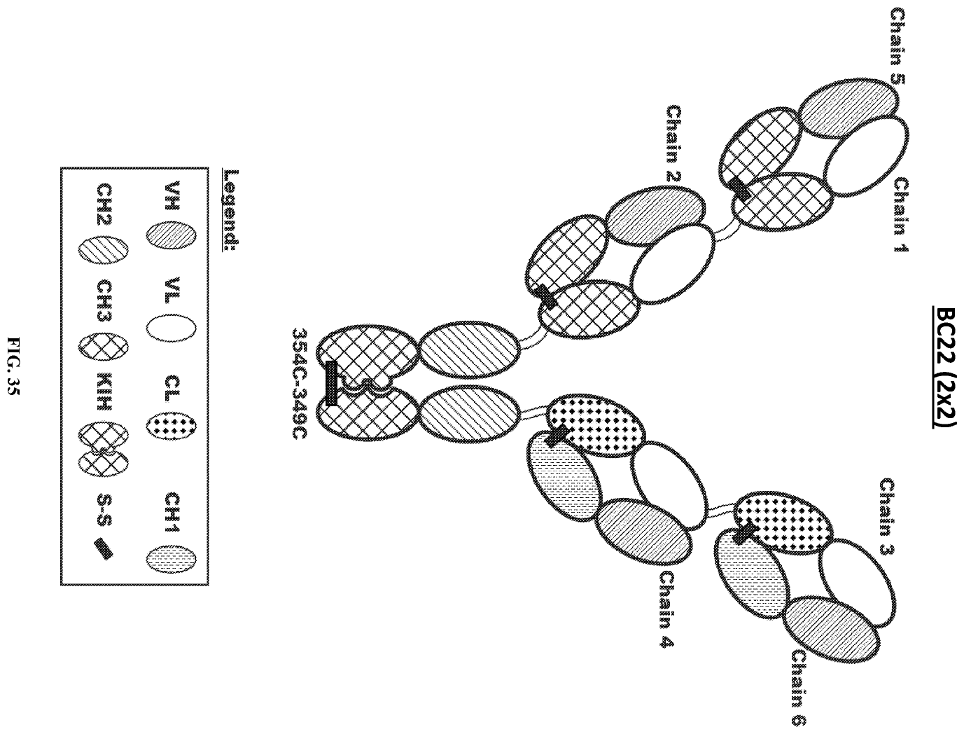

With reference to FIG. 34, in a further series of embodiments, the binding molecules further comprise a fifth and a sixth polypeptide chain, wherein (a) the first polypeptide chain further comprises a domain N and a domain O, wherein the domains are arranged, from N-terminus to C-terminus, in a N-O-A-B-D-E orientation; (b) the third polypeptide chain further comprises a domain R and a domain S, wherein the domains are arranged, from N-terminus to C-terminus, in a R-S-H-I-J-K orientation; (c) the binding molecule further comprises a fifth and a sixth polypeptide chain, wherein the fifth polypeptide chain comprises a domain P and a domain Q, wherein the domains are arranged, from N-terminus to C-terminus, in a P-Q orientation, and the sixth polypeptide chain comprises a domain T and a domain U, wherein the domains are arranged, from N-terminus to C-terminus, in a T-U orientation; and (d) the first and the fifth polypeptides are associated through an interaction between the N and the P domains and an interaction between the O and the Q domains, and the third and the sixth polypeptides are associated through an interaction between the R and the T domains and an interaction between the S and the U domains to form the binding molecule.

In a variety of embodiments, the domain O is connected to domain A through a peptide linker and the domain S is connected to domain H through a peptide linker. In a preferred embodiment, the peptide linker connecting domain O to domain A and connecting domain S to domain H is a 6 amino acid GSGSGS peptide sequence, as described in more detail in Section 6.4.19.6.

### 6.4.18.1. Tetravalent 2×2 Bispecific Constructs

With reference to FIG. 34, in a series of tetravalent 2×2 bispecific binding molecules, the amino acid sequences of domain N and domain A are identical, the amino acid sequences of domain H and domain R are identical, the amino acid sequences of domain O and domain B are identical, the amino acid sequences of domain I and domain S are identical, the amino acid sequences of domain P and domain F are identical, the amino acid sequences of domain L and domain T are identical, the amino acid sequences of domain Q and domain G are identical, the amino acid sequences of domain M and domain U are identical; and wherein the interaction between the A domain and the F domain form a first antigen binding site specific for a first antigen, the domain N and domain P form a second antigen binding site specific for the first antigen, the interaction between the H domain and the L domain form a third antigen binding site specific for a second antigen, and the interaction between the R domain and the T domain form a fourth antigen binding site specific for the second antigen.

With reference to FIG. 34, in another series of tetravalent 2×2 bispecific binding molecules, the amino acid sequences of domain H and domain A are identical, the amino acid sequences of domain N and domain R are identical, the amino acid sequences of domain I and domain B are identical, the amino acid sequences of domain O and domain S are identical, the amino acid sequences of domain L and domain F are identical, the amino acid sequences of domain P and domain T are identical, the amino acid sequences of domain M and domain G are identical, the amino acid sequences of domain Q and domain U are identical; and wherein the interaction between the A domain and the F domain form a first antigen binding site specific for a first antigen, the domain N and domain P form a second antigen binding site specific for a second antigen, the interaction between the H domain and the L domain form a third antigen binding site specific for the first antigen, and the interaction between the R domain and the T domain form a fourth antigen binding site specific for the second antigen.

### 6.4.19. Domain Junctions

### 6.4.19.1. Junctions Connecting VL and CH3 Domains

In a variety of embodiments, the amino acid sequence that forms a junction between the C-terminus of a VL domain and the N-terminus of a CH3 domain is an engineered sequence. In certain embodiments, one or more amino acids are deleted or added in the C-terminus of the VL domain. In certain embodiments, the junction connecting the C-terminus of a VL domain and the N-terminus of a CH3 domain is one of the sequences described in Table 2 in Section 6.15.7. In particular embodiments, A111 is deleted in the C-terminus of the VL domain. In certain embodiments, one or more amino acids are deleted or added in the N-terminus of the CH3 domain. In particular embodiments, P343 is deleted in the N-terminus of the CH3 domain. In particular embodiments, P343 and R344 are deleted in the N-terminus of the CH3 domain. In certain embodiments, one or more amino acids are deleted or added to both the C-terminus of the VL domain and the N-terminus of the CH3 domain. In particular embodiments, A111 is deleted in the C-terminus of the VL domain and P343 is deleted in the N-terminus of the CH3 domain. In a preferred embodiment, A111 and V110 are deleted in the C-terminus of the VL domain. In another preferred embodiment, A111 and V110 are deleted in the C-terminus of the VL domain and the N-terminus of the CH3 domain has a P343V mutation.

### 6.4.19.2. Junctions Connecting VII and CH3 Domains

In a variety of embodiments, the amino acid sequence that forms a junction between the C-terminus of a VH domain and the N-terminus of a CH3 domain is an engineered sequence. In certain embodiments, one or more amino acids are deleted or added in the C-terminus of the VH domain. In certain embodiments, the junction connecting the C-terminus of a VH domain and the N-terminus of the CH3 domain is one of the sequences described in Table 3 in Section 6.15.7. In particular embodiments, K117 and G118 are deleted in the C-terminus of the VH domain. In certain embodiments, one or more amino acids are deleted or added in the N-terminus of the CH3 domain. In particular embodiments, P343 is deleted in the N-terminus of the CH3 domain. In particular embodiments, P343 and R344 are deleted in the N-terminus of the CH3 domain. In particular embodiments, P343, R344, and E345 are deleted in the N-terminus of the CH3 domain. In certain embodiments, one or more amino acids are deleted or added to both the C-terminus of the VH domain and the N-terminus of the CH3 domain. In a preferred embodiment, T116, K117, and G118 are deleted in the C-terminus of the VH domain.

### 6.4.19.3. Junctions Connecting CH3 C-Terminus to CH2 N-Terminus (Hinge)

In the binding molecules described herein, the N-terminus of the CH2 domain has a “hinge” region amino acid sequence. As used herein, hinge regions are sequences of an antibody heavy chain that link the N-terminal variable domain-constant domain segment of an antibody and a CH2 domain of an antibody. In addition, the hinge region typically provides both flexibility between the N-terminal variable domain-constant domain segment and CH2 domain, as well as amino acid sequence motifs that form disulfide bridges between heavy chains (e.g. the first and the third polypeptide chains). As used herein, the hinge region amino acid sequence is SEQ ID NO: 56.

In a variety of embodiments, a CH3 amino acid sequence is extended at the C-terminus at the junction between the C-terminus of the CH3 domain and the N-terminus of a CH2 domain. In certain embodiments, a CH3 amino acid sequence is extended at the C-terminus at the junction between the C-terminus of the CH3 domain and a hinge region, which in turn is connected to the N-terminus of a CH2 domain. In a preferred embodiment, the CH3 amino acid sequence is extended by inserting a CH3 amino acid extension sequence (“CH3 linker sequence” or “CH3 linker”). In some embodiments, the CH3 amino acid extension sequence is followed by the DKTHT motif of an IgG1 hinge region. In some embodiments, the CH3 amino acid extension sequence is 3-10 amino acids in length. In some embodiments, the CH3 amino acid extension sequence is 3-8 amino acids in length. In some embodiments, the CH3 amino acid extension sequence is 3-6 amino acids in length.

In some embodiments, the CH3 amino acid extension sequence is a PGK tripeptide. In some embodiments, the CH3 amino acid extension sequence is an AGC tripeptide. In some embodiments, the CH3 amino acid extension sequence is a GEC tripeptide. In some embodiments, the CH3 amino acid extension sequence is AGKC (SEQ ID NO:96). In some embodiments, the CH3 amino acid extension sequence is PGKC (SEQ ID NO:97). In some embodiments, the CH3 amino acid extension sequence is AGKGC (SEQ ID NO:98). In some embodiments, the CH3 amino acid extension sequence is AGKGSC (SEQ ID NO:99).

In a particular embodiment, the extension at the C-terminus of the CH3 domain incorporates amino acid sequences that can form a disulfide bond with orthogonal C-terminal extension of another CH3 domain. In a preferred embodiment, the extension at the C-terminus of the CH3 domain incorporates a KSC tripeptide sequence that is followed by the DKTHT motif of an IgG1 hinge region that forms a disulfide bond with orthogonal C-terminal extension of another CH3 domain that incorporates a GEC motif of a kappa light chain.

In some embodiments of a binding molecule wherein domains B and G comprise CH3 amino acid sequences, domain B comprises a first CH3 linker sequence and domain G comprises a second CH3 linker sequence. In some embodiments, the first CH3 linker sequence associates with the second CH3 linker sequence by formation of a disulfide bridge between cysteine residues of the first and second CH3 linker sequences. In some embodiments, the first CH3 linker and the second CH3 linker are identical. In some embodiments, the first CH3 linker and second CH3 linker are non-identical. In some embodiments, the first CH3 linker and second CH3 linker differ in length by 1-6 amino acids. In some embodiments, the first CH3 linker and second CH3 linker differ in length by 1-3 amino acids.

In some embodiments, the first CH3 linker and the second CH3 linker are provided in Table 13, as described in Section 6.15.27.

In preferred embodiments, the first CH3 linker is AGC and the second CH3 linker is AGKGSC. In some embodiments, the first CH3 linker is AGKGC and the second CH3 linker is AGC. In some embodiments, the first CH3 linker is AGKGSC and the second CH3 linker is AGC. In some embodiments, the first CH3 linker is AGKC and the second CH3 linker is AGC.

### 6.4.19.4. Junctions Connecting CL C-Terminus and CH2 N-Terminus (Hinge)

In a variety of embodiments, a CL amino acid sequence is connected through its C-terminus to a hinge region, which in turn is connected to the N-terminus of a CH2 domain. Hinge region sequences are described in more detail in Section 6.4.19.3. In a preferred embodiment, the hinge region amino acid sequence is SEQ ID NO:56.

### 6.4.19.5. Junctions Connecting CH2 C-Terminus to Constant Region Domain

In a variety of embodiments, a CH2 amino acid sequence is connected through its C-terminus to the N-terminus of a constant region domain. Constant regions are described in more detail in Section 6.4.4. In a preferred embodiment, the CH2 sequence is connected to a CH3 sequence via its endogenous sequence. In other embodiments, the CH2 sequence is connected to a CH1 or CL sequence. Examples discussing connecting a CH2 sequence to a CH1 or CL sequence are described in more detail in U.S. Pat. No. 8,242,247, which is hereby incorporated in its entirety.

### 6.4.19.6. Junctions Connecting Domain O to Domain A or Domain S to Domain H on Trivalent and Tetravalent Molecules

In a variety of embodiments, heavy chains of antibodies (e.g. the first and third polypeptide chains) are extended at their N-terminus to include additional domains that provide additional ABSs. With reference to FIG. 21, FIG. 26, and FIG. 34, in certain embodiments, the C-terminus of the constant region domain amino acid sequence of a domain O and/or a domain S is connected to the N-terminus of the variable region domain amino acid sequence of a domain A and/or a domain H, respectively. In some preferred embodiments, the constant region domain is a CH3 amino acid sequence and the variable region domain is a VL amino acid sequence. In some preferred embodiments, the constant region domain is a CL amino acid sequence and the variable region domain is a VL amino acid sequence. In certain embodiments, the constant region domain is connected to the variable region domain through a peptide linker. In a preferred embodiment, the peptide linker is a 6 amino acid GSGSGS peptide sequence.

In a variety of embodiments, light chains of antibodies (e.g. the second and fourth polypeptide chains) are extended at their N-terminus to include additional variable domain-constant domain segments of an antibody. In certain embodiments, the constant region domain is a CH1 amino acid sequence and the variable region domain is a VH amino acid sequence.

### 6.5. Exemplary Bivalent Binding Molecules

In a further aspect, bivalent binding molecules are provided.

With reference to FIG. 3, in a series of embodiments the binding molecules comprise a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a VL amino acid sequence, domain B has a CH3 amino acid sequence, domain D has a CH2 amino acid sequence, and domain E has a constant region domain amino acid sequence; (b) the second polypeptide chain comprises a domain F and a domain G, wherein the domains are arranged, from N-terminus to C-terminus, in a F-G orientation, and wherein domain F has a VH amino acid sequence and domain G has a CH3 amino acid sequence; (c) the third polypeptide chain comprises a domain H, a domain I, a domain J, and a domain K, wherein the domains are arranged, from N-terminus to C-terminus, in a H-I-J-K orientation, and wherein domain H has a variable region domain amino acid sequence, domain I has a constant region domain amino acid sequence, domain J has a CH2 amino acid sequence, and K has a constant region domain amino acid sequence; (d) the fourth polypeptide chain comprises a domain L and a domain M, wherein the domains are arranged, from N-terminus to C-terminus, in a L-M orientation, and wherein domain L has a variable region domain amino acid sequence and domain M has a constant region domain amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; and (g) the first and the third polypeptides are associated through an interaction between the D and the J domains and an interaction between the E and the K domains to form the binding molecule.

In a preferred embodiment, domain E has a CH3 amino acid sequence, domain H has a VL amino acid sequence, domain I has a CL amino acid sequence, domain K has a CH3 amino acid sequence, domain L has a VH amino acid sequence, and domain M has a CH1 amino acid sequence.

In certain embodiments, the interaction between the A domain and the F domain form a first antigen binding site specific for a first antigen, and the interaction between the H domain and the L domain form a second antigen binding site specific for a second antigen, and the binding molecule is a bispecific bivalent binding molecule. In certain embodiments, the interaction between the A domain and the F domain form a first antigen binding site specific for a first antigen, and the interaction between the H domain and the L domain form a second antigen binding site specific for the first antigen, and the binding molecule is a monospecific bivalent binding molecule.

### 6.5.1. Bivalent Bispecific B-Body “BC1”

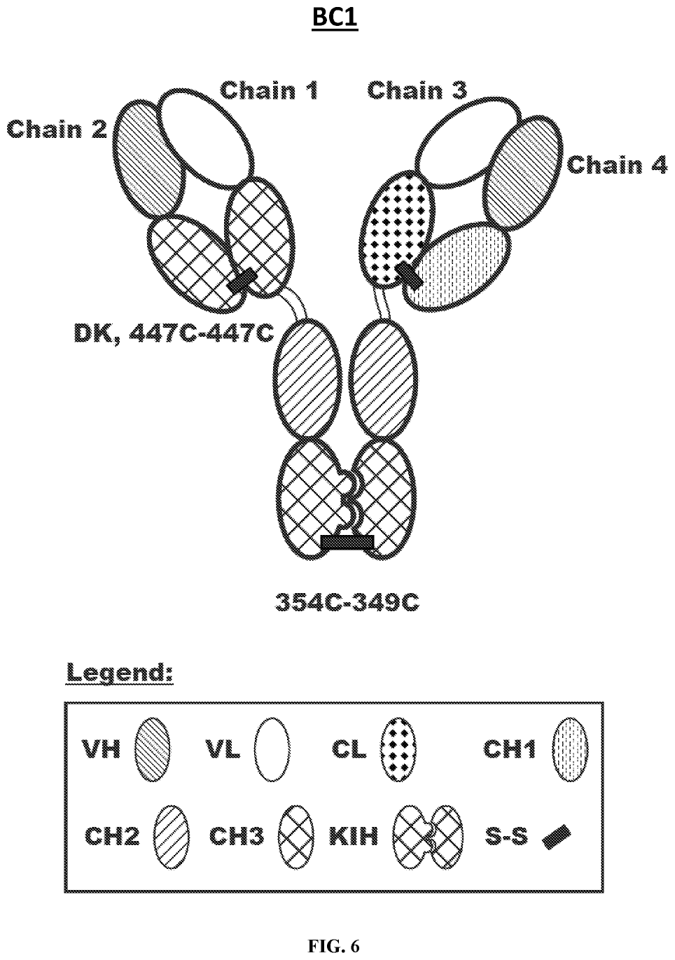

With reference to FIG. 3 and FIG. 6, in a series of embodiments, the binding molecule has a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a first VL amino acid sequence, domain B has a human IgG1 CH3 amino acid sequence with a T366K mutation and a C-terminal extension incorporating a KSC tripeptide sequence that is followed by the DKTHT motif of an IgG1 hinge region, domain D has a human IgG1 CH2 amino acid sequence, and domain E has human IgG1 CH3 amino acid with a S354C and T366W mutation; (b) the second polypeptide chain has a domain F and a domain G, wherein the domains are arranged, from N-terminus to C-terminus, in a F-G orientation, and wherein domain F has a first VH amino acid sequence and domain G has a human IgG1 CH3 amino acid sequence with a L351D mutation and a C-terminal extension incorporating a GEC amino acid disulfide motif; (c) the third polypeptide chain has a domain H, a domain I, a domain J, and a domain K, wherein the domains are arranged, from N-terminus to C-terminus, in a H-I-J-K orientation, and wherein domain H has a second VL amino acid sequence, domain I has a human CL kappa amino acid sequence, domain J has a human IgG1 CH2 amino acid sequence, and K has a human IgG1 CH3 amino acid sequence with a Y349C, a D356E, a L358M, a T366S, a L368A, and a Y407V mutation; (d) the fourth polypeptide chain has a domain L and a domain M, wherein the domains are arranged, from N-terminus to C-terminus, in a L-M orientation, and wherein domain L has a second VH amino acid sequence and domain M has a human IgG1 CH1 amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the binding molecule; (h) domain A and domain F form a first antigen binding site specific for a first antigen; and (i) domain H and domain L form a second antigen binding site specific for a second antigen.

In preferred embodiments, the first polypeptide chain has the sequence SEQ ID NO:8, the second polypeptide chain has the sequence SEQ ID NO:9, the third polypeptide chain has the sequence SEQ ID NO:10, and the fourth polypeptide chain has the sequence SEQ ID NO:11.

### 6.5.2. Bivalent Bispecific B-Body “BC6”

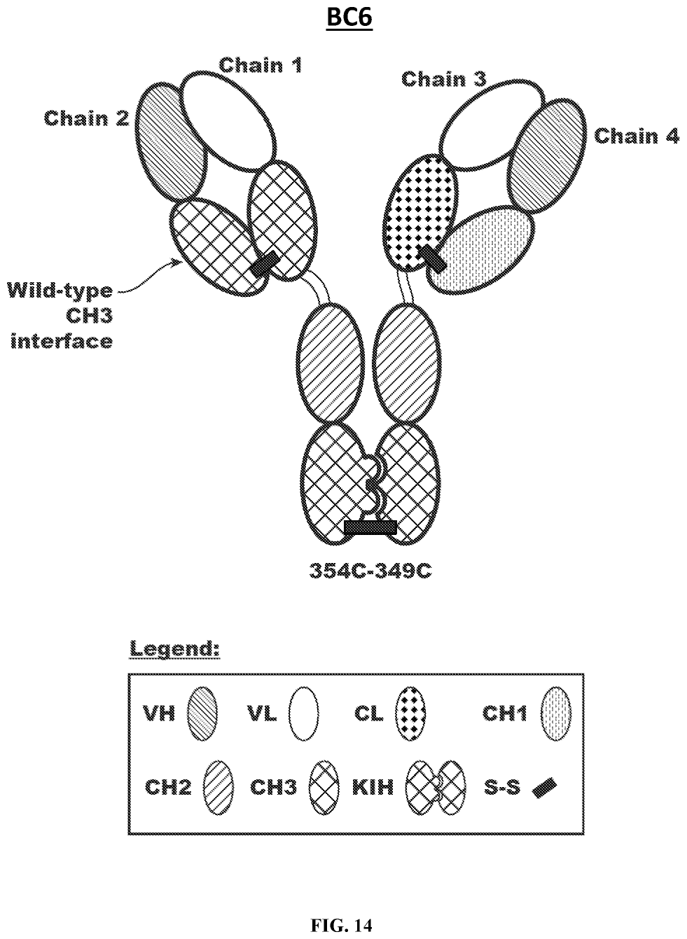

With reference to FIG. 3 and FIG. 14, in a series of embodiments, the binding molecule has a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a first VL amino acid sequence, domain B has a human IgG1 CH3 amino acid sequence with a C-terminal extension incorporating a KSC tripeptide sequence that is followed by the DKTHT motif of an IgG1 hinge region, domain D has a human IgG1 CH2 amino acid sequence, and domain E has human IgG1 CH3 amino acid with a S354C and a T366W mutation; (b) the second polypeptide chain has a domain F and a domain G, wherein the domains are arranged, from N-terminus to C-terminus, in a F-G orientation, and wherein domain F has a first VH amino acid sequence and domain G has a human IgG1 CH3 amino acid sequence with a C-terminal extension incorporating a GEC amino acid disulfide motif; (c) the third polypeptide chain has a domain H, a domain I, a domain J, and a domain K, wherein the domains are arranged, from N-terminus to C-terminus, in a H-I-J-K orientation, and wherein domain H has a second VL amino acid sequence, domain I has a human CL kappa amino acid sequence, domain J has a human IgG1 CH2 amino acid sequence, and K has a human IgG1 CH3 amino acid sequence with a Y349C, a D356E, a L358M, a T366S, a L368A, and a Y407V mutation; (d) the fourth polypeptide chain has a domain L and a domain M, wherein the domains are arranged, from N-terminus to C-terminus, in a L-M orientation, and wherein domain L has a second VH amino acid sequence and domain M has a human IgG1 amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the binding molecule; (h) domain A and domain F form a first antigen binding site specific for a first antigen; and (i) domain H and domain L form a second antigen binding site specific for a second antigen.

### 6.5.3. Bivalent Bispecific B-Body “BC28”

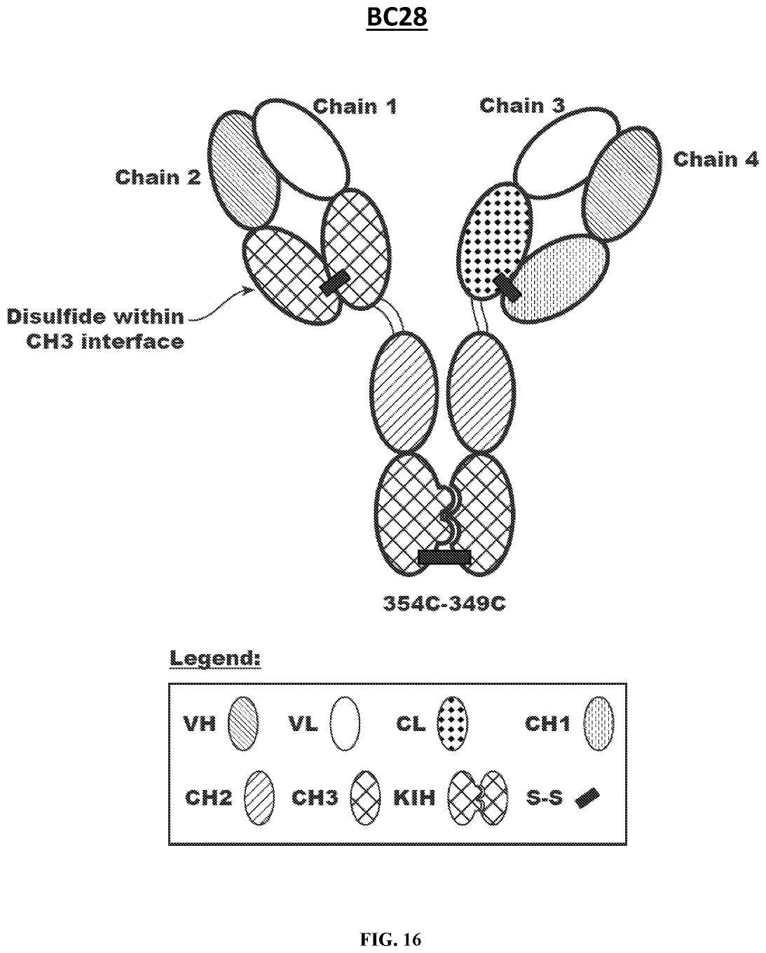

With reference to FIG. 3 and FIG. 16, in a series of embodiments, the binding molecule has a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a first VL amino acid sequence, domain B has a human IgG1 CH3 amino acid sequence with a Y349C mutation and a C-terminal extension incorporating a PGK tripeptide sequence that is followed by the DKTHT motif of an IgG1 hinge region, domain D has a human IgG1 CH2 amino acid sequence, and domain E has a human IgG1 CH3 amino acid with a S354C and a T366W mutation; (b) the second polypeptide chain has a domain F and a domain G, wherein the domains are arranged, from N-terminus to C-terminus, in a F-G orientation, and wherein domain F has a first VH amino acid sequence and domain G has a human IgG1 CH3 amino acid sequence with a S354C mutation and a C-terminal extension incorporating a PGK tripeptide sequence; (c) the third polypeptide chain has a domain H, a domain I, a domain J, and a domain K, wherein the domains are arranged, from N-terminus to C-terminus, in a H-I-J-K orientation, and wherein domain H has a second VL amino acid sequence, domain I has a human CL kappa amino acid sequence, domain J has a human IgG1 CH2 amino acid sequence, and K has a human IgG1 CH3 amino acid sequence with a Y349C, a D356E, a L358M, a T366S, a L368A, and a Y407V; (d) the fourth polypeptide chain has a domain L and a domain M, wherein the domains are arranged, from N-terminus to C-terminus, in a L-M orientation, and wherein domain L has a second VH amino acid sequence and domain M has a human IgG1 CH1 amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the binding molecule; (h) domain A and domain F form a first antigen binding site specific for a first antigen; and (i) domain H and domain L form a second antigen binding site specific for a second antigen.

In preferred embodiments, the first polypeptide chain has the sequence SEQ ID NO:24, the second polypeptide chain has the sequence SEQ ID NO:25, the third polypeptide chain has the sequence SEQ ID NO:10, and the fourth polypeptide chain has the sequence SEQ ID NO:11.

### 6.5.4. Bivalent Bispecific B-Body “BC44”

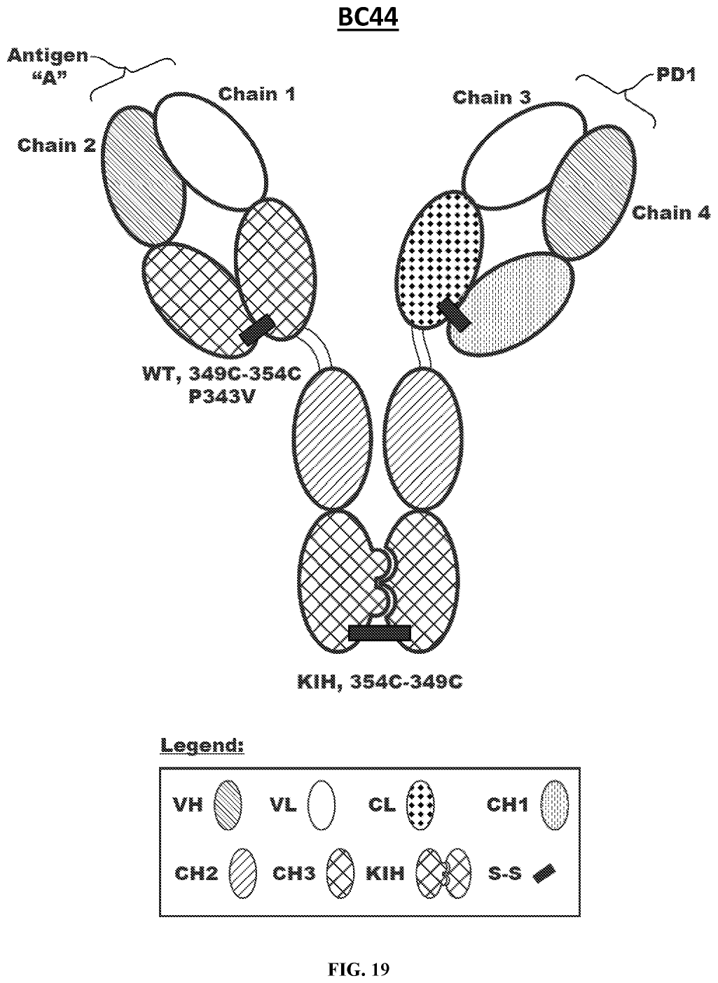

With reference to FIG. 3 and FIG. 19, in a series of embodiments, the binding molecule has a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a first VL amino acid sequence, domain B has a human IgG1 CH3 amino acid sequence with a Y349C mutation, a P343V mutation, and a C-terminal extension incorporating a PGK tripeptide sequence that is followed by the DKTHT motif of an IgG1 hinge region, domain D has a human IgG1 CH2 amino acid sequence, and domain E has human IgG1 CH3 amino acid with a S354C mutation and a T366W mutation; (b) the second polypeptide chain has a domain F and a domain G, wherein the domains are arranged, from N-terminus to C-terminus, in a F-G orientation, and wherein domain F has a first VH amino acid sequence and domain G has a human IgG1 CH3 amino acid sequence with a S354C mutation and a C-terminal extension incorporating a PGK tripeptide sequence; (c) the third polypeptide chain has a domain H, a domain I, a domain J, and a domain K, wherein the domains are arranged, from N-terminus to C-terminus, in a H-I-J-K orientation, and wherein domain H has a second VL amino acid sequence, domain I has a human CL kappa amino acid sequence, domain J has a human IgG1 CH2 amino acid sequence, and K has a human IgG1 CH3 amino acid sequence with a Y349C, T366S, L368A, and aY407V; (d) the fourth polypeptide chain has a domain L and a domain M, wherein the domains are arranged, from N-terminus to C-terminus, in a L-M orientation, and wherein domain L has a second VH amino acid sequence and domain M has a human IgG1 amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; and (g) the first and the third polypeptides are associated through an interaction between the D and the J domains and an interaction between the E and the K domains to form the binding molecule; (h) domain A and domain F form a first antigen binding site specific for a first antigen; and (i) domain H and domain L form a second antigen binding site specific for a second antigen.

In preferred embodiments, the first polypeptide chain has the sequence SEQ ID NO:32, the second polypeptide chain has the sequence SEQ ID NO:25, the third polypeptide chain has the sequence SEQ ID NO:10, and the fourth polypeptide chain has the sequence SEQ ID NO:11.

### 6.5.5. Exemplary Bivalent Binding Molecules with IgA-CH3 Domain Pairs

With reference to FIG. 3, in a series of embodiments, the binding molecule has a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a variable region amino acid sequence, domain B has a human IgA CH3 amino acid sequence, domain D has a human IgG1 CH2 amino acid sequence, and domain E has human IgG1 CH3 amino acid sequence; (b) the second polypeptide chain has a domain F and a domain G, wherein the domains are arranged, from N-terminus to C-terminus, in a F-G orientation, and wherein domain F has a variable region amino acid sequence and domain G has a human IgA CH3 amino acid sequence; (c) the third polypeptide chain has a domain H, a domain I, a domain J, and a domain K, wherein the domains are arranged, from N-terminus to C-terminus, in a H-I-J-K orientation, and wherein domain H has a variable region amino acid sequence, domain I has a constant region amino acid sequence, domain J has a human IgG1 CH2 amino acid sequence, and domain K has a human IgG1 CH3 amino acid sequence; (d) the fourth polypeptide chain has a domain L and a domain M, wherein the domains are arranged, from N-terminus to C-terminus, in a L-M orientation, and wherein domain L has a variable region amino acid sequence and domain M has a constant region amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the binding molecule. In some embodiments, domain A and domain F form a first antigen binding site specific for a first antigen; and domain H and domain L form a second antigen binding site specific for a second antigen.

In some embodiments, domain A comprises a VH amino acid sequence, domain F comprises a VL amino acid sequence, domain H comprises a VH amino acid sequence, domain I comprises a CH1 amino acid sequence, domain L comprises a VL amino acid sequence, and domain M comprises a CL amino acid sequence. In some embodiments, domain A comprises a first VH amino acid sequence and domain F comprises a first VL amino acid sequence, domain H comprises a second VH amino acid sequence and domain L comprises a second VL amino acid sequence.

In preferred embodiments, domain A comprises a VL amino acid sequence, domain F comprises a VH amino acid sequence, domain H comprises a VL amino acid sequence, domain L comprises a VH amino acid sequence, domain I comprises a CL amino acid sequence, and domain M comprises a CH1 amino acid sequence. In some embodiments, the CL amino acid sequence is a CL-kappa sequence. In some embodiments, domain A comprises a first VL amino acid sequence and domain F comprises a first VH amino acid sequence, domain H comprises a second VL amino acid sequence and domain L comprises a second VH amino acid sequence.

In some embodiments, domain E further comprises a S354C and T366W mutation in the human IgG1 CH3 amino acid sequence. In some embodiments, domain K further comprises a Y349C, a D356E, a L358M, a T366S, a L368A, and a Y407V mutation in the human IgG1 CH3 amino acid sequence.

In some embodiments, domain B comprises a first CH3 linker sequence as described in Section 6.4.19.3 that is followed by the DKTHT motif of an IgG1 hinge region; and domain G comprises a second CH3 linker sequence as described in Section 6.4.19.3. In some embodiments, the first CH3 linker sequence associates with the second CH3 linker sequence by formation of a disulfide bridge between cysteine residues of the first and second CH3 linker sequences.

In some embodiments, the first CH3 linker and the second CH3 linker are identical. In some embodiments, the first CH3 linker and second CH3 linker are non-identical. In some embodiments, the first CH3 linker and second CH3 linker differ in length by 1-6 amino acids. In some embodiments, the first CH3 linker and second CH3 linker differ in length by 1-3 amino acids. In some embodiments, the first CH3 linker is AGC and the second CH3 linker is AGKGSC. In some embodiments, the first CH3 linker is AGKGC and the second CH3 linker is AGC. In some embodiments, the first CH3 linker is AGKGSC and the second CH3 linker is AGC. In some embodiments, the first CH3 linker is AGKC and the second CH3 linker is AGC.

In some embodiments, the binding molecule further comprises one or more CH1/CL modifications as described in Sections 6.3.1.1. and 6.3.1.2.

In some embodiments, the binding molecule further comprises a modification that reduces effector function as described in Section 6.10.4.

### 6.6. Exemplary Trivalent Binding Molecules

### 6.6.1. Trivalent 1×2 Bispecific B-Body “BC28-1×2”

With reference to Section 6.5.3. and FIG. 26, in a series of embodiments, the binding molecules further comprise a sixth polypeptide chain, wherein (a) the third polypeptide chain further comprises a domain R and a domain S, wherein the domains are arranged, from N-terminus to C-terminus, in a R-S-H-I-J-K orientation, and wherein domain R has the first VL amino acid sequence and domain S has a human IgG1 CH3 amino acid sequence with a Y349C mutation and a C-terminal extension incorporating a PGK tripeptide sequence that is followed by GSGSGS linker peptide connecting domain S to domain H; (b) the binding molecule further comprises a sixth polypeptide chain, comprising: a domain T and a domain U, wherein the domains are arranged, from N-terminus to C-terminus, in a T-U orientation, and wherein domain T has the first VH amino acid sequence and domain U has a human IgG1 CH3 amino acid sequence with a S354C mutation and a C-terminal extension incorporating a PGK tripeptide sequence; (c) the third and the sixth polypeptides are associated through an interaction between the R and the T domains and an interaction between the S and the U domains to form the binding molecule, and (d) domain R and domain T form a third antigen binding site specific for the first antigen.

In preferred embodiments, the first polypeptide chain has the sequence SEQ ID NO:24, the second polypeptide chain has the sequence SEQ ID NO:25, the third polypeptide chain has the sequence SEQ ID NO:37, the fourth polypeptide chain has the sequence SEQ ID NO:11, and the sixth polypeptide chain has the sequence SEQ ID NO:25.

### 6.6.2. Trivalent 1×2 Trispecific B-Body “BC28-1×1×1a”

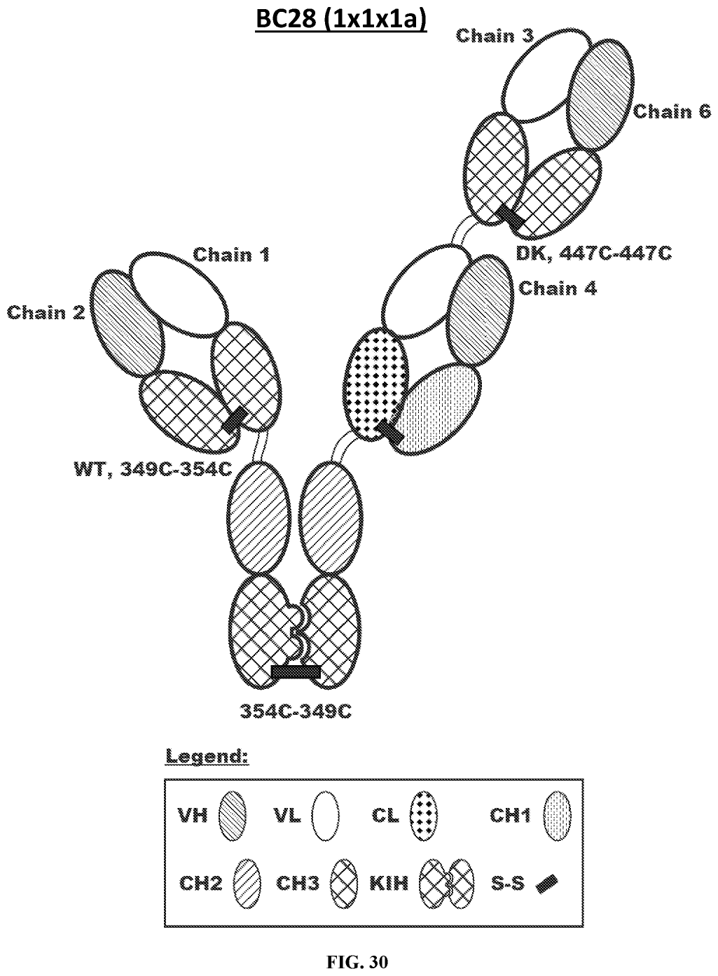

With reference to Section 6.5.3. and FIG. 26 and FIG. 30, in a series of embodiments, the binding molecules further comprise a sixth polypeptide chain, wherein (a) the third polypeptide chain further comprises a domain R and a domain S, wherein the domains are arranged, from N-terminus to C-terminus, in a R-S-H-I-J-K orientation, and wherein domain R has a third VL amino acid sequence and domain S has a human IgG1 CH3 amino acid sequence with a T366K mutation and a C-terminal extension incorporating a KSC tripeptide sequence that is followed by GSGSGS linker peptide connecting domain S to domain H; (b) the binding molecule further comprises a sixth polypeptide chain, comprising: a domain T and a domain U, wherein the domains are arranged, from N-terminus to C-terminus, in a T-U orientation, and wherein domain T has a third VH amino acid sequence and domain U has a human IgG1 CH3 amino acid sequence with a L351D mutation and a C-terminal extension incorporating a GEC amino acid disulfide motif; and (c) the third and the sixth polypeptides are associated through an interaction between the R and the T domains and an interaction between the S and the U domains to form the binding molecule, and (d) domain R and domain T form a third antigen binding site specific for a third antigen.

In preferred embodiments, the first polypeptide chain has the sequence SEQ ID NO:24, the second polypeptide chain has the sequence SEQ ID NO:25, the third polypeptide chain has the sequence SEQ ID NO:45, the fourth polypeptide chain has the sequence SEQ ID NO:11, and the sixth polypeptide chain has the sequence SEQ ID NO: 53.

### 6.6.3. Trivalent Molecules with CH/CL, IgA-CH3 and IgG-CH3

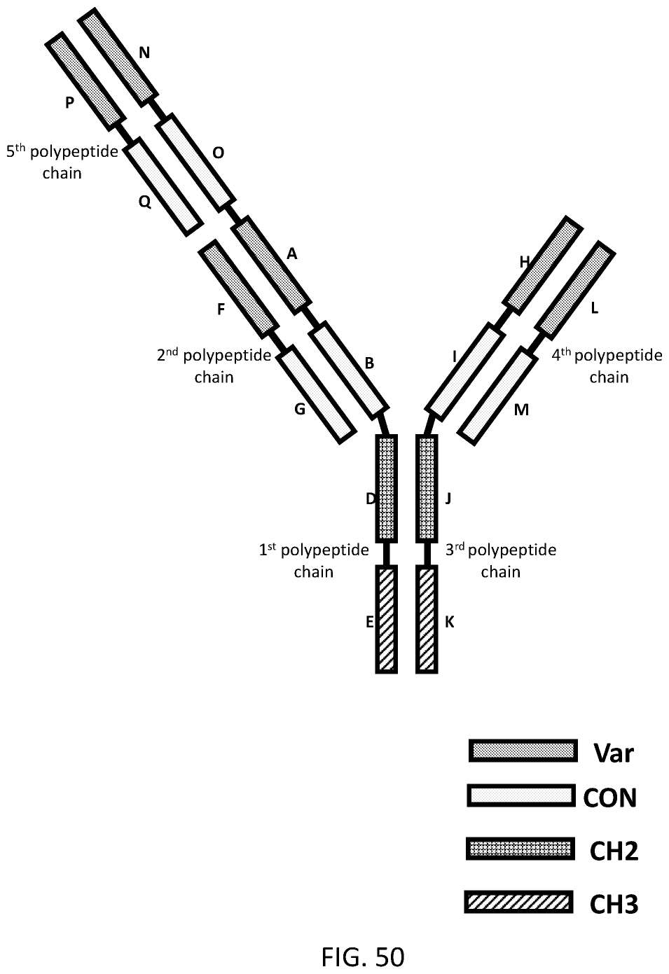

FIG. 50 depicts an exemplary structure of a trivalent binding molecule. With reference to FIG. 50, in various embodiments, the binding molecules comprises a first polypeptide chain, a second polypeptide chain, a third polypeptide chain, a fourth polypeptide chain, and a fifth polypeptide chain, wherein (a) the first polypeptide chain comprises a domain N, a domain O, a domain A, a domain B, a domain D, and a domain E, wherein the domains are arranged, from N-terminus to C-terminus, in a N-O-A-B-D-E orientation, and wherein domain N has a variable region amino acid sequence, domain O has a constant region amino acid sequence; domain A has a variable region amino acid sequence, domain B has a constant region amino acid sequence, domain D has a CH2 sequence, and domain E has a CH3 sequence; (b) the second polypeptide chain comprises a domain F and a domain G, wherein the domains are arranged, from N-terminus to C-terminus, in a F-G orientation, and wherein domain F has a variable region domain amino acid sequence and domain G has a constant region domain amino acid sequence; (c) the third polypeptide chain comprises a domain H, a domain I, a domain J, and a domain K, wherein the domains are arranged, from N-terminus to C-terminus, in a H-I-J-K orientation, and wherein domain H has a variable region domain amino acid sequence, domain I has a constant region amino acid sequence, domain J has a CH2 sequence, and domain K has a CH3 sequence; (d) the fourth polypeptide chain comprises a domain L and a domain M, wherein the domains are arranged, from N-terminus to C-terminus, in a L-M orientation, and wherein domain L has a variable region domain amino acid sequence and domain M comprises a constant region amino acid sequence; (e) the fifth polypeptide chain comprises a domain P and a domain Q, wherein the domains are arranged, from N-terminus to C-terminus, in a P-Q orientation, and wherein domain P comprises a variable region amino acid sequence and domain Q comprises a constant region amino acid sequence; (f) domains B and G form a first domain pair of associated constant region domains (“first domain pair”), domains I and M form a second domain pair of associated constant region domains (“second domain pair”), and domains Q and O form a third domain pair of associated constant region domains (“third domain pair”); (g) at least one of the first, second, and third domain pairs is a CH1/CL pair; (h) at least one of the first, second, and third domain pairs is an IgG-CH3/IgG-CH3 pair; and (i) at least one of the first, second, and third domain pairs is an IgA-CH3/IgA-CH3 pair. In some embodiments, the binding molecule is formed by domain interactions, including but not necessarily exclusive to interactions of the first domain pair, interactions of the second domain pair, interactions of the third domain pair, an association between domains D and J, and an association between domains E and K.

In some embodiments, domains A and F associate to form a first antigen binding site; domains H and L associate to form a second antigen binding site; and domains N and P associate to form a third antigen binding site.

In some embodiments, the first domain pair is an IgA-CH3/IgA-CH3 pair, the second domain pair is an IgG-CH3/IgG-CH3 pair, and the third domain pair is a CH1/CL pair. In some embodiments, the first domain pair is an IgA-CH3/IgA-CH3 pair, the second domain pair is a CH1/CL pair, and the third domain pair is an IgG-CH3/IgG-CH3 pair. In some embodiments, the first domain pair is an IgG-CH3/IgG-CH3 pair, the second domain pair is an IgA-CH3/IgA-CH3 pair, and the third domain pair is a CH1/CL pair. In some embodiments, the first domain pair is an IgG-CH3/IgG-CH3 pair, the second domain pair is a CH1/CL pair, and the third domain pair is an IgA-CH3/IgA-CH3 pair. In some embodiments, the first domain pair is a CH1/CL pair, the second domain pair is an IgA-CH3/IgA-CH3 pair, and the third domain pair is an IgG-CH3/IgG-CH3 pair. In some embodiments, the first domain pair is a CH1/CL pair, the second domain pair is an IgG-CH3/IgG-CH3 pair, and the third domain pair is an IgA-CH3/IgA-CH3 pair.

In some embodiments, an association between domains A and F form a first antigen binding site, an association between domains H and L form a second antigen binding site, and an association between domains N and P form a third antigen binding site. In some embodiments, the first antigen binding site, the second antigen binding site, and the third antigen binding site bind to the same antigen. In some embodiments, the first antigen binding site and second antigen binding site bind to a first antigen, and the third antigen binding site binds to a second antigen. In some embodiments, the first antigen binding site and third antigen binding site bind to a first antigen, and the second antigen binding site binds to a second antigen. In some embodiments, the first antigen binding site binds to a first antigen, and the second antigen binding site and third antigen binding site binds to a second antigen. In some embodiments, the first antigen binding site binds to a first antigen, the second antigen binding site binds to a second antigen, and the third antigen binding site binds to a third antigen.

In some embodiments, domains E and K comprise a knob-in-hole orthogonal modification, as described in Section 6.4.14.2.

In some embodiments, the CH1/CL pair comprises one or more CH1/CL orthogonal modifications as described in Sections 6.3.1.1 and 6.3.1.2.

In some embodiments, the IgG-CH3/IgG-CH3 pair comprises one or more orthogonal modifications described in Sections 6.4.14.1, 6.4.14.2, and 6.4.14.3.

In some embodiments, the Fc region of the binding molecule comprises one or more mutations in CH2 which reduce effector function. Such mutations are described in Section 6.10.4.

### 6.7. Exemplary Binding Molecules with CH1/CL Modifications

In some embodiments, a binding molecule described herein comprises one or more orthogonal CH1/CL modifications described above. In some embodiments, the binding molecule, generally comprising an architecture as described in FIG. 3, 21, 26, 30 or 34, comprises one or more orthogonal modifications in one or more CH1/CL domain-associated pairs. In some cases, the one or more CH1/CL domain-associated pairs comprising the one or more orthogonal CH1/CL modifications have non-identical sets of CH1/CL orthogonal modifications as compared to the other CH1/CL domain-associated pairs. For example, the binding molecule may comprise one or more orthogonal modifications in a CH1/CL pair of one arm of the Y-shaped structure. For another example, a binding molecule having a general Y-shaped architecture as described in FIG. 3, 21, 26, 30 or 34, may comprise one or more orthogonal modifications in CH1/CL pairs in both arms of the Y-shaped structure, wherein each CH1/CL pair comprises non-identical CH1/CL orthogonal modifications as compared to the other CH1/CL pairs. In some embodiments, a binding molecule having a general architecture as described in FIG. 21, 26, 30 or 34, comprises at least a first CH1/CL pair and a second CH1/CL pair in one arm of the Y-shaped structure, wherein the first CH1/CL pair comprise non-identical CH1/CL orthogonal modifications as compared to the second CH1/CL pair.

In some embodiments, the first CH1/CL pair comprises a first charged-pair orthogonal mutation and the second CH1/CL pair comprises a second charged-pair orthogonal mutation, in the same amino acid position, wherein the second charged-pair orthogonal mutation is oppositely charged as compared to the first charged-pair orthogonal mutation. In some embodiments, the first CH1/CL pair comprises a first charged-pair orthogonal mutation that introduces a positively-charged residue in an amino acid position of CH1 and a negatively-charged residue in the orthogonal CL position, and the second CH1/CL pair comprises a second charged-pair orthogonal mutation that introduces a negatively-charged residue in the same amino acid position of CH1 and a positively-charged residue in the orthogonal CL position. In some embodiments, the first CH1/CL pair comprises a first charged-pair orthogonal mutation that introduces a negatively-charged residue in an amino acid position of CH1 and a positively-charged residue in the orthogonal CL position, and the second CH1/CL pair comprises a second charged-pair orthogonal mutation that introduces a positively-charged residue in the same amino acid position of CH1 and a negatively-charged residue in the orthogonal CL position. For example, the first CH1/CL pair may comprise a CH1 domain comprising a G166D mutation and a CL domain comprising a N138K mutation, and the second CH1/CL pair may comprise a CH1 domain comprising a G166K mutation and a CL domain comprising a N138D mutation. For other example, the first CH1/CL pair may comprise a CH1 domain comprising a G166K mutation and a CL domain comprising a N138D mutation, and the second CH1/CL pair may comprise a CH1 domain comprising a G166D mutation and a CL domain comprising a N138K mutation. In some embodiments, the first or second CH1/CL pair may further comprise an engineered disulfide bridge described in Table 6 herein. In some embodiments, the engineered disulfide bridge comprises an orthogonal L128C mutation in CH1 and F118C mutation in CL.

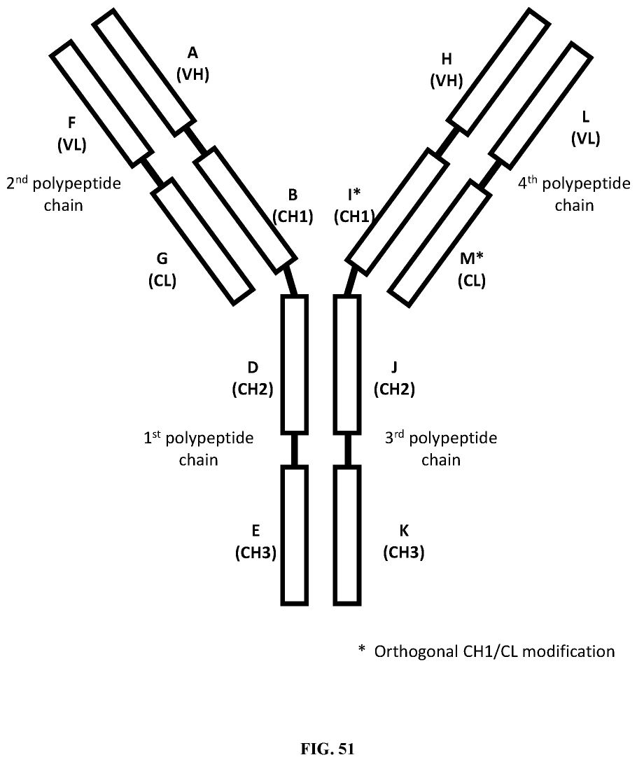

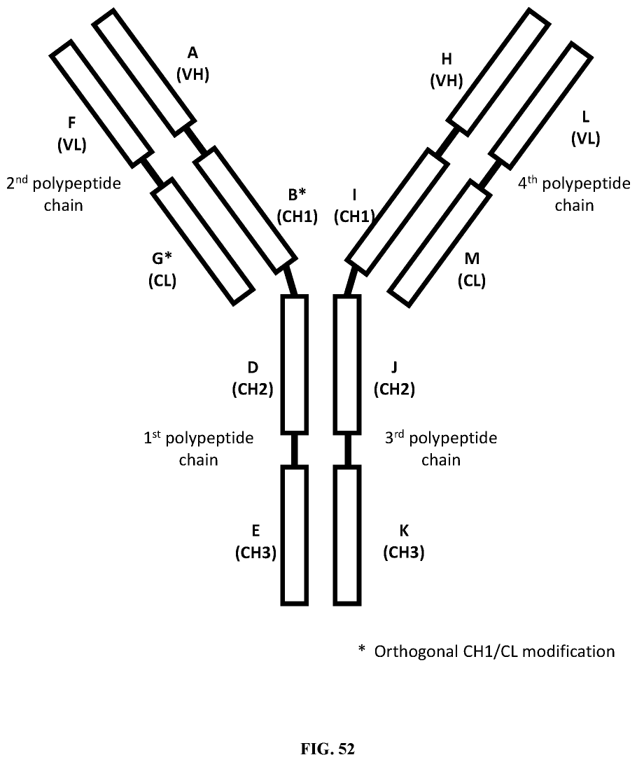

In some embodiments, the binding molecule is structured as described in paragraph [0126], wherein domain B comprises CH1 and domain G comprises CL, thereby forming a first CH1/CL associated domain pair; domain I comprises CH1 and domain M comprises CL, thereby forming a second CH1/CL associated domain pair, and wherein the first and second CH1/CL pairs each comprise non-identical sets of CH1/CL orthogonal modifications. In some embodiments, the first CH1/CL pair comprises an L128C/F118C engineered disulfide bridge and a G166D/N138K orthogonal charged-pair mutation, and the second CH1/CL pair comprises a G166K/N138D orthogonal charged-pair mutation. In some embodiments, the first CH1/CL pair comprises an L128C/F118C engineered disulfide bridge and a G162K/N138D orthogonal charged-pair mutation, and the second CH1/CL pair comprises a G162D/N138K orthogonal charged-pair mutation. In some cases, the binding molecule further comprises a knob-in-hole orthogonal modification described herein. Exemplary binding molecule are depicted in FIGS. 51, 52, and 53.

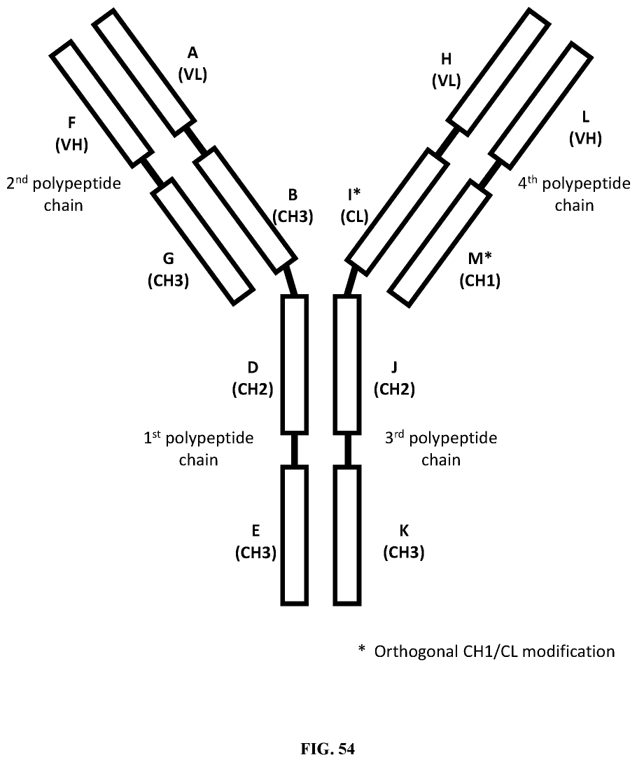

In some embodiments, the binding molecule is a B-Body™. B-Body™ binding molecules are described in International patent Application No. PCT/US2017/057268. In some embodiments, the binding molecule is structured as described in [0126], wherein A is VL, B is CH3, D is CH2, E is CH3, F is VH, G is CH3, H is VL, I is CL, J is CH2, K is CH3, L is VH, and M is CH1, and wherein domain pair I and M comprise one or more CH1/CL orthogonal modification as described in Tables 6 and 7. An exemplary binding molecule is depicted in FIG. 54.

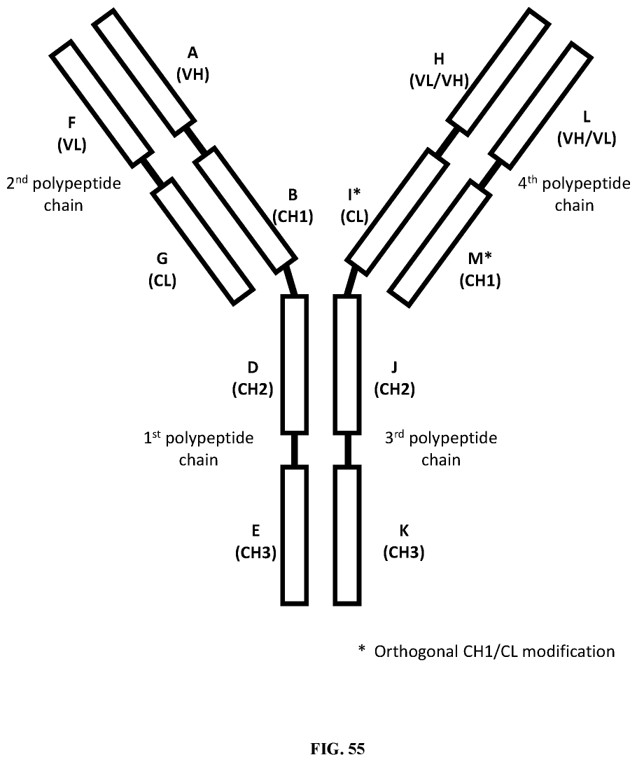

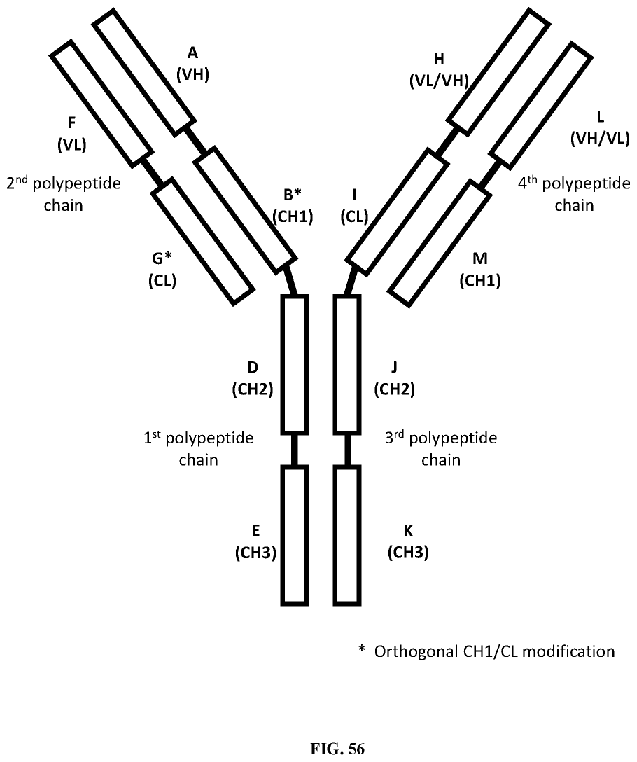

In some embodiments, the binding molecule is a CrossMab™ antibody comprising one or more CH1/CL orthogonal modifications described in Tables 6 and 7. CrossMab™ antibodies are described in U.S. Pat. Nos. 8,242,247; 9,266,967; and 8,227,577, U.S. Patent Application Pub. No. 20120237506, U.S. Patent Application Pub. No. US20090162359, WO2016016299, WO2015052230. In some embodiments, the binding molecule is a bivalent, bispecific antibody, comprising: a) the light chain and heavy chain of an antibody specifically binding to a first antigen; and b) the light chain and heavy chain of an antibody specifically binding to a second antigen, wherein constant domains CL and CH1 from the antibody specifically binding to a second antigen are replaced by each other; wherein constant domains CL and CH1 of) the light chain and heavy chain of an antibody specifically binding to the first or second antigen comprises one or more CH1/CL orthogonal modifications described in Tables 6 and 7. In some embodiments, the binding molecule is structured as described in paragraph [0126], wherein A is VH, B is CH1, D is CH2, E is CH3, F is VL, G is CL, H is VL or VH, I is CL, J is CH2, K is CH3, L is VH or VL, and M is CH1, and wherein at least one of domain pairs B and G, and I and M, comprise one or more CH1/CL orthogonal modification as described in Tables 6 and 7. In some cases, domain pair B and G comprise one or more CH1/CL orthogonal modifications and domain pair I and M does not. In other cases, domain pair I and M comprise one or more CH1/CL orthogonal modifications and domain pair B and G does not. In yet other cases, both domain pair B and G and domain pair I and M comprise non-identical sets of one or more CH1/CL orthogonal modifications. Exemplary binding molecules are depicted in FIGS. 55 and 56.

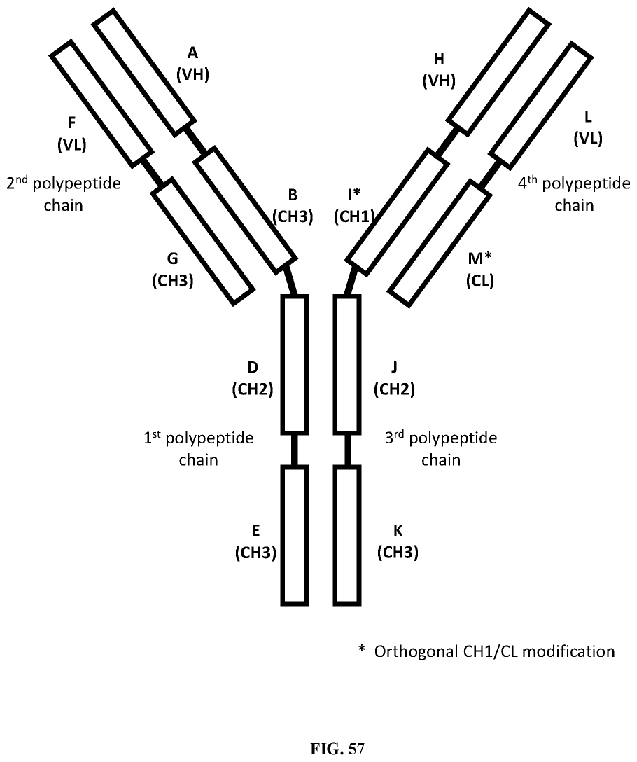

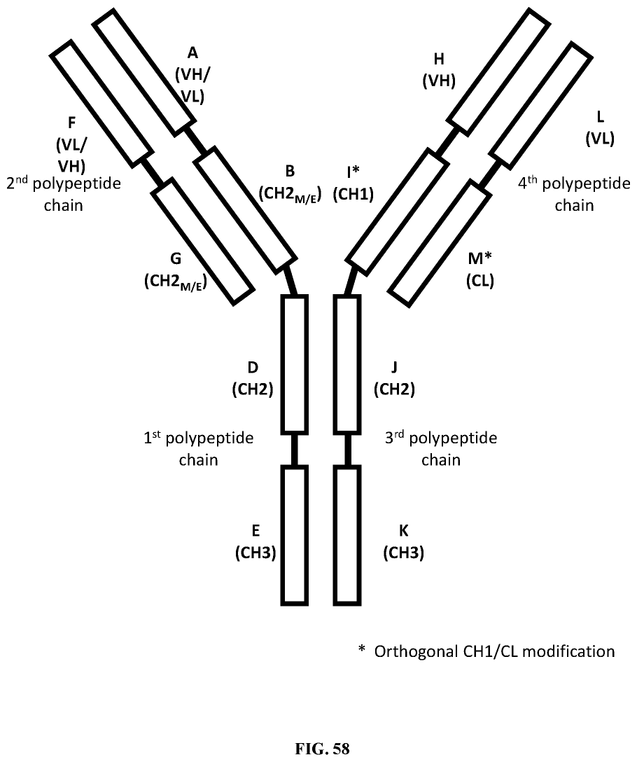

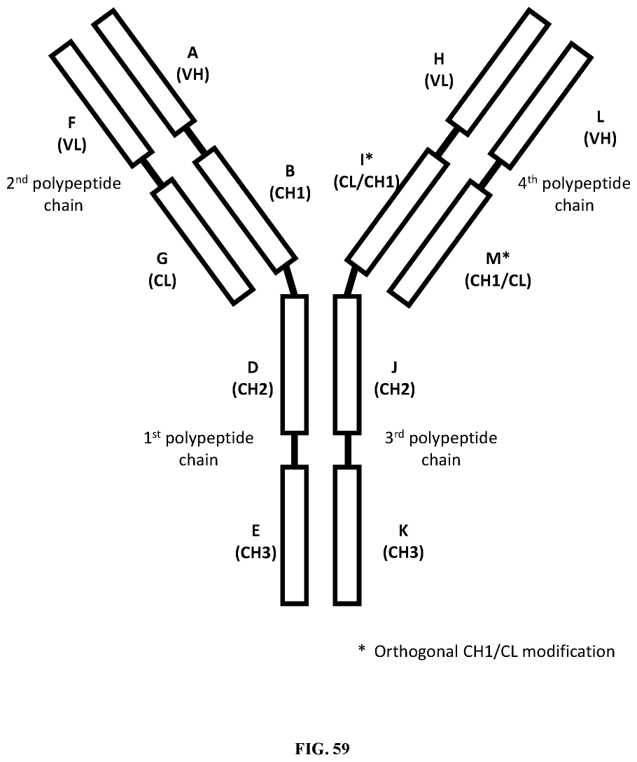

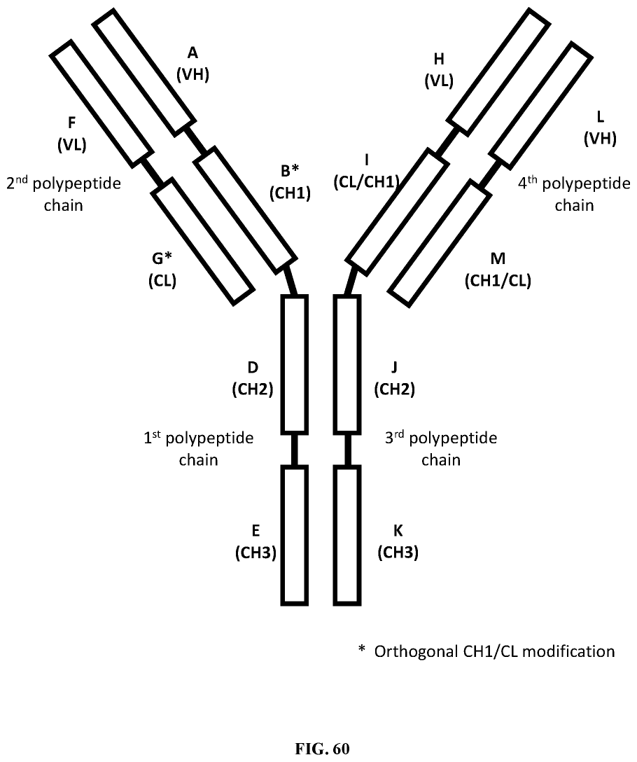

In some embodiments, the binding molecule is an antibody having a general architecture described in U.S. Pat. No. 8,871,912 and WO2016087650. In some embodiments, the binding molecule is a domain-exchanged antibody comprising a light chain (LC) composed of VL-CH3, and a heavy chain (HC) comprising VH-CH3-CH2-CH3, wherein the VL-CH3 of the LC dimerizes with the VH-CH3 of the HC thereby forming a domain-exchanged LC/HC dimer comprising a CH3LC/CH3HC domain pair, wherein the antibody further comprises an additional light chain composed of VL-CL and an additional heavy chain composed of VH-CH1-CH2-CH3, and wherein the CH1 and CL comprise one or more CH1/CL orthogonal modifications described in Tables 6 and 7. In some embodiments, the binding molecule is structured as described in paragraph [0126], wherein A is VH, B is CH3, D is CH2, E is CH3, F is VL, G is CH3, H is VH, I is CH1, J is CH2, K is CH3, L is VL, and M is CL, and wherein domain pair I and M comprise one or more CH1/CL orthogonal modifications as described in Tables 6 and 7. An exemplary binding molecule is depicted in FIG. 57.

In some embodiments, the binding molecule is as described in WO2017011342. In some embodiments, the binding molecule is structured as described in paragraph [0126], wherein A is VH or VL, B is CH2 from IgM or IgE, D is CH2, E is CH3, F is VL or VH, G is CH2 from IgM or IgE, H is VH, I is CH1, J is CH2, K is CH3, L is VL, and M is CL, and wherein domain pair I and M comprise one or more CH1/CL orthogonal modification as described in Tables 6 and 7. An exemplary binding molecule is depicted in FIG. 58.

In some embodiments, the binding molecule is as described in WO2006093794. In some embodiments, the binding molecule is structured as described in paragraph [0126], wherein A is VH, B is CH1, D is CH2, E is CH3, F is VL, G is CL, H is VL, I is CL or CH1, J is CH2, K is CH3, L is VH, and M is CH1 or CL, and wherein at least one of domain pairs B and G, and I and M, comprise one or more CH1/CL orthogonal modification as described in Tables 6 and 7. In some cases, domain pair B and G comprise one or more CH1/CL orthogonal modifications and domain pair I and M does not. In other cases, domain pair I and M comprise one or more CH1/CL orthogonal modifications and domain pair B and G does not. In yet other cases, both domain pair B and G and domain pair I and M comprise non-identical sets of one or more CH1/CL orthogonal modifications. Exemplary binding molecules are depicted in FIGS. 59 and 60.

It is contemplated that binding molecules comprising one or more CH1/CL modifications described herein may further comprise modifications of one or more other domains. For example, any of the binding molecules comprising one or more CH1/Cl modifications, described herein may further comprise knob-in-hole mutations, described in Section 6.4.14.2, mutations that reduce effector function, as described in Section 6.10.4, and/or IgA-CH3 domain paring as described in Section 6.4.14.4.

### 6.8. Other Binding Molecule Platforms

The various antibody platforms described above are not limiting. The antigen binding sites described herein, including specific CDR subsets, can be formatted into any binding molecule platform including, but not limited to, full-length antibodies, Fab fragments, Fvs, scFvs, tandem scFvs, Diabodies, scDiabodies, DARTs, tandAbs, minibodies, camelid VHH, and other antibody fragments or formats known to those skilled in the art. Exemplary antibody and antibody fragment formats are described in detail in Brinkmann et al. (MABS, 2017, Vol. 9, No. 2, 182-212), herein incorporated by reference for all that it teaches. Furthermore, any of the modifications and mutations described herein, can be formatted into any binding molecule platform described herein.

### 6.9. Antigen Specificities

Antigen binding sites potentially relevant to the binding molecules described herein may be chosen to specifically bind a wide variety of molecular targets. For example, an antigen binding site or sites may specifically bind E-Cad, CLDN7, FGFR2b, N-Cad, Cad-11, FGFR2c, ERBB2, ERBB3, FGFR1, FOLR1, IGF-Ira, GLP1R, PDGFRa, PDGFRb, EPHB6, ABCG2, CXCR4, CXCR7, Integrin-avb3, SPARC, VCAM, ICAM, Annexin, TNFα, CD137, angiopoietin 2, angiopoietin 3, BAFF, beta amyloid, C5, CA-125, CD147, CD125, CD147, CD152, CD19, CD20, CD22, CD23, CD24, CD25, CD274, CD28, CD3, CD30, CD33, CD37, CD4, CD40, CD44, CD44v4, CD44v6, CD44v7, CD50, CD51, CD52, CEA, CSF1R, CTLA-2, DLL4, EGFR, EPCAM, HER3, GD2 ganglioside, GDF-8, Her2/neu, CD2221, IL-17A, IL-12, IL-23, IL-13, IL-6, IL-23, an integrin, CD11a, MUC1, Notch, TAG-72, TGFβ, TRAIL-R2, VEGF-A, VEGFR-1, VEGFR2, VEGFc, hematopoietins (four-helix bundles) (such as EPO (erythropoietin), IL-2 (T-cell growth factor), IL-3 (multicolony CSF), IL-4 (BCGF-1, BSF-1), IL-5 (BCGF-2), IL-6 IL-4 (IFN-β2, BSF-2, BCDF), IL-7, IL-8, IL-9, IL-11, IL-13 (P600), G-CSF, IL-15 (T-cell growth factor), GM-CSF (granulocyte macrophage colony stimulating factor), OSM (OM, oncostatin M), and LIF (leukemia inhibitory factor)); interferons (such as IFN-γ, IFN-α, and IFN-β); immunoglobin superfamily (such as B7.1 (CD80), and B7.2 (B70, CD86)); TNF family (such as TNF-α (cachectin), TNF-β (lymphotoxin, LT, LT-α), LT-β, Fas, CD27, CD30, and 4-1BBL); and those unassigned to a particular family (such as TGF-β, IL 1α, IL-1β, IL-1 RA, IL-10 (cytokine synthesis inhibitor F), IL-12 (NK cell stimulatory factor), MIF, IL-16, IL-17 (mCTLA-8), and/or IL-18 (IGIF, interferon-γ inducing factor)); in embodiments relating to bispecific antibodies, the antibody may for example bind two of these targets. Furthermore, the Fc portion of the heavy chain of an antibody may be used to target Fc receptor-expressing cells such as the use of the Fc portion of an IgE antibody to target mast cells and basophils. An antigen binding site or sites may be chosen that specifically binds the TNF family of receptors including, but not limited to, TNFR1 (also known as CD120a and TNFRSF1A), TNFR2 (also known as CD120b and TNFRSF1B), TNFRSF3 (also known as LTβR), TNFRSF4 (also known as OX40 and CD134), TNFRSF5 (also known as CD40), TNFRSF6 (also known as FAS and CD95), TNFRSF6B (also known as DCR3), TNFRSF7 (also known as CD27), TNFRSF8 (also known as CD30), TNFRSF9 (also known as 4-1BB), TNFRSF10A (also known as TRAILR1, DR4, and CD26), TNFRSF10B (also known as TRAILR2, DR5, and CD262), TNFRSF10C (also known as TRAILR3, DCR1, CD263), TNFRSF10D (also known as TRAILR4, DCR2, and CD264), TNFRSF11A (also known as RANK and CD265), TNFRSF11B (also known as OPG), TNFRSF12A (also known as FN14, TWEAKR, and CD266), TNFRSF13B (also known as TACI and CD267), TNFRSF13C (also known as BAFFR, BR3, and CD268), TNFRSF14 (also known as HVEM and CD270), TNFRSF16 (also known as NGFR, p75NTR, and CD271), or TNFRSF17 (also known as BCMA and CD269), TNFRSF18 (also known as GITR and CD357), TNFRSF19 (also known as TROY, TAJ, and TRADE), TNFRSF21 (also known as CD358), TNFRSF25 (also known as Apo-3, TRAMP, LARD, or WS-1), EDA2R (also known as XEDAR).

An antigen binding site or sites may be chosen that specifically binds immune-oncology targets including, but not limited to, checkpoint inhibitor targets such as PD1, PDL1, CTLA-4, PDL2, B7-H3, B7-H4, BTLA, TIM3, GAL9, LAG3, VISTA, KIR, 2B4, BY55, and CGEN-15049.

In particular embodiments, the trivalent trispecific binding molecule has antigen binding sites that specifically bind two tumor associated antigens and a T cell surface expressed molecule. In a specific embodiment, the trivalent trispecific binding molecule has antigen binding sites that specifically bind two tumor associated antigens and the T cell surface expressed protein CD3. Without wishing to be bound by theory, the trivalent trispecific binding molecule that specifically binds tumor-associated antigen and the T cell surface expressed molecule (i.e., CD3) can direct T cell mediated killing (cytotoxicity) of cells expressing the two tumor associated antigens through redirecting T cells to the tumor associated antigens expressing cells (i.e., target cells). T cell mediated killing using bispecific anti-CD3 molecules is described in detail in U.S. Pub. No. 2006/0193852, herein incorporated by reference in its entirety. In some embodiments, the T cell surface expressed molecule is selected from any molecule capable of redirecting T cells to a target cell. In some embodiments, the one or more affinities of individual ABSs for the two tumor associated antigens do not have has a K_{D} value that qualifies as specifically binding their respective antigens or epitopes on their own, but the avidity of the trivalent trispecific binding molecule for a specific target cell expressing the two tumor associated antigens has a K_{D} value such that the interaction is a specific binding interaction.

In a series of embodiments, an antigen binding site or sites may be chosen that specifically target tumor-associated cells. In various embodiments, the antigen binding site or sites specifically target tumor associated immune cells. In certain embodiments, the antigen binding site or sites specifically target tumor associated regulatory T cells (Tregs). In specific embodiments, a binding molecule has antigen binding sites specific for antigens selected from one or more of CD25, OX40, CTLA-4, and NRP1 such that the binding molecule specifically targets tumor associated regulatory T cells. In specific embodiments, a binding molecule has antigen binding sites that specifically bind CD25 and OX40, CD25 and CTLA-4, CD25 and NRP1, OX40 and CTLA-4, OX40 and NRP1, or CTLA-4 and NRP1 such that the binding molecule specifically targets tumor associated regulatory T cells. In preferred embodiments, a bispecific bivalent binding molecule has antigen binding sites that specifically bind CD25 and OX40, CD25 and CTLA-4, CD25 and NRP1, OX40 and CTLA-4, OX40 and NRP1, or CTLA-4 and NRP1 such that the binding molecule specifically targets tumor associated regulatory T cells. In specific embodiments, the specific targeting of the tumor associated regulatory T cells results in depletion (e.g. killing) of the regulatory T cells. In preferred embodiments, the depletion of the regulatory T cells is mediated by an antibody-drug conjugate (ADC) modification, such as an antibody conjugated to a toxin, as discussed in more detail in Section 6.10.1.

### 6.10. Further Modifications

In a further series of embodiments, the binding molecule has additional modifications.

### 6.10.1. Antibody-Drug Conjugates

In various embodiments, the binding molecule is conjugated to a therapeutic agent (i.e. drug) to form a binding molecule-drug conjugate. Therapeutic agents include, but are not limited to, chemotherapeutic agents, imaging agents (e.g. radioisotopes), immune modulators (e.g. cytokines, chemokines, or checkpoint inhibitors), and toxins (e.g. cytotoxic agents). In certain embodiments, the therapeutic agents are attached to the binding molecule through a linker peptide, as discussed in more detail in Section 6.10.3.

Methods of preparing antibody-drug conjugates (ADCs) that can be adapted to conjugate drugs to the binding molecules disclosed herein are described, e.g., in U.S. Pat. No. 8,624,003 (pot method), U.S. Pat. No. 8,163,888 (one-step), U.S. Pat. No. 5,208,020 (two-step method), U.S. Pat. Nos. 8,337,856, 5,773,001, 7,829,531, 5,208,020, 7,745,394, WO 2017/136623, WO 2017/015502, WO 2017/015496, WO 2017/015495, WO 2004/010957, WO 2005/077090, WO 2005/082023, WO 2006/065533, WO 2007/030642, WO 2007/103288, WO 2013/173337, WO 2015/057699, WO 2015/095755, WO 2015/123679, WO 2015/157286, WO 2017/165851, WO 2009/073445, WO 2010/068759, WO 2010/138719, WO 2012/171020, WO 2014/008375, WO 2014/093394, WO 2014/093640, WO 2014/160360, WO 2015/054659, WO 2015/195925, WO 2017/160754, Storz (MAbs. 2015 November-December; 7(6): 989-1009), Lambert et al. (Adv Ther, 2017 34: 1015), Diamantis et al. (British Journal of Cancer, 2016, 114, 362-367), Carrico et al. (Nat Chem Biol, 2007. 3: 321-2), We et al. (Proc Natl Acad Sci USA, 2009. 106: 3000-5), Rabuka et al. (Curr Opin Chem Biol., 2011 14: 790-6), Hudak et al. (Angew Chem Int Ed Engl., 2012: 4161-5), Rabuka et al. (Nat Protoc., 2012 7:1052-67), Agarwal et al. (Proc Natl Acad Sci USA., 2013, 110: 46-51), Agarwal et al. (Bioconjugate Chem., 2013, 24: 846-851), Barfield et al. (Drug Dev. and D., 2014, 14:34-41), Drake et al. (Bioconjugate Chem., 2014, 25:1331-41), Liang et al. (J Am Chem Soc., 2014, 136:10850-3), Drake et al. (Curr Opin Chem Biol., 2015, 28:174-80), and York et al. (BMC Biotechnology, 2016, 16(1):23), each of which is hereby incorporated by reference in its entirety for all that it teaches.

### 6.10.2. Additional Binding Moieties

In various embodiments, the binding molecule has modifications that comprise one or more additional binding moieties. In certain embodiments the binding moieties are antibody fragments or antibody formats including, but not limited to, full-length antibodies, Fab fragments, Fvs, scFvs, tandem scFvs, Diabodies, scDiabodies, DARTs, tandAbs, minibodies, camelid VHH, and other antibody fragments or formats known to those skilled in the art. Exemplary antibody and antibody fragment formats are described in detail in Brinkmann et al. (MABS, 2017, Vol. 9, No. 2, 182-212), herein incorporated by reference for all that it teaches.

In particular embodiments, the one or more additional binding moieties are attached to the C-terminus of the first or third polypeptide chain. In particular embodiments, the one or more additional binding moieties are attached to the C-terminus of both the first and third polypeptide chain. In particular embodiments, the one or more additional binding moieties are attached to the C-terminus of both the first and third polypeptide chains. In certain embodiments, individual portions of the one or more additional binding moieties are separately attached to the C-terminus of the first and third polypeptide chains such that the portions form the functional binding moiety.

In particular embodiments, the one or more additional binding moieties are attached to the N-terminus of any of the polypeptide chains (e.g. the first, second, third, fourth, fifth, or sixth polypeptide chains). In certain embodiments, individual portions of the additional binding moieties are separately attached to the N-terminus of different polypeptide chains such that the portions form the functional binding moiety.

In certain embodiments, the one or more additional binding moieties are specific for a different antigen or epitope of the ABSs within the binding molecule. In certain embodiments, the one or more additional binding moieties are specific for the same antigen or epitope of the ABSs within the binding molecule. In certain embodiments, wherein the modification is two or more additional binding moieties, the additional binding moieties are specific for the same antigen or epitope. In certain embodiments, wherein the modification is two or more additional binding moieties, the additional binding moieties are specific for different antigens or epitopes.

In certain embodiments, the one or more additional binding moieties are attached to the binding molecule using in vitro methods including, but not limited to, reactive chemistry and affinity tagging systems, as discussed in more detail in Section 6.10.3. In certain embodiments, the one or more additional binding moieties are attached to the binding molecule through Fc-mediated binding (e.g. Protein A/G). In certain embodiments, the one or more additional binding moieties are attached to the binding molecule using recombinant DNA techniques, such as encoding the nucleotide sequence of the fusion product between the binding molecule and the additional binding moieties on the same expression vector (e.g. plasmid).

### 6.10.3. Functional/Reactive Groups

In various embodiments, the binding molecule has modifications that comprise functional groups or chemically reactive groups that can be used in downstream processes, such as linking to additional moieties (e.g. drug conjugates and additional binding moieties, as discussed in more detail in Sections 6.10.1. and 6.10.2.) and downstream purification processes.

In certain embodiments, the modifications are chemically reactive groups including, but not limited to, reactive thiols (e.g. maleimide based reactive groups), reactive amines (e.g. N-hydroxysuccinimide based reactive groups), “click chemistry” groups (e.g. reactive alkyne groups), and aldehydes bearing formylglycine (FGly). In certain embodiments, the modifications are functional groups including, but not limited to, affinity peptide sequences (e.g. HA, HIS, FLAG, GST, MBP, and Strep systems etc.). In certain embodiments, the functional groups or chemically reactive groups have a cleavable peptide sequence. In particular embodiments, the cleavable peptide is cleaved by means including, but not limited to, photocleavage, chemical cleavage, protease cleavage, reducing conditions, and pH conditions. In particular embodiments, protease cleavage is carried out by intracellular proteases. In particular embodiments, protease cleavage is carried out by extracellular or membrane associated proteases. ADC therapies adopting protease cleavage are described in more detail in Choi et al. (Theranostics, 2012; 2(2): 156-178.), the entirety of which is hereby incorporated by reference for all it teaches.

### 6.10.4. Reduced Effector Function

In certain embodiments, the binding molecule has one or more engineered mutations in an amino acid sequence of an antibody domain that reduce the effector functions generally associated with antibody binding. Effector functions include, but are not limited to, cellular functions that result from an Fc receptor binding to an Fc portion of an antibody, such as antibody dependent cellular cytotoxicity (ADCC), complement fixation (e.g. C1q binding), antibody dependent cellular-mediated phagocytosis (ADCP), opsonization. Engineered mutations that reduce the effector functions are described in more detail in U.S. Pub. No. 2017/0137530, Armour, et al. (Eur. J. Immunol. 29(8) (1999) 2613-2624), Shields, et al. (J. Biol. Chem. 276(9) (2001) 6591-6604), and Oganesyan, et al. (Acta Cristallographica D64 (2008) 700-704), each herein incorporated by reference in their entirety.

In specific embodiments, the binding molecule has one or more engineered mutations in an amino acid sequence of an antibody domain that reduce binding of an Fc portion of the binding molecule by FcR receptors. In some embodiments, the FcR receptors are FcRγ receptors. In some embodiments, the FcR receptors are FcRγR1 receptors. In some embodiments, the FcR receptors are FcγRIIa receptors. In some embodiments, the FcR receptors are FcγRIIIA receptors.

In specific embodiments, the one or more engineered mutations that reduce effector function are mutations in a CH2 domain of an antibody. In various embodiments, the one or more engineered mutations comprise a mutation at position L234 of the CH2 domain. In some embodiments, the mutation at position L234 is L234A. In some embodiments, the mutation at position L234 is L234G. In various embodiments, the one or more engineered mutations comprise a mutation at position L235 of the CH2 domain. In some embodiments, the mutation at position L235 is L235A. In some embodiments, the mutation at position L235 is L235G. In various embodiments, the one or more engineered mutations comprise mutations at positions L234 and L235 of the CH2 domain. In some embodiments, the mutations at positions L234 and L235 of the CH2 domain are L234A and L235A. In some embodiments, the mutations at positions L234 and L235 of the CH2 domain are L234G and L235G.

In various embodiments, the one or more engineered mutations comprise a mutation at position P329 of the CH2 domain. In some embodiments, the mutation at position P329 of the CH2 domain is P329A. In some embodiments, the mutation at position P329 of the CH2 domain is P329G. In some embodiments, the mutation at position P329 of the CH2 domain is P329K.

In other embodiments, the one or more engineered mutations are at positions L234, L235, and P329 of the CH2 domain. In particular embodiments, the one or more engineered mutations are L234A, L235A, and P329A of the CH2 domain. In particular embodiments, the one or more engineered mutations are L234A, L235A, and P329G of the CH2 domain. In preferred embodiments, the one or more engineered mutations are L234A, L235A, and P329K of the CH2 domain. In particular embodiments, the one or more engineered mutations are L234G, L235G, and P329A of the CH2 domain. In particular embodiments, the one or more engineered mutations are L234G, L235G, and P329G of the CH2 domain. In particular embodiments, the one or more engineered mutations are L234G, L235G, and P329K of the CH2 domain.

### 6.10.4.1. Exemplary Binding Molecules with Reduced Effector Function

In some embodiments, the binding molecule is structured as described in [0126], wherein domains A and H are VH, domains B and I are CH1, domains D and J are CH2, domains E and K are CH3, domains F and L are VL, and domains G and M are CL, and wherein at least one CH2 sequence of the binding molecule comprises one or more mutations that reduce effector function as described in Section 6.10.4. In some cases, the one or more CH2 mutations comprise L234A, L235A, and P329K. In some cases, the one or more CH2 mutations comprise L234A, L235A, and P329A. In some cases, the one or more CH2 mutations comprise L234A, L235A, and P329G. In some cases, the one or more CH2 mutations comprise L234G, L235G, and P329A. In some cases, the one or more CH2 mutations comprise L234G, L235G, and P329G. In some cases, the one or more CH2 mutations comprise L234G, L235G, and P329K. In some cases, the binding molecule further comprises one or more CH1/CL orthogonal modifications as described herein. In some cases, the binding molecule further comprises a knob-in-hole orthogonal modification described herein.

In some embodiments, the binding molecule is a B-Body™. B-Body™ binding molecules are described in International patent Application No. PCT/US2017/057268. In some embodiments, the binding molecule is structured as described in Paragraph [0126], wherein A is VL, B is CH3, D is CH2, E is CH3, F is VH, G is CH3, H is VL, I is CL, J is CH2, K is CH3, L is VH, and M is CH1, and wherein at least one CH2 sequence of the binding molecule comprises one or more mutations that reduce effector function as described in Section 6.10.4. In some embodiments, the binding molecule is a trivalent binding molecule as described in Sections 6.4.17, 6.6.1, and 6.6.2, wherein at least one CH2 sequence of the binding molecule comprises one or more mutations that reduce effector function as described in Section 6.10.4.

In some embodiments, the binding molecule is a CrossMab™ antibody comprising one or more CH1/CL orthogonal modifications described in Tables 6 and 7. CrossMab™ antibodies are described in U.S. Pat. Nos. 8,242,247; 9,266,967; and 8,227,577, U.S. Patent Application Pub. No. 20120237506, U.S. Patent Application Pub. No. US20090162359, WO2016016299, WO2015052230. In some embodiments, the binding molecule is a bivalent, bispecific antibody, comprising: a) the light chain and heavy chain of an antibody specifically binding to a first antigen; and b) the light chain and heavy chain of an antibody specifically binding to a second antigen, wherein constant domains CL and CH1 from the antibody specifically binding to a second antigen are replaced by each other; and wherein the binding molecule comprises one or more mutations that reduce effector function as described in Section 6.10.4. In some embodiments, the binding molecule is structured as described in Paragraph [0126], wherein A is VH, B is CH1, D is CH2, E is CH3, F is VL, G is CL, H is VL or VH, I is CL, J is CH2, K is CH3, L is VH or VL, and M is CH1, and wherein the binding molecule comprises one or more mutations that reduce effector function as described in Section 6.10.4.

In some embodiments, the binding molecule is an antibody having a general architecture described in U.S. Pat. No. 8,871,912 and WO2016087650. In some embodiments, the binding molecule is a domain-exchanged antibody comprising a light chain (LC) composed of VL-CH3, and a heavy chain (HC) comprising VH-CH3-CH2-CH3, wherein the VL-CH3 of the LC dimerizes with the VH-CH3 of the HC thereby forming a domain-exchanged LC/HC dimer comprising a CH3LC/CH3HC domain pair, wherein the antibody further comprises an additional light chain composed of VL-CL and an additional heavy chain composed of VH-CH1-CH2-CH3, and wherein the binding molecule comprises one or more mutations that reduce effector function as described in Section 6.10.4. In some embodiments, the binding molecule is structured as described in Paragraph [0126], wherein A is VH, B is CH3, D is CH2, E is CH3, F is VL, G is CH3, H is VH, I is CH1, J is CH2, K is CH3, L is VL, and M is CL, and wherein the binding molecule comprises one or more mutations that reduce effector function as described in Section 6.10.4.

In some embodiments, the binding molecule is as described in WO2017011342. In some embodiments, the binding molecule is structured as described in Paragraph [0126], wherein A is VH or VL, B is CH2 from IgM or IgE, D is CH2, E is CH3, F is VL or VH, G is CH2 from IgM or IgE, H is VH, I is CH1, J is CH2, K is CH3, L is VL, and M is CL, and wherein the binding molecule comprises one or more mutations that reduce effector function as described in Section 6.10.4.

In some embodiments, the binding molecule is as described in WO2006093794. In some embodiments, the binding molecule is structured as described in Paragraph [0126], wherein A is VH, B is CH1, D is CH2, E is CH3, F is VL, G is CL, H is VL, I is CL or CH1, J is CH2, K is CH3, L is VH, and M is CH1 or CL and wherein the binding molecule comprises one or more mutations that reduce effector function as described in Section 6.10.4.

It is contemplated that binding molecules comprising one or more mutations that reduce effector function, described herein may further comprise modifications of one or more other domains. For example, any of the binding molecules in this section may further comprise knob-in-hole mutations, described in Section 6.4.14.2 and/or CH1/CL orthogonal modifications as described in Sections 6.3.1.1 and 6.3.1.2.

It is to be understood that any of the modifications described in this application are not limited to the exemplary embodiments listed above, but are instead applicable to any binding molecule platform, including but not limited to the binding molecule platforms described in Section 6.8. In addition, it is contemplated that binding molecules may include any combination of the modifications described herein.

### 6.11. Methods of Purification

A method of purifying a binding molecule comprising a B-body platform is provided herein.

In a series of embodiments, the method comprises the steps of: i) contacting a sample comprising the binding molecule with a CH1 binding reagent, wherein the binding molecule comprises at least a first, a second, a third, and a fourth polypeptide chain associated in a complex, wherein the complex comprises at least one CH1 domain, or portion thereof, and wherein the number of CH1 domains in the complex is at least one fewer than the valency of the complex, and wherein the contacting is performed under conditions sufficient for the CH1 binding reagent to bind the CH1 domain, or portion thereof; and ii) purifying the complex from one or more incomplete complexes, wherein the incomplete complexes do not comprise the first, the second, the third, and the fourth polypeptide chain.

In a typical, naturally occurring, antibody, two heavy chains are associated, each of which has a CH1 domain as the second domain, numbering from N-terminus to C-terminus. Thus, a typical antibody has two CH1 domains. CH1 domains are described in more detail in Section 6.3. In a variety of the binding molecules described herein, the CH1 domain typically found in the protein has been substituted with another domain, such that the number of CH1 domains in the protein is effectively reduced. In a non-limiting illustrative example, the CH1 domain of a typical antibody can be substituted with a CH3 domain, generating an antigen-binding protein having only a single CH1 domain.

Binding molecules can also refer to molecules based on antibody architectures that have been engineered such that they no longer possess a typical antibody architecture. For example, an antibody can be extended at its N or C terminus to increase the valency (described in more detail in Section 6.4.13.1) of the antigen-binding protein, and in certain instances the number of CH1 domains is also increased beyond the typical two CH1 domains. Such molecules can also have one or more of their CH1 domains substituted, such that the number of CH1 domains in the protein is at least one less than the valency of the antigen-binding protein. In some embodiments, the number of CH1 domains that are substituted by other domains generates a binding molecule having only a single CH1 domain. In other embodiments, the number of CH1 domains substituted by another domain generates a binding molecule having two or more CH1 domains, but at least one fewer than the valency of the antigen-binding protein. In particular embodiments, where a binding molecule has two or more CH1 domains, the multiple CH1 domains can all be in the same polypeptide chain. In other particular embodiments, where a binding molecule has two or more CH1 domains, the multiple CH1 domains can be a single CH1 domain in multiple copies of the same polypeptide chain present in the complete complex.

### 6.11.1. C111 Binding Reagents

In exemplary non-limiting methods of purifying binding molecules, a sample comprising the binding molecules is contacted with CH1 binding reagents. CH1 binding reagents, as described herein, can be any molecule that specifically binds a CH1 epitope. The various CH1 sequences that provide the CH1 epitope are described in more detail in Section 6.3, and specific binding is described in more detail in Section 6.4.13.1.

In some embodiments, CH1 binding reagents are derived from immunoglobulin proteins and have an antigen binding site (ABS) that specifically binds the CH1 epitope. In particular embodiments, the CH1 binding reagent is an antibody, also referred to as an “anti-CH1 antibody.” The anti-CH1 antibody can be derived from a variety of species. In particular embodiments, the anti-CH1 antibody is a mammalian antibody, including, but not limited to mouse, rat, hamster, rabbit, camel, donkey, goat, and human antibodies. In specific embodiments, the anti-CH1 antibody is a single-domain antibody. Single-domain antibodies, as described herein, have a single variable domain that forms the ABS and specifically binds the CH1 epitope. Exemplary single-domain antibodies include, but are not limited to, heavy chain antibodies derived from camels and sharks, as described in more detail in international application WO 2009/011572, herein incorporated by reference for all it teaches. In a preferred embodiment, the anti-CH1 antibody is a camel derived antibody (also referred to as a “camelid antibody”). Exemplary camelid antibodies include, but are not limited to, human IgG-CH1 CaptureSelect™ (ThermoFisher, #194320010) and human IgA-CH1 (ThermoFisher, #194311010). In some embodiments, the anti-CH1 antibody is a monoclonal antibody. Monoclonal antibodies are typically produced from cultured antibody-producing cell lines. In other embodiments, the anti-CH1 antibody is a polyclonal antibody, i.e., a collection of different anti-CH1 antibodies that each recognize the CH1 epitope. Polyclonal antibodies are typically produced by collecting the antibody containing serum of an animal immunized with the antigen of interest, or fragment thereof, here CH1.

In some embodiments, CH1 binding reagents are molecules not derived from immunoglobulin proteins. Examples of such molecules include, but are not limited to, aptamers, peptoids, and affibodies, as described in more detail in Perret and Boschetti (Biochimie, February 2018, Vol 145:98-112).

### 6.11.2. Solid Supports

In exemplary non-limiting methods of purifying binding molecules, the CH1 binding reagent can be attached to a solid support in various embodiments of the invention. Solid supports, as described herein, refers to a material to which other entities can be attached or immobilized, e.g., the CH1 binding reagent. Solid supports, also referred to as “carriers,” are described in more detail in international application WO 2009/011572.

In specific embodiments, the solid support comprises a bead or nanoparticle. Examples of beads and nanoparticles include, but are not limited to, agarose beads, polystyrene beads, magnetic nanoparticles (e.g., Dynabeads™, ThermoFisher), polymers (e.g., dextran), synthetic polymers (e.g., Sepharose™), or any other material suitable for attaching the CH1 binding reagent. In particular embodiments, the solid support is modified to enable attachment of the CH1 binding reagent. Example of solid support modifications include, but are not limited to, chemical modifications that form covalent bonds with proteins (e.g., activated aldehyde groups) and modifications that specifically pair with a cognate modification of a CH1 binding reagent (e.g., biotin-streptavidin pairs, disulfide linkages, polyhistidine-nickel, or “click-chemistry” modifications such as azido-alkynyl pairs).

In certain embodiments, the CH1 binding reagent is attached to the solid support prior to the CH1 binding reagent contacting the binding molecules, herein also referred to as an “anti-CH1 resin.” In some embodiments, anti-CH1 resins are dispersed in a solution. In other embodiments, anti-CH1 resins are “packed” into a column. The anti-CH1 resin is then contacted with the binding molecules and the CH1 binding reagents specifically bind the binding molecules.

In other embodiments, the CH1 binding reagent is attached to the solid support after the CH1 binding reagent contacts the binding molecules. As a non-limiting illustration, a CH1 binding reagent with a biotin modification can be contacted with the binding molecules, and subsequently the CH1 binding reagent/binding molecule mixture can be contacted with streptavidin modified solid support to attach the CH1 binding reagent to the solid support, including CH1 binding reagents specifically bound to the binding molecules.

In methods wherein the CH1 binding reagents are attached to solid supports, in a variety of embodiments, the bound binding molecules are released, or “eluted,” from the solid support forming an eluate having the binding molecules. In some embodiments, the bound binding molecules are released through reversing the paired modifications (e.g., reduction of the disulfide linkage), adding a reagent to compete off the binding molecules (e.g., adding imidazole that competes with a polyhistidine for binding to nickel), cleaving off the binding molecules (e.g., a cleavable moiety can be included in the modification), or otherwise interfering with the specific binding of the CH1 binding reagent for the binding molecule. Methods that interfere with specific binding include, but are not limited to, contacting binding molecules bound to CH1 binding reagents with a low-pH solution. In preferred embodiment, the low-pH solution comprises 0.1 M acetic acid pH 4.0. In other embodiments, the bound binding molecules can be contacted with a range of low-pH solutions, i.e., a “gradient.”

### 6.11.3. Further Purification

In some embodiments of the exemplary non-limiting methods, a single iteration of the method using the steps of contacting the binding molecules with the CH1 binding reagents, followed by eluting the binding molecules, is used to purify the binding molecules from the one or more incomplete complexes. In particular embodiments, no other purifying step is performed. In other embodiments, one or more additional purification steps are performed to further purify the binding molecules from the one or more incomplete complexes. The one or more additional purification steps include, but are not limited to, purifying the binding molecules based on other protein characteristics, such as size (e.g., size exclusion chromatography), charge (e.g., ion exchange chromatography), or hydrophobicity (e.g., hydrophobicity interaction chromatography). In a preferred embodiment, an additional cation exchange chromatograph is performed. Additionally, the binding molecules can be further purified repeating contacting the binding molecules with the CH1 binding reagents as described above, as well as modifying the CH1 purification method between iterations, e.g., using a step elution for the first iteration and a gradient elution for a subsequent elution.

### 6.11.4. Assembly and Purity of Complexes

In the embodiments of the present invention, at least four distinct polypeptide chains associate together to form a complete complex, i.e., the binding molecule. However, incomplete complexes can also form that do not contain the at least four distinct polypeptide chains. For example, incomplete complexes may form that only have one, two, or three of the polypeptide chains. In other examples, an incomplete complex may contain more than three polypeptide chains, but does not contain the at least four distinct polypeptide chains, e.g., the incomplete complex inappropriately associates with more than one copy of a distinct polypeptide chain. The method of the invention purifies the complex, i.e., the completely assembled binding molecule, from incomplete complexes.

Methods to assess the efficacy and efficiency of the purification steps are well known to those skilled in the art and include, but are not limited to, SDS-PAGE analysis, ion exchange chromatography, size exclusion chromatography, and mass spectrometry. Purity can also be assessed according to a variety of criteria. Examples of criterion include, but are not limited to: 1) assessing the percentage of the total protein in an eluate that is provided by the completely assembled binding molecule, 2) assessing the fold enrichment or percent increase of the method for purifying the desired products, e.g., comparing the total protein provided by the completely assembled binding molecule in the eluate to that in a starting sample, 3) assessing the percentage of the total protein or the percent decrease of undesired products, e.g., the incomplete complexes described above, including determining the percent or the percent decrease of specific undesired products (e.g., unassociated single polypeptide chains, dimers of any combination of the polypeptide chains, or trimers of any combination of the polypeptide chains). Purity can be assessed after any combination of methods described herein. For example, purity can be assessed after a single iteration of using the anti-CH1 binding reagent, as described herein, or after additional purification steps, as described in more detail in Section 6.11.3. The efficacy and efficiency of the purification steps may also be used to compare the methods described using the anti-CH1 binding reagent to other purification methods known to those skilled in the art, such as Protein A purification.

### 6.12. Methods of Manufacturing

The binding molecules described herein can readily be manufactured by expression using standard cell free translation, transient transfection, and stable transfection approaches currently used for antibody manufacture. In specific embodiments, Expi293 cells (ThermoFisher) can be used for production of the binding molecules using protocols and reagents from ThermoFisher, such as ExpiFectamine, or other reagents known to those skilled in the art, such as polyethylenimine as described in detail in Fang et al. (Biological Procedures Online, 2017, 19:11), herein incorporated by reference for all it teaches.

As further described in the Examples below, the expressed proteins can be readily separated from undesired proteins and protein complexes using a CH1 affinity resin, such as the CaptureSelect CH1 resin and provided protocol from ThermoFisher. Other purification strategies include, but are not limited to, use of Protein A, Protein G, or Protein A/G reagents. Further purification can be affected using ion exchange chromatography as is routinely used in the art.

### 6.13. Pharmaceutical Compositions

In another aspect, pharmaceutical compositions are provided that comprise a binding molecule as described herein and a pharmaceutically acceptable carrier or diluent. In typical embodiments, the pharmaceutical composition is sterile.

In various embodiments, the pharmaceutical composition comprises the binding molecule at a concentration of 0.1 mg/ml-100 mg/ml. In specific embodiments, the pharmaceutical composition comprises the binding molecule at a concentration of 0.5 mg/ml, 1 mg/ml, 1.5 mg/ml, 2 mg/ml, 2.5 mg/ml, 5 mg/ml, 7.5 mg/ml, or 10 mg/ml. In some embodiments, the pharmaceutical composition comprises the binding molecule at a concentration of more than 10 mg/ml. In certain embodiments, the binding molecule is present at a concentration of 20 mg/ml, 25 mg/ml, 30 mg/ml, 35 mg/ml, 40 mg/ml, 45 mg/ml, or even 50 mg/ml or higher. In particular embodiments, the binding molecule is present at a concentration of more than 50 mg/ml.

In various embodiments, the pharmaceutical compositions are described in more detail in U.S. Pat. Nos. 8,961,964, 8,945,865, 8,420,081, 6,685,940, 6,171,586, 8,821,865, 9,216,219, U.S. application Ser. No. 10/813,483, WO 2014/066468, WO 2011/104381, and WO 2016/180941, each of which is incorporated herein in its entirety.

### 6.14. Methods of Treatment

In another aspect, methods of treatment are provided, the methods comprising administering a binding molecule as described herein to a patient in an amount effective to treat the patient.

In some embodiments, an antibody of the present disclosure may be used to treat a cancer. The cancer may be a cancer from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In some embodiments, the cancer may be a neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

An antibody of the present disclosure may be administered to a subject per se or in the form of a pharmaceutical composition for the treatment of, e.g., cancer, autoimmunity, transplantation rejection, post-traumatic immune responses, graft-versus-host disease, ischemia, stroke, and infectious diseases, for example by targeting viral antigens, such as gp 120 of HIV.

### 6.15. Examples

The following examples are provided by way of illustration, not limitation.

### 6.15.1. Methods

Non-limiting, illustrative methods for the purification of the various antigen-binding proteins and their use in various assays are described in more detail below.

### 6.15.1.1. Expi293 Expression

The various antigen-binding proteins tested were expressed using the Expi293 transient transfection system according to manufacturer's instructions. Briefly, four plasmids coding for four individual chains were mixed at 1:1:1:1 mass ratio, unless otherwise stated, and transfected with ExpiFectamine 293 transfection kit to Expi 293 cells. Cells were cultured at 37° C. with 8% CO2, 100% humidity and shaking at 125 rpm. Transfected cells were fed once after 16-18 hours of transfections. The cells were harvested at day 5 by centrifugation at 2000 g for 10 munities. The supernatant was collected for affinity chromatography purification.

### 6.15.1.2. Protein A and Anti-CH1 Purification

Cleared supernatants containing the various antigen-binding proteins were separated using either a Protein A (ProtA) resin or an anti-CH1 resin on an AKTA Purifier FPLC. In examples where a head-to-head comparison was performed, supernatants containing the various antigen-binding proteins were split into two equal samples. For ProtA purification, a 1 mL Protein A column (GE Healthcare) was equilibrated with PBS (5 mM sodium potassium phosphate pH 7.4, 150 mM sodium chloride). The sample was loaded onto the column at 5 ml/min. The sample was eluted using 0.1 M acetic acid pH 4.0. The elution was monitored by absorbance at 280 nm and the elution peaks were pooled for analysis. For anti-CH1 purification, a 1 mL CaptureSelect™ XL column (ThermoFisher) was equilibrated with PBS. The sample was loaded onto the column at 5 ml/min. The sample was eluted using 0.1 M acetic acid pH 4.0. The elution was monitored by absorbance at 280 nm and the elution peaks were pooled for analysis.

### 6.15.1.3. SDS-Page Analysis

Samples containing the various separated antigen-binding proteins were analyzed by reducing and non-reducing SDS-PAGE for the presence of complete product, incomplete product, and overall purity. 2 μg of each sample was added to 15 μL SDS loading buffer. Reducing samples were incubated in the presence of 10 mM reducing agent at 75° C. for 10 minutes. Non-reducing samples were incubated at 95° C. for 5 minutes without reducing agent. The reducing and non-reducing samples were loaded into a 4-15% gradient TGX gel (BioRad) with running buffer and run for 30 minutes at 250 volts. Upon completion of the run, the gel was washed with DI water and stained using GelCode Blue Safe Protein Stain (ThermoFisher). The gels were destained with DI water prior to analysis. Densitometry analysis of scanned images of the destained gels was performed using standard image analysis software to calculate the relative abundance of bands in each sample.

### 6.15.1.4. IEX Chromatography

Samples containing the various separated antigen-binding proteins were analyzed by cation exchange chromatography for the ratio of complete product to incomplete product and impurities. Cleared supernatants were analyzed with a 5-ml MonoS (GE Lifesciences) on an AKTA Purifier FPLC. The MonoS column was equilibrated with buffer A 10 mM MES pH 6.0. The samples were loaded onto the column at 2 ml/min. The sample was eluted using a 0-30% gradient with buffer B (10 mM IVIES pH 6.0, 1 M sodium chloride) over 6 CV. The elution was monitored by absorbance at 280 nm and the purity of the samples were calculated by peak integration to identify the abundance of the monomer peak and contaminants peaks. The monomer peak and contaminant peaks were separately pooled for analysis by SDS-PAGE as described above.

### 6.15.1.5. Analytical SEC Chromatography

Samples containing the various separated antigen-binding proteins were analyzed by analytical size exclusion chromatography for the ratio of monomer to high molecular weight product and impurities. Cleared supernatants were analyzed with an industry standard TSK G3000SW×1 column (Tosoh Bioscience) on an Agilent 1100 HPLC. The TSK column was equilibrated with PBS. 25 μL of each sample at 1 mg/mL was loaded onto the column at 1 ml/min. The sample was eluted using an isocratic flow of PBS for 1.5 CV. The elution was monitored by absorbance at 280 nm and the elution peaks were analyzed by peak integration.

### 6.15.1.6. Mass Spec

Samples containing the various separated antigen-binding proteins were analyzed by mass spectrometry to confirm the correct species by molecular weight. All analysis was performed by a third-party research organization. Briefly, samples were treated with a cocktail of enzymes to remove glycosylation. Samples were both tested in the reduced format to specifically identify each chain by molecular weight. Samples were all tested under non-reducing conditions to identify the molecular weights of all complexes in the samples. Mass spec analysis was used to identify the number of unique products based on molecular weight.

### 6.15.1.7. Antibody Discovery by Phage Display

Phage display of human Fab libraries are carried out using standard protocols. Phage clones are screened for the ability to bind an antigen of interest by phage ELISA using standard protocols. Briefly, Fab-formatted phage libraries were constructed using expression vectors capable of replication and expression in phage (also referred to as a phagemid). Both the heavy chain and the light chain were encoded for in the same expression vector, where the heavy chain was fused to a truncated variant of the phage coat protein pIII. The light chain and heavy chain are expressed as a separate polypeptides, and the light chain and heavy chain-pIII fusion assemble in the bacterial periplasm, where the redox potential enables disulfide bond formation, to form the antibody containing the candidate ABS.

The library was created using sequences derived from a specific human heavy chain variable domain (VH3-23) and a specific human light chain variable domain (Vk-1). Light chain variable domains within the screened library were generated with diversity was introduced into the VL CDR3 (L3) and where the light chain VL CDR1 (L1) and CDR2 (L2) remained the human germline sequence. For the screened library, all three CDRs of the VH domain were diversified to match the positional amino acid frequency by CDR length found in the human antibody repertoire. The phage display heavy chain (SEQ ID NO:74) and light chain (SEQ ID NO:75) scaffolds used in the library are listed below, where a lower case “x” represents CDR amino acids that were varied to create the library, and bold italic represents the CDR sequences that were constant.

Diversity was created through Kunkel mutagenesis using primers to introduce diversity into VL CDR3 and VH CDR1 (H1), CDR2 (H2) and CDR3 (H3) to mimic the diversity found in the natural antibody repertoire, as described in more detail in Kunkel, TA (PNAS Jan. 1, 1985. 82 (2) 488-492), herein incorporated by reference in its entirety. Briefly, single-stranded DNA were prepared from isolated phage using standard procedures and Kunkel mutagenesis carried out. Chemically synthesized DNA was then electroporated into TG1 cells, followed by recovery. Recovered cells were sub-cultured and infected with M13K07 helper phage to produce the phage library.

Phage panning was performed using standard procedures. Briefly, the first round of phage panning was performed with target immobilized on streptavidin magnetic beads which were subjected to −5×10¹² phages from the prepared library in a volume of 1 mL in PBST-2% BSA. After a one-hour incubation, the bead-bound phage were separated from the supernatant using a magnetic stand. Beads were washed three times to remove non-specifically bound phage and were then added to ER2738 cells (5 mL) at OD₆₀₀˜0.6. After 20 minutes, infected cells were sub-cultured in 25 mL 2×YT+Ampicillin and M13K07 helper phage and allowed to grow overnight at 37° C. with vigorous shaking. The next day, phage were prepared using standard procedures by PEG precipitation. Pre-clearance of phage specific to SAV-coated beads was performed prior to panning. The second round of panning was performed using the KingFisher magnetic bead handler with 100 nM bead-immobilized antigen using standard procedures. In total, 3-4 rounds of phage panning were performed to enrich in phage displaying Fabs specific for the target antigen. Target-specific enrichment was confirmed using polyclonal and monoclonal phage ELISA. DNA sequencing was used to determine isolated Fab clones containing a candidate ABS.

To measure binding affinity in discovery campaigns, the VL and VH domains are formatted into a bivalent monospecific native human full-length IgG1 architecture and immobilized to a biosensor on an Octet (Pall ForteBio) biolayer interferometer. Soluble antigens are then added to the system and binding measured.

For experiments performed using the B-Body format, VL variable regions of individual clones are formatted into Domain A and/or H, and VH region into Domain F and/or L of a bivalent 1×1 B-Body “BC1” scaffold shown below and with reference to FIG. 3.

“BC1” Scaffold:
1^{st} polypeptide chain (SEQ ID NO:78)
   Domain A=Antigen 1 B-Body Domain A/H Scaffold (SEQ ID NO:76)
   Domain B=CH3 (T366K; 445K, 446S, 447C tripeptide insertion)
   Domain D=CH2
   Domain E=CH3 (T366W, S354C)
2^{nd} polypeptide chain (SEQ ID NO:79):
   Domain F=Antigen 1 B-Body Domain F/L Scaffold (SEQ ID NO:77)
   Domain G=CH3 (L351D; 445G, 446E, 447C tripeptide insertion)
3^{rd} polypeptide chain (SEQ ID NO:80):
   Domain H=Antigen 2 B-Body Domain A/H Scaffold (SEQ ID NO:76)
   Domain I=CL (Kappa)
   Domain J=CH2
   Domain K=CH3 (Y349C, D356E, L358M, T366S, L368A, Y407V)
4^{th} polypeptide chain (SEQ ID NO:81):
   Domain L=Antigen 2 B-Body Domain F/L Scaffold (SEQ ID NO:77)
   Domain M=CH1.

For BC1 1×2 formats, the variable domains were formatted into the 1(A)×2(B-A) format described in Section 6.4.17.4. Polypeptide Chain 2 and Chain 6 are identical in the 1(A)×2(B-A) format.

### 6.15.1.8. NFκB GFP Jurkat T Cell Stimulation Assay

The NFκB/Jurkat/GFP transcriptional reporter cell line was purchased from System Biosciences (Cat #TR850-1). The anti-CD28 antibody used for co-stimulation was purchased from BD Pharmingen (Cat 555725). The Solution C background suppression dye was purchased from Life Technologies (K1037). Briefly, the Jurkat cells (effector cells, E) were mixed with the tumor cells (T) at an E:T ratio of 2:1 to 4:1 in the presence of a dilution series of B-body™ antibodies and an anti-CD28 antibody at 1 μg/mL in a 96 well black walled clear bottom plate. The plate was incubated at 37° C./5% CO2 for 6 hours, following which a 6× solution of Solution C background suppressor was added to the plate and GFP fluorescence was read out on a plate reader. EC50 values, referring to the concentration of antibody that gives the half-maximal response, were determined from the dilution series.

### 6.15.1.9. Primary T Cell Cytotoxicity Assay

Cells expressing the target tumor antigen (T) and effector cells (E) were mixed at an E:T ratio ranging from 3:1 to 10:1. Effector cells used include PBMCs or isolated cytotoxic CD8+ T cells. The candidate redirecting T cell antibody was added in a dilution series to the cells. Controls included media only controls, tumor cell only controls, and untreated E:T cell controls. The mixed cells and control conditions were incubated at 37° C./5% CO2 for 40-50 hours. The Cytotoxicity Detection Kit Plus (LDH) was purchased from Sigma (Cat 4744934001) and the manufacturer's directions were followed. Briefly, lysis solution added to tumor cells served as the 100% cytotoxicity control and untreated E:T cells served as the 0% cytotoxicity control. The level of lactate dehydrogenase (LDH) in each sample was determined via absorbance at 490 nm and normalize to the 100% and 0% controls. EC50 values, referring to the concentration of antibody that gives the half-maximal response, were determined from the dilution series.

### 6.15.2. Example 1: Bivalent Monospecific Construct and Bivalent Bispecific Construct

A bivalent monospecific B-Body recognizing TNFα was constructed with the following architecture (VL(Certolizumab)-CH3(Knob)-CH2-CH3/VH(Certolizumab)-CH3(Hole)) using standard molecular biology procedures. In this construct,
1st polypeptide chain (SEQ ID NO:1)
   Domain A=VL (certolizumab)
   Domain B=CH3 (IgG1) (knob: S354C+T366W)
   Domain D=CH2 (IgG1)
   Domain E=CH3 (IgG1)
2nd polypeptide chain (SEQ ID NO:2)
   Domain F=VH (certolizumab)
   Domain G=CH3 (IgG1) (hole:Y349C, T366'S, L368A, Y407V)
3rd polypeptide chain:
   identical to the 1st polypeptide chain
4th polypeptide chain:
   identical to the 2nd polypeptide chain.

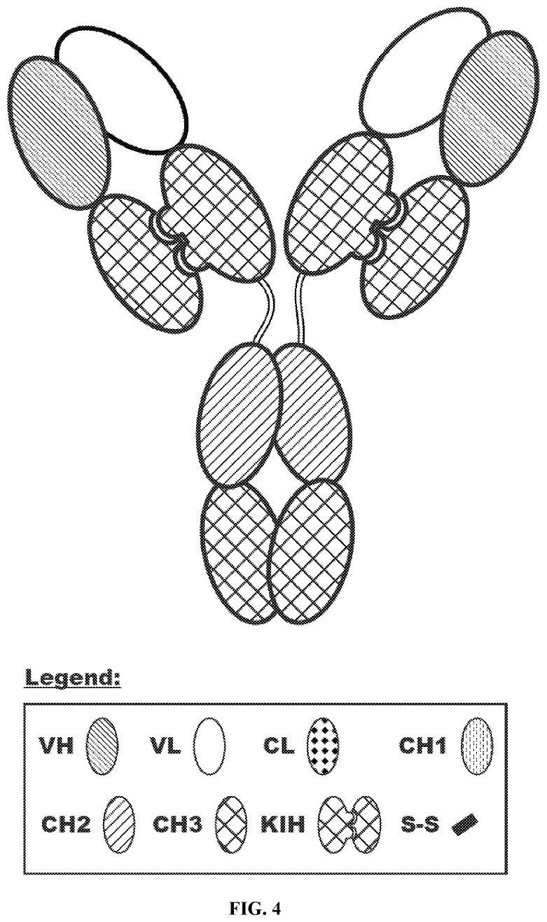

Domain and polypeptide chain references are in accordance with FIG. 3. The overall construct architecture is illustrated in FIG. 4. The sequence of the first polypeptide chain, with domain A identified in shorthand as “(VL)”, is provided in SEQ ID NO:1. The sequence of the second polypeptide chain, with domain F identified in shorthand as “(VH)”, is provided in SEQ ID NO:2.

The full-length construct was expressed in an E. coli cell free protein synthesis expression system for ˜18 hours at 26° C. with gentle agitation. Following expression, the cell-free extract was centrifuged to pellet insoluble material and the supernatant was diluted 2× with 10× Kinetic Buffer (Forte Bio) and used as the analyte for biolayer interferometry.

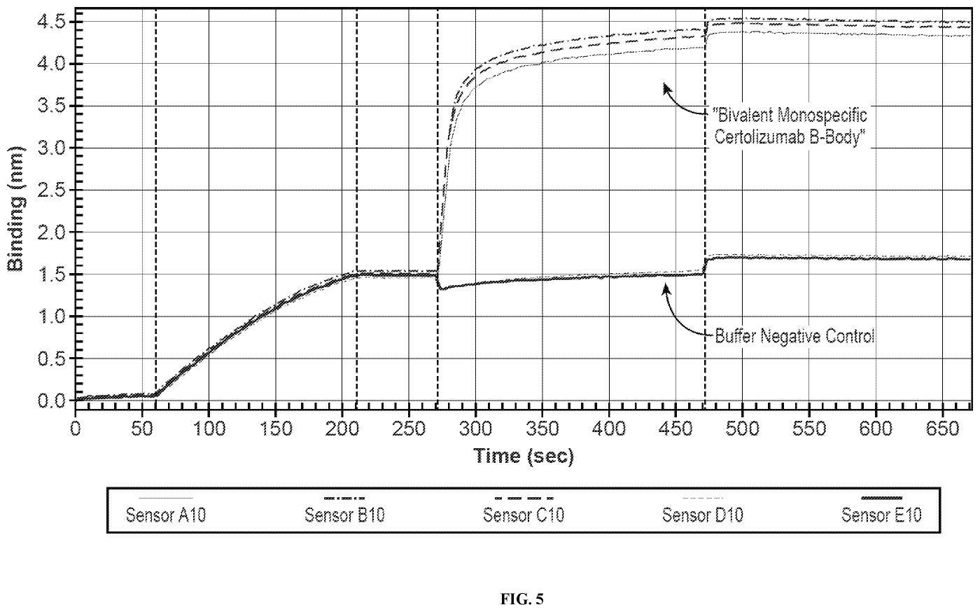

Biotinylated TNFα was immobilized on a streptavidin sensor to give a wave shift response of ˜1.5 nm. After establishing a baseline with 10× kinetic buffer, the sensor was dipped into the antibody construct analyte solution. The construct gave a response of ˜3 nm, comparable to the traditional IgG format of certolizumab, demonstrating the ability of the bivalent monospecific construct to assemble into a functional, full-length antibody. Results are shown in FIG. 5.

We also constructed a bivalent bispecific antibody with the following domain architecture:
1^{st} polypeptide chain: VL-CH3-CH2-CH3(Knob)
2^{nd} polypeptide chain: VH-CH3
3^{rd} polypeptide chain: VL-CL-CH2-CH3(Hole)
4^{th} polypeptide chain VH-CH1.

The sequences (except for the variable region sequences) are provided respectively in SEQ ID NO:3 (1st polypeptide chain), SEQ ID NO:4 (2nd polypeptide chain), SEQ ID NO:5 (3rd polypeptide chain), SEQ ID NO:6 (4th polypeptide chain).

### 6.15.3. Example 2: Bivalent Bispecific B-Body “BC1”

We constructed a bivalent bispecific construct, termed “BC1”, specific for PD1 and a second antigen, “Antigen A”). Salient features of the “BC1” architecture are illustrated in FIG. 6.

In greater detail, with domain and polypeptide chain references in accordance with FIG. 3 and modifications from native sequence indicated in parentheses, the architecture was:
1^{st} polypeptide chain (SEQ ID NO:8)
   Domain A=VL (“Antigen A”)
   Domain B=CH3 (T366K; 445K, 446S, 447C tripeptide insertion)
   Domain D=CH2
   Domain E=CH3 (T366W, S354C)
2nd polypeptide chain (SEQ ID NO:9):
   Domain F=VH (“Antigen A”)
   Domain G=CH3 (L351D; 445G, 446E, 447C tripeptide insertion)
3rd polypeptide chain (SEQ ID NO:10):
   Domain H=VL (“Nivo”)
   Domain I=CL (Kappa)
   Domain J=CH2
   Domain K=CH3 (Y349C, D356E, L358M, T366S, L368A, Y407V)
4th polypeptide chain (SEQ ID NO:11):
   Domain L=VH (“Nivo”)
   Domain M=CH1.

The A domain (SEQ ID NO: 12) and F domain (SEQ ID NO: 16) form an antigen binding site (A:F) specific for “Antigen A”. The H domain has the VH sequence from nivolumab and the L domain has the VL sequence from nivolumab; H and L associate to form an antigen binding site (H:L) specific for human PD1.

The B domain (SEQ ID NO:13) has the sequence of human IgG1 CH3 with several mutations: T366K, 445K, 446S, and 447C insertion. The T366K mutation is a charge pair cognate of the L351D residue in Domain G. The “447C” residue on domain B comes from the C-terminal KSC tripeptide insertion.

Domain D (SEQ ID NO: 14) has the sequence of human IgG1 CH2

Domain E (SEQ ID NO: 15) has the sequence of human IgG1 CH3 with the mutations T366W and S354C. The 366W is the “knob” mutation. The 354C introduces a cysteine that is able to form a disulfide bond with the cognate 349C mutation in Domain K.

Domain G (SEQ ID NO: 17) has the sequence of human IgG1 CH3 with the following mutations: L351D, and 445G, 446E, 447C tripeptide insertion. The L351D mutation introduces a charge pair cognate to the Domain B T366K mutation. The “447C” residue on domain G comes from the C-terminal GEC tripeptide insertion.

Domain I (SEQ ID NO: 19) has the sequence of human C kappa light chain (Cκ)

Domain J [SEQ ID NO: 20] has the sequence of human IgG1 CH2 domain, and is identical to the sequence of domain D.

Domain K [SEQ ID NO: 21] has the sequence of human IgG1 CH3 with the following changes: Y349C, D356E, L358M, T366S, L368A, Y407V. The 349C mutation introduces a cysteine that is able to form a disulfide bond with the cognate 354C mutation in Domain E. The 356E and L358M introduce isoallotype amino acids that reduce immunogenicity. The 366S, 368A, and 407V are “hole” mutations.

Domain M [SEQ ID NO: 23] has the sequence of the human IgG1 CH1 region.

“BC1” could readily be expressed at high levels using mammalian expression at concentrations greater than 100 μg/ml.

We found that the bivalent bispecific “BC1” protein could easily be purified in a single step using a CH1-specific CaptureSelect™ affinity resin from ThermoFisher.

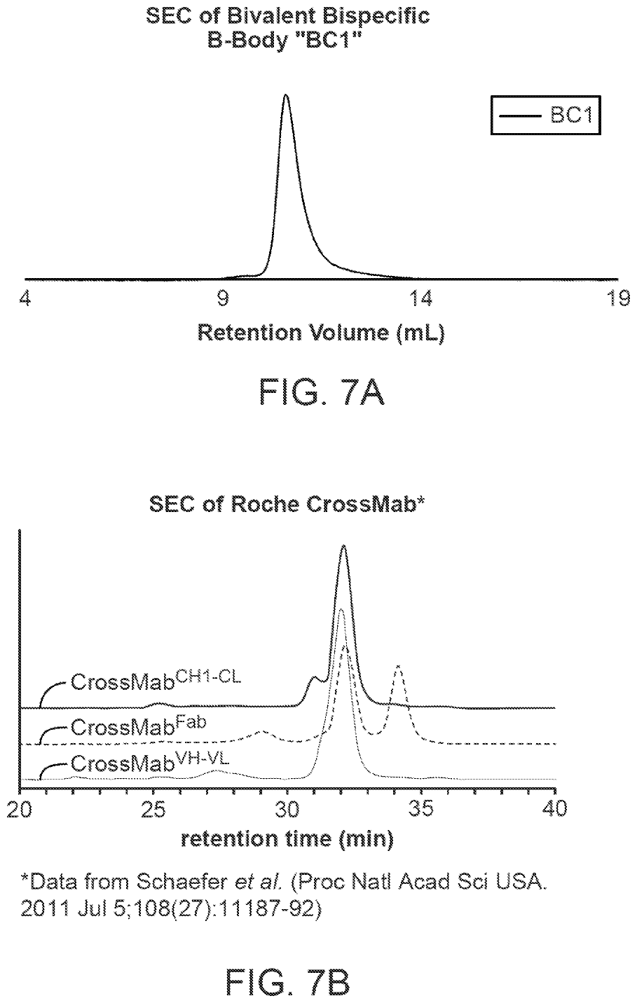

As shown in FIG. 7A, SEC analysis demonstrates that a single-step CH1 affinity purification step yields a single, monodisperse peak via gel filtration in which >98% is monomer. FIG. 7B shows comparative literature data of SEC analysis of a CrossMab bivalent antibody construct.

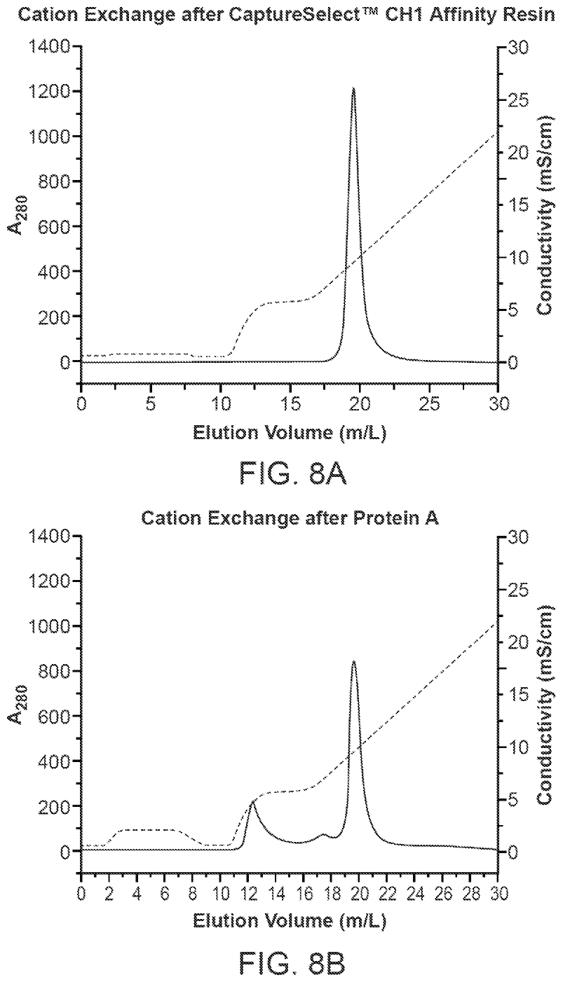

FIG. 8A is a cation exchange chromatography elution profile of “BC1” following one-step purification using the CaptureSelect™ CH1 affinity resin, showing a single tight peak. FIG. 8B is a cation exchange chromatography elution profile of “BC1” following purification using standard Protein A purification, showing additional elution peaks consistent with the co-purification of incomplete assembly products.

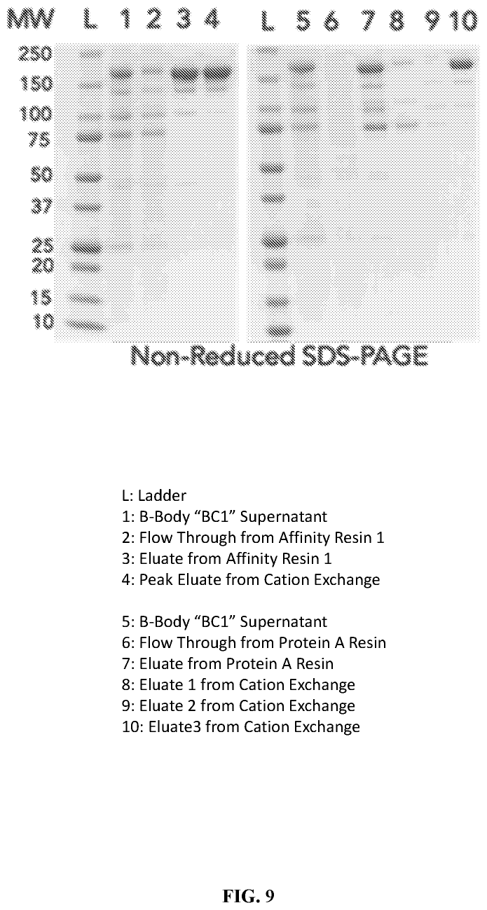

FIG. 9 shows SDS-PAGE gels under non-reducing conditions. As seen in lane 3, single-step purification of “BC1” with CH1 affinity resin provides a nearly homogeneous single band, with lane 4 showing minimal additional purification with a subsequent cation exchange polishing step. Lane 7, by comparison, shows less substantial purification using standard Protein A purification, with lanes 8-10 demonstrating further purification of the Protein A purified material using cation exchange chromatography.

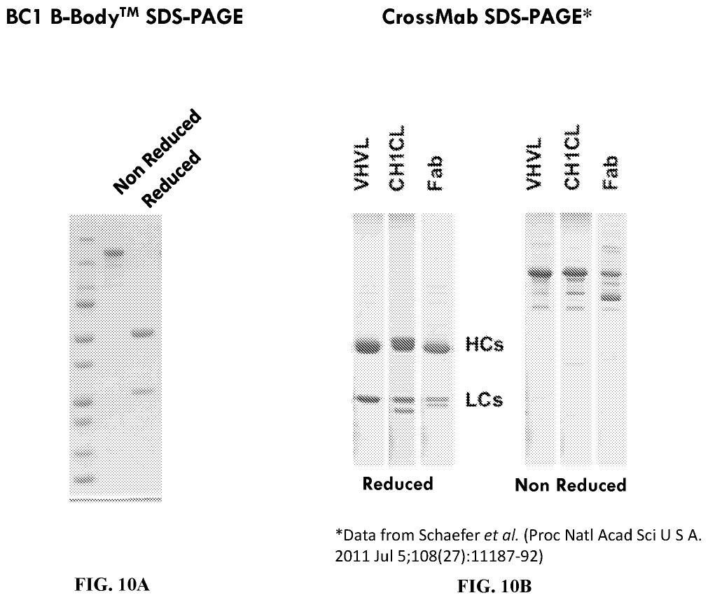

FIG. 10 compares SDS-PAGE gels of “BC1” after single-step CH1-affinity purification, under both non-reducing and reducing conditions (Panel A) with SDS-PAGE gels of a CrossMab bispecific antibody under non-reducing and reducing conditions as published in the referenced literature (Panel B).

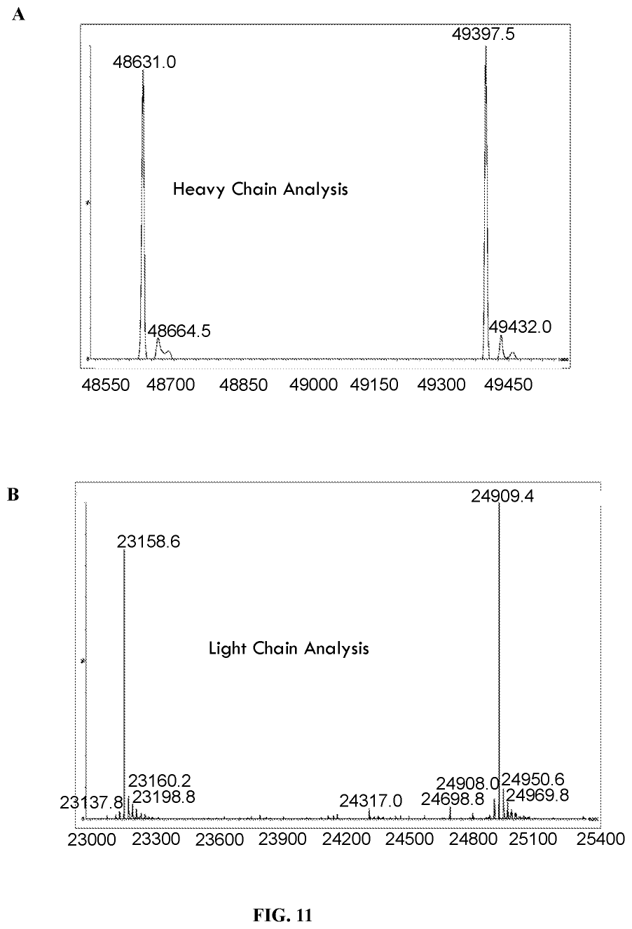

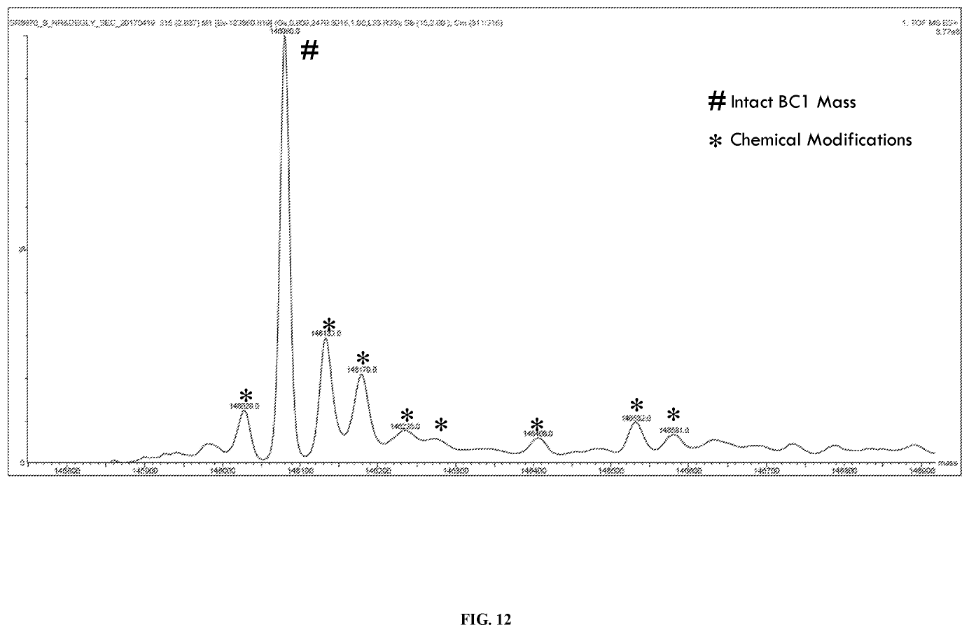

FIG. 11 shows mass spec analysis of “BC1”, demonstrating two distinct heavy chains (FIG. 11A) and two distinct light chains (FIG. 11B) under reducing conditions. The mass spectrometry data in FIG. 12 confirms the absence of incomplete pairing after purification.

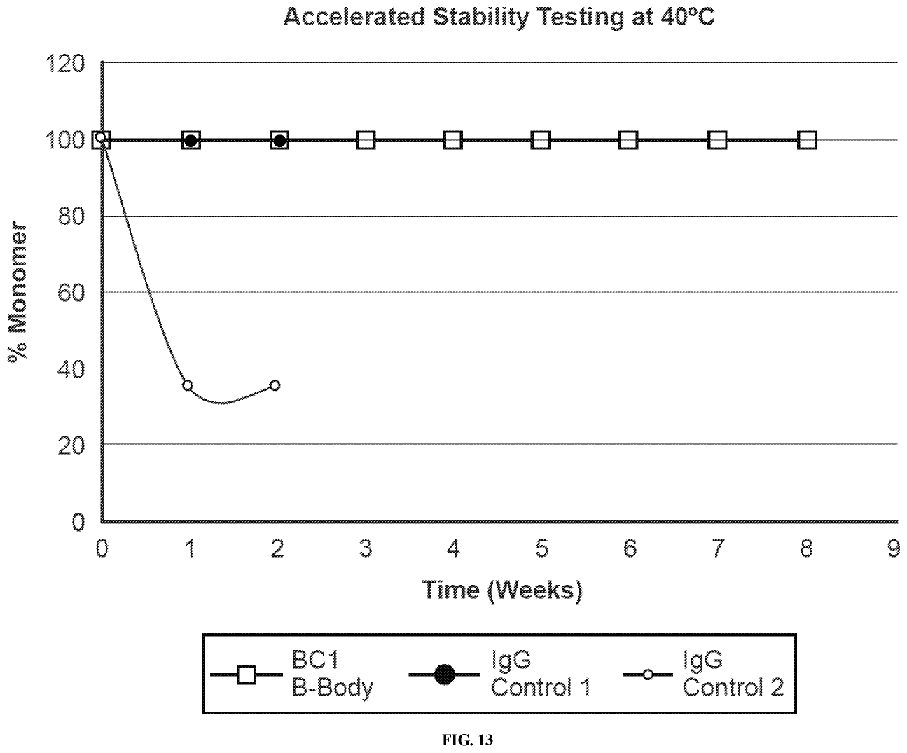

Accelerated stability testing was performed to evaluate the long-term stability of the “BC1” B-Body design. The purified B-Body was concentrated to 8.6 mg/ml in PBS buffer and incubated at 40° C. The structural integrity was measured weekly using analytical size exclusion chromatography (SEC) with a Shodex KW-803 column. The structural integrity was determined by measuring the percentage of intact monomer (% Monomer) in relation to the formation of aggregates. Data are shown in FIG. 13. The IgG Control 1 is a positive control with good stability properties. IgG Control 2 is a negative control that is known to aggregate under the incubation conditions. The “BC1” B-Body has been incubated for 8 weeks without any loss of structural integrity as determined by the analytical SEC.

We have also determined that “BC1” has high thermostability, with a TM of the bivalent construct of ˜72° C.

Table 1 compares “BC1” to CrossMab in key developability characteristics:

TABLE 1

| | | Roche | |
|---|---|---|---|
| Parameter | Unit | CrossMab* | “BC1” |
| Purification yield after protein | mg/L | 58.5 | 300 |
| A/SEC | | | |
| Homogeneity After purification | % SEC Area | 50-85 | 98 |
| Denaturation Temp (Tm) | degrees C. | 69.2 | 72 |

*Data from Schaefer et al. (Proc Natl Acad Sci USA. 2011 Jul. 5; 108(27): 11187-92)

### 6.15.4. Example 3: Bivalent Bispecific B-Body “BC6”

We constructed a bivalent bispecific B-Body, termed “BC6”, that is identical to “BC1” but for retaining wild type residues in Domain B at residue 366 and Domain G at residue 351. “BC6” thus lacks the charge-pair cognates T366K and L351D that had been designed to facilitate correct pairing of domain B and domain G in “BC1”. Salient features of the “BC6” architecture are illustrated in FIG. 14.

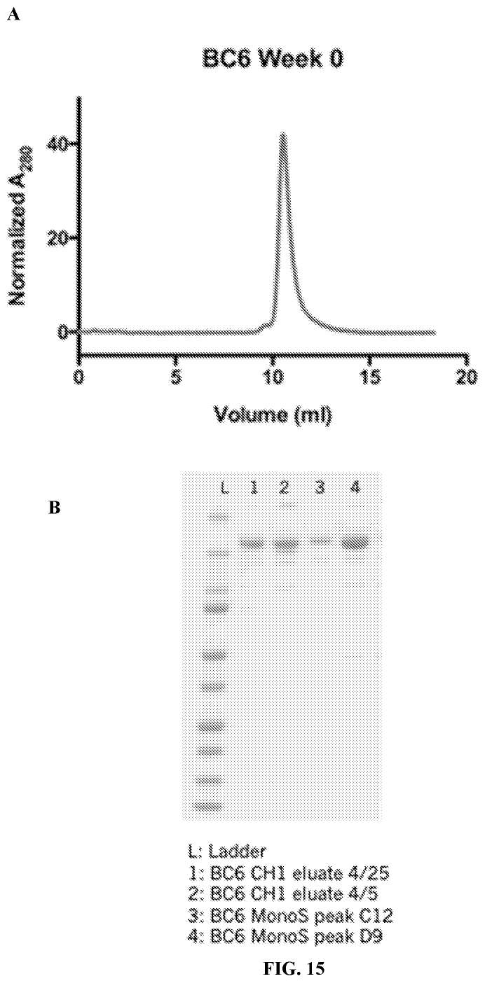

Notwithstanding the absence of the charge-pair residues present in “BC1”, we found that a single step purification of “BC6” using CH1 affinity resin resulted in a highly homogeneous sample. FIG. 15A shows SEC analysis of “BC6” following one-step purification using the CaptureSelect™ CH1 affinity resin. The data demonstrate that the single step CH1 affinity purification yields a single monodisperse peak, similar to what we observed with “BC1”, demonstrating that the disulfide bonds between polypeptide chains 1 and 2 and between polypeptide chains 3 and 4 are intact. The chromatogram also shows the absence of non-covalent aggregates.

FIG. 15B shows a SDS-PAGE gel under non-reducing conditions, with lane 1 loaded with a first lot of “BC6” after a single-step CH1 affinity purification, lane 2 loaded with a second lot of “BC6” after a single-step CH1 affinity purification. Lanes 3 and 4 demonstrate further purification can be achieved with ion exchange chromatography subsequent to CH1 affinity purification.

### 6.15.5. Example 4: Bivalent Bispecific B-Bodies “BC28”, “BC29”, “BC30”, “BC31”

We constructed bivalent 1×1 bispecific B-Bodies “BC28”, “BC29”, “BC30” and “BC31” having an engineered disulfide within the CH3 interface in Domains B and G as an alternative S-S linkage to the C-terminal disulfide present in “BC1” and “BC6”. Literature indicates that CH3 interface disulfide bonding is insufficient to enforce orthogonality in the context of Fc CH3 domains. The general architecture of these B-Body constructs is schematized in FIG. 16 with salient features of “BC28” summarized below:

Polypeptide chain 1: “BC28” chain 1 (SEQ ID NO:24)
Domain A=VL (Antigen “A”)
Domain B=CH3 (Y349C; 445P, 446G, 447K insertion)
Domain D=CH2
Domain E=CH3 (S354C, T366W)
Polypeptide chain 2: “BC28” chain 2 (SEQ ID NO:25)
   Domain F=VH (Antigen “A”)
   Domain G=CH3 (S354C; 445P, 446G, 447K insertion)
Polypeptide chain 3: “BC1” chain 3 (SEQ ID NO:10)
   Domain H=VL (“Nivo”)
   Domain I=CL (Kappa)
   Domain J=CH2
   Domain K=CH3 (Y349C, D356E, L358M, T366S, L368A, Y407V)
Polypeptide chain 4: “BC1” chain 4 (SEQ ID NO:11)
   Domain L=VH (“Nivo”)
   Domain M=CH1.

The “BC28” A:F antigen binding site is specific for “Antigen A”. The “BC28” H:L antigen binding site is specific for PD1 (nivolumab sequences). “BC28” domain B has the following changes as compared to wild type CH3: Y349C; 445P, 446G, 447K insertion. “BC28” domain E has the following changes as compared to wild type CH3: S354C, T366W. “BC28” domain G has the following changes as compared to wild type: S354C; 445P, 446G, 447K insertion.

“BC28” thus has an engineered cysteine at residue 349C of Domain B and engineered cysteine at residue 354C of domain G (“349C-354C”).

“BC29” has engineered cysteines at residue 351C of Domain B and 351C of Domain G (“351C-351C”). “BC30” has an engineered cysteine at residue 354C of Domain B and 349C of Domain G (“354C-349C”). BC31 has an engineered cysteine at residue 394C and engineered cysteine at 394C of Domain G (“394C-394C”). BC32 has engineered cysteines at residue 407C of Domain B and 407C of Domain G (“407C-407C”).

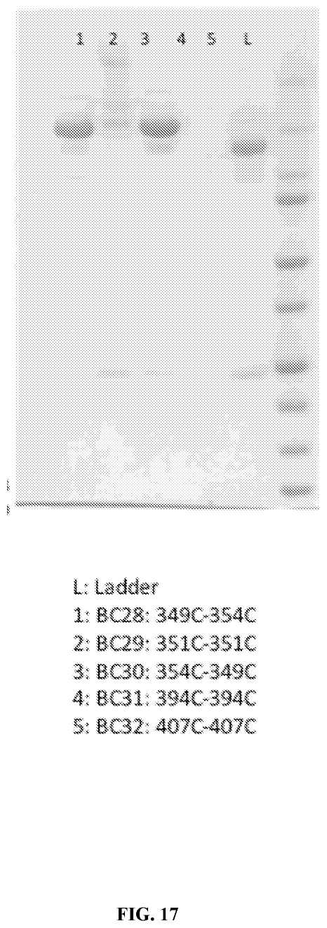

FIG. 17 shows SDS-PAGE analysis under non-reducing conditions following one-step purification using the CaptureSelect™ CH1 affinity resin. Lanes 1 and 3 show high levels of expression and substantial homogeneity of intact “BC28” (lane 1) and “BC30” (lane 3). Lane 2 shows oligomerization of BC29. Lanes 4 and 5 show poor expression of BC31 and BC32, respectively, and insufficient linkage in BC32. Another construct, BC9, which had cysteines introduced at residue 392 in domain B and 399 in Domain G (“392C-399C”), a disulfide pairing reported by Genentech, demonstrated oligomerization on SDS PAGE (data not shown).

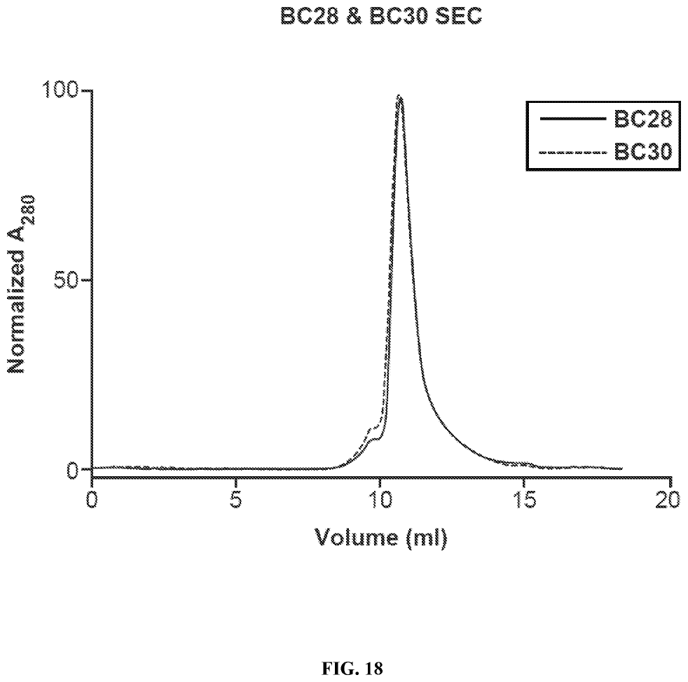

FIG. 18 shows SEC analysis of “BC28” and “BC30” following one-step purification using the CaptureSelect™ CH1 affinity resin. We have also demonstrated that “BC28” can readily be purified using a single step purification using Protein A resin (results not shown).

### 6.15.6. Example 5: Bivalent Bispecific B-Body “BC44”

FIG. 19 shows the general architecture of the bivalent bispecific 1×1 B-Body “BC44”, our currently preferred bivalent bispecific 1×1 construct.
first polypeptide chain (′BC44″ chain 1) (SEQ ID NO:32)
   Domain A=VL (Antigen “A”)
   Domain B=CH3 (P343V; Y349C; 445P, 446G, 447K insertion)
   Domain D=CH2
   Domain E=CH3 (S354C, T366W)
second polypeptide chain (=“BC28” polypeptide chain 2) (SEQ NO:25)
   Domain F=VH (Antigen “A”)
   Domain G=CH3 (S354C; 445P, 446G, 447K insertion)
third polypeptide chain (=“BC1” polypeptide chain 3) (SEQ ID NO:10)
   Domain H=VL (“Nivo”)
   Domain I=CL (Kappa)
   Domain J=CH2
   Domain K=CH3 (Y349C, D356E, L358M, T366S, L368A, Y407V)
fourth polypeptide chain (=“BC1” polypeptide chain 4) (SEQ ID NO:11)
   Domain L=VH (“Nivo”)
   Domain M=CH1.

### 6.15.7. Example 6: Variable-CH3 Junction Engineering

We produced a series of variants in which we mutated the VL-CH3 junction between Domains A and B and the VH-CH3 junction between domains F and G to assess the expression level, assembly and stability of bivalent 1×1 B-Body constructs. Although there are likely many solutions, to reduce introduction of T cell epitopes we chose to only use residues found naturally within the VL, VH and CH3 domains. Structural assessment of the domain architecture further limits desirable sequence combinations. Table 2 and Table 3 below show junctions for several junctional variants based on “BC1” and other bivalent constructs.

TABLE 2
Variants of Variable Domain/Constant Domain Junctions for 1^{st} Polypeptide Chain

| | VL | | | | | | CH3 | | | | |
|---|---|---|---|---|---|---|---|---|---|---|---|
| Variant | 106 | 107 | 108 | 109 | 110 | 111 | 343 | 344 | 345 | 346 | Sequence |
| BC1 | I | K | R | T | | | P | R | E | P | IKRTPREP |
| | | | | | | | | | | | (SEQ ID NO: 57) |
| BC13 | I | K | R | T | | | P | R | E | P | IKRTPREP |
| | | | | | | | | | | | (SEQ ID NO: 57) |
| BC14 | I | K | R | T | | | P | R | E | P | IKRTPREP |
| | | | | | | | | | | | (SEQ ID NO: 57) |
| BC15 | I | K | R | T | V | | | R | E | P | IKRTVREP |
| | | | | | | | | | | | (SEQ ID NO: 58) |
| BC16 | I | K | R | T | | | | R | E | P | IKRTREP |
| | | | | | | | | | | | (SEQ ID NO: 59) |
| BC17 | I | K | R | T | V | | P | R | E | P | IKRTVPREP |
| | | | | | | | | | | | (SEQ ID NO: 60) |
| BC24 | I | K | R | T | | | P | R | E | P | IKRTPREP |
| | | | | | | | | | | | (SEQ ID NO: 57) |
| BC25 | I | K | R | T | | | P | R | E | P | IKRTPREP |
| | | | | | | | | | | | (SEQ ID NO: 57) |
| BC26 | I | K | R | T | V | A | | | E | P | IKRTVAEP |
| | | | | | | | | | | | (SEQ ID NO: 61) |
| BC27 | I | K | R | T | V | A | P | R | E | P | IKRTVAPREP |
| | | | | | | | | | | | (SEQ ID NO: 62) |
| BC44 | I | K | R | T | | | V | R | E | P | IKRTVREP |
| | | | | | | | | | | | (SEQ ID NO: 58) |
| BC45 | I | K | R | T | | | P | R | E | P | IKRTPREP |
| | | | | | | | | | | | (SEQ ID NO: 57) |
| BC5 | I | K | R | T | | | P | R | E | P | IKRTPREP |
| | | | | | | | | | | | (SEQ ID NO: 57) |
| BC6 | I | K | R | T | | | P | R | E | P | IKRTPREP |
| | | | | | | | | | | | (SEQ ID NO: 57) |
| BC28 | I | K | R | T | | | P | R | E | P | IKRTPREP |
| | | | | | | | | | | | (SEQ ID NO: 57) |
| BC30 | I | K | R | T | | | P | R | E | P | IKRTPREP |
| | | | | | | | | | | | (SEQ ID NO: 57) |

TABLE 3
Variants of Variable Domain/Constant Domain Junctions for 2^{nd} Polypeptide Chain

| | |
|---|---|
| VL | CH3 |

| Variant | 112 | 113 | 114 | 115 | 116 | 117 | 118 | 343 | 344 | 345 | 346 | Sequence |
|---|---|---|---|---|---|---|---|---|---|---|---|---|
| BC1 | S | S | A | S | | | | P | R | E | P | SSASPREP |
| | | | | | | | | | | | | (SEQ ID NO: 63) |
| BC13 | S | S | A | S | T | | | | R | E | P | SSASTREP |
| | | | | | | | | | | | | (SEQ ID NO: 64) |
| BC14 | S | S | A | S | T | | | P | R | E | P | SSASTRPREP |
| | | | | | | | | | | | | (SEQ OD NO: 65) |
| BC15 | S | S | A | S | | | | P | R | E | P | SSASPREP |
| | | | | | | | | | | | | (SEQ ID NO: 63) |
| BC16 | S | S | A | S | | | | P | R | E | P | SSASPREP |
| | | | | | | | | | | | | (SEQ ID NO: 63) |
| BC17 | S | S | A | S | | | | P | R | E | P | SSASPREP |
| | | | | | | | | | | | | (SEQ ID NO: 63) |
| BC24 | S | S | A | S | T | K | G | | | E | P | SSASTKGEP |
| | | | | | | | | | | | | (SEQ ID NO: 66) |
| BC25 | S | S | A | S | T | K | G | | R | E | P | SSASTKGREP |
| | | | | | | | | | | | | (SEQ ID NO: 67) |
| BC26 | S | S | A | S | | | | P | R | E | P | SSASPREP |
| | | | | | | | | | | | | (SEQ ID NO: 63) |
| BC27 | S | S | A | S | | | | P | R | E | P | SSASPREP |
| | | | | | | | | | | | | (SEQ ID NO: 63) |
| BC44 | S | S | A | S | | | | P | R | E | P | SSASPREP |
| | | | | | | | | | | | | (SEQ ID NO: 63) |
| BC45 | S | S | A | S | | | | P | R | E | P | SSASPREP |
| | | | | | | | | | | | | (SEQ ID NO: 63) |
| BC5 | S | S | A | S | | | | P | R | E | P | SSASPREP |
| | | | | | | | | | | | | (SEQ ID NO: 63) |
| BC6 | S | S | A | S | | | | P | R | E | P | SSASPREP |
| | | | | | | | | | | | | (SEQ ID NO: 63) |
| BC28 | S | S | A | S | | | | P | R | E | P | SSASPREP |
| | | | | | | | | | | | | (SEQ ID NO: 63) |
| BC30 | S | S | A | S | | | | P | R | E | P | SSASPREP |
| | | | | | | | | | | | | (SEQ ID NO: 63) |

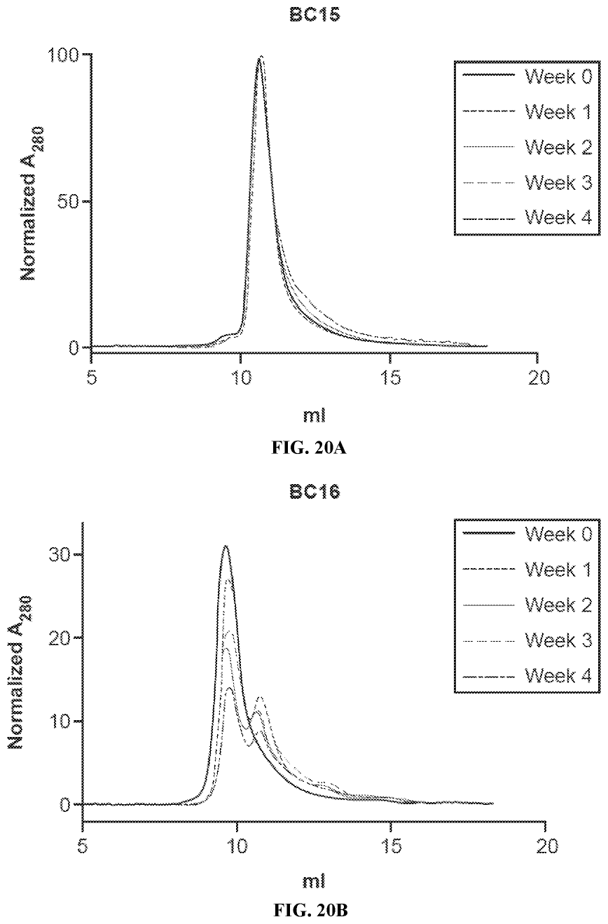

FIG. 20 shows size exclusion chromatography of “BC15” and “BC16” samples at the indicated week of an accelerated stability testing protocol at 40° C. “BC15” remained stable; “BC16” proved to be unstable over time.

### 6.15.8. Example 7: Trivalent 2×1 Bispecific B-Body Construct (“BC1-2×1”)

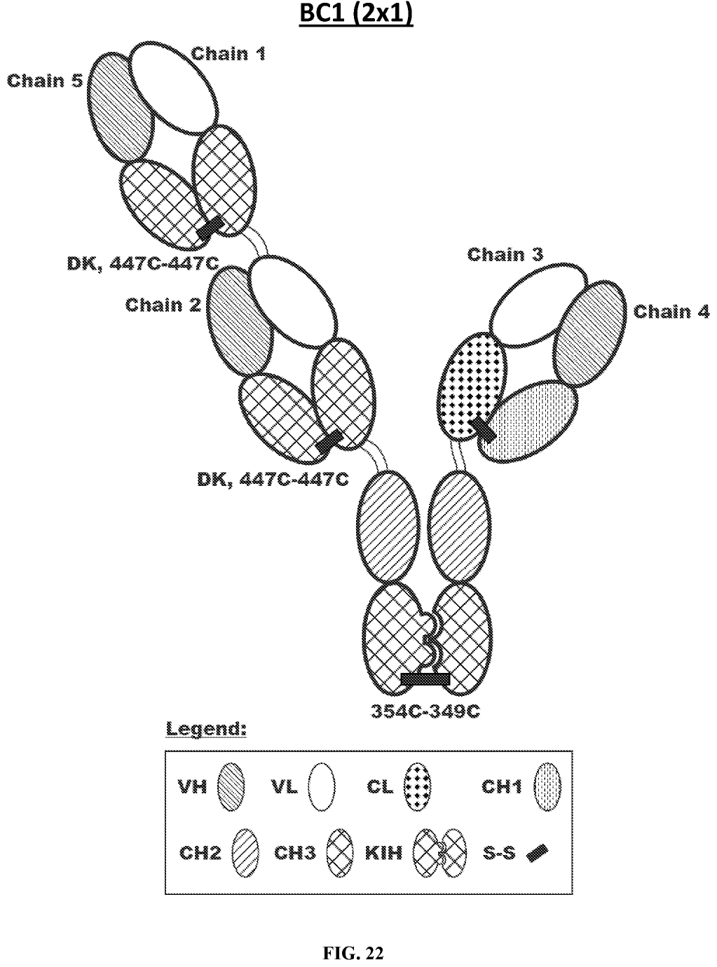

We constructed a trivalent 2×1 bispecific B-Body “BC1-2×1” based on “BC1”. Salient features of the architecture are illustrated in FIG. 22.

In greater detail, using the domain and polypeptide chain references summarized in FIG. 21,
1^{st} polypeptide chain
   Domain N=VL (“Antigen A”)
   Domain O=CH3 (T366K, 447C)
   Domain A=VL (“Antigen A”)
   Domain B=CH3 (T366K, 447C)
   Domain D=CH2
   Domain E=CH3 (Knob, 354C)
5^{th} polypeptide chain (=“BC1” chain 2)
   Domain P=VH (“Antigen A”)
   Domain Q=CH3 (L351D, 447C)
2^{nd} polypeptide chain (=“BC1” chain 2)
   Domain F=VH (“Antigen A”)
   Domain G=CH3 (L351D, 447C)
3^{rd} polypeptide chain (=“BC1” chain 3)
   Domain H=VL (“Nivo”)
   Domain I=CL (Kappa)
   Domain J=CH2
   Domain K=CH3 (Hole, 349C)
4^{th} polypeptide chain (=“BC1” chain 4)
   Domain L=VH (“Nivo”)
   Domain M=CH1.

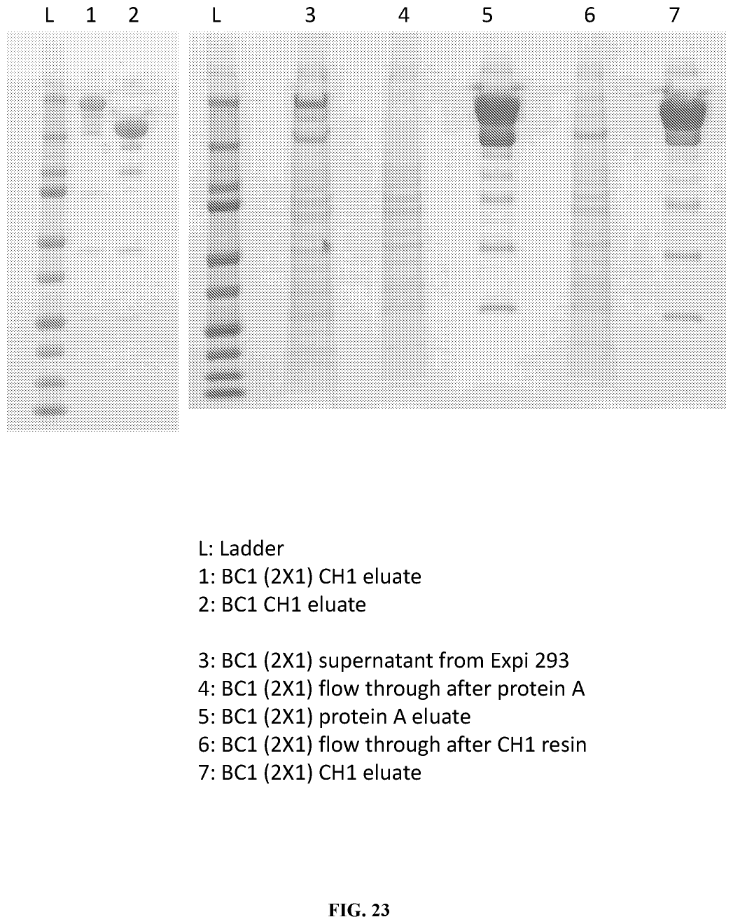

FIG. 23 shows non-reducing SDS-PAGE of protein expressed using the ThermoFisher Expi293 transient transfection system.

Lane 1 shows the eluate of the trivalent 2×1 “BC1-2×1” protein following one-step purification using the CaptureSelect™ CH1 affinity resin. Lane 2 shows the lower molecular weight, faster migrating, bivalent “BC1” protein following one-step purification using the CaptureSelect™ CH1 affinity resin. Lanes 3-5 demonstrate purification of “BC1-2×1” using protein A. Lanes 6 and 7 show purification of “BC1-2×1” using CH1 affinity resin.

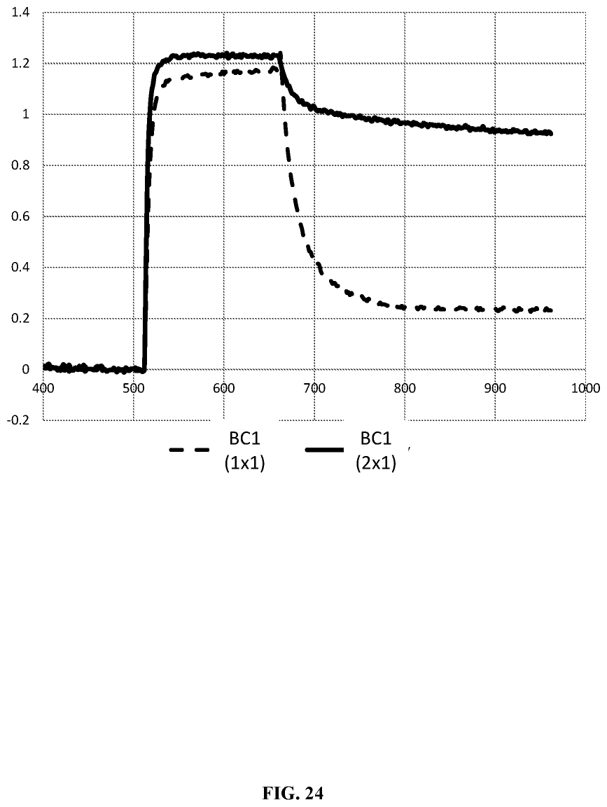

FIG. 24 compares the avidity of the bivalent “BC1” construct to the avidity of the trivalent 2×1 “BC1-2×1” construct using an Octet (Pall ForteBio) analysis. Biotinylated antigen “A” is immobilized on the surface, and the antibody constructs are passed over the surface for binding analysis.

### 6.15.9. Example 8: Trivalent 2×1 Trispecific B-Body Construct (“TB111”)

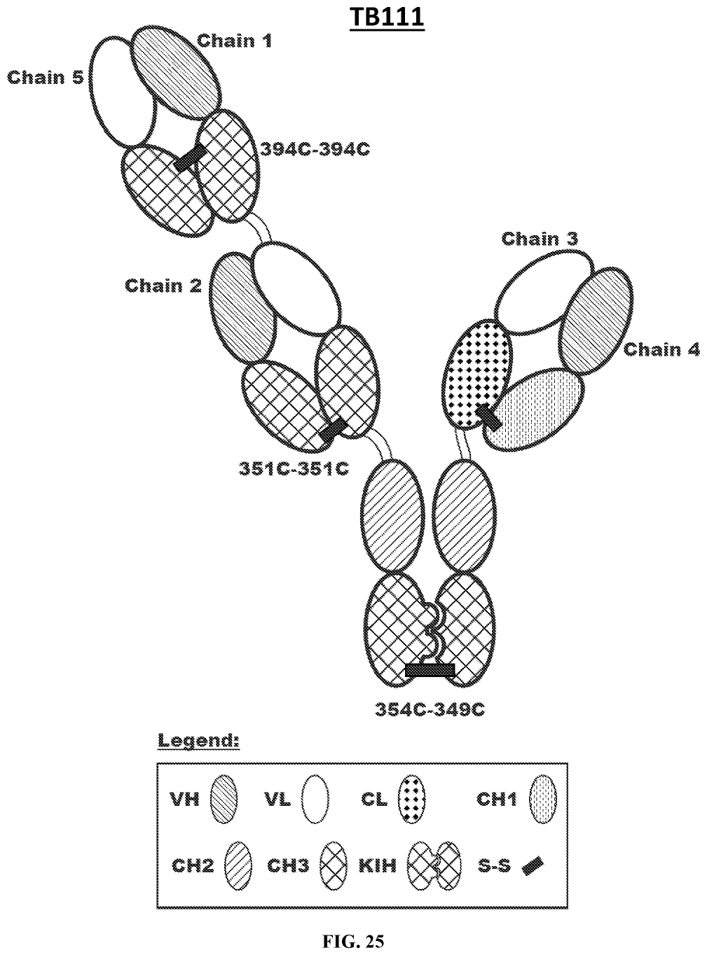

We designed a trivalent 2×1 trispecific molecule, “TB111”, having the architecture schematized in FIG. 25. With reference to the domain naming conventions set forth in FIG. 21, TB111 has the following architecture (“Ada” indicates a V region from adalimumab):
polypeptide chain 1
   Domain N: VH (“Ada”)
   Domain O: CH3 (T366K, 394C)
   Domain A: VL (“Antigen A”)
   Domain B: CH3 (T366K, 349C)
   Domain D: CH2
   Domain E: CH3 (Knob, 354C) polypeptide chain 5
   Domain P: VL (“Ada”)
   Domain Q: CH3 (L351D, 394C)
polypeptide chain 2
   Domain F: VH (“Antigen A”)
   Domain G: CH3 (L351D, 351C)
polypeptide chain 3
   Domain H: VL (“Nivo”)
   Domain I: CL (kappa)
   Domain J: CH2
   Domain K: CH3 (Hole, 349C)
polypeptide chain 4 (=“BC1” chain 4)
   Domain L: VH (“Nivo”)
   Domain M: CH1

This construct did not express.

### 6.15.10. Example 9: Trivalent 1×2 Bispecific Construct (“BC28-1×2”)

We constructed a trivalent 1×2 bispecific B-Body having the following domain structure:
1^{st} polypeptide chain (=“BC28” chain 1) (SEQ ID NO:24)
   Domain A=VL (Antigen “A”)
   Domain B=CH3 (Y349C; 445P, 446G, 447K insertion)
   Domain D=CH2
   Domain E=CH3 (S354C, T366W)
2^{nd} polypeptide chain (=“BC28” chain 2) (SEQ ID NO:25)
   Domain F=VH (Antigen “A”)
   Domain G=CH3 (S354C; 445P, 446G, 447K insertion)
3^{rd} polypeptide chain (SEQ ID NO:37)
   Domain R=VL (Antigen “A”)
   Domain S=CH3 (Y349C; 445P, 446G, 447K insertion)
   Linker=GSGSGS
   Domain H=VL (“Nivo”)
   Domain I=CL
   Domain J=CH2
   Domain K=CH3 (Y349C, D356E, L358M, T366S, L368A, Y407V)
4^{th} polypeptide chain (=“BC1” chain 4) (SEQ ID NO:11):
   Domain L=VH (“Nivo”)
   Domain M=CH1.
6^{th} polypeptide chain (=“BC28” chain 2) (SEQ ID NO:25)
   Domain T=VH (Antigen “A”)
   Domain U=CH3 (S354C; 445P, 446G, 447K insertion)

The A:F antigen binding site is specific for “Antigen A”, as is the H:L binding antigen binding site. The R:T antigen binding site is specific for PD. The specificity of this construct is thus Antigen “A”×(PD1-Antigen “A”).

### 6.15.11. Example 10: Trivalent 1×2 Bispecific Construct (“CTLA4-4× Nivo×CTLA4-4”)

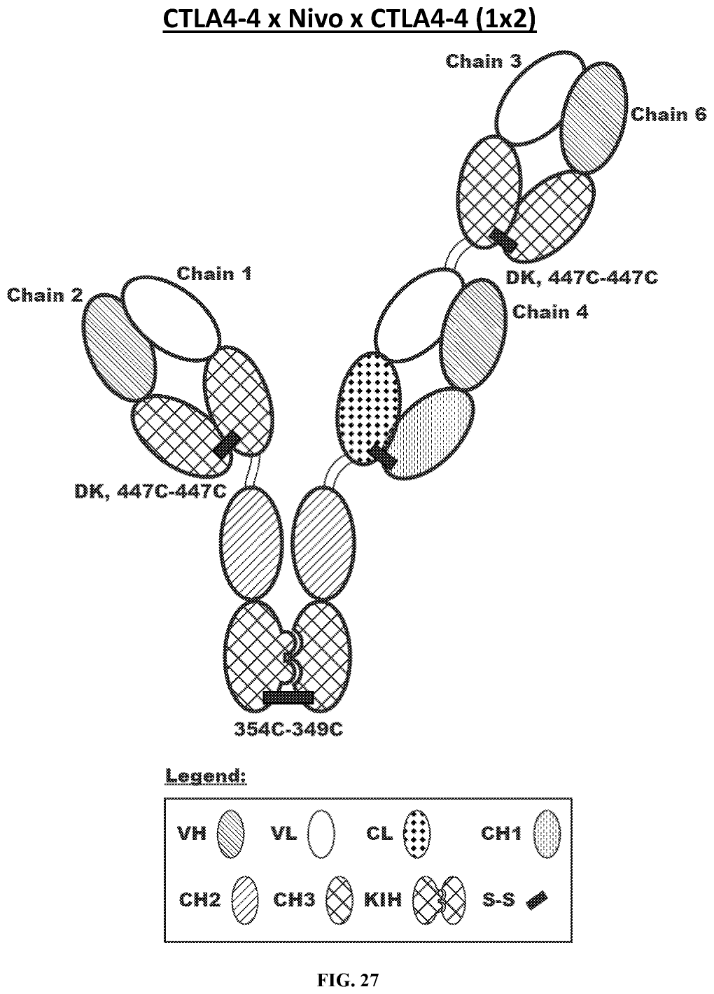

We constructed a trivalent 1×2 bispecific molecule having the general structure schematized in FIG. 27 (“CTLA4-4×Nivo×CTLA4-4”). Domain nomenclature is set forth in FIG. 26.

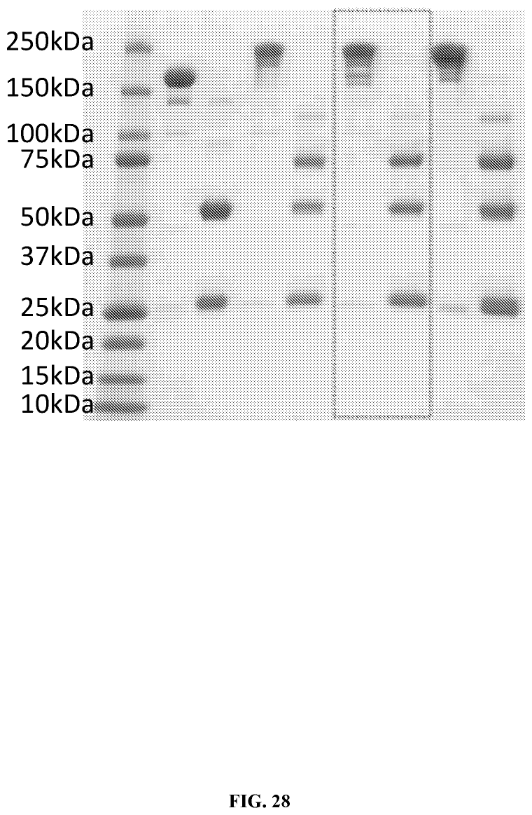

FIG. 28 is a SDS-PAGE gel in which the lanes showing the “CTLA4-4×Nivo×CTLA4-4” construct under non-reducing and reducing conditions have been boxed.

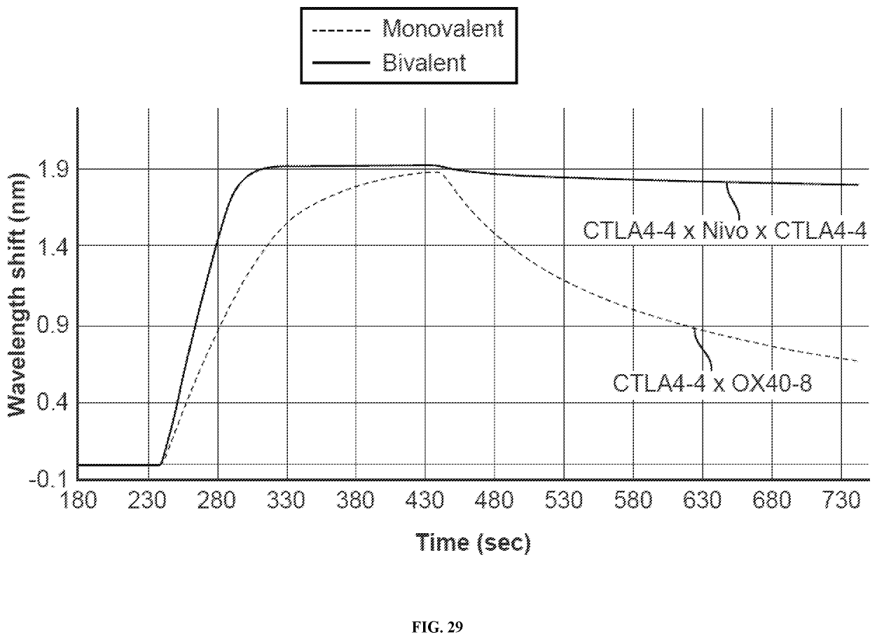

FIG. 29 compares antigen binding of two antibodies: “CTLA4-4×OX40-8” and “CTLA4-4×Nivo×CTLA4-4”. “CTLA4-4×OX40-8” binds to CTLA4 monovalently; while “CTLA4-4×Nivo×CTLA4-4” bind to CTLA4 bivalently.

### 6.15.12. Example 11: Trivalent 1×2 Trispecific Construct “BC28-1×1×1a”

We constructed a trivalent 1×2 trispecific molecule having the general structure schematized in FIG. 30. With reference to the domain nomenclature set forth in FIG. 26,
1^{st} polypeptide chain (=“BC28” chain1) [SEQ ID NO:24]
   Domain A=VL (Antigen “A”)
   Domain B=CH3 (Y349C; 445P, 446G, 447K insertion)
   Domain D=CH2
   Domain E=CH3 (S354C, T366W)
2^{nd} polypeptide chain (=“BC28” chain 2) (SEQ ID NO:25)
   Domain F=VH (Antigen “A”)
   Domain G=CH3 (S354C; 445P, 446G, 447K insertion)
3^{rd} polypeptide chain (SEQ ID NO:45)
   Domain R=VL (CTLA4-4)
   Domain S=CH3 (T366K; 445K, 446S, 447C insertion)
   Linker=GSGSGS
   Domain H=VL (“Nivo”)
   Domain I=CL
   Domain J=CH2
   Domain K=CH3 (Y349C, D356E, L358M, T366S, L368A, Y407V)
4^{th} polypeptide chain (=“BC1” chain 4) (SEQ ID NO:11)
   Domain L=VH (“Nivo”)
   Domain M=CH1.
6^{th} polypeptide chain (=hCTLA4-4 chain2) (SEQ ID NO:53)
   Domain T=VH (CTLA4)
   Domain U=CH3 (L351D, 445G, 446E, 447C insertion)

The antigen binding sites of this trispecific construct were:

Antigen binding site A:F was specific for “Antigen A”

Antigen binding site H:L was specific for PD1 (nivolumab sequence) Antigen binding site R:T was specific for CTLA4.

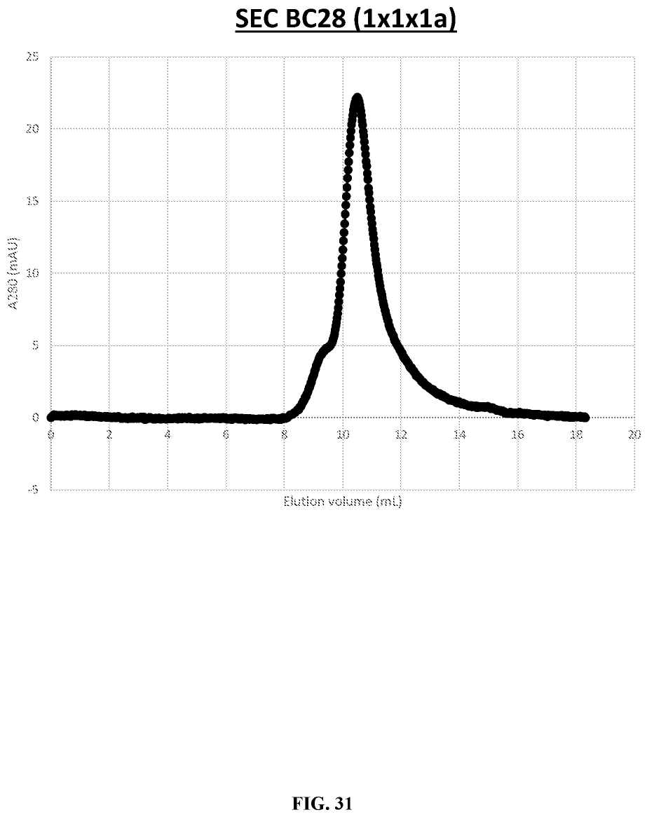

FIG. 31 shows size exclusion chromatography with “BC28-1×1×1a” following transient expression and one-step purification using the CaptureSelect™ CH1 affinity resin, demonstrating a single well-defined peak.

### 6.15.13. Example 12: SDS-PAGE Analysis of Bivalent and Trivalent Constructs

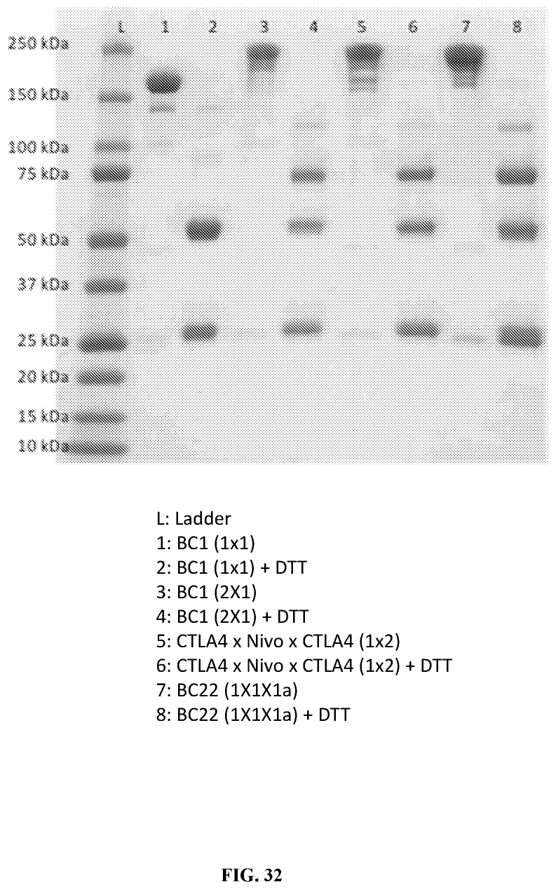

FIG. 32 shows a SDS-PAGE gel with various constructs, each after transient expression and one-step purification using the CaptureSelect™ CH1 affinity resin, under non-reducing and reducing conditions.

Lanes 1 (nonreducing conditions) and 2 (reducing conditions, +DTT) are the bivalent 1×1 bispecific construct “BC1”. Lanes 3 (nonreducing) and 4 (reducing) are the trivalent bispecific 2×1 construct “BC1-2×1” (see Example 7). Lanes 5 (nonreducing) and 6 (reducing) are the trivalent 1×2 bispecific construct “CTLA4-4×Nivo×CTLA4-4” (see Example 10). Lanes 7 (nonreducing) and 8 (reducing) are the trivalent 1×2 trispecific “BC28-1×1×1a” construct described in Example 11.

The SDS-PAGE gel demonstrates the complete assembly of each construct, with the predominant band in the non-reducing gel appearing at the expected molecular weight for each construct.

### 6.15.14. Example 13: Binding Analysis

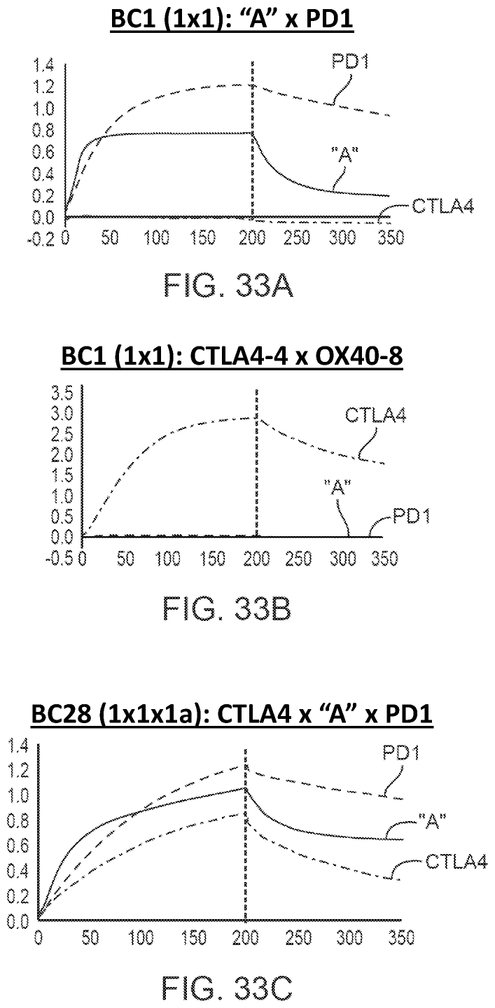

FIG. 33 shows Octet binding analyses to 3 antigens: PD1, Antigen “A”, and CTLA-4. In each instance, the antigen is immobilized and the B-Body is the analyte. For reference, 1×1 bispecifics “BC1” and “CTLA4-4×OX40-8” were also compared to demonstrate 1×1 B-Bodies bind specifically only to antigens for which the antigen binding sites were selected.

FIG. 33A shows binding of “BC1” to PD1 and to Antigen “A”, but not CTLA4. FIG. 33B shows binding of a bivalent bispecific 1×1 construct “CTLA4-4×OX40-8” to CTLA4, but not to Antigen “A” or PD1. FIG. 33C shows the binding of the trivalent trispecific 1×2 construct, “BC28-1×1×1a” to PD1, Antigen “A”, and CTLA4.

### 6.15.15. Example 14: Tetravalent Constructs

FIG. 35 shows the overall architecture of a 2×2 tetravalent bispecific construct “BC22-2×2”. The 2×2 tetravalent bispecific was constructed with “BC1” scaffold by duplicating each variable domain-constant domain segment. Domain nomenclature is schematized in FIG. 34.

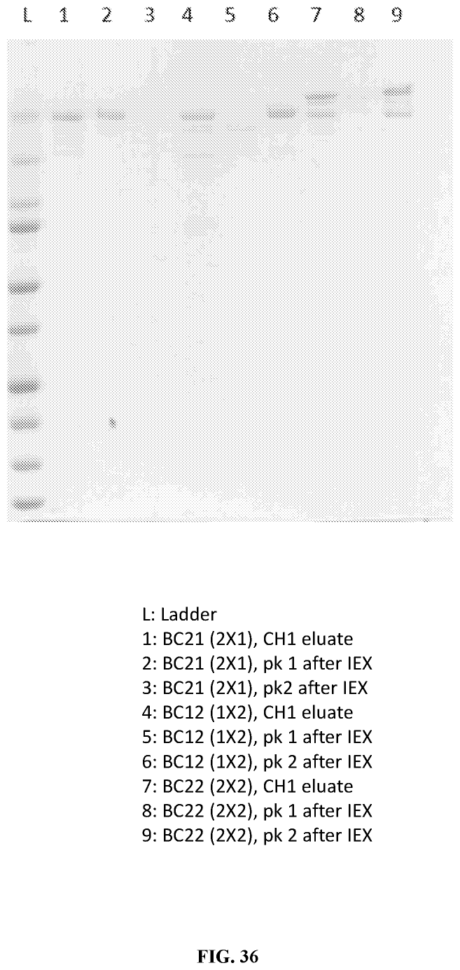

FIG. 36 is a SDS-PAGE gel. Lanes 7-9 show the “BC22-2×2” tetravalent construct respectively following one-step purification using the CaptureSelect™ CH1 affinity resin (“CH1 eluate”), and after an additional ion exchange chromatography purification (lane 8, “pk 1 after IEX”; lane 9, “pk 2 after IEX”). Lanes 1-3 are the trivalent 2×1 construct “BC21-2×1” after CH1 affinity purification (lane 1) and, in lanes 2 and 3, subsequent ion exchange chromatography. Lanes 4-6 are the 1×2 trivalent construct “BC12-1×2”.

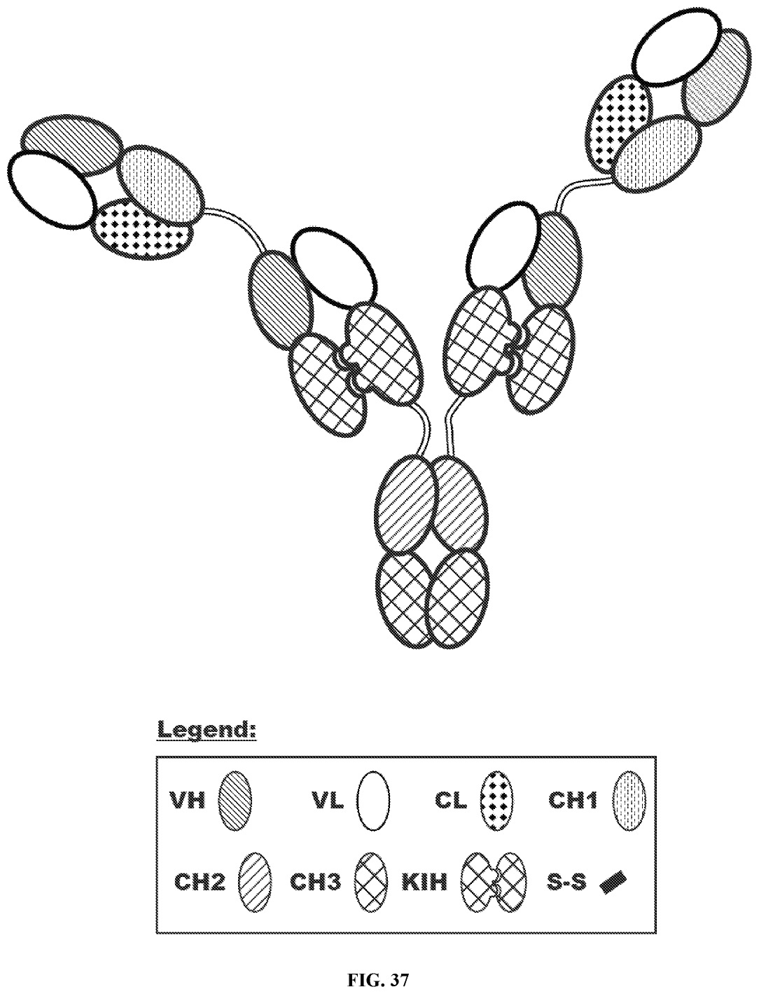

FIG. 37 shows the overall architecture of a 2×2 tetravalent construct.

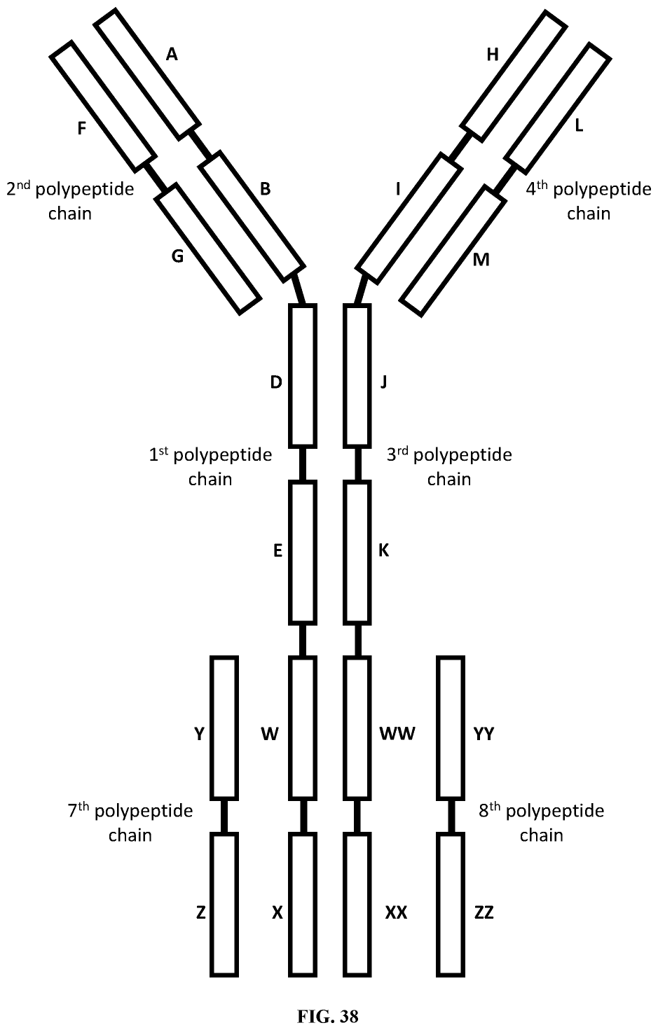

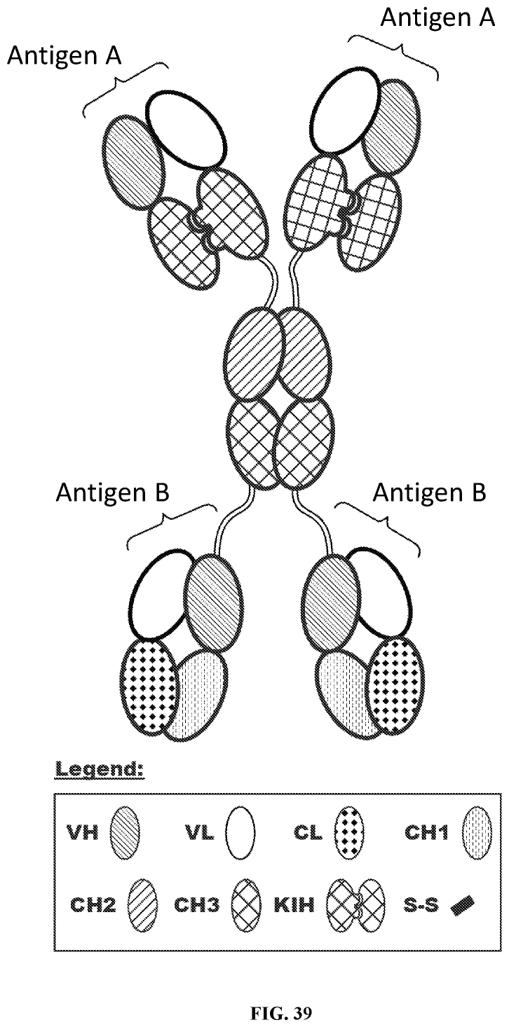

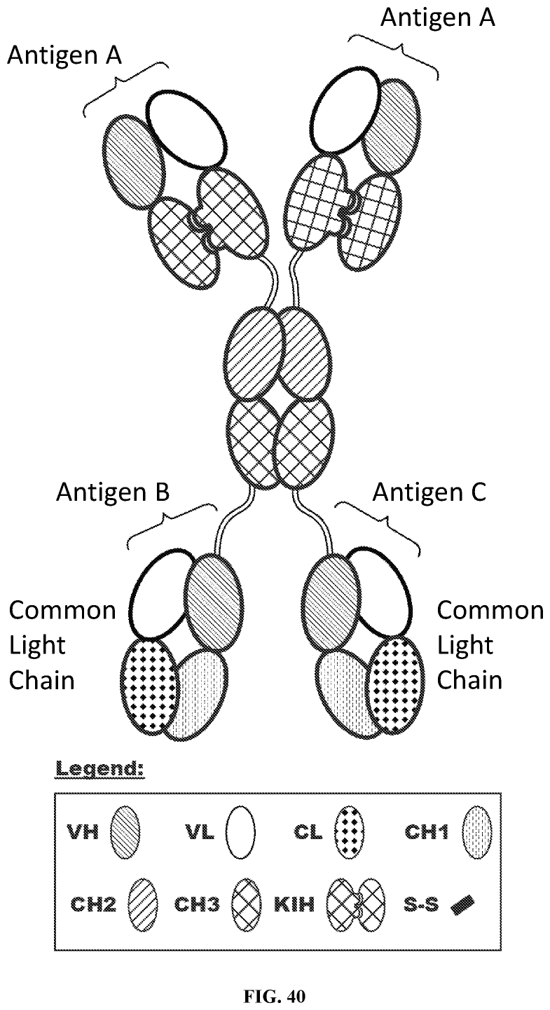

FIGS. 39 and 40 schematize tetravalent constructs having alternative architectures. Domain nomenclature is presented in FIG. 38.

### 6.15.16. Example 15: Bispecific Antigen Engagement by B-Body

A tetravalent bispecific 2×2 B-Body “B-Body-IgG 2×2” was constructed. In greater detail, using the domain and polypeptide chain references summarized in FIG. 38,
1^{st} polypeptide chain
   Domain A=VL (Certolizumab)
   Domain B=CH3 (IgG1, knob)
   Domain D=CH2 (IgG1)
   Domain E=CH3 (IgG1)
   Domain W=VH (Antigen “A”)
   Domain X=CH1 (IgG1)
3^{rd} polypeptide chain (identical to first polypeptide chain)
   Domain H=VL (Certolizumab)
   Domain I=CH3 (IgG1, knob)
   Domain J=CH2 (IgG1)
   Domain K=CH3 (IgG1)
   Domain WW=VH (Antigen “A”)
   Domain XX=CH1 (IgG1)
2^{nd} polypeptide chain
   Domain F=VH (Certolizumab)
   Domain G=CH3 (IgG1, hole)
4^{th} polypeptide chain (identical to third polypeptide chain)
   Domain F=VH (Certolizumab)
   Domain G=CH3 (IgG1, hole)
7^{th} polypeptide chain
   Domain Y=VH (“Antigen A”)
   Domain Z=CL Kappa
8^{th} polypeptide chain (identical to seventh polypeptide chain)
   Domain YY=VH (“Antigen A”)
   Domain ZZ=CL Kappa.

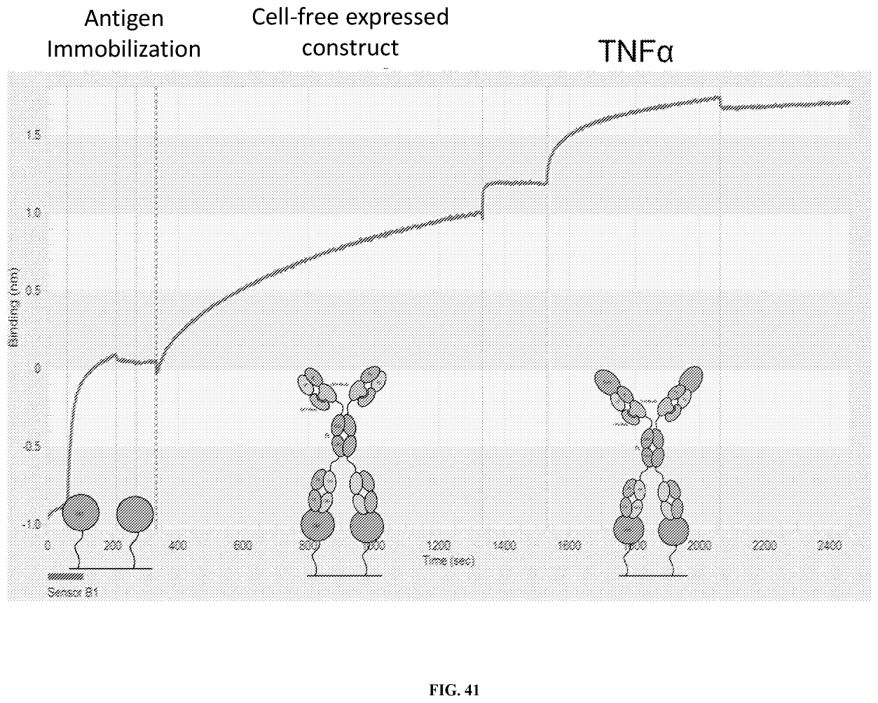

This was cloned and expressed as described in Example 1. Here, the BLI experiment consisted of immobilization of biotinylated antigen “A” on a streptavidin sensor, followed by establishing baseline with 10× kinetic buffer. The sensor was then dipped in cell-free expressed “B-Body-IgG 2×2” followed by establishment of a new baseline. Finally, the sensor was dipped in 100 nM TNFα where a second binding event was observed, confirming the bispecific binding of both antigens by a single “B-Body-IgG 2×2” construct. Results are shown in FIG. 41.

### 6.15.17. Example 16: Antigen-Specific Cell Binding of “BB-IgG 2×2”

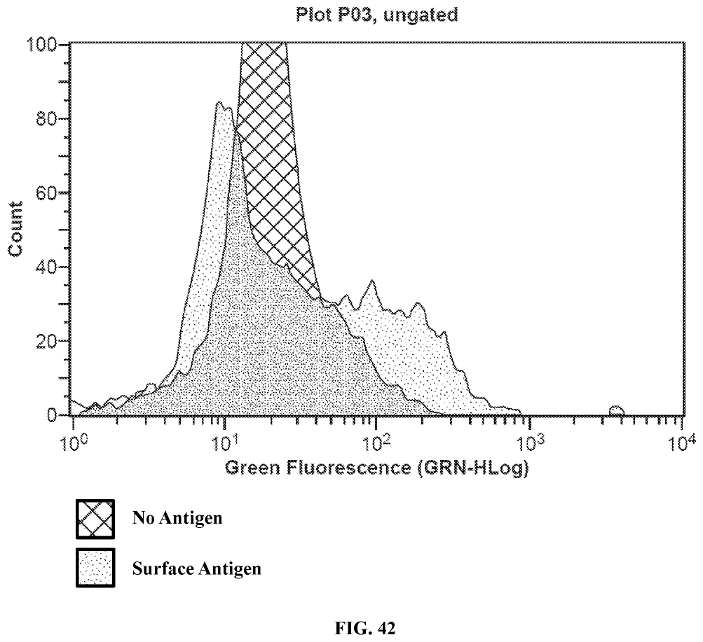

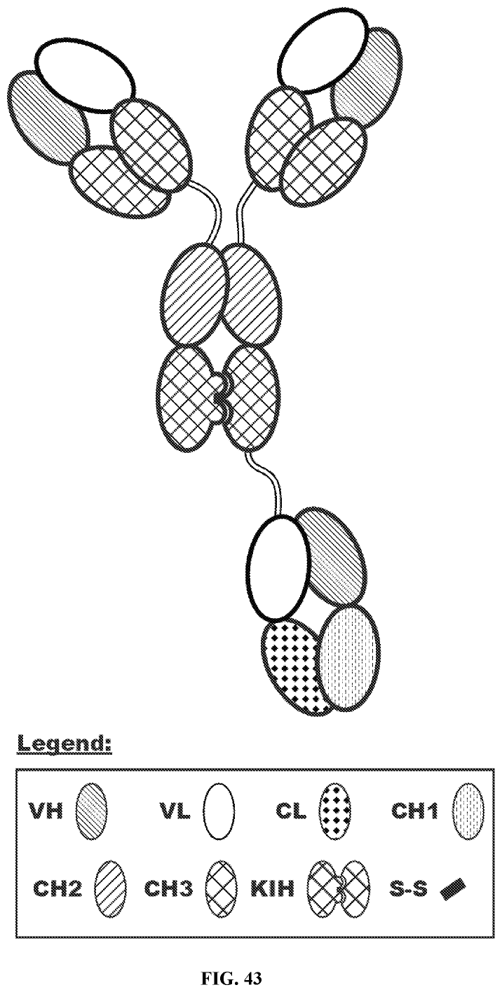

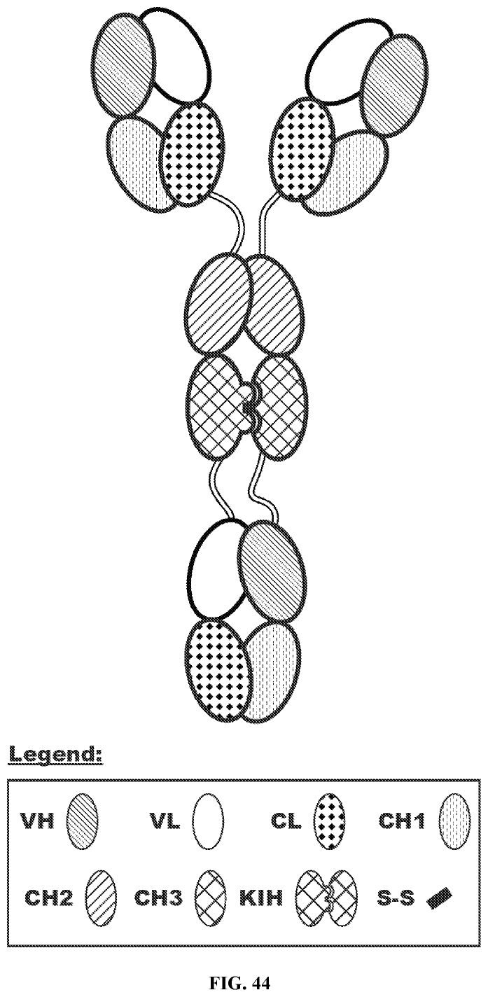

Expi-293 cells were either mock transfected or transiently transfected with Antigen “B” using the Expi-293 Transfection Kit (Life Technologies). Forty-eight hours after transfection, the Expi-293 cells were harvested and fixed in 4% paraformaldehyde for 15 minutes at room temperature. The cells were washed twice in PBS. 200,000 Antigen B or Mock transfected Expi-293 cells were placed in a V-bottom 96 well plate in 100 μL of PBS. The cells were incubated with the “B-Body-IgG 2×2” at a concentration of 3 μg/mL for 1.5 hours at room temperature. The cells were centrifuged at 300×G for 7 minutes, washed in PBS, and incubated with 100 μL of FITC labeled goat-anti human secondary antibody at a concentration of 8 μg/mL for 1 hour at room temperature. The cells were centrifuged at 300×G for 7 minutes, washed in PBS, and cell binding was confirmed by flow cytometry using a Guava easyCyte. Results are shown in FIG. 42.

### 6.15.18. Example 17: SDS-PAGE Analysis of Bivalent and Trivalent Constructs

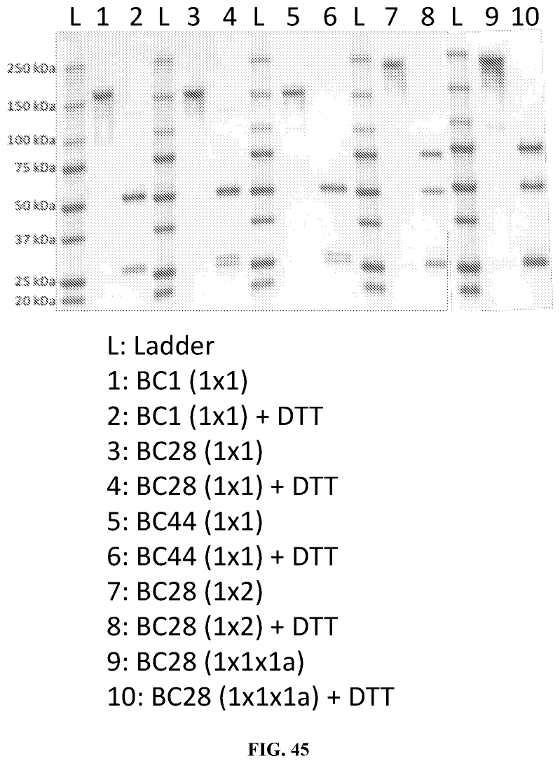

FIG. 45 shows a SDS-PAGE gel with various constructs, each after transient expression and one-step purification using the CaptureSelect™ CH1 affinity resin, under non-reducing and reducing conditions.

Lanes 1 (nonreducing conditions) and 2 (reducing conditions, +DTT) are the bivalent 1×1 bispecific construct “BC1”. Lanes 3 (nonreducing) and 4 (reducing) are the bivalent 1×1 bispecific construct “BC28” (see Example 4). Lanes 5 (nonreducing) and 6 (reducing) are the bivalent 1×1 bispecific construct “BC44” (see Example 5). Lanes 7 (nonreducing) and 8 (reducing) are the trivalent 1×2 bispecific “BC28-1×2” construct (see Example 9). Lanes 9 (nonreducing) and 10 (reducing) are the trivalent 1×2 trispecific “BC28-1×1×1a” construct described in Example 11.

The SDS-PAGE gel demonstrates the complete assembly of each construct, with the predominant band in the non-reducing gel appearing at the expected molecular weight for each construct.

### 6.15.19. Example 18: Stability Analysis of Variable-CH3 Junction Engineering

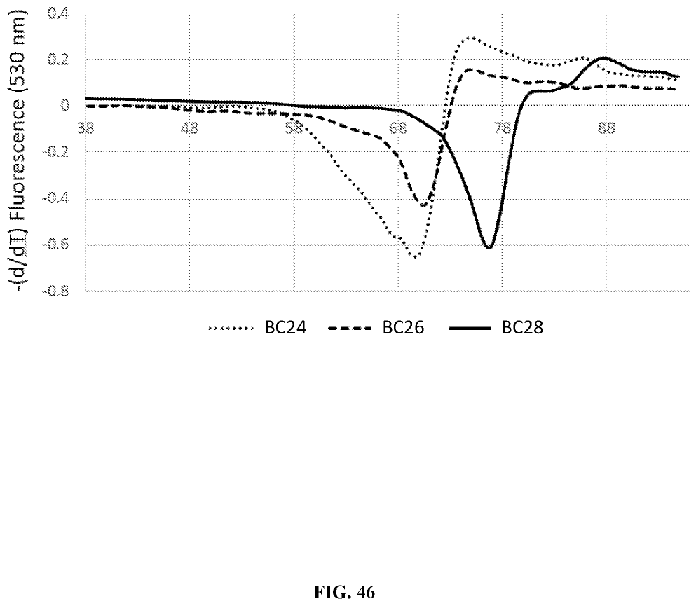

Pairing stability between various junctional variant combinations was assessed. Differential scanning fluorimetry was performed to determine the melting temperature of various junctional variant pairings between VL-CH3 polypeptides from Chain 1 (domains A and B) and VH-CH3 polypeptides from 2 (domains F and G). Junctional variants “BC6jv”, “BC28jv”, “BC30jv”, “BC44jv”, and “BC45jv”, each having the corresponding junctional sequences of “BC6”, “BC28”, “BC30”, “BC44”, and “BC45” found in Table 2 and Table 3 above, demonstrate increased pairing stability with Tm's in the 76-77 degree range (see Table 4). FIG. 46 shows differences in the thermal transitions for “BC24jv”, “BC26jv”, and “BC28jv”, with “BC28jv” demonstrating the greatest stability of the three. The x-axis of the figure is temperature and the y-axis is the change in fluorescence divided by the change in temperature (−dFluor/dTemp). Experiments were performed as described in Niesen et al. (Nature Protocols, (2007) 2, 2212-2221), which is hereby incorporated by reference for all it teaches.

TABLE 4
Melting Temperatures of Junctional Variant Pairs

| JUNCTIONAL VARIANT | MELTING TEMP | MELTING TEMP |
|---|---|---|
| PAIR | #1 (° C.) | #2 (° C.) |
| BC1jv | 69.7 | 55.6 |
| BC5jv | 71.6 | |
| BC6jv | 77 | |
| BC15jv | 68.2 | 54 |
| BC16jv | 65.9 | |
| BC17jv | 68 | |
| BC24jv | 69.7 | |
| BC26jv | 70.3 | |
| BC28jv | 76.7 | |
| BC30jv | 76.8 | |
| BC44jv | 76.2 | |
| BC45jv | 76 | |

### 6.15.20. Example 19: CD3 Candidate Binding Molecules

Various CD3 antibodies were constructed and tested as described below.

### 6.15.20.1. CD3 Binding Arm

A series of CD3 binding arm variants based on a humanized version of the SP34 anti-CD3 antibody (SP34-89, SEQ ID NOs:68 and 69) were engineered with point mutations in either the VH or VL amino acid sequences (SEQ ID Nos:70-73). The various VH and VL sequences were paired together as described in Table 5.

TABLE 5
Anti-CD3 SP34 Binding Arm Variants

| | SP34-89 VL-WT | SP34-89 VL-W57G |
|---|---|---|
| VL/VH Variants | (SEQ ID NO: 69) | (SEQ ID NO: 73) |
| SP34-89 VH-WT | SP34-89 VL-WT/ | SP34-89 VL-W57G/ |
| SEQ ID NO: 68 | SP34-89 VH-WT | SP34-89 VH-WT |
| SP34-89 VH-N30S | SP34-89 VL-WT/ | SP34-89 VL-W57G/ |
| SEQ ID NO: 70 | SP34-89 VH-N30S | SP34-89 VH-N30S |
| SP34-89 VH-G65D | SP34-89 VL-WT/ | SP34-89 VL-W57G/ |
| SEQ ID NO: 71 | SP34-89 VH-G65D | SP34-89 VH-G65D |
| SP34-89 VH-S68T | SP34-89 VL-WT/ | SP34-89 VL-W57G/ |
| SEQ ID NO: 72 | SP34-89 VH-S68T | SP34-89 VH-S68T |

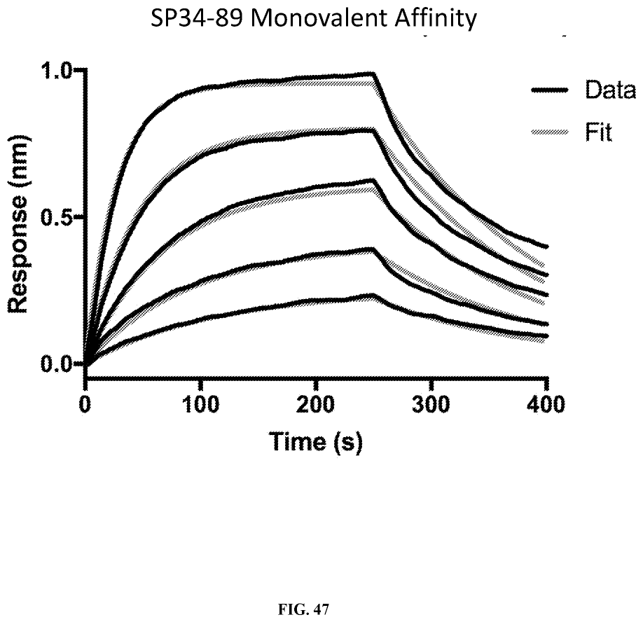

The VL and VH variants were cloned into one arm of a 1×1 BC1 B-Body, while the other arm contained an irrelevant antigen binding site. FIG. 47 demonstrates binding affinity of the non-mutagenized SP34-89 monovalent B-Body as determined by Octet (Pall ForteBio) biolayer interferometry analysis. A two-fold serial dilution (200-12.5 nM) of the construct was used to determine a binding affinity of 23 nM for SP34-89 (kₒₙ=3×10⁵M⁻¹s⁻¹, k_{off}=7.1×10⁻³s⁻¹), matching the affinity for other SP34 variants in the literature. The kinetic affinity also matched the equilibrium binding affinity.

### 6.15.20.2. CD3 Binding Arm Discovery

A chemically synthetic Fab phage library with diversity introduced into the Fab CDRs was screened against CD3 antigens using a monoclonal phage ELISA format where plate-immobilized CD3 variants were assessed for binding to phage, as described above. Phage clones expressing Fabs that recognized CD3 antigens were sequenced. The following table lists CD3 antigen binding site candidates. CD3-8 interestingly was cross-reactive with human and cyno CD3 antigen.

TABLE 8
CD3 Antigen Binding Site Candidates

| NAME | CDR H1 | CDR H2 | CDR H3 | CDR L1 | CDR L2 | CDR L3 |
|---|---|---|---|---|---|---|
| 12B-4 | FYTYSI | YISSYSSYTY | IRLDVL | RASQSVSSAVA | SASSLYS | STSTPY |
| 12B-5 | FSSYYI | SIASLSGQTS | GAEGGM | RASQSVSSAVA | SASSLYS | WGSSLA |
| 12B-6 | FSYYFI | SIDPDFGSTY | AFFTVM | RASQSVSSAVA | SASSLYS | SSSTPR |
| 12B-8 | FKSYYI | GITSYDGYTS | GYYGGM | RASQSVSSAVA | SASSLYS | WGRLLW |
| 12B-9 | FSLYYI | GIWPHAGYTY | SYSISVL | RASQSVSSAVA | SASSLYS | AYTYPY |
| 12B-11 | FSHYSI | YIYPQDDYTE | SIGYGAM | RASQSVSSAVA | SASSLYS | FRSYLR |
| 12B-12 | FYSYYI | SIDPYFGDTT | AHFLAYGL | RASQSVSSAVA | SASSLYS | RSSDLY |
| 12B-13 | FSSYLI | WIYPYDDYTY | DFGFMHGF | RASQSVSSAVA | SASSLYS | WYSSLS |
| 12B-14 | FSSYYI | YIYPYDGYTK | YSYGSFGL | RASQSVSSAVA | SASSLYS | WYSSPV |
| 12B-16 | FLFYRI | QIYPQSGYTS | ADSYRSAF | RASQSVSSAVA | SASSLYS | SWDDLR |
| 12B-17 | FSSYYI | WIYSSGSYTS | GIFSGYGL | RASQSVSSAVA | SASSLYS | RYTSLV |
| 12B-18 | FSYYYI | YISSYRGSTA | SSSYLRIRY | RASQSVSSAVA | SASSLYS | AITSLL |
| 12B-19 | FSSYRI | YIAPYAGYTY | GYYLGQGAF | RASQSVSSAVA | SASSLYS | AISTLW |
| 12B-20 | FVYYYI | WIYSTGGGTS | GYYLTYTGL | RASQSVSSAVA | SASSLYS | LDDSLF |
| 12B-21 | FSYYSI | YISPYKGYTY | YTSYSREAM | RASQSVSSAVA | SASSLYS | SKRVPL |
| 12B-22 | FGYYDI | YISPGGGSTG | LGSLRYYVF | RASQSVSSAVA | SASSLYS | WYSSLL |
| 12B-24 | FSSYGI | GIDPYGTYTS | HFGTGRYGGL | RASQSVSSAVA | SASSLYS | SDSVPL |
| 12B-25 | FSSYGI | YIYPSWGYTV | YRPGVYMYGL | RASQSVSSAVA | SASSLYS | AHSSLP |
| 12B-26 | FTGYYI | SIYPDGGSTI | GGAGSRLVV | RASQSVSSAVA | SASSLYS | LYSSLW |
| 12B-27 | FSSYYI | WISSSGSHTS | GSSHTFFDAL | RASQSVSSAVA | SASSLYS | RGSSLL |
| 12B-29 | FRFYDI | AIYPTRSYTW | GAPFSGYSGM | RASQSVSSAVA | SASSLYS | ASRIPL |
| 12B-30 | FKSYYI | LIDPYSGITT | PSGASALQAM | RASQSVSSAVA | SASSLYS | APSWLA |
| 12B-31 | FPYYLI | VIQPYSSYTA | ESAGYFYGGL | RASQSVSSAVA | SASSLYS | FGSTLY |
| 12B-33 | FPGYSI | YIYPYGGYTY | FSRSRYGVGM | RASQSVSSAVA | SASSLYS | TDSLPL |
| 12B-34 | FDSYII | YITPDIDITY | ASSWTFFEAF | RASQSVSSAVA | SASSLYS | SITSLS |
| 12B-35 | FRSYYI | EISPYTGYTY | GRLVTYSGAL | RASQSVSSAVA | SASSLYS | FDFTLA |
| 12B-37 | FSSYYI | YIYSYDRYTY | GGYYYVVRVM | RASQSVSSAVA | SASSLYS | SSSGLR |
| 12B-42 | FTRYII | SIDPSRGYTK | GLVYYYHYGL | RASQSVSSAVA | SASSLYS | LTLHLS |
| 12B-43 | FSRYAI | YIWPYTGTTI | VAHSSHVGQAM | RASQSVSSAVA | SASSLYS | GKTSPL |
| CD3-3 | FSKYVI | AISSSGGYTL | TIYVPGM | RASQSVSSAVA | SASSLYS | LDYSPW |
| CD3-8 | FPWYA | GISPGTGYTY | GGRYYAM | RASQSVSSAVA | SASSLYS | AWETPL |

### 6.15.21. Example 20: Single Step Purification of Bispecific Antibodies Having CH1 in Both Arms

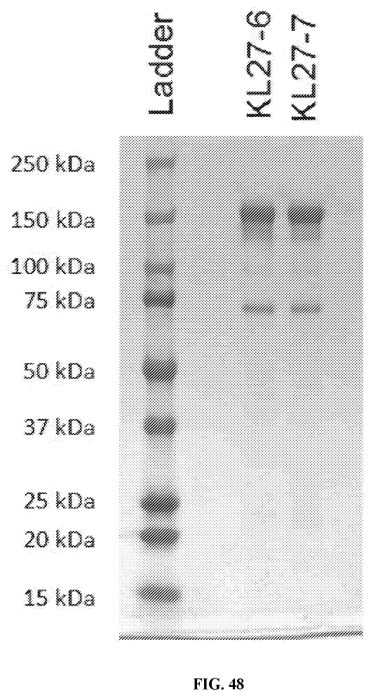

Anti-CH1 purification efficiency of bispecific antibodies was also tested for binding molecules having only standard knob-hole orthogonal mutations introduced into CH3 domains found in their native positions within the Fc portion of the bispecific antibody with no other domain modifications. Therefore, the two antibodies tested, KL27-6 and KL27-7, each contained two CH1 domains, one on each arm of the antibody. As described in more detail in Section 6.15.1, each bispecific antibody was expressed, purified from undesired protein products on an anti-CH1 column, and run on an SDS-PAGE gel. As shown in FIG. 48, a significant band at 75 kDa representing an incomplete bispecific antibody was present, interpreted as a complex containing only (i) a first and second or (ii) third and fourth polypeptide chains with reference to FIG. 3. Thus, methods using anti-CH1 to purify complete bispecific molecules that have a CH1 domain in each arm resulted in background contamination due to incomplete antibody complexes.

### 6.15.22. Example 21: Fc Mutations Reducing Effector Function

A series of engineered Fc variants were generated in the monoclonal IgG1 antibody trastuzumab (Herceptin, “WT-IgG1”) with mutations at positions L234, L235, and P329 of the CH2 domain. The specific mutations for the variants tested are described in Table 9 below and include sFc1 (PALALA), sFc7 (PGLALA), and sFc10 (PKLALA). All variants were produced by Expi293 expression as described herein.

TABLE 9
Fc variants

| Variant | L234 | L235 | P329 |
|---|---|---|---|
| sFc1 | A | A | P |
| sFc2 | G | G | P |
| sFc3 | L | L | A |
| sFc4 | A | A | A |
| sFc5 | G | G | A |
| sFc6 | L | L | G |
| sFc7 | A | A | G |
| sFc8 | G | G | G |
| sFc9 | L | L | K |
| sFc10 | A | A | K |
| sFc11 | G | G | K |
| wt IgG1 | L | L | P |

Stability Analysis

The protein melting temperature was determined using the Protein Thermal Shift Dye Kit (Thermo Fisher), Briefly, proteins of interest were brought to a concentration of 1 mg/ml. Thermal shift dye mix (water, Thermal shift buffer, and Thermal Shift Dye) was added to the protein of interest. The protein/thermal dye mix was added to glass capillary tubes and analyzed using a thermal gradient on a Roche Light Cycler. Proteins were incubated at 37° C. for 2 minutes before initiating a thermal gradient from 37° C. to 99° C. with a temperature increase rate of 0.1° C./sec. Fluorescence increase was measured over time and used to calculate the thermal melting temperature.

Table 10 depicts results from the Protein Thermal Shift experiment above. All variants showed comparable stability as the wild-type IgG.

TABLE 10
melting temperature analysis

| | | | | TM1 | TM2 |
|---|---|---|---|---|---|
| Variant | L234 | L235 | P329 | (° C.) | (° C.) |
| sFc1 | A | A | P | 68.1 | 81.8 |
| sFc2 | G | G | P | 69.7 | 81.7 |
| sFc3 | L | L | A | 65.6 | 81.7 |
| sFc4 | A | A | A | 66.4 | 81.9 |
| sFc5 | G | G | A | 67.5 | 81.5 |
| sFc6 | L | L | G | 64.6 | 81.4 |
| sFc7 | A | A | G | 65.7 | 81.8 |
| sFc8 | G | G | G | 66.2 | 81.8 |
| sFc9 | L | L | K | 64.5 | 81.2 |
| sFc10 | A | A | K | 65.3 | 81.1 |
| sFc11 | G | G | K | 66.2 | 81.5 |
| wt IgG1 | L | L | P | 66.2 | 81.1 |

Interaction of the trastuzumab Fc variants with CD64 was assessed using bio-layer interferometry (Octet/FORTEBIO®). Briefly, Her2 antigen or anti-CH1 antibody was immobilized onto the biosensor tip surface. Antibody solutions comprising the Fc variants listed in Table 9 were flowed over the biosensor, followed by one wash for baseline equilibration followed by an analyte solution containing 200 nM CD64 Response profiles were generated in real time.

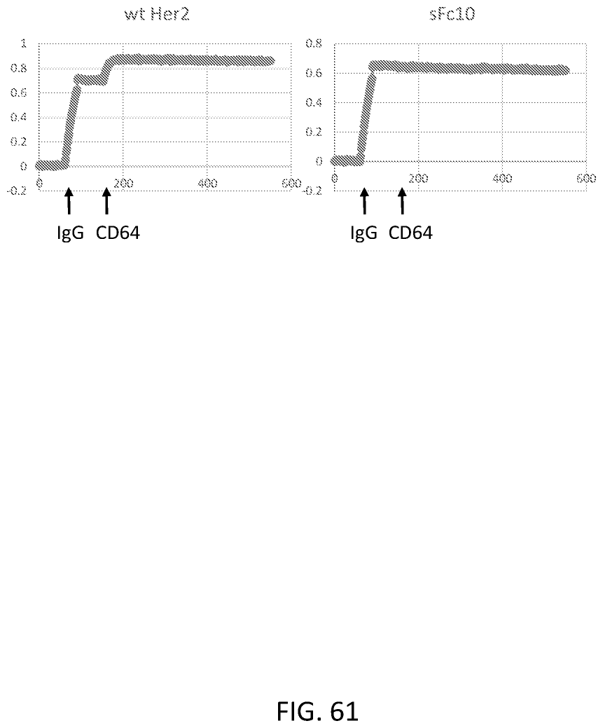

FIG. 61 depicts an exemplary Octet binding analysis. The native trastuzumab antibody (WT Her2) condition revealed a first shift when the antibody solution was flowed over the biosensor, and a second shift when CD64 was added. By contrast, the sFc10 variant condition, while revealing a first shift when the antibody solution was flowed over the biosensor, revealed no shift when CD64 was added.

TABLE 11
summary of Octet analysis

| Variant | L234 | L235 | P329 | Her2 Binding | CD64 Binding? |
|---|---|---|---|---|---|
| sFc1 | A | A | P | Yes (strong) | Yes (weak) |
| sFc2 | G | G | P | Yes (strong) | No |
| sFc3 | L | L | A | Yes (strong) | Yes (strong) |
| sFc4 | A | A | A | Yes (strong) | No |
| sFc5 | G | G | A | Yes (strong) | No |
| sFc6 | L | L | G | Yes (strong) | Yes (strong) |
| sFc7 | A | A | G | Yes (strong) | No |
| sFc8 | G | G | G | Yes (strong) | No |
| sFc9 | L | L | K | Yes (strong) | Yes (weak) |
| sFc10 | A | A | K | Yes (strong) | No |
| sFc11 | G | G | K | Yes (strong) | No |
| wt IgG1 | L | L | P | Yes (strong) | Yes (strong) |

Antibody-Dependent Cell-Mediated Cytotoxicity (ADCC) Assay

The impact of selected Fc mutations on Freia effector function was assessed using the ADCC Bioreporter Assay Kit (Promega). Briefly, a serial dilution of each variant was incubated with SKBR3 cells. The reactions were then incubated at 37° C. in a humidified CO2 incubator with the ADCC Bioassay effector cells according to the manufacturer's protocol and incubated for 6 to 24 hrs. After incubation, the Bio-Glo™ Luciferase Assay Reagent was added to each sample and the luminescent signal was measured with a plate reader with glow-type luminescence read capabilities.

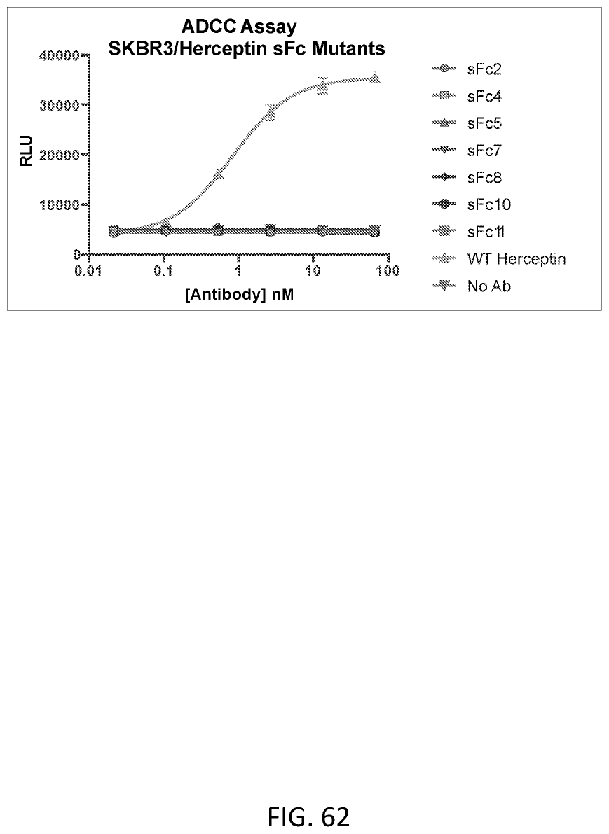

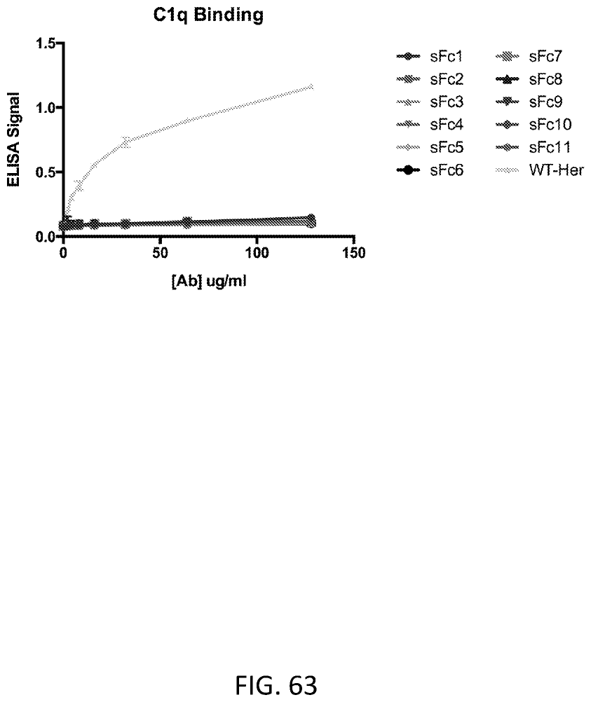

FIG. 62 depicts results from the above ADCC assay. The native trastuzumab antibody (WT Herceptin) exhibited robust ADCC activity. By contrast, the Fc variants exhibited no ADCC activity.

C1q Binding Profiles

The impact of Fc mutations on C1q binding was assessed using an ELISA assay. Up to 128 μg/ml IgG was immobilized for each of the variants. Here the ELISA was performed with 12 μg/ml C1q, 1/400 dilution of the C1q-HRP secondary antibody. Washes and samples were diluted in PBST-BSA (1%).

FIG. 63 depicts results from the above C1q ELISA assay. The native trastuzumab antibody exhibited robust binding to C1q. By contrast, all of the Fc variants exhibited virtually no detectable binding to C1q.

### 6.15.23. Example 22: Construction of a 1×1×1 Trispecific Antibody with Orthogonal CH1/CL Modifications

Antibody Construction

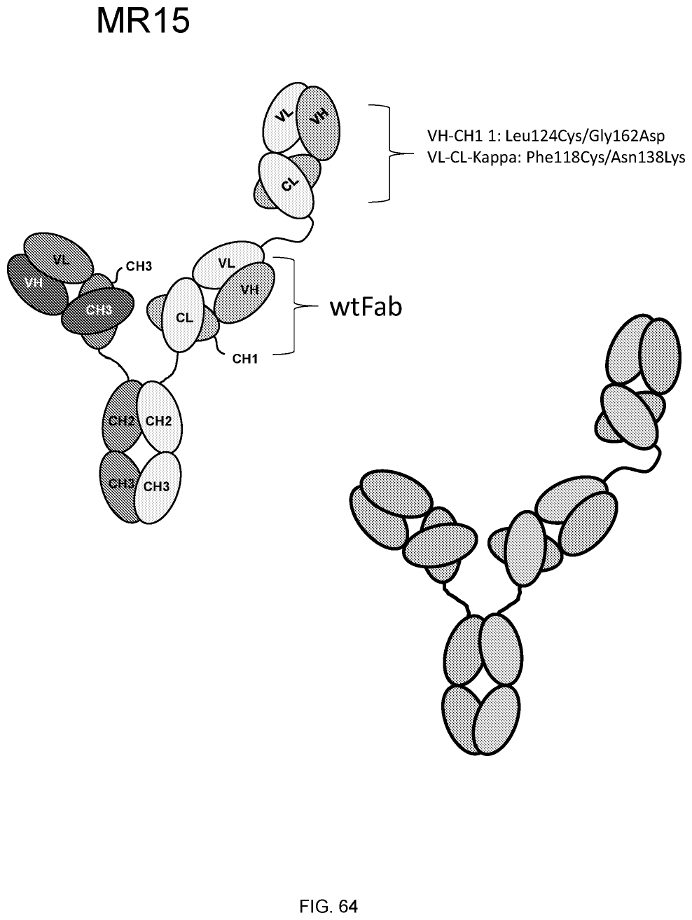

We constructed binding molecule “MR-15” having the architecture depicted in FIG. 64. MR-15 has a first, second, third, fourth, and fifth polypeptide chain, wherein (a) the first polypeptide chain comprises, from N-terminus to C-terminus, a first VL amino acid sequence, a human IgG1 CH3 amino acid sequence with a Y349C mutation and a C-terminal extension incorporating a PGK tripeptide sequence that is followed by the DKTHT motif of an IgG1 hinge region, a human IgG1 CH2 amino acid sequence, and a human IgG1 CH3 amino acid with a S354C and a T366W mutation; (b) the second polypeptide comprises, from N-terminus to C-terminus, a first VH amino acid sequence and a human IgG1 CH3 amino acid sequence with a S354C mutation and a C-terminal extension incorporating a PGK tripeptide sequence; (c) the third polypeptide comprises, from N-terminus to C-terminus, a second VL amino acid sequence which is a wtFab sequence, a first CL kappa amino acid sequence comprising Phe118Cys and Asn138Lys mutations, a third VL amino acid sequence which is a wtFab sequence, a second CL kappa amino acid sequence which is a wtFab sequence; (d) the fourth polypeptide comprises, from N-terminus to C-terminus, a second VH sequence which is a wtFab sequence, a first CH1 sequence comprising Leu128Phe118Cys and Asn138Lys mutations; and (e) the fifth polypeptide comprises, from N-terminus to C-terminus and a third VH sequence which is a wtFab sequence, and a second CH1 sequence which is a wtFab sequence.

SDS-PAGE Analysis

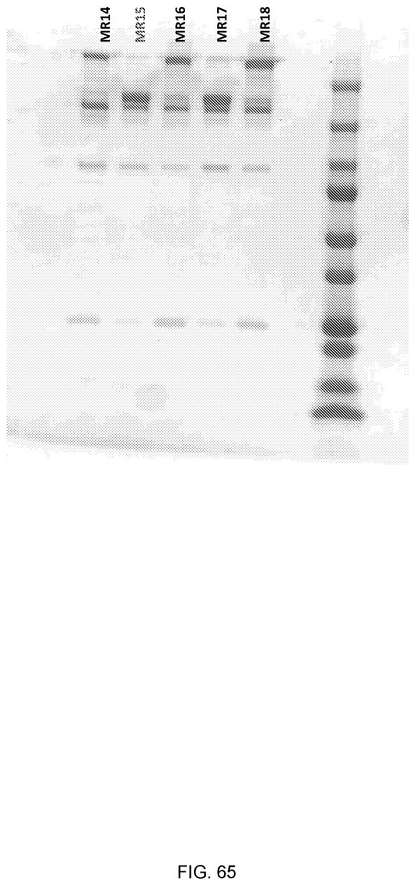

Purified variants were analyzed by non-reducing SDS-PAGE for the presence of complete product, incomplete product, and overall purity. 2 μg of each sample was added to 15 μL SDS loading buffer. Non-reducing samples were incubated at 95° C. for 5 minutes without reducing agent. The reducing and non-reducing samples were loaded into a 4-15% gradient TGX gel (BioRad) with running buffer and run for 30 minutes at 250 volts. Upon completion of the run, the gel was washed with DI water and stained using GelCode Blue Safe Protein Stain (ThermoFisher). The gels were destained with DI water prior to analysis. FIG. 65 depicts an image of the destained gel, showing that MR15 largely assembles into the intended full-length construct with minimal incomplete assembly.

Mass Spectrometry Analysis

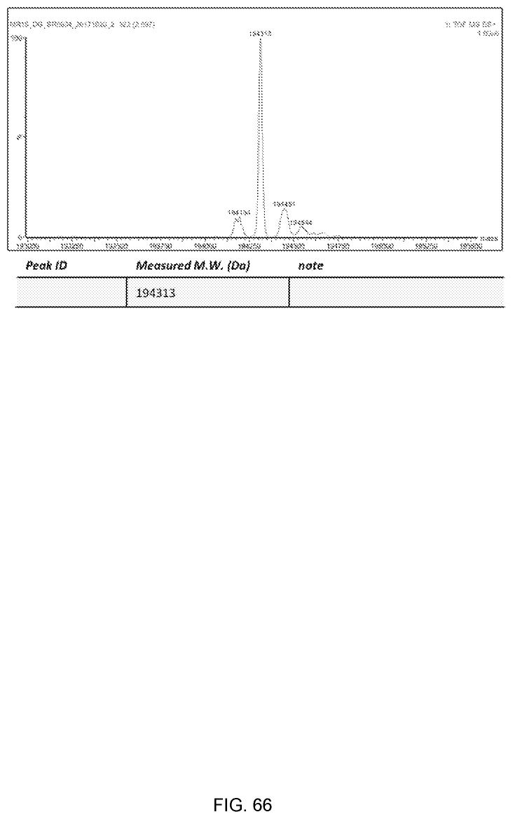

MR variants, including MR15, were purified and analyzed by mass spectrometry to confirm the correct species by molecular weight. Briefly, samples were treated with a cocktail of enzymes to remove glycosylation. Samples were both tested in the reduced format to specifically identify each chain by molecular weight. Samples were also tested under non-reducing conditions to identify the molecular weights of all complexes in the purified samples. FIG. 66 depicts mass spectrogram results, indicating a lack of incompletely assembled variants.

### 6.15.24. Example 23: Bivalent, Bispecific Antibody with Distinct Orthogonal Pair CH1/CL Modifications in Each Arm Improves Full Antibody Assembly

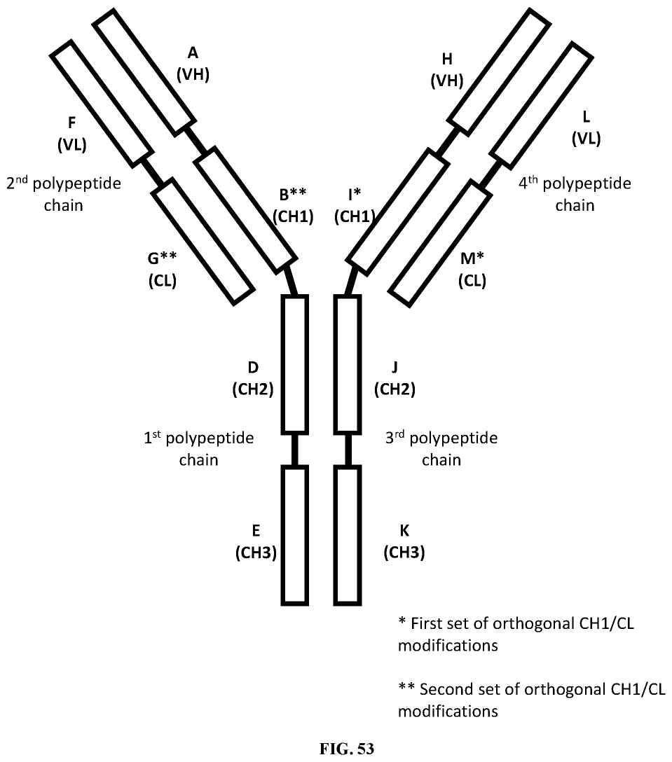

A bivalent, bispecific antibody having distinct orthogonal CH1/CL modifications in each arm, and having a knob-in-hole orthogonal mutation in CH3, was constructed. With reference to FIG. 53, the bivalent, bispecific antibody had the following architecture and amino acid substitutions:
Polypeptide 1: A-B-D-E, wherein
   A=VH
   B=CH1, Leu128Cys/Gly166Asp
   D=CH2
   E=CH3, Ser354Cys/Thr366Trp
Polypeptide 2: F-G, wherein
   F=VL
   G=CL, Phe118Cys/Asn138Lys
Polypeptide 3: H-I-J-K, wherein
   H=VH
   I=CH1, Gly166Lys
   J=CH2
   K=CH3, Tyr349Cys/Thr366Ser/Leu368A1a/Tyr407Val
Polypeptide 4: L-M, wherein
   L=VL
   M=CL, Asn138Asp

Briefly, the four polypeptides were transfected together in Expi293 cells. The supernatants were collected after five days. Antibodies were purified with Protein-A using standard procedures in step 1. In a subsequent step Mono-S ion exchange chromatography was used to isolate the intact, full-length species. Fractions 1, 2, and 3 were collected.

Purified antibodies were analyzed by non-reducing SDS-PAGE for the presence of complete product, incomplete product, and overall purity. 2 μg of each sample was added to 15 μL SDS loading buffer. Non-reducing samples were incubated at 95° C. for 5 minutes without reducing agent. The non-reducing samples were loaded into a 4-15% gradient TGX gel (BioRad) with running buffer and run for 30 minutes at 250 volts. Upon completion of the run, the gel was washed with DI water and stained using GelCode Blue Safe Protein Stain (ThermoFisher). The gels were destained with DI water prior to analysis.

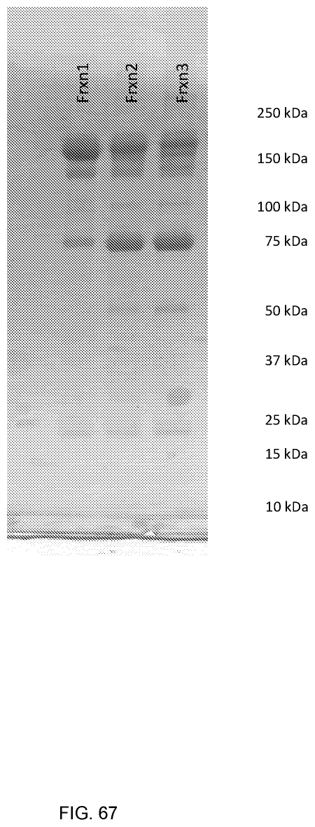

FIG. 67 depicts an image of the destained gel, showing that the fully assembled antibody is largely isolated in the first fraction, and that the second and third fractions generally contain incomplete product.

### 6.15.25. Example 24: Bivalent, Bispecific Antibody with Distinct Orthogonal Pair CH1/CL Modifications in Each Arm Improves Yield, Full Antibody Assembly, and Correct Heavy/Light Chain Pairings in Each Arm

Bivalent, bispecific antibodies having distinct orthogonal CH1/CL modifications in each arm, and having a knob-in-hole orthogonal modification in CH3/CH3, were constructed. With reference to FIG. 53, the bivalent, bispecific antibodies had the following architecture and amino acid substitutions:
Polypeptide 1: A-B-D-E, wherein
   A=VH
   B=CH1, Gly166Asp
   D=CH2
   E=CH3, Ser354Cys/Thr366Trp
Polypeptide 2: F-G, wherein
   F=VL
   G=CL, Asn138Lys
Polypeptide 3: H-I-J-K, wherein
   H=VH
   I=CH1, Gly166Lys
   J=CH2
   K=CH3, Tyr349Cys/Thr366Ser/Leu368A1a/Tyr407Val
Polypeptide 4: L-M, wherein
   L=VL
   M=CL, Asn138Asp

Three different antibodies were expressed by transfection in Expi293 cells. MR30 expressed polypeptides 1, 2, 3, and 4. MR31 expressed polypeptides 1, 3, and 4. MR32 expressed polypeptides 1, 2, and 3. Briefly, the four polypeptides were transfected together in Expi293 cells. The supernatants were collected after five days. Antibodies were purified with Protein-A using standard procedures in step 1.

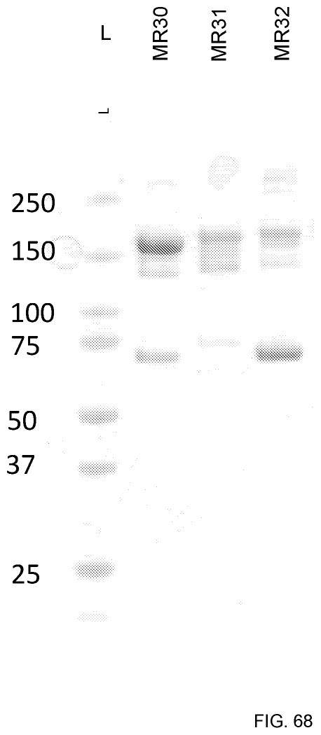

Purified antibodies were analyzed by SDS-PAGE analysis as described in Example A2. Protein concentration was estimated measuring the absorbance at 280 nm which revealed high antibody yields when all four polypeptides were expressed. MR30 yielded 2.5 mg antibody, MR31 yielded 250 μg antibody, and MR32 yielded 2.7 mg antibody. Further, SDS-PAGE analysis, as depicted in FIG. 68, revealed that single-step Protein A purification resulted in overall higher purity of fully-assembled antibody. The MR31 and MR32 lanes revealed that polypeptide 1 interacts poorly with polypeptide 4, and that polypeptide 3 interacts poorly with polypeptide 2. These results indicate that full assembly of the bispecific antibody is driven by the correct pairings of polypeptides 1 and 2, and polypeptides 3 and 4, respectively.

### 6.15.26. Example 25: Bivalent Bispecific B-Body “BA1” with IgA-CH3

### 6.15.26.1. Construction of BA Binding Molecules

We constructed a bivalent bispecific B-Body, termed “BA”, specific for a first antigen PD1 and a second antigen (“Antigen A”). Salient features of the general BA architecture are illustrated in FIG. 3. In greater detail, with domain and polypeptide chain references in accordance with FIG. 3, the architecture of binding molecule “BA” was:
1^{st} polypeptide chain:
   Domain A=VL (“Antigen A”)
   Domain B=C_{H3} (IgA, first CH3 linker)
   Domain D=C_{H2}
   Domain E=C_{H3} (Knob T366W, 354C)
2^{nd} polypeptide chain:
   Domain F=V_{H} (“Antigen A”)
   Domain G=C_{H3} (IgA, second C_{H3} linker)
3^{rd} polypeptide chain:
   Domain H=V_{L} (“Nivo”)
   Domain I=C_{L} (Kappa)
   Domain J=C_{H2}
   Domain K=C_{H3} (Hole, 349C)
4^{th} polypeptide chain:
   Domain L=V_{H} (“Nivo”)
   Domain M=CH1.

The A domain (SEQ ID NO: 12) and F domain (SEQ ID NO: 16) form an antigen binding site (A:F) specific for “Antigen A”. Domain H has the VL sequence from nivolumab (“Nivo”), and domain L has the VH sequence from “Nivo”. H and L associate to form an antigen binding site (H:L) specific for human PD1.

Domain B has the human IgA CH3 sequence, (SEQ ID NO:184), with a first CH3 linker sequence which connects the C-terminus of CH3 to the N-terminus of CH2 (domain D).

Domain G has the human IgA CH3 sequence, (SEQ ID NO:184), with a second CH3 linker sequence which forms a disulfide bridge with the first CH3 linker sequence.

Domain D has the sequence of human IgG1 CH2 domain (SEQ ID NO:20), with a CH2 hinge sequence (SEQ ID NO: 56) appended to the N-terminus of the CH2 domain.

Domain E (SEQ ID NO:15) has the sequence of human IgG1 CH3 with the mutations T366W and S354C.

Domain I (SEQ ID NO:19) has the sequence of human C kappa light chain (Cκ)

Domain J [SEQ ID NO:20] has the sequence of human IgG1 CH2 domain.

Domain K [SEQ ID NO: 21] has the sequence of human IgG1 CH3 with the following changes: Y349C, D356E, L358M, T366S, L368A, Y407V. The 349C mutation introduces a cysteine that is able to form a disulfide bond with the cognate 354C mutation in

Domain E. The 356E and L358M introduce isoallotype amino acids that reduce immunogenicity. The 366S, 368A, and 407V are “hole” mutations.

Domain M [SEQ ID NO: 23] has the sequence of the human IgG1 CH1 region.

### 6.15.26.2. Construction of First BA Variant “BA1”

A first variant of binding molecule BA, “BA1” was constructed as above, wherein the first CH3 linker sequence that connects domain B to domain D is GEC, and wherein the second CH3 linker sequence is GEC.

The four polypeptide chains of BA1 were designed to comprise the following amino acid sequences in Table 12, below.

TABLE 12
BA1 sequences

| BA Chain # | Sequence | SED ID NO: |
|---|---|---|
| BA1.chain 1 | DIQMTQSPSSLSASVGDRVTITCRASQDVNTAVAWYQQ | 100 |
| | KPGKAPKLLIYSASFLYSGVPSRFSGSRSGTDFTLTISSLQ | |
| | PEDFATYYCQQHYTTPPTFGQGTKVEIKRTTFRPEVHLL | |
| | PPPSEELALNELVTLTCLARGFSPKDVLVRWLQGSQELP | |
| | REKYLTWASRQEPSQGTTTFAVTSILRVAAEDWKKGDT | |
| | FSCMVGHEALPLAFTQKTIDRLGECDKTHTCPPCPAPEL | |
| | LGGPSVFLFPPKPKDTLMISRTPEVTCVVVDVSHEDPEV | |
| | KFNWYVDGVEVHNAKTKPREEQYNSTYRVVSVLTVLH | |
| | QDWLNGKEYKCKVSNKALPAPIEKTISKAKGQPREPQV | |
| | YTLPPCRDELTKNQVSLWCLVKGFYPSDIAVEWESNGQ | |
| | PENNYKTTPPVLDSDGSFFLYSKLTVDKSRWQQGNVFS | |
| | CSVMHEALHNHYTQKSLSLSPGK | |
| BA1.chain 2 | EVQLVESGGGLVQPGGSLRLSCAASGFTFSDYDIHWVR | 101 |
| | QAPGKGLEWVGDITPYDGTTNYADSVKGRFTISADTSK | |
| | NTAYLQMNSLRAEDTAVYYCARLVGEIATGFDYWGQG | |
| | TLVTVSSASTFRPEVHLLPPPSEELALNELVTLTCLARGF | |
| | SPKDVLVRWLQGSQELPREKYLTWASRQEPSQGTTTFA | |
| | VTSILRVAAEDWKKGDTFSCMVGHEALPLAFTQKTIDR | |
| | LGEC | |
| BA(all).chain 3 | EIVLTQSPATLSLSPGERATLSCRASQSVSSYLAWYQQK | 102 |
| | PGQAPRLLIYDASNRATGIPARFSGSGSGTDFTLTISSLEP | |
| | EDFAVYYCQQSSNWPRTFGQGTKVEIKRTVAAPSVFIFP | |
| | PSDEQLKSGTASVVCLLNNFYPREAKVQWKVDNALQS | |
| | GNSQESVTEQDSKDSTYSLSSTLTLSKADYEKHKVYAC | |
| | EVTHQGLSSPVTKSFNRGECDKTHTCPPCPAPELLGGPS | |
| | VFLFPPKPKDTLMISRTPEVTCVVVDVSHEDPEVKFNW | |
| | YVDGVEVHNAKTKPREEQYNSTYRVVSVLTVLHQDWL | |
| | NGKEYKCKVSNKALPAPIEKTISKAKGQPREPQVCTLPP | |
| | SREEMTKNQVSLSCAVKGFYPSDIAVEWESNGQPENNY | |
| | KTTPPVLDSDGSFFLVSKLTVDKSRWQQGNVFSCSVMH | |
| | EALHNHYTQKSLSLSPGK | |
| BA(all).chain 4 | QVQLVESGGGVVQPGRSLRLDCKASGITFSNSGMHWV | 103 |
| | RQAPGKGLEWVAVIWYDGSKRYYADSVKGRFTISRDN | |
| | SKNTLFLQMNSLRAEDTAVYYCATNDDYWGQGTLVT | |
| | VSSASTKGPSVFPLAPSSKSTSGGTAALGCLVKDYFPEP | |
| | VTVSWNSGALTSGVHTFPAVLQSSGLYSLSSVVTVPSSS | |
| | LGTQTYICNVNHKPSNTKVDKKVEPPKSC | |

Antibodies were produced by Expi293 expression as described herein.

### 6.15.26.3. BA1 Exhibits Specific Binding to Both PD1 and Antigen A

Interaction of BA1 with PD1 and Antigen A was assessed using bio-layer interferometry)(Octet/FORTEBIO®). Briefly, biotinylated PD1 or biotinylated Antigen A (magenta) was immobilized on streptavidin sensor. BA1 was then added, followed by a dissociation step. Response profiles were generated in real time.

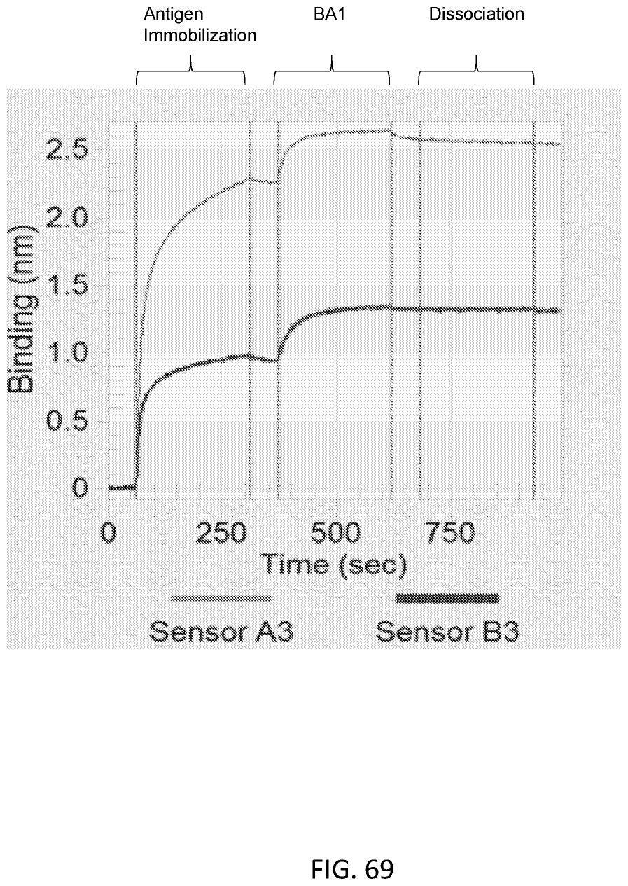

FIG. 69 depicts results from the Octet assay. BA1 contained both binding sites for PD1 and Antigen A.

### 6.15.27. Example 26: Optimization of CH3 Linker Sequences

Variants of BA were constructed as described in Section 6.15.26.1, where the first CH3 linker sequence and second CH3 linker sequence were varied according to the following table. For all BA variants, polypeptides 3 (SEQ ID NO:102) and 4 (SEQ ID NO:103) were unchanged.

Table 13 provides the amino acid sequences for the first CH3 linker and second CH3 linkers used in constructing the BA variants.

TABLE 13
CH3 linker sequences for BA variants

| | First CH3 | Second CH3 |
|---|---|---|
| BA variant # | linker sequence | linker sequence |
| BA1 | GEC | GEC |
| BA2 | AGC | AGKGSC |
| BA3 | AGKGC | AGC |
| BA4 | AGKGSC | AGC |
| BA5 | AGKC | AGC |
| BA9 | AGC | AGC |
| BA10 | AGC | AGKGC |
| BA11 | AGC | AGKGSC |
| BA12 | AGC | AGKC |
| BA13 | AGC | GEC |
| BA14 | AGC | PGKC |
| BA15 | AGKGC | AGC |
| BA16 | AGKGC | AGKGC |
| BA17 | AGKGC | AGKGSC |
| BA18 | AGKGC | AGKC |
| BA19 | AGKGC | GEC |
| BA20 | AGKGC | PGKC |
| BA21 | AGKGSC | AGC |
| BA22 | AGKGSC | AGKGC |
| BA23 | AGKGSC | AGKGSC |
| BA24 | AGKGSC | AGKC |
| BA25 | AGKGSC | GEC |
| BA26 | AGKGSC | PGKC |
| BA27 | AGKC | AGC |
| BA28 | AGKC | AGKGC |
| BA29 | AGKC | AGKGSC |
| BA30 | AGKC | AGKC |
| BA31 | AGKC | GEC |
| BA32 | AGKC | PGKC |
| BA33 | GEC | AGC |
| BA34 | GEC | AGKGC |
| BA35 | GEC | AGKGSC |
| BA36 | GEC | AGKC |
| BA37 | GEC | GEC |
| BA38 | GEC | PGKC |
| BA39 | PGKC | AGC |
| BA40 | PGKC | AGKGC |
| BA41 | PGKC | AGKGSC |
| BA42 | PGKC | AGKC |
| BA43 | PGKC | GEC |
| BA44 | PGKC | PGKC |

Table 14 provides the amino acid sequences for polypeptide 1 of the additional BA variants.

TABLE 14
additional BA variants, chain 1 sequences

| BA# | Chain 1 Sequence | SEQ ID NO: |
|---|---|---|
| BA2 | DIQMTQSPSSLSASVGDRVTITCRASQDVNTAVAWYQQKPGKAPKLLIYSASFL | 104 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGC | |
| BA3 | DIQMTQSPSSLSASVGDRVTITCRASQDVNTAVAWYQQKPGKAPKLLIYSASFL | 105 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGC | |
| BA4 | DIQMTQSPSSLSASVGDRVTITCRASQDVNTAVAWYQQKPGKAPKLLIYSASFL | 106 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGSC | |
| BA5 | DIQMTQSPSSLSASVGDRVTITCRASQDVNTAVAWYQQKPGKAPKLLIYSASFL | 107 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKC | |
| BA9 | DIQMTQSPSSLSASVGDRVTITCRASQDVNTAVAWYQQKPGKAPKLLIYSASFL | 108 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGC | |
| BA10 | DIQMTQSPSSLSASVGDRVTITCRASQDVNTAVAWYQQKPGKAPKLLIYSASFL | 109 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGC | |
| BA11 | DIQMTQSPSSLSASVGDRVTITCRASQDVNTAVAWYQQKPGKAPKLLIYSASFL | 110 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGC | |
| BA12 | DIQMTQSPSSLSASVGDRVTITCRASQDVNTAVAWYQQKPGKAPKLLIYSASFL | 111 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGC | |
| BA13 | DIQMTQSPSSLSASVGDRVTITCRASQDVNTAVAWYQQKPGKAPKLLIYSASFL | 112 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGC | |
| BA14 | DIQMTQSPSSLSASVGDRVTITCRASQDVNTAVAWYQQKPGKAPKLLIYSASFL | 113 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGC | |
| BA15 | DIQMTQSPSSLSASVGDRVTITCRASQDVNTAVAWYQQKPGKAPKLLIYSASFL | 114 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGC | |
| BA16 | DIQMTQSPSSLSASVGDRVTITCRASQDVNTAVAWYQQKPGKAPKLLIYSASFL | 115 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGC | |
| BA17 | DIQMTQSPSSLSASVGDRVTITCRASQDVNTAVAWYQQKPGKAPKLLIYSASFL | 116 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGC | |
| BA18 | DIQMTQSPSSLSASVGDRVTITCRASQDVNTAVAWYQQKPGKAPKLLIYSASFL | 117 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGC | |
| BA19 | DIQMTQSPSSLSASVGDRVTITCRASQDVNTAVAWYQQKPGKAPKLLIYSASFL | 118 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGC | |
| BA20 | DIQMTQSPSSLSASVGDRVTITCRASQDVNTAVAWYQQKPGKAPKLLIYSASFL | 119 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGC | |
| BA21 | DIQMTQSPSSLSASVGDRVTITCRASQDVNTAVAWYQQKPGKAPKLLIYSASFL | 120 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGSC | |
| BA22 | DIQMTQSPSSLSASVGDRVTITCRASQDVNTAVAWYQQKPGKAPKLLIYSASFL | 121 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGSC | |
| BA23 | DIQMTQSPSSLSASVGDRVTITCRASQDVNTAVAWYQQKPGKAPKLLIYSASFL | 122 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGSC | |
| BA24 | DIQMTQSPSSLSASVGDRVTITCRASQDVNTAVAWYQQKPGKAPKLLIYSASFL | 123 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGSC | |
| BA25 | DIQMTQSPSSLSASVGDRVTITCRASQDVNTAVAWYQQKPGKAPKLLIYSASFL | 124 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGSC | |
| BA26 | DIQMTQSPSSLSASVGDRVTITCRASQDVNTAVAWYQQKPGKAPKLLIYSASFL | 125 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKGSC | |
| BA27 | DIQMTQSPSSLSASVGDRVTITCRASQDVNTAVAWYQQKPGKAPKLLIYSASFL | 126 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKC | |
| BA28 | DIQMTQSPSSLSASVGDRVTITCRASQDVNTAVAWYQQKPGKAPKLLIYSASFL | 127 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKC | |
| BA29 | DIQMTQSPSSLSASVGDRVTITCRASQDVNTAVAWYQQKPGKAPKLLIYSASFL | 128 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKC | |
| BA30 | DIQMTQSPSSLSASVGDRVTITCRASQDVNTAVAWYQQKPGKAPKLLIYSASFL | 129 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKC | |
| BA31 | DIQMTQSPSSLSASVGDRVTITCRASQDVNTAVAWYQQKPGKAPKLLIYSASFL | 130 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKC | |
| BA32 | DIQMTQSPSSLSASVGDRVTITCRASQDVNTAVAWYQQKPGKAPKLLIYSASFL | 131 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | AGKC | |
| BA33 | DIQMTQSPSSLSASVGDRVTITCRASQDVNTAVAWYQQKPGKAPKLLIYSASFL | 132 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | GEC | |
| BA34 | DIQMTQSPSSLSASVGDRVTITCRASQDVNTAVAWYQQKPGKAPKLLIYSASFL | 133 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | GEC | |
| BA35 | DIQMTQSPSSLSASVGDRVTITCRASQDVNTAVAWYQQKPGKAPKLLIYSASFL | 134 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | GEC | |
| BA36 | DIQMTQSPSSLSASVGDRVTITCRASQDVNTAVAWYQQKPGKAPKLLIYSASFL | 135 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | GEC | |
| BA37 | DIQMTQSPSSLSASVGDRVTITCRASQDVNTAVAWYQQKPGKAPKLLIYSASFL | 136 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | GEC | |
| BA38 | DIQMTQSPSSLSASVGDRVTITCRASQDVNTAVAWYQQKPGKAPKLLIYSASFL | 137 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | GEC | |
| BA39 | DIQMTQSPSSLSASVGDRVTITCRASQDVNTAVAWYQQKPGKAPKLLIYSASFL | 138 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | PGKC | |
| BA40 | DIQMTQSPSSLSASVGDRVTITCRASQDVNTAVAWYQQKPGKAPKLLIYSASFL | 139 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | PGKC | |
| BA41 | DIQMTQSPSSLSASVGDRVTITCRASQDVNTAVAWYQQKPGKAPKLLIYSASFL | 140 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | PGKC | |
| BA42 | DIQMTQSPSSLSASVGDRVTITCRASQDVNTAVAWYQQKPGKAPKLLIYSASFL | 141 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | PGKC | |
| BA43 | DIQMTQSPSSLSASVGDRVTITCRASQDVNTAVAWYQQKPGKAPKLLIYSASFL | 142 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | PGKC | |
| BA44 | DIQMTQSPSSLSASVGDRVTITCRASQDVNTAVAWYQQKPGKAPKLLIYSASFL | 143 |
| | YSGVPSRFSGSRSGTDFTLTISSLQPEDFATYYCQQHYTTPPTFGQGTKVEIKR | |
| | TTFRPEVHLLPPPSEELALNELVTLTCLARGFSPKDVLVRWLQGSQELPREKYLT | |
| | WASRQEPSQGTTTFAVTSILRVAAEDWKKGDTFSCMVGHEALPLAFTQKTIDRL | |
| | PGKC | |

Table 15 provides the amino acid sequences for polypeptide 2 of the additional BA variants.

TABLE 15
additional BA variants, chain 2 sequences

| BA# | Chain 2 Sequence (base + linker) | SEQ ID NO: |
|---|---|---|
| BA2 | EVQLVESGGGLVQPGGSLRLSCAASGFTFSDYDIHWVRQAPGKGLEWVGDITPY | 144 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKGSC | |
| BA3 | EVQLVESGGGLVQPGGSLRLSCAASGFTFSDYDIHWVRQAPGKGLEWVGDITPY | 145 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGC | |
| BA4 | EVQLVESGGGLVQPGGSLRLSCAASGFTFSDYDIHWVRQAPGKGLEWVGDITPY | 146 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGC | |
| BA5 | EVQLVESGGGLVQPGGSLRLSCAASGFTFSDYDIHWVRQAPGKGLEWVGDITPY | 147 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGC | |
| BA9 | EVQLVESGGGLVQPGGSLRLSCAASGFTFSDYDIHWVRQAPGKGLEWVGDITPY | 148 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGC | |
| BA10 | EVQLVESGGGLVQPGGSLRLSCAASGFTFSDYDIHWVRQAPGKGLEWVGDITPY | 149 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKGC | |
| BA11 | EVQLVESGGGLVQPGGSLRLSCAASGFTFSDYDIHWVRQAPGKGLEWVGDITPY | 150 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKGSC | |
| BA12 | EVQLVESGGGLVQPGGSLRLSCAASGFTFSDYDIHWVRQAPGKGLEWVGDITPY | 151 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKC | |
| BA13 | EVQLVESGGGLVQPGGSLRLSCAASGFTFSDYDIHWVRQAPGKGLEWVGDITPY | 152 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLGEC | |
| BA14 | EVQLVESGGGLVQPGGSLRLSCAASGFTFSDYDIHWVRQAPGKGLEWVGDITPY | 153 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLPGKC | |
| BA15 | EVQLVESGGGLVQPGGSLRLSCAASGFTFSDYDIHWVRQAPGKGLEWVGDITPY | 154 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGC | |
| BA16 | EVQLVESGGGLVQPGGSLRLSCAASGFTFSDYDIHWVRQAPGKGLEWVGDITPY | 155 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKGC | |
| BA17 | EVQLVESGGGLVQPGGSLRLSCAASGFTFSDYDIHWVRQAPGKGLEWVGDITPY | 156 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKGSC | |
| BA18 | EVQLVESGGGLVQPGGSLRLSCAASGFTFSDYDIHWVRQAPGKGLEWVGDITPY | 157 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKC | |
| BA19 | EVQLVESGGGLVQPGGSLRLSCAASGFTFSDYDIHWVRQAPGKGLEWVGDITPY | 158 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLGEC | |
| BA20 | EVQLVESGGGLVQPGGSLRLSCAASGFTFSDYDIHWVRQAPGKGLEWVGDITPY | 159 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLPGKC | |
| BA21 | EVQLVESGGGLVQPGGSLRLSCAASGFTFSDYDIHWVRQAPGKGLEWVGDITPY | 160 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGC | |
| BA22 | EVQLVESGGGLVQPGGSLRLSCAASGFTFSDYDIHWVRQAPGKGLEWVGDITPY | 161 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKGC | |
| BA23 | EVQLVESGGGLVQPGGSLRLSCAASGFTFSDYDIHWVRQAPGKGLEWVGDITPY | 162 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKGSC | |
| BA24 | EVQLVESGGGLVQPGGSLRLSCAASGFTFSDYDIHWVRQAPGKGLEWVGDITPY | 163 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKC | |
| BA25 | EVQLVESGGGLVQPGGSLRLSCAASGFTFSDYDIHWVRQAPGKGLEWVGDITPY | 164 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLGEC | |
| BA26 | EVQLVESGGGLVQPGGSLRLSCAASGFTFSDYDIHWVRQAPGKGLEWVGDITPY | 165 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLPGKC | |
| BA27 | EVQLVESGGGLVQPGGSLRLSCAASGFTFSDYDIHWVRQAPGKGLEWVGDITPY | 166 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGC | |
| BA28 | EVQLVESGGGLVQPGGSLRLSCAASGFTFSDYDIHWVRQAPGKGLEWVGDITPY | 167 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKGC | |
| BA29 | EVQLVESGGGLVQPGGSLRLSCAASGFTFSDYDIHWVRQAPGKGLEWVGDITPY | 168 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKGSC | |
| BA30 | EVQLVESGGGLVQPGGSLRLSCAASGFTFSDYDIHWVRQAPGKGLEWVGDITPY | 169 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKC | |
| BA31 | EVQLVESGGGLVQPGGSLRLSCAASGFTFSDYDIHWVRQAPGKGLEWVGDITPY | 170 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLGEC | |
| BA32 | EVQLVESGGGLVQPGGSLRLSCAASGFTFSDYDIHWVRQAPGKGLEWVGDITPY | 171 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLPGKC | |
| BA33 | EVQLVESGGGLVQPGGSLRLSCAASGFTFSDYDIHWVRQAPGKGLEWVGDITPY | 172 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGC | |
| BA34 | EVQLVESGGGLVQPGGSLRLSCAASGFTFSDYDIHWVRQAPGKGLEWVGDITPY | 173 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKGC | |
| BA35 | EVQLVESGGGLVQPGGSLRLSCAASGFTFSDYDIHWVRQAPGKGLEWVGDITPY | 174 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKGSC | |
| BA36 | EVQLVESGGGLVQPGGSLRLSCAASGFTFSDYDIHWVRQAPGKGLEWVGDITPY | 175 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKC | |
| BA37 | EVQLVESGGGLVQPGGSLRLSCAASGFTFSDYDIHWVRQAPGKGLEWVGDITPY | 176 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLGEC | |
| BA38 | EVQLVESGGGLVQPGGSLRLSCAASGFTFSDYDIHWVRQAPGKGLEWVGDITPY | 177 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLPGKC | |
| BA39 | EVQLVESGGGLVQPGGSLRLSCAASGFTFSDYDIHWVRQAPGKGLEWVGDITPY | 178 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGC | |
| BA40 | EVQLVESGGGLVQPGGSLRLSCAASGFTFSDYDIHWVRQAPGKGLEWVGDITPY | 179 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRL AGKGC | |
| BA41 | EVQLVESGGGLVQPGGSLRLSCAASGFTFSDYDIHWVRQAPGKGLEWVGDITPY | 180 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLAGKGSC | |
| BA42 | EVQLVESGGGLVQPGGSLRLSCAASGFTFSDYDIHWVRQAPGKGLEWVGDITPY | 181 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRL AGKC | |
| BA43 | EVQLVESGGGLVQPGGSLRLSCAASGFTFSDYDIHWVRQAPGKGLEWVGDITPY | 182 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLGEC | |
| BA44 | EVQLVESGGGLVQPGGSLRLSCAASGFTFSDYDIHWVRQAPGKGLEWVGDITPY | 183 |
| | DGTTNYADSVKGRFTISADTSKNTAYLQMNSLRAEDTAVYYCARLVGEIATGFDY | |
| | WGQGTLVTVSSASTFRPEVHLLPPPSEELALNELVTLTCLARGFSPKDVLVRWLQ | |
| | GSQELPREKYLTWASRQEPSQGTTTFAVTSILRVAAEDWKKGDTFSCMVGHEAL | |
| | PLAFTQKTIDRLPGKC | |

CH1 Purification and SDS-PAGE Analysis

Protein was expressed by transient expression in Expi 293 cells as described above, and purified by one-step affinity chromatography using CH1 resin.

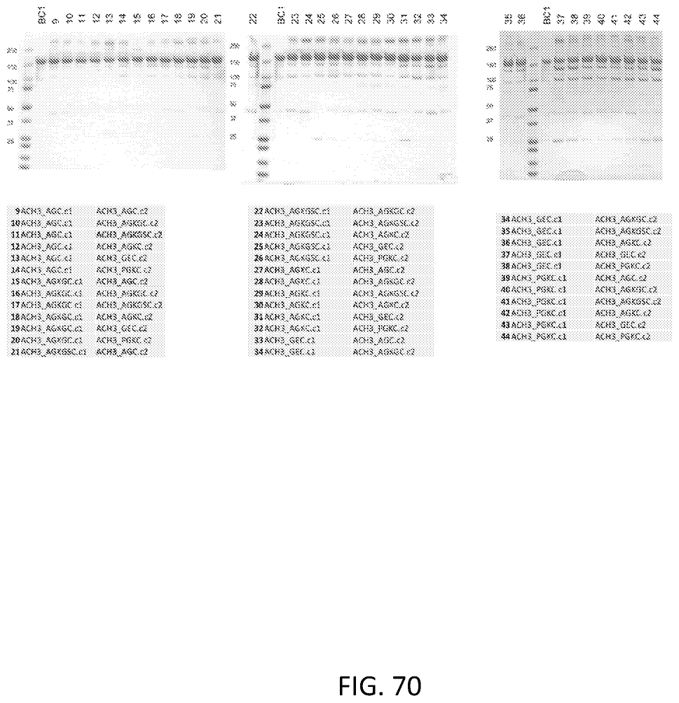

FIG. 70 depicts SDS-PAGE analysis of BC1 and variants BA9-44. Variants exhibiting high yield from transient transfection, low levels of low molecular weight contaminants, and low levels of high molecular weight contaminants, were selected for further optimization. It was observed that use of non-identical CH3 linkers for domains B and G resulted in improved yield and reduced contaminants. Variants BA11, BA15, BA21, and BA27 in particular exhibited high yield and high-fidelity assembly.

### 6.15.28. Example 27: Further Optimization and Characterization of CH3 Linker Sequences

In a further set of experiments, bivalent bispecific binding molecule variants, also termed “BA” specific for a first antigen (“Antigen A”) and second antigen (“Antigen B”) was constructed.

Salient features of the general BA architecture are illustrated in FIG. 3. In greater detail, with domain and polypeptide chain references in accordance with FIG. 3, the architecture of binding molecule “BA” was:
1^{st} polypeptide chain:

Domain A=V_{L} (“Antigen A”)

Domain B=C_{H3} (IgA, optional first CH3 linker)

Domain D=C_{H2}

Domain E=C_{H3} (Knob T366W, 354C)
2^{nd} polypeptide chain:
   Domain F=V_{H} (“Antigen A”)
   Domain G=C_{H3} (IgA, optional second CH3 linker)
3^{rd} polypeptide chain:
   Domain H=V_{L} (“Antigen B”)
   Domain I=C_{L} (Kappa)
   Domain J=C_{H2}
   Domain K=IgG1 CH3 (Y349C, D356E, L358M, T366S, L368A, Y407V)
4^{th} polypeptide chain:
   Domain L=VH (“Antigen B”)
   Domain M=CH1.

The A domain (SEQ ID NO: 12) and F domain (SEQ ID NO: 16) form an antigen binding site (A:F) specific for “Antigen A”. Domain H and domain L form an antigen binding site (H:L) specific for “Antigen B”).

Domain B has the human IgA CH3 sequence, (SEQ ID NO:184), with an optional first CH3 linker sequence or modification which connects the C-terminus of CH3 to the N-terminus of CH2 (domain D).

Domain G has the human IgA CH3 sequence, (SEQ ID NO:184), with a second CH3 linker sequence or modification which forms a disulfide bridge with the first CH3 linker sequence.

Domain D has the sequence of human IgG1 CH2 domain, with a CH2 hinge sequence (SEQ ID NO: 56) appended to the N-terminus of the CH2 domain.

Domain E (SEQ ID NO:15) has the sequence of human IgG1 CH3 with the mutations T366W and S354C.

Domain I (SEQ ID NO:19) has the sequence of human C kappa light chain (CIO

Domain J has the sequence of human IgG1 CH2 domain.

Domain K [SEQ ID NO: 21] has the sequence of human IgG1 CH3 with the following changes: Y349C, D356E, L358M, T366S, L368A, Y407V. The 349C mutation introduces a cysteine that is able to form a disulfide bond with the cognate 354C mutation in

Domain E. The 356E and L358M introduce isoallotype amino acids that reduce immunogenicity. The 366S, 368A, and 407V are “hole” mutations.

Domain M has the sequence of the human IgG1 CH1 region.

The BA variants in this experiment comprise the architecture stated above and comprise different sets of first and second CH3 linkers in the first and second polypeptide chains, respectively (see Table 16 below). The first CH3 linker attaches domain B to domain D. the second CH3 linker comprises an engineered cysteine that forms a disulfide bond with a cysteine in the first CH3 linker.

TABLE 16
Additional BA variants with CH3 linkers

| | Chain 1 | Chain 2 |
|---|---|---|
| | (first CH3 | (second |
| | linker) | CH3 linker) |
| BA11 (see Table 13) | AGC.c1 | AGKGSC.c2 |
| BA15 | AGKGC.c1 | AGC.c2 |
| BA11 (see Table 13) | | |
| BA45 | H350C | P355C |
| | amino acid | amino acid |
| | substitution | substitution |
| BA46 | P355C | H350C |
| | amino acid | amino acid |
| | substitution | substitution |

For clarity, the residue designated “H350” in the IgA-CH3 domain sequence is the underlined “H” residue in the following endogenous IgA-CH3 sequence:

TFRPEVHLLPPPSEELALNELVTLTCLARGFSPKDVLVRWLQGSQELPRE
KYLTWASRQEPSQGTTTFAVTSILRVAAEDWKKGDTFSCMVGHEALPLAF
TQKTIDRL.

By way of example, an IgA-CH3 amino acid domain sequence with a “H350C” mutation in an otherwise endogenous IgA-CH3 domain has the following sequence:

TFRPEVCLLPPPSEELALNELVTLTCLARGFSPKDVLVRWLQGSQELPRE
KYLTWASRQEPSQGTTTFAVTSILRVAAEDWKKGDTFSCMVGHEALPLAF
TQKTIDRL.

For clarity, the residue designated “P355” in the IgA-CH3 domain sequence is the underlined “P” residue in the following endogenous IgA-CH3 sequence:

TFRPEVHLLPPPSEELALNELVTLTCLARGFSPKDVLVRWLQGSQELPRE
KYLTWASRQEPSQGTTTFAVTSILRVAAEDWKKGDTFSCMVGHEALPLAF
TQKTIDRL.

By way of example, an IgA-CH3 amino acid domain sequence with a “P355C” mutation in an otherwise endogenous IgA-CH3 domain has the following sequence:

TFRPEVHLLPPCSEELALNELVTLTCLARGFSPKDVLVRWLQGSQELPRE
KYLTWASRQEPSQGTTTFAVTSILRVAAEDWKKGDTFSCMVGHEALPLAF
TQKTIDRL.

Protein was expressed by transient expression in Expi 293 cells as described above, and purified by one-step affinity chromatography using CH1 resin.

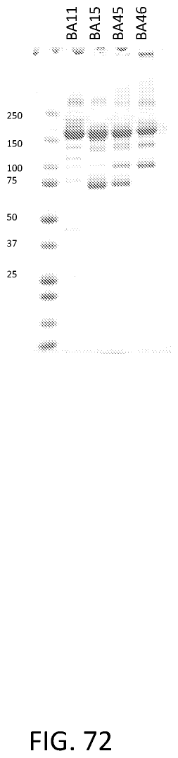

FIG. 72 depicts SDS-PAGE analysis of the BA variants in Table 16. All constructs exhibited full assembly and overall greater yield of fully assembled binding molecules as compared to incomplete products. Of the four variants, BA11 exhibited particularly high-fidelity assembly and high yield.

### 6.15.29. Example 28: Trivalent Constructs with IgA-CH3 Domain Swaps

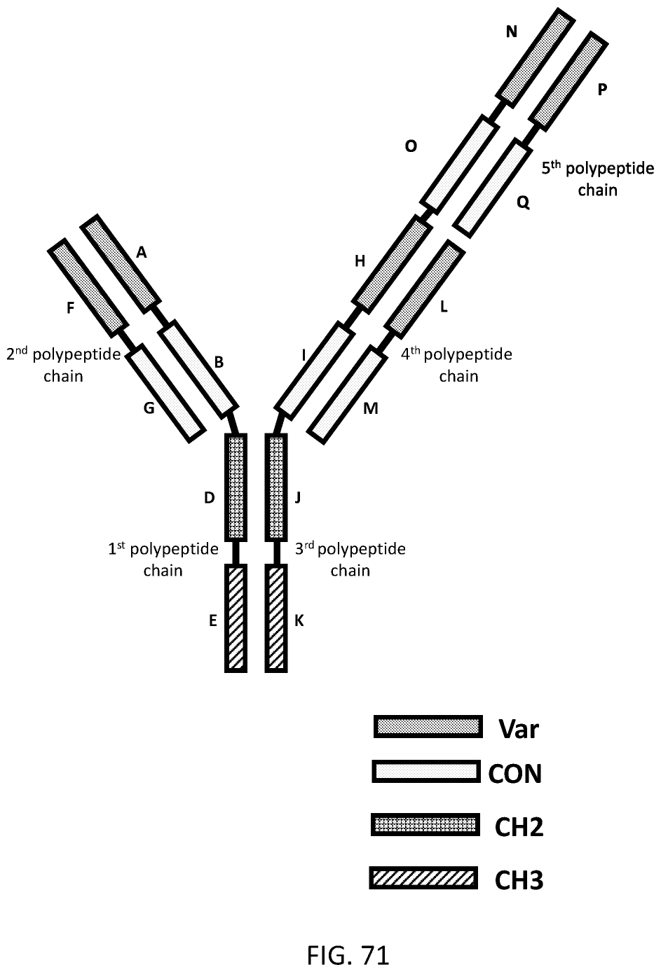

Trivalent binding molecules were constructed according to the general architecture depicted in FIG. 71. The binding molecules comprise a first polypeptide chain, a second polypeptide chain, a third polypeptide chain, a fourth polypeptide chain, and a fifth polypeptide chain.

The first polypeptide chain comprises a domain A, a domain B, a domain D, and a domain E, wherein the domains are arranged, from N-terminus to C-terminus, in an A-B-D-E orientation. Domain A has a variable region amino acid sequence, domain B has a constant region amino acid sequence, domain D has a CH2 sequence, and domain E has a CH3 sequence.

The second polypeptide chain comprises a domain F and a domain G, wherein the domains are arranged, from N-terminus to C-terminus, in a F-G orientation. Domain F has a variable region domain amino acid sequence and domain G has a constant region domain amino acid sequence.

The third polypeptide chain comprises a domain N, a domain O, a domain H, a domain I, a domain J, and a domain K, wherein the domains are arranged, from N-terminus to C-terminus, in a N-O-H-I-J-K orientation. Domain N has a variable region amino acid sequence, domain O has a constant region amino acid sequence, domain H has a variable region domain amino acid sequence, domain I has a constant region amino acid sequence, domain J has a CH2 sequence, and domain K has a CH3 sequence.

The fourth polypeptide chain comprises a domain L and a domain M, wherein the domains are arranged, from N-terminus to C-terminus, in a L-M orientation. Domain L has a variable region domain amino acid sequence and domain M comprises a constant region amino acid sequence.

The fifth polypeptide chain comprises a domain P and a domain Q, wherein the domains are arranged, from N-terminus to C-terminus, in a P-Q orientation. Domain P comprises a variable region amino acid sequence and domain Q comprises a constant region amino acid sequence.

Domains A and F associate to form a first antigen binding site; domains H and L associate to form a second antigen binding site; and domains N and P associate to form a third antigen binding site.

Domains B and G form a first domain pair of associated constant region domains (“first domain pair”), domains I and M form a second domain pair of associated constant region domains (“second domain pair”), and domains Q and O form a third domain pair of associated constant region domains (“third domain pair”). At least one of the first, second, and third domain pairs is a pair of associated IgA-CH3 domains.

The architecture of exemplary constructed trivalent molecules is shown in the Table below. A non-exhaustive list of optional modifications are shown in italics.

TABLE 17
trivalent binding molecules comprising IgA-CH3/IgA-CH3 domain pairs

| Construct | | | | | |
|---|---|---|---|---|---|
| Name | Chain 1 | Chain 2 | Chain 3 | Chain 4 | Chain 5 |
| T26 | A = VL | F= VH | N = VL | L = VH | P = VH |
| | B = IgA-CH3 | G = IgA-CH3 | O = IgA-CH3 | M = CH1 | Q = IgA-CH3 |
| | H350C | P355C | AGKGSC | | AGC |
| | D = CH2 | | linker | | linker |
| | L234A, L235A, | | H = VL | | |
| | P329K | | I = CL | | |
| | E = IgG CH3 | | J = CH2 | | |
| | Y349C, | | L234A, L235A, | | |
| | D356E, | | P329K | | |
| | L358M, | | K = IgG CH3 | | |
| | T366S, L368A, | | S354C, | | |
| | Y407V | | T366W | | |
| T27 | A = VL | F = VH | N = VL | L = VH | P = VH |
| | B = IgA-CH3 | G = IgA-CH3 | O = IgA-CH3 | M = CH1 | Q = IgA-CH3 |
| | AGC linker | AGKGSC | H350C | | P355C |
| | D = CH2 | linker | H = VL | | |
| | L234A, L235A, | | I = CL | | |
| | P329K | | J = CH2 | | |
| | E = IgG CH3 | | L234A, L235A, | | |
| | Y349C, | | P329K | | |
| | D356E, | | K = IgG CH3 | | |
| | L358M, | | S354C, | | |
| | T366S, L368A, | | T366W | | |
| | Y407V | | | | |
| T28 | A = VL | F = VH | N = VL | L = VH | P = VH |
| | B = IgA-CH3 | G = IgA-CH3 | O = IgA-CH3 | M = CH1 | Q = IgA-CH3 |
| | H350C | P355C | AGC linker | | AGKGSC |
| | D = CH2 | | H = VL | | linker |
| | L234A, L235A, | | I = CL | | |
| | P329K | | J = CH2 | | |
| | E = IgG CH3 | | L234A, L235A, | | |
| | Y349C, | | P329K | | |
| | D356E, | | K = IgG CH3 | | |
| | L358M, | | S354C, | | |
| | T366S, L368A, | | T366W | | |
| | Y407V | | | | |
| T33 | A = VL | F = VH | N = VL | L = VH | P = VH |
| | B = IgG-CH3 | G = IgG-CH3 | O = IgA-CH3 | M = CH1 | Q = IgA-CH3 |
| | T366K, 447C | L351D, 447C | H350C | | P355C |
| | D = CH2 | | H = VL | | |
| | L234A, L235A, | | I = CL | | |
| | P329K | | J = CH2 | | |
| | E = IgG CH3 | | L234A, L235A, | | |
| | Y349C, | | P329K | | |
| | D356E, | | K = IgG CH3 | | |
| | L358M, | | S354C, | | |
| | T366S, L368A, | | T366W | | |
| | Y407V | | | | |
| T34 | A = VL | F = VH | N = VL | L = VH | P = VH |
| | B = IgA-CH3 | G = IgA-CH3 | O = IgG-CH3 | M = CH1 | Q = IgG-CH3 |
| | H350C | P355C | T366K, 447C | | L351D, |
| | D = CH2 | | H = VL | | 447C |
| | L234A, L235A, | | I = CL | | |
| | P329K | | J = CH2 | | |
| | E = IgG CH3 | | L234A, L235A, | | |
| | Y349C, | | P329K | | |
| | D356E, | | K = IgG CH3 | | |
| | L358M, | | S354C, | | |
| | T366S, L368A, | | T366W | | |
| | Y407V | | | | |
| T35 | A = VL | F = VH | N = VL | L = VH | P = VH |
| | B = IgG-CH3 | G = IgG-CH3 | O = IgA-CH3 | M = CH1 | Q = IgA-CH3 |
| | T366K, 447C | L351D, 447C | AGC linker | | AGKGSC |
| | D = CH2 | | H = VL | | linker |
| | L234A, L235A, | | I = CL | | |
| | P329K | | J = CH2 | | |
| | E = IgG CH3 | | L234A, L235A, | | |
| | Y349C, | | P329K | | |
| | D356E, | | K = IgG CH3 | | |
| | L358M, | | S354C, | | |
| | T366S, L368A, | | T366W | | |
| | Y407V | | | | |
| T36 | A = VL | F = VH | N = VL | L = VH | P = VH |
| | B = IgA-CH3 | G = IgA-CH3 | O = IgG-CH3 | M = CH1 | Q = IgG-CH3 |
| | AGC linker | AGKGSC | T366K, 447C | | L351D, |
| | D = CH2 | linker | H = VL | | 447C |
| | L234A, L235A, | | I = CL | | |
| | P329K | | J = CH2 | | |
| | E = IgG CH3 | | L234A, L235A, | | |
| | Y349C (for | | P329K | | |
| | engineered | | K = IgG CH3 | | |
| | disulfide | | S354C, | | |
| | bridge), | | T366W | | |
| | D356E, | | | | |
| | L358M, | | | | |
| | T366S, L368A, | | | | |
| | Y407V | | | | |
| T37 | A = VL | F = VH | N = VL | L = VH | P = VH |
| | B = IgG-CH3 | G = IgG-CH3 | O = IgA-CH3 | M = CH1 | Q = IgA-CH3 |
| | [P343V; | (S354C; 445P, | AGC linker | | AGKGSC |
| | Y349C; 445P, | 446G, 447K | H = VL | | linker |
| | 446G, 447K | insertion) | I = CL | | |
| | insertion] | | J = CH2 | | |
| | D = CH2 | | L234A, L235A, | | |
| | L234A, L235A, | | P329K | | |
| | P329K | | K = IgG CH3 | | |
| | E = IgG CH3 | | S354C, | | |
| | Y349C, | | T366W | | |
| | D356E, | | | | |
| | L358M, | | | | |
| | T366S, L368A, | | | | |
| | Y407V | | | | |
| T38 | A = VH | F = VL | N = VH | L = VL | P = VL |
| | B = IgG-CH3 | G = IgG-CH3 | O = IgA-CH3 | M = C1 | Q = IgA-CH3 |
| | P343V; | (S354C; 445P, | AGC linker | | AGKGSC |
| | Y349C; 445P, | 446G, 447K | H = VH | | linker |
| | 446G, 447K | insertion) | I = CH1 | | |
| | insertion | | J = CH2 | | |
| | D = CH2 | | L234A, L235A, | | |
| | L234A, L235A, | | P329K | | |
| | P329K | | K = IgG-CH3 | | |
| | E = IgG-CH3 | | S354C, | | |
| | Y349C, | | T366W | | |
| | D356E, | | | | |
| | L358M, | | | | |
| | T366S, L368A, | | | | |
| | Y407V | | | | |

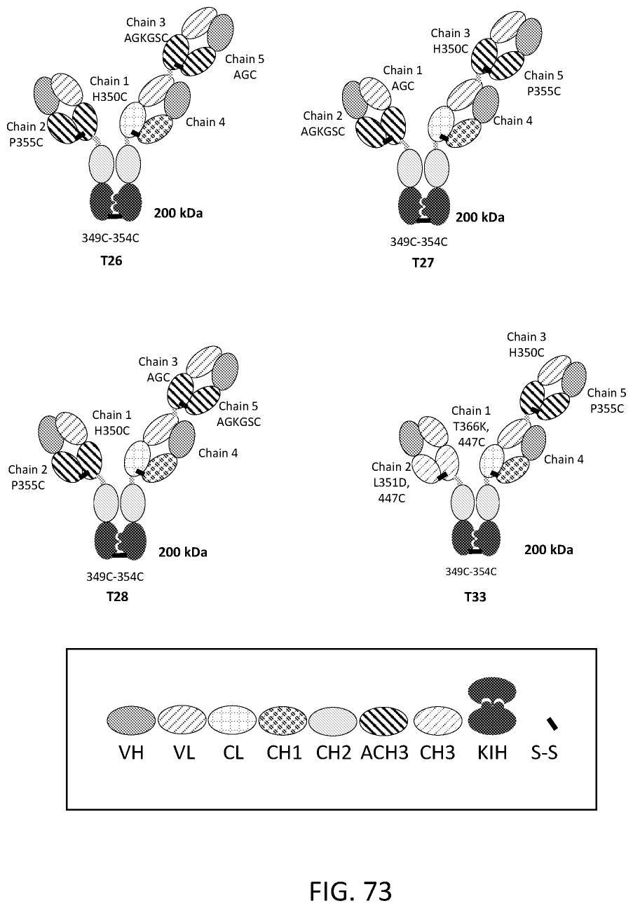

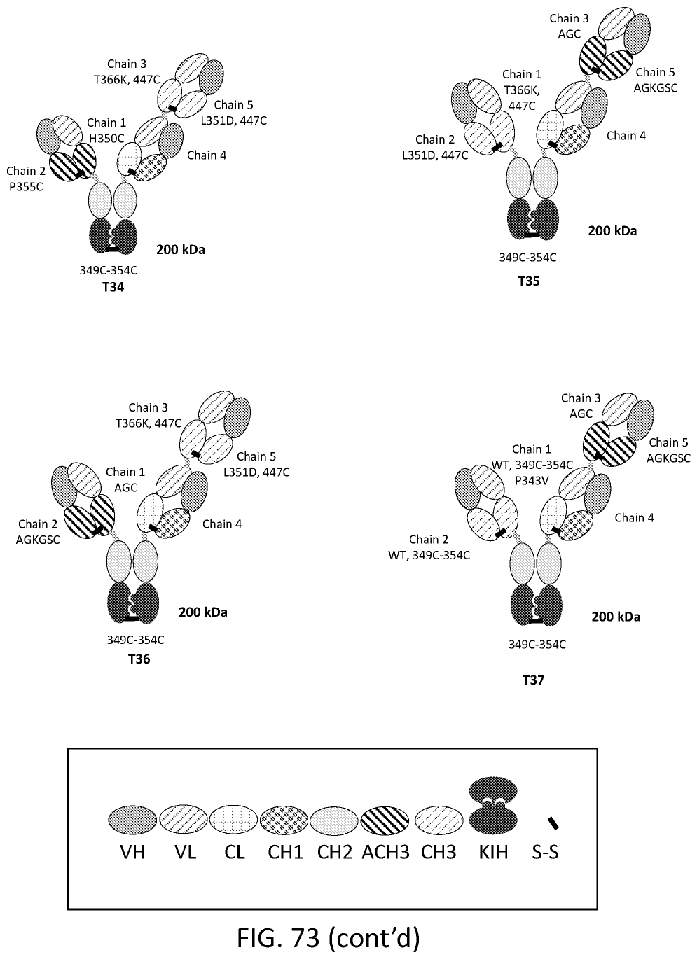

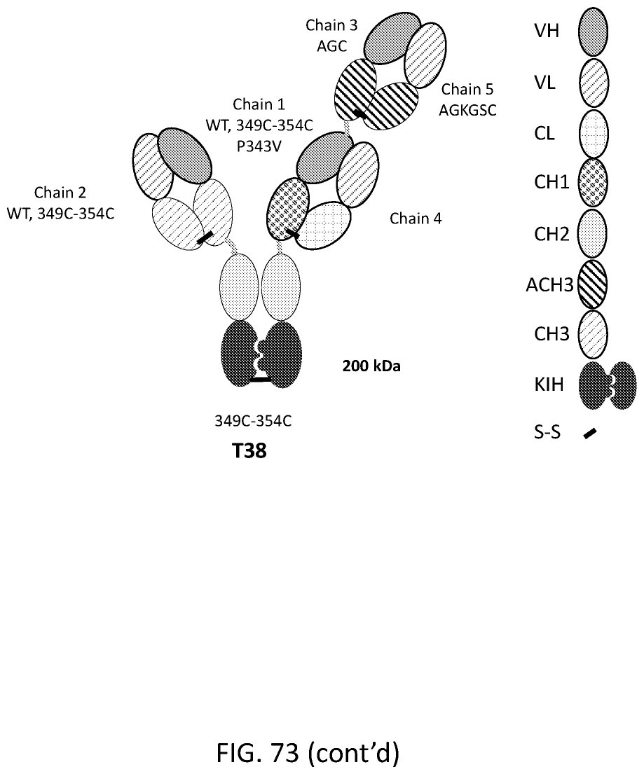

FIG. 73 depicts architectures of the various trivalent molecules (“T26,” “T27,” “T28,” “T33,” “T34,” “T35,” “T36”, “T37,” and “T38”).

Polypeptide chain amino acid sequences of the trivalent molecules T27 and T36 are included in the Sequences section.

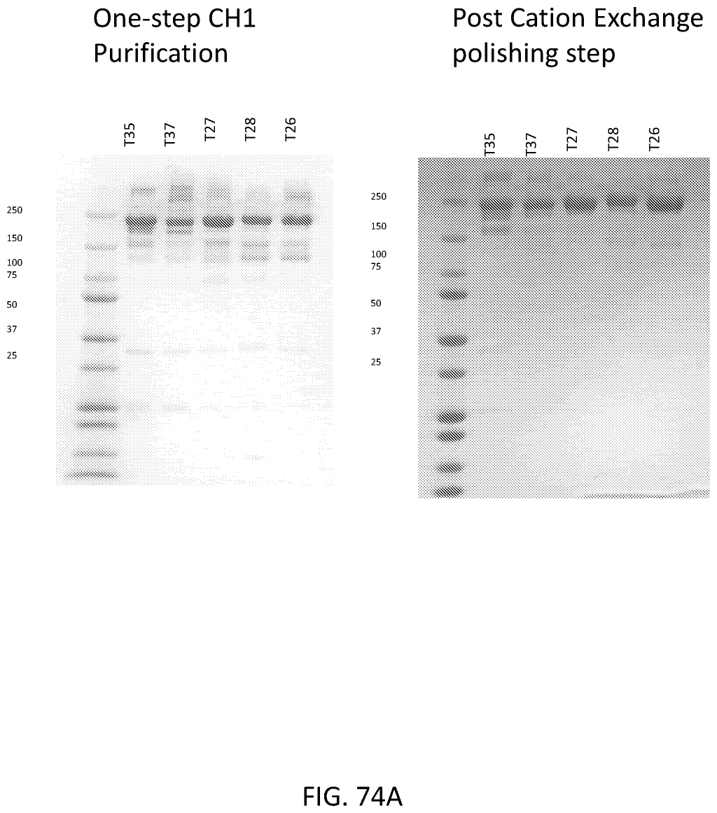

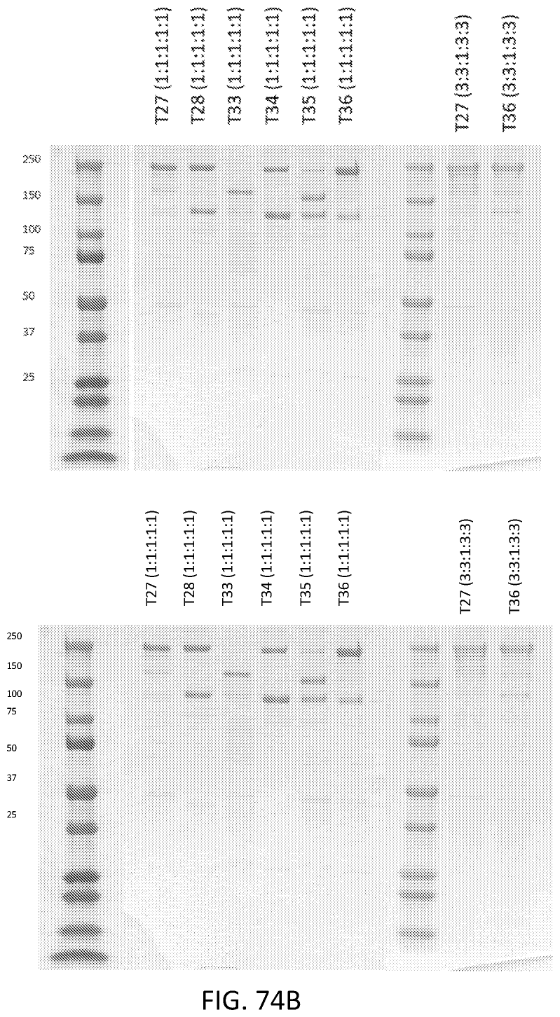

All constructs were expressed using the Expi293 system and isolated using CH1 purification as described herein. In some cases, the resulting products from CH1 purification were subjected to further purification using cation exchange polishing (IEX Chromatography), as described herein in Example 1. The resulting products were subjected to SDS-PAGE analysis. SDS-PAGE gels are shown in FIG. 74A. All constructs exhibited full assembly and overall greater yield of fully assembled binding molecules as compared to incomplete products, both with one-step CH1 purification and two-step purification.

In a further experiment, constructs T27, T28, T33, T34, T35, and T36 were expressed using varying ratios of polypeptide chains (by mass). For clarification, chain ratios are expressed as Chain 1: Chain 2: Chain 3: Chain 4: Chain 5 ratios. By way of example only, a chain ratio of (1:1:1:1:1) describes an experiment in which equal masses of Chain 1: Chain 2: Chain 3: Chain 4: Chain 5 were expressed. Constructs were expressed using the Expi293 system and then purified using one-step CH1 purification as described herein. The resulting SDS-PAGE gel is shown in FIG. 74B. T27 and T37 exhibited particularly high fidelity assembly with a 1:1:1:1:1 chain ratio, and even higher fidelity assembly with a 3:3:1:3:3 chain ratio.

### 6.16. Sequences

>Example 1, bivalent monospecific construct CHAIN 1 [SEQ ID NO: 1]
(VL)~VEIKRTPREPQVYTLPPCRDELTKNQVSLWCLVKGFYPSDIAVEWESNGQPEN
NYKTTPPVLDSDGSFFLYSKLTVDKSRWQQGNVFSCSVMHEALHNHYTQKSLSLSPG
KDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPI
EKTISKAKGQPREPQVYTLPPCRDELTKNQVSLWCLVKGFYPSDIAVEWESNGQPEN
NYKTTPPVLDSDGSFFLYSKLTVDKSRWQQGNVFSCSVMHEALHNHYTQKSLSLSPG
K
>Example 1, bivalent monospecific construct CHAIN 2 [SEQ ID NO: 2]
(VH)~VTVSSASPREPQVYTLPPCRDELTKNQVSLWCLVKGFYPSDIAVEWESNGQPE
NNYKTTPPVLDSDGSFFLYSKLTVDKSRWQQGNVFSCSVMHEALHNHYTQKSLSLSP
GK
>Example 1, bivalent, bispecific construct CHAIN 1 [SEQ ID NO: 31]
(VL)~VEIKRTPREPQVYTLPPCRDELTKNQVSLWCLVKGFYPSDIAVEWESNGQPEN
NYKTTPPVLDSDGSFFLYSKLTVDKSRWQQGNVFSCSVMHEALHNHYTQKSLSLSPG
KDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKA
LPAPIEKTISKAKGQPREPQVYTLPPCRDELTKIVQVSLWCLVKGFYPSDIAVEWESNGQ
PENNYKTTPPVLDSDGSFFLYSKLTVDKSRWQQGNVFSCSVMHEALHNHYTQKSLSLSPG
K
VL- CH3- Hinge- CH2- CH3 (knob)
>Example 1, bivalent, bispecific construct CHAIN 2 [SEQ ID NO: 4]
(VH)~VTVSSASPREPQVYTLPPCRDELTKNQVSLWCLVKGFYPSDIAVEWESNGQPE
NNYKTTPPVLDSDGSFFLYSKLTVDKSRWQQGNVFSCSVMHEALHNHYTQKSLSLSP
GK
VH- CH3
>Example 1, bivalent, bispecific construct CHAIN 3_[SEQ ID NO: 5]
(VL)~VEIKRTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVTKSFNRGE
CDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKA
LPAPIEKTISKAKGQPREPQVCTLPPSREEMTKNQVSLSCAVKGFYPSDIAVEWESNGQP
ENNYKTTPPVLDSDGSFFLVSKLTVDKSRWQQGNVFSCSVMHEALHNHYTQKSLSLSPGK
VL- CL- Hinge- CH2- CH3 (hole)
>Example 1, bivalent, bispecific construct CHAIN 4 [SEQ ID NO: 6]
(VH)~VTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPPKSC
VH- CH1
>Fc Fragment of Human IgG1 [SEQ ID NO: 7]
GQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSP
>BC1 chain 1 [SEQ ID NO: 8]
DIQMTQSPSSLSASVGDRVTITCRASQDVNTAVAWYQQKPGKAPKLLIYSASF
LYSGVPSRFSGSRSGTDFTLTISSLQPEDFATYYCQQHYTTPPTFGQGTKVEIK
RTPREPQVYTLPPSRDELTKNQVSLKCLVKGFYPSDIAVEWESNGQPENNYK
TTPPVLDSDGSFFLYSKLTVDKSRWQQGNVFSCSVMHEALHNHYTQKSLSLS
KSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNG
KEYKCKVSNKALPAPIEKTISKAKGQPREPQVYTLPPCRDELTKNQVSLWCLV
KGFYPSDIAVEWESNGQPENNYKTTPPVLDSDGSFFLYSKLTVDKSRWQQGNFFS
CSMHEALHNHYTQKSLSLSPGK
Domain arrangement:
A- B- Hinge- D- E
VL- CH3- Hinge- CH2- CH3 (knob)
Mutations in first CH3 (Domain B):
T366K; 445K, 446S, 447C insertion
Mutations in second CH3 (Domain E):
S354C, T366W
>BC1 chain 2 [SEQ ID NO: 9]
EVQLVESGGGLVQPGGSLRLSCAASGFTFSDYDIHWVRQAPGKGLEWVGDIT
PYDGTTNYADSVKGRFTISADTSKNTAYLQMNSLRAEDTAVYYCARLVGEIA
TGFDYWGQGTLVTVSSASPREPQVYTDPPSRDELTKNQVSLTCLVKGFYPSDI
AVEWESNGQPENNYKTTPPVLDSDGSFFLYSKLTVDKSRWQQGNVFSCSVM
HEALHNHYTQKSLSLSGEC
Domain arrangement:
F- G
VH- CH3
Mutations in CH3 (Domain G):
L351D; 445G, 446E, 447C insertion
>BC1 chain 3 [SEQ ID NO: 10]
EIVLTQSPATLSLSPGERATLSCRASQSVSSYLAWYQQKPGQAPRLLIYDASN
RATGIPARFSGSGSGTDFTLTISSLEPEDFAVYYCQQSSNWPRTFGQGTKVEIK
RTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNR
GECDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNG
KEYKCKVSNKALPAPIEKTISKAKGQPREPQVCTLPPSREEMTKNQVSLSCAV
KGFYPSDIAVEWESNGQPENNYKTTPPVLDSDGSFFLVSKLTVDKSRWQQGNFFS
CSMHEALHNHYTQKSLSLSPGK
Domain arrangement:
H- I- Hinge- J- K
VL- CL- Hinge- CH2- CH3 (hole)
Mutations in CH3 (domain K):
Y349C, D356E, L358M, T366S, L368A, Y407V
>BC1 chain 4 [SEQ ID NO: 11]
QVQLVESGGGVVQPGRSLRLDCKASGITFSNSGMHWVRQAPGKGLEWVAVI
WYDGSKRYYADSVKGRFTISRDNSKNTLFLQMNSLRAEDTAVYYCATNDDY
WGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKV
DKKVEPPKSC
Domain arrangement:
L- M
VH- CH1
>BC1, BA1 Domain A [SEQ ID NO: 12]
DIQMTQSPSSLSASVGDRVTITCRASQDVNTAVAWYQQKPGKAPKLLIYSASF
LYSGVPSRFSGSRSGTDFTLTISSLQPEDFATYYCQQHYTTPPTFGQGTKVEIK
RT
>BC1 Domain B [SEQ ID NO: 13]
PREPQVYTLPPSRDELTKNQVSLKCLVKGFYPSDIAVEWESNGQPENNYKTTP
PVLDSDGSFFLYSKLTVDKSRWQQGNVFSCSVMHEALHNHYTQKSLSLSKSC
>BC1 Domain D [SEQ ID NO: 14]
APELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIE
KTISKAK
>BC1, BA1 Domain E [SEQ ID NO: 15]
GQPREPQVYTLPPCRDELTKNQVSLWCLVKGFYPSDIAVEWESNGQPENNYK
TTPPVLDSDGSFFLYSKLTVDKSRWQQGNVFSCSVMHEALHNHYTQKSLSLS
PGK
>BA1, BC1 Domain F, [SEQ ID NO: 16]
EVQLVESGGGLVQPGGSLRLSCAASGFTFSDYDIHWVRQAPGKGLEWVGDIT
PYDGTTNYADSVKGRFTISADTSKNTAYLQMNSLRAEDTAVYYCARLVGEIA
TGFDYWGQGTLVTVSSAS
>BC1 Domain G [SEQ ID NO: 17]
PREPQVYTDPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSGEC
>BC1 Domain H [SEQ ID NO: 18]
EIVLTQSPATLSLSPGERATLSCRASQSVSSYLAWYQQKPGQAPRLLIYDASN
RATGIPARFSGSGSGTDFTLTISSLEPEDFAVYYCQQSSNWPRTFGQGTKVEIK
>BC1, BA1 Domain I [SEQ ID NO: 19]
RTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNR
GEC
>BC1 Domain J [SEQ ID NO: 20]
APELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIE
KTISKAK
>BC1, BA1 Domain K [SEQ ID NO: 21]
GQPREPQVCTLPPSREEMTKNQVSLSCAVKGFYPSDIAVEWESNGQPENNYK
TTPPVLDSDGSFFLVSKLTVDKSRWQQGNVFSCSVMHEALHNHYTQKSLSLS
PGK
>BC1 Domain L [SEQ ID NO: 22]
QVQLVESGGGVVQPGRSLRLDCKASGITFSNSGMHWVRQAPGKGLEWVAVI
WYDGSKRYYADSVKGRFTISRDNSKNTLFLQMNSLRAEDTAVYYCATNDDY
WGQGTLVTVSS
>BC1, BA1 Domain M [SEQ ID NO: 23]
ASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPPKSC
>BC28 chain 1 [SEQ ID NO: 24]
DIQMTQSPSSLSASVGDRVTITCRASQDVNTAVAWYQQKPGKAPKLLIYSASF
LYSGVPSRFSGSRSGTDFTLTISSLQPEDFATYYCQQHYTTPPTFGQGTKVEIK
RTPREPQVCTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSLSP
GKDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGK
EYKCKVSNKALPAPIEKTISKAKGQPREPQVYTLPPCRDELTKNQVSLWCLVK
GFYPSDIAVEWESNGQPENNYKTTPPVLDSDGSFFLYSKLTVDKSRWQQGNVFSC
SVMHEALHNHYTQKSLSLSPGK
A- B- Hinge- D- E
VL- CH3- Hinge- CH2- CH3(knob)
Mutations in domain B:
Y349C; 445P, 446G, 447K insertion
Mutations in domain E:
S354C, T366W
>BC28 chain 2 [SEQ ID NO: 25]
EVQLVESGGGLVQPGGSLRLSCAASGFTFSDYDIHWVRQAPGKGLEWVGDIT
PYDGTTNYADSVKGRFTISADTSKNTAYLQMNSLRAEDTAVYYCARLVGEIA
TGFDYWGQGTLVTVSSASPREPQVYTLPPCRDELTKNQVSLTCLVKGFYPSDI
AVEWESNGQPENNYKTTPPVLDSDGSFFLYSKLTVDKSRWQQGNVFSCSVM
HEALHNHYTQKSLSLSPGK
Domain arrangement:
F- G
VH- CH3
Mutations in domain G:
S354C; 445P, 446G, 447K insertion
>BC28 domain A [SEQ ID NO: 26]
DIQMTQSPSSLSASVGDRVTITCRASQDVNTAVAWYQQKPGKAPKLLIYSASF
LYSGVPSRFSGSRSGTDFTLTISSLQPEDFATYYCQQHYTTPPTFGQGTKVEIK
RT
>BC28 domain B [SEQ ID NO: 27]
PREPQVCTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSPGK
>BC28 domain D [SEQ ID NO: 28]
APELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIE
KTISKAK
>BC28 domain E [SEQ ID NO: 29]
GQPREPQVYTLPPCRDELTKNQVSLWCLVKGFYPSDIAVEWESNGQPENNYK
TTPPVLDSDGSFFLYSKLTVDKSRWQQGNVFSCSVMHEALHNHYTQKSLSLS
PGK
>BC28 domain F [SEQ ID NO: 30]
EVQLVESGGGLVQPGGSLRLSCAASGFTFSDYDIHWVRQAPGKGLEWVGDIT
PYDGTTNYADSVKGRFTISADTSKNTAYLQMNSLRAEDTAVYYCARLVGEIA
TGFDYWGQGTLVTVSSAS
>BC28 domain G [SEQ ID NO: 31]
PREPQVYTLPPCRDELTKNQVSLTCLVKGFYPSDIAVEWESNGQPENNYKTTP
PVLDSDGSFFLYSKLTVDKSRWQQGNVFSCSVMHEALHNHYTQKSLSLSPGK
>BC44 chain 1 [SEQ ID NO: 32]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTVREPQVC
TLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGKDKTHTCPPCPAPELLG
GPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQP
REPQVYTLPPCRDELTKNQVSLWCLVKGFYPSDIAVEWESNGQPENNYKTTPPVLDSDGS
FFLYSKLTVDKSRWQQGNFFSCSVMHEALHNHYTQKSLSLSPGK
Domain arrangement:
A- B- Hinge- D- E
VL- CH3- Hinge- CH2- CH3(knob)
Mutations in domain B:
P343V, Y349C; 445P, 446G, 447K insertion
Mutations in domain E:
S354C, T366W
>BC44 Domain A [SEQ ID NO: 33]
DIQMTQSPSSLSASVGDRVTITCRASQDVNTAVAWYQQKPGKAPKLLIYSASF
LYSGVPSRFSGSRSGTDFTLTISSLQPEDFATYYCQQHYTTPPTFGQGTKVEIK
RT
>BC44 Domain B [SEQ ID NO: 34]
VREPQVCTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSPGK
>BC44 Domain D [SEQ ID NO: 35]
APELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIE
KTISKAK
>BC44 Domain E [SEQ ID NO: 36]
GQPREPQVYTLPPCRDELTKNQVSLWCLVKGFYPSDIAVEWESNGQPENNYK
TTPPVLDSDGSFFLYSKLTVDKSRWQQGNVFSCSVMHEALHNHYTQKSLSLS
PGK
>BC28 bivalent chain 3 equivalent to SEQ ID NO: 10
>BC28 bivalent chain 4 equivalent to SEQ ID NO: 11
>BC28 1x2 chain 3 [SEQ ID NO: 37]
DIQMTQSPSSLSASVGDRVTITCRASQDVNTAVAWYQQKPGKAPKLLIYSASF
LYSGVPSRFSGSRSGTDFTLTISSLQPEDFATYYCQQHYTTPPTFGQGTKVEIK
RTPREPQVCTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSLSP
GKGSGSGS

[symbol]

[symbol]

[symbol]

[symbol]

[symbol]

[symbol]

[symbol]

[symbol]

RTVAAPSVFIFPPSDEQLKSGTASVVCLLNNFYPREAKVQWKV
DNALQSGNSQESVTEQDSKDSTYSLSSTLTLSKADYEKIIKVYACEVTHQG
LSSPVTKSFNRGECDKTHTCPPCPAPELLGGPSVFLFPPKPKDTLMISRTPE
VTCVVVDVSHEDPEVKFNWYVDGVEVHNAKTKPREEQYNSTYRVVSVL
TVLHQDWLNGKEYKCKVSNKALPAPIEKTISKAKGQPREPQVCTLPPSREE
MTKNQVSLSCAVKGFYPSDIAVEWESNGQPENNYKTTPPVLDSDGSFELVSKLTVD
KSRWQQGNVESCSMHEALHNHYTQKSLSLSPGK
Domain arrangement:
R- S- linker- H- I- Hinge- J- K-
VL- CH3- linker- VL- CL- Hinge- CH2- CH3(hole)
Mutations in domain S:
Y349C; 445P, 446G, 447K insertion
Six amino acids linker insertion: GSGSGS
Mutations in domain K:
Y349C, D356E, L358M, T3665, L368A, Y407V
>BC28 1x2 domain R [SEQ ID NO: 38]
DIQMTQSPSSLSASVGDRVTITCRASQDVNTAVAWYQQKPGKAPKLLIYSASF
LYSGVPSRFSGSRSGTDFTLTISSLQPEDFATYYCQQHYTTPPTEGQGTKVEIK
RT
>BC28 1x2 domain S [SEQ ID NO: 39]
PREPQVCTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSPGK
>BC28 1x2 linker [SEQ ID NO: 40]
GSGSGS
>BC28 1x2 domain H [SEQ ID NO: 41]
EIVLTQSPATLSLSPGERATLSCRASQSVSSYLAWYQQKPGQAPRLLIYDASN
RATGIPARFSGSGSGTDFTLTISSLEPEDFAVYYCQQSSNWPRTFGQGTKVEIK
>BC28 1x2 domain I [SEQ ID NO: 42]
RTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNR
GEC
>BC28 1x2 domain J [SEQ ID NO: 43]
APELLGGPSVFLEPPKPKDTLMISRTPEVTCVVVDVSHEDPEVKFNWYVDGV
EVHNAKTKPREEQYNSTYRVVSVLTVLHQDWLNGKEYKCKVSNKALPAPIE
KTISKAK
>BC28 1x2 domain K [SEQ ID NO: 44]
GQPREPQVCTLPPSREEMTKNQVSLSCAVKGFYPSDIAVEWESNGQPENNYK
TTPPVLDSDGSFFLVSKLTVDKSRWQQGNVFSCSVMHEALHNHYTQKSLSLS
PGK
>BC28-1x1x1a chain 3 [SEQ ID NO: 45]
DIQMTQSPSSLSASVGDRVTITCRASQSVSSAVAWYQQKPGKAPKLLIYSASS
LYSGVPSRFSGSRSGTDFTLTISSLQPEDFATYYCQQRDSYLWTFGQGTKVEIK
RTPREPQVYTLPPSRDELTKNQVSLKCLVKGFYPSDIAVEWESNGQPENNYK
TTPPVLDSDGSFFLYSKLTVDKSRWQQGNVFSCSVMHEALHNHYTQKSLSLS
KSCGSGSGS

[symbol]

[symbol]

[symbol]

[symbol]

[symbol]

[symbol]

[symbol]

[symbol]

RTVAAPSVFIFPPSDEQLKSGTASVVCLLNNFYPREAKVQWK
VDNALQSGNSQESVTEQDSKDSTYSLSSTLTLSKADYEKIIKVYACEVTHQ
GLSSPVTKSFNRGECDKTHTCPPCPAPELLGGPSVFLFPPKPKDTLMISRTP
EVTCVVVDVSHEDPEVKFNWYVDGVEVHNAKTKPREEQYNSTYRVVSV
LTVLHQDWLNGKEYKCKVSNKALPAPIEKTISKAKGQPREPQVCTLPPSRE
EMTKNQVSLSCAVKGFYPSDIAVEWESNGQPENNYKTTPPVLDSDGSFFLVSKLTV
DKSRWQQGNVFSCSVMHEALHNHYTQKSLSLSPGK
Domain arrangement:
R- S- linker- H- I- Hinge- J- K-
VL- CH3- linker- VL- CL- Hinge- CH2- CH3(hole)
Mutations in domain S:
T366K; 445K, 446S, 447C insertion
Six amino acids linker insertion: GSGSGS
Mutations in domain K:
Y349C, D356E, L358M, T3665, L368A, Y407V
>BC28-1x1x1a domain R [SEQ ID NO: 46]
DIQMTQSPSSLSASVGDRVTITCRASQSVSSAVAWYQQKPGKAPKLLIYSASS
LYSGVPSRFSGSRSGTDFTLTISSLQPEDFATYYCQQRDSYLWTEGQGTKVEIK
RT
>BC28-1x1x1a domain S [SEQ ID NO: 47]
PREPQVYTLPP SRDELTKNQVSLKCLVKGFYPSDIAVEWESNGQPENNYKTTP
PVLDSDGSFELYSKLTVDKSRWQQGNVESCSVMHEALHNHYTQKSLSLSKSC
>BC28-1x1x1a linker [SEQ ID NO: 48]
GSGSGS
>BC28-1x1x1a domain H [SEQ ID NO: 49]
EIVLTQSPATLSLSPGERATLSCRASQSVSSYLAWYQQKPGQAPRLLIYDASN
RATGIPARFSGSGSGTDFTLTISSLEPEDFAVYYCQQSSNWPRTFGQGTKVEIK
>BC28-1x1x1a domain I [SEQ ID NO: 50]
RTVAAPSVFIFPPSDEQLKSGTASVVCLLNNEYPREAKVQWKVDNALQSGNS
QESVTEQDSKDSTYSLSSTLTLSKADYEKHKVYACEVTHQGLSSPVTKSENR
GEC
>BC28-1x1x1a domain J [SEQ ID NO: 51]
APELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIE
KTISKAK
>BC28-1x1x1a domain K [SEQ ID NO: 52]
GQPREPQVCTLPPSREEMTKNQVSLSCAVKGFYPSDIAVEWESNGQPENNYK
TTPPVLDSDGSFFLVSKLTVDKSRWQQGNVFSCSVMHEALHNHYTQKSLSLS
PGK
>hCTLA4-4.chain 2 [SEQ ID NO: 53]
EVQLVESGGGLVQPGGSLRLSCAASGFTFSTYYIHWVRQAPGKGLEWVAVIY
PYTGFTYYADSVKGRFTISADTSKNTAYLQMNSLRAEDTAVYYCARGEYTV
LDYWGQGTLVTVSSASPREPQVYTDPPSRDELTKNQVSLTCLVKGFYPSDIA
VEWESNGQPENNYKTTPPVLDSDGSFFLYSKLTVDKSRWQQGNVFSCSVMH
EALHNHYTQKSLSLSGEC
Domain arrangement:
F- G
VH- CH3
Mutations in domain G
L351D, 445G, 446E, 447C insertion
>hCTLA4-4 domain F [SEQ ID NO: 54]
EVQLVESGGGLVQPGGSLRLSCAASGFTFSTYYIHWVRQAPGKGLEWVAVIY
PYTGFTYYADSVKGRFTISADTSKNTAYLQMNSLRAEDTAVYYCARGEYTV
LDYWGQGTLVTVSSAS
>hCTLA4-4 domain G [SEQ ID NO: 55]
PREPQVYTDPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSGEC
Other Sequences:
>Hinge: DKTHTCPPCP [SEQ ID NO: 56]
>BC1-Polypeptide 1 Domain Junction: IKRTPREP [SEQ ID NO: 57]
>BC15-Polypeptide 1 Domain Junction: IKRTVREP [SEQ ID NO: 58]
>BC16-Polypeptide 1 Domain Junction: IKRTREP [SEQ ID NO: 59]
>BC17-Polypeptide 1 Domain Junction: IKRTVPREP [SEQ ID NO: 60]
>BC26-Polypeptide 1 Domain Junction: IKRTVAEP [SEQ ID NO: 61]
>BC27-Polypeptide 1 Domain Junction: IKRTVAPREP [SEQ ID NO: 62]
>BC1-Polypeptide 2 Domain Junction: SSASPREP [SEQ ID NO: 63]
>BC13-Polypeptide 2 Domain Junction: SSASTREP [SEQ ID NO: 64]
>BC14-Polypeptide 2 Domain Junction: SSASTPREP [SEQ ID NO: 65]
>BC24-Polypeptide 2 Domain Junction: SSASTKGEP [SEQ ID NO: 66]1
>BC25-Polypeptide 2 Domain Junction: SSASTKGREP [SEQ ID NO: 67]
>SP34-89 VH [SEQ ID NO: 68]
EVQLVESGGGLVQPGGSLRLSCAASGFTFNTYAMNWVRQAPGKGLEWVARIRSKYNNYAT
YYADSVKGRFSISRDDSKNTAYLQMNSLRAEDTAVYYCVRHGNFGNSYVSWFAYWGQGTL
VTV
>SP34-89 VL [SEQ ID NO: 69]
QAVVTQEPSLTVSPGGTVTLTCRSSTGAVTTSNYANWVQQKPGQAPRGLIGGTNKRAPWTP
ARFSGSLLGGKAALTITGAQAEDEADYYCALWYSNLWVFGGGTKLTVL
>SP34-89 VH-N30S VH [SEQ ID NO: 70] lower case denotes mutation
EVQLVESGGGLVQPGGSLRLSCAASGFTFsTYAMNWVRQAPGKGLEWVARIRSKYNNYATY
YADSVKGRFSISRDDSKNTAYLQMNSLRAEDTAVYYCVRHGNFGNSYVSWFAYWGQGTLV
TV
>SP34-89 VH-G65D VH [SEQ ID NO: 71] lower case denotes mutation
EVQLVESGGGLVQPGGSLRLSCAASGFTFNTYAMNWVRQAPGKGLEWVARIRSKYNNYAT
YYADSVKdRFSISRDDSKNTAYLQMNSLRAEDTAVYYCVRHGNFGNSYVSWFAYWGQGTL
VTV
>SP34-89 VH-S68T VH [SEQ ID NO: 72] lower case denotes mutation
EVQLVESGGGLVQPGGSLRLSCAASGFTFNTYAMNWVRQAPGKGLEWVARIRSKYNNYAT
YYADSVKGRFtISRDDSKNTAYLQMNSLRAEDTAVYYCVRHGNFGNSYVSWFAYWGQGTL
VTV
>SP34-89 VL-W57G VL [SEQ ID NO: 73] lower case denotes mutation
QAVVTQEPSLTVSPGGTVTLTCRSSTGAVTTSNYANWVQQKPGQAPRGLIGGTNKRAPgTPA
RFSGSLLGGKAALTITGAQAEDEADYYCALWYSNLWVFGGGTKLTVL
>Phage display heavy chain [SEQ ID NO: 74]:
EVQLVESGGGLVQPGGSLRLSCAASGFTExxxx [symbol] WVRQAPGKGLEWVAxxxx
xxxxxxx [symbol] RFTISADTSKNTAYLQMNSLRAEDTAVYYCARxxxxxxxxxx
xxx [symbol] WGQGTLVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPS
NTKVDKKVEPKSCDKTHTCPPCPAPELLGGPSVFLEPPKPKDTLMISRTPEVTC
VVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQV
SLTCLVKGEYPSDIAVEWESNGQPENNYKTTPPVLDSDGSFFLYSKLTVDKSR
WQQGNVFSCSVMHEALHNHYTQKSLSLSPGK
>Phage display light chain [SEQ ID NO: 75]:
DIQMTQSPSSLSASVGDRVTITC [symbol] VAWYQQKPGKAPKLLIY [symbol] [symbol]
GVPSRFSGSRSGTDFTLTISSLQPEDFATYYC [symbol] xxxxxx [symbol] GQGTKVEIKRT
VAAPSVFIFPPSDSQLKSGTASVVCLLNNFYPREAKVQWKVDNALQSGNSQE
SVTEQDSKDSTYSLSSTLTLSKADYEKHKVYACEVTHQGLSSPVTKSFNRGEC
>B-Body Domain A/H Scaffold [SEQ ID NO: 76]:
DIQMTQSPSSLSASVGDRVTITC [symbol] VAWYQQKPGKAPKLLIY [symbol] [symbol]
GVPSRFSGSRSGTDFTLTISSLQPEDFATYYC [symbol] xxxxxx [symbol] GQGTKVEIKRT
>B-Body Domain F/L Scaffold [SEQ ID NO: 77]:
EVQLVESGGGLVQPGGSLRLSCAASGFTExxxx [symbol] WVRQAPGKGLEWVAxxxx
xxxxxxx [symbol] RFTISADTSKNTAYLQMNSLRAEDTAVYYCARxxxxxxxxxx
xxx [symbol] WGQGTLVTVSSAS
>BC1 Chain 1 Scaffold [SEQ ID NO: 78]
DIQMTQSPSSLSASVGDRVTITC [symbol] VAWYQQKPGKAPKWY [symbol] [symbol]
GVPSRFSGSRSGTDFTLTISSLQPEDFATYYC [symbol] xxxxxx [symbol] GQGTKVEIKRTP
REPQVYTLPPSRDELTKNQVSLKCLVKGFYPSDIAVEWESNGQPENNYKTTPP
VLDSDGSFFLYSKLTVDKSRWQQGNVFSCSVMHEALHNHYTQKSLSLSKSC
DKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKE
YKCKVSNKALPAPIEKTISKAKGQPREPQVYTLPPCRDELTKNQVSLWCLVKG
FYPSDIAVEWESNGQPENNYKTTPPVLDSDGSFFLYSKLTVDKSRWQQGNVFSCSV
MHEALHNHYTQKSLSLSPGK
″x″ represents CDR amino acids that were varied to create the library, and bold
italic represents the CDR sequences that were constant
Domain arrangement:
A- B- Hinge- D- E
VL- CH3- Hinge- CH2- CH3(knob)
Mutations in first CH3 (Domain B):
T366K; 445K, 446S, 447C insertion
Mutations in second CH3 (Domain E):
S354C, T366W
>BC1 Chain 2 Scaffold [SEQ ID NO: 79]
EVQLVESGGGLVQPGGSLRLSCAASGFTExxxx [symbol] WVRQAPGKGLEWVAxxxx
xxxxxxx [symbol] RFTISADTSKNTAYLQMNSLRAEDTAVYYCARxxxxxxxxxx
xxx [symbol] WGQGTLVTVSSASPREPQVYTDPPSRDELTKNQVSLTCLVKGFYPSDI
AVEWESNGQPENNYKTTPPVLDSDGSFFLYSKLTVDKSRWQQGNVFSCSVM
HEALHNHYTQKSLSLSGEC
″x″ represents CDR amino acids that were varied to create the library, and bold
italic represents the CDR sequences that were constant
Domain arrangement:
F- G
VH- CH3
Mutations in CH3 (Domain G):
L351D; 445G, 446E, 447C insertion
>BC1 Chain 3 Scaffold [SEQ ID NO: 80]
DIQMTQSPSSLSASVGDRVTITC [symbol] VAWYQQKPGKAPKLLIY [symbol] [symbol]
GVPSRFSGSRSGTDFTLTISSLQPEDFATYYC [symbol] xxxxxx [symbol] GQGTKVEIKRT
VAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC
DKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKE
YKCKVSNKALPAPIEKTISKAKGQPREPQVCTLPPSREEMTKNQVSLSCAVKG
FYPSDIAVEWESNGQPENNYKTTPPVLDSDGSFFLVSKLTVDKSRWQQGNFFSCS
VMHEALHNHYTQKSLSLSPGK
″x″ represents CDR amino acids that were varied to create the library, and bold
italic represents the CDR sequences that were constant
Domain arrangement:
H- I- Hinge- J- K
VL- CL- Hinge- CH2- CH3(hole)
Mutations in CH3 (domain K):
Y349C, D356E, L358M, T3665, L368A, Y407V
>BC1 Chain 4 Scaffold [SEQ ID NO: 81]
EVQLVESGGGLVQPGGSLRLSCAASGFTFxxxx [symbol] WVRQAPGKGLEWVAxxxx
xxxxxxx [symbol] RFTISADTSKNTAYLQMNSLRAEDTAVYYCARxxxxxxxxxx
xxx [symbol] WGQGTLVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPS
NTKVDKKVEPPKSC
″x″ represents CDR amino acids that were varied to create the library, and bold
italic represents the CDR sequences that were constant
Domain arrangement:
L- M
VH- CH1
>BC1 Chain 3 1(A)x2(B-A) SP34-89 Scaffold [SEQ ID NO: 82]
DIQMTQSPSSLSASVGDRVTITC [symbol] VAWYQQKPGKAPKWY [symbol] [symbol]
GVPSRFSGSRSGTDFTLTISSLQPEDFATYYC [symbol] xxxxxx [symbol] GQGTKVEIKRTP
REPQVYTLPPSRDELTKNQVSLKCLVKGFYPSDIAVEWESNGQPENNYKTTPP
VLDSDGSFFLYSKLTVDKSRWQQGNVFSCSVMHEALHNHYTQKSLSLSKSCT
ASSGGSSSGQAVVTQEPSLTVSPGGTVTLTCRSSTGAVTTSNYANWVQQKPG
QAPRGLIGGTNKRAPWTPARFSGSLLGGKAALTITGAQAEDEADYYCALWY
SNLWVFGGGTKLTVLGRTVAAPSVFIFPPSDEQLKSGTASVVCLLNNFYPR
EAKVQWKVDNALQSGNSQESVTEQDSKDSTYSLSSTLTLSKADYEKHKV
″YACEVTHQGLSSPVTKSFNRGECDKTHTCPPCPAPEAAGGPSVFLFPPKPK
DTLMISRTPEVTCVVVDVSHEDPEVKFNWYVDGVEVHNAKTKPREEQYN
STYRVVSVLTVLHQDWLNGKEYKCKVSNKALKAPIEKTISKAKGQPREP
QVCTLPPSREEMTKNQVSLSCAVKGFYPSDIAVEWESNGQPENNYKTTPPVLDSD
GSFFLVSKLTVDKSRWQQGNYFSCSVMHEALHNHYTQKSLSLSPGK
″x″ represents CDR amino acids that were varied to create the library, and bold
italic represents the CDR sequences that were constant
Domain arrangement:
R- S- linker- H- I- Hinge- J- K
VL- CH3- linker- SP34- CL- Hinge- CH2- CH3(hole)
Mutations in domain S:
T366K; 445K, 446S, 447C insertion
Ten amino acids linker insertion: TASSGGSSSG
Mutations in Domain J:
L234A, L235A, and P329K
Mutations in domain K:
Y349C, D356E, L358M, T366S, L368A, Y407V
>BC1 Chain 3 1(A)x2(B-A) SP34-89 S-H Junction [SEQ ID NO: 83]
TASSGGSSSG
>Library Parent Heavy Chain [SEQ ID NO: 84]
EVQLVESGGGLVQPGGSLRLSCAASGFTFxxxx [symbol] WVRQAPGKGLEWV
Axxxxxxxxxxx [symbol] RFTISADTSKNTAYLQMNSLRAEDTAVYYCARxxxxx
xxxxxxxx [symbol] WGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHK
PSNTKVDKKVEPKSCDKTHTCPPCPAPELLGGPSVFLFPPKP
(SEQ ID NO: 96)
AGKC
(SEQ ID NO: 97)
PGKC
(SEQ ID NO: 98)
AGKGC
(SEQ ID NO: 99)
AGKGSC
>BA1 chain 1 [SEQ ID NO: 100]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLGECDKTHTCPPCP
APELLGGPSVFLEPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQP
REPQVYTLPPCRDELTKNQVSLWCLVKGFYPSDIAVEWESNGQPENNYKTTPPVLDS
DGSFFLYSKLTVDKSRWQQGNVFSCSVMHEALHNHYTQKSLSLSPGK
>BA1 chain 2 [SEQ ID NO: 101]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLGEC
>BA(all) chain 3 [SEQ ID NO: 102]
EIVLTQSPATLSLSPGERATLSCRASQSVSSYLAWYQQKPGQAPRLLIYDASNRATGI
PARFSGSGSGTDFTLTISSLEPEDFAVYYCQQSSNWPRTFGQGTKVEIKRTVAAPSVFI
FPPSDEQLKSGTASVVCLLNNFYPREAKVQWKVDNALQSGNSQESVTEQDSKDSTY
SLSSTLTLSKADYEKHKVYACEVTHQGLSSPVTKSFNRGECDKTHTCPPCPAPELLG
GPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQV
CTLPPSREEMTKNQVSLSCAVKGFYPSDIAVEWESNGQPENNYKTTPPVLDSDGSFF
LVSKLTVDKSRWQQGNVFSCSVMHEALHNHYTQKSLSLSPGK
>BA(all) chain 4 [SEQ ID NO: 103]
QVQLVESGGGVVQPGRSLRLDCKASGITFSNSGMHWVRQAPGKGLEWVAVIWYDG
SKRYYADSVKGRFTISRDNSKNTLFLQMNSLRAEDTAVYYCATNDDYWGQGTLVT
VSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPPKSC
>BA2 [SEQ ID NO: 104]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGC
>BA3 [SEQ ID NO: 105]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGC
>BA4 [SEQ ID NO: 106]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGSC
>BA5 [SEQ ID NO: 107]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKC
>BA9 [SEQ ID NO: 108]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGC
>BA10 [SEQ ID NO: 109]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGC
>BA11 [SEQ ID NO: 110]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGC
>BA12 [SEQ ID NO: 111]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGC
>BA13 [SEQ ID NO: 112]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGC
>BA14 [SEQ ID NO: 113]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGC
>BA15 [SEQ ID NO: 114]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGC
>BA16 [SEQ ID NO: 115]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGC
>BA17 [SEQ ID NO: 116]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGC
>BA18 [SEQ ID NO: 117]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGC
>BA19 [SEQ ID NO: 118]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGC
>BA20 [SEQ ID NO: 119]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGC
>BA21 [SEQ ID NO: 120]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGSC
>BA22 [SEQ ID NO: 121]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGSC
>BA23 [SEQ ID NO: 122]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGSC
>BA24 [SEQ ID NO: 123]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGSC
>BA25 [SEQ ID NO: 124]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGSC
>BA26 [SEQ ID NO: 125]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKGSC
>BA27 [SEQ ID NO: 126]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKC
>BA28 [SEQ ID NO: 127]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKC
>BA29 [SEQ ID NO: 128]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKC
>BA30 [SEQ ID NO: 129]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKC
>BA31 [SEQ ID NO: 130]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKC
>BA32 [SEQ ID NO: 131]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKC
>BA33 [SEQ ID NO: 132]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLGEC
>BA34 [SEQ ID NO: 133]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLGEC
>BA35 [SEQ ID NO: 134]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLGEC
>BA36 [SEQ ID NO: 135]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLGEC
>BA37 [SEQ ID NO: 136]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLGEC
>BA38 [SEQ ID NO: 137]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLGEC
>BA39 [SEQ ID NO: 138]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLPGKC
>BA40 [SEQ ID NO: 139]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLPGKC
>BA41 [SEQ ID NO: 140]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLPGKC
>BA42 [SEQ ID NO: 141]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLPGKC
>BA43 [SEQ ID NO: 142]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLPGKC
>BA44 [SEQ ID NO: 143]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLPGKC
>BA2 [SEQ ID NO: 144]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKGSC
>BA3 [SEQ ID NO: 145]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGC
>BA4 [SEQ ID NO: 146]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGC
>BA5 [SEQ ID NO: 147]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGC
>BA9 [SEQ ID NO: 148]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGC
>BA10 [SEQ ID NO: 149]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKGC 1\
>BA11 [SEQ ID NO: 150]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKGSC
>BA12 [SEQ ID NO: 151]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKC
>BA13 [SEQ ID NO: 152]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLGEC
>BA14 [SEQ ID NO: 153]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLPGKC
>BA15 [SEQ ID NO: 154]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGC
>BA16 [SEQ ID NO: 155]
EVQLVESGGGLVQPGGSLRLSCAASGETFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKGC
>BA17 [SEQ ID NO: 156]
EVQLVESGGGLVQPGGSLRLSCAASGETFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKGSC
>BA18 [SEQ ID NO: 157]
EVQLVESGGGLVQPGGSLRLSCAASGETFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKC
>BA19 [SEQ ID NO: 158]
EVQLVESGGGLVQPGGSLRLSCAASGETFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLGEC
>BA20 [SEQ ID NO: 159]
EVQLVESGGGLVQPGGSLRLSCAASGETFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLPGKC
>BA21 [SEQ ID NO: 160]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGC
>BA22 [SEQ ID NO: 161]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKGC
>BA23 [SEQ ID NO: 162]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKGSC
>BA24 [SEQ ID NO: 163]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKC
>BA25 [SEQ ID NO: 164]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLGEC
>BA26 [SEQ ID NO: 165]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLPGKC
>BA27 [SEQ ID NO: 166]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGC
>BA28 [SEQ ID NO: 167]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKGC
>BA29 [SEQ ID NO: 168]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKGSC
>BA30 [SEQ ID NO: 169]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKC
>BA31 [SEQ ID NO: 170]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLGEC
>BA32 [SEQ ID NO: 171]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLPGKC
>BA33 [SEQ ID NO: 172]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGC
>BA34 [SEQ ID NO: 173]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKGC
>BA35 [SEQ ID NO: 174]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSAS
TFRPEVHLLPPPSEELALNELVTLTCLARGFSPKDVLVRWLQGSQELPREKYLTWAS
RQEPSQGTTTFAVTSILRVAAEDWKKGDTFSCMVGHEALPLAFTQKTIDRLAGKGSC
>BA36 [SEQ ID NO: 175]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKC
>BA37 [SEQ ID NO: 176]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLGEC
>BA38 [SEQ ID NO: 177]1
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLPGKC
>BA39 [SEQ ID NO: 178]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGC
>BA40 [SEQ ID NO: 179]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKGC
>BA41 [SEQ ID NO: 180]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKGSC
>BA42 [SEQ ID NO: 181]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLAGKC
>BA43 [SEQ ID NO: 182]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLGEC
>BA44 [SEQ ID NO: 183]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTFRPEVHLLPPPSEELALNELVTLTCLARGFSPKDVLVRWLQGSQELP
REKYLTWASRQEPSQGTTTFAVTSILRVAAEDWKKGDTFSCMVGHEALPLAFTQKTI
DRLPGKC 1
>Human IgA CH3 sequence [SEQ ID NO: 184]
TFRPEVHLLPPPSEELALNELVTLTCLARGFSPKDVLVRWLQGSQELPREKYLTWAS
RQEPSQGTTTFAVTSILRVAAEDWKKGDTFSCMVGHEALPLAFTQKTIDRL
>T27, T36 chain 1 [SEQ ID NO: 185]
ELVMTQSPSSLTVTAGEKVTMSCKSSQSLLNSGNQKNYLTWYQQKPGQPPKLLIYW
ASTRESGVPDRFTGSGSGTDFTLTISSVQAEDLAVYYCQNDYSYPLTFGAGTKLEIKR
TTFRPEVHLLPPPSEELALNELVTLTCLARGFSPKDVLVRWLQGSQELPREKYLTWA
SRQEPSQGTTTFAVTSILRVAAEDWKKGDTFSCMVGHEALPLAFTQKTIDRLAGCDK
THTCPPCPAPEAAGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALK
APIEKTISKAKGQPREPQVCTLPPSREEMTKNQVSLSCAVKGFYPSDIAVEWESNGQ
PENNYKTTPPVLDSDGSFFLVSKLTVDKSRWQQGNVFSCSVMHEALHNHYTQKSLS
LSPGK
Domain arrangement
A B Hinge D E
VL CH3 Hinge CH2 CH3
Mutations in first CH3 (Domain B)
Substitution of IgG-CH3 with IgA-CH3, C-terminus AGC linker addition
Mutations in CH2 (Domain D)
L234A, L235A, P329K
Mutations in second CH3 (Domain E)
Y349C, D356E, L358M, T3665, L368A, Y407V
>T27, T36 chain 2 [SEQ ID NO: 186]
EVQLLEQSGAELVRPGTSVKISCKASGYAFTNYWLGWVKQRPGHGLEWIGDIFPGS
GNIHYNEKFKGKATLTADKSSSTAYMQLSSLTFEDSAVYFCARLRNWDEPMDYWG
QGTTVTVSSASTFRPEVHLLPPPSEELALNELVTLTCLARGFSPKDVLVRWLQGSQEL
PREKYLTWASRQEPSQGTTTFAVTSILRVAAEDWKKGDTFSCMVGHEALPLAFTQK
TIDRLAGKGSC
Domain arrangement
F G
VH CH3
Mutations in CH3 (Domain G)
Substitution of IgG-CH3 with IgA-CH3, C-terminus AGKGSC linker addition
>T27 chain 3 [SEQ ID NO: 187]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTTFRPEVC
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLTASSGGSSSGQAVV
TQEPSLTVSPGGTVTLTCRSSTGAVTTSNYANWVQQKPGQAPRGLIGGTNKRAP
WTPARFSGSLLGGKAALTITGAQAEDEADYYCALWYSNLWVFGGGTKLTVLG
RTVAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGECDKTHTCP
PCPAPEAAGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALKAPIEKTISKAK
GQPREPQVYTLPPCRDELTKNQVSLWCLVKGFYPSDIAVEWESNGQPENNYKT
TPPVLDSDGSFFLYSKLTVDKSRWQQGNVFSCSVMHEALHNHYTQKSLSLSPGK
Domain arrangement
N O Hinge H I Hinge 2 J K
VL CH3 Hinge VL CL Hinge 2 CH2 CH3
Mutations in first CH3 (Domain O)
Substitution of IgG-CH3 with IgA-CH3, H350C
Mutations in CH2 (Domain J)
L234A, L235A, P329K
Mutations in second CH3 (Domain K)
S354C, T366W
>T27 chain 4 [SEQ ID NO: 188]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPPKSC
Domain arrangement
L M
VH CH1
>T27 chain 5 [SEQ ID NO: 161
EVQLVESGGGLVQPGGSLRLSCAASGFNIKDTYIHWVRQAPGKGLEWVARTYPTNG
YTRYADSVKGRFTISADTSKNTAYLQMNSLRAEDTAVYYCSRWGGDGFYAMDYW
GQGTLVTVSSTFRPEVHLLPPCSEELALNELVTLTCLARGFSPKDVLVRWLQGSQELP
REKYLTWASRQEPSQGTTTFAVTSILRVAAEDWKKGDTFSCMVGHEALPLAFTQKTI
DRL
Domain arrangement
P Q
VH CH3
Mutations in CH3 (Domain Q)
Substitution of IgG-CH3 with IgA-CH3, P355C
>T36 chain 3 [SEQ ID NO: 189]
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTPREPQVY
TLPPSRDELTKNQVSLKCLVKGFYPSDIAVEWESNGQPENNYKTTPPVLDSDGSFFLY
SKLTVDKSRWQQGNVFSCSVMHEALHNHYTQKSLSLSKSCTASSGGSSSGQAVVTQ
EPSLTVSPGGTVTLTCRSSTGAVTTSNYANWVQQKPGQAPRGLIGGTNKRAPW
TPARFSGSLLGGKAALTITGAQAEDEADYYCALWYSNLWVFGGGTKLTVLGRT
VAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGECDKTHTCPPC
PAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALKAPIEKTISKAKG
QPREPQVYTLPPCRDELTKNQVSLWCLVKGFYPSDIAVEWESNGQPENNYKTT
PPVLDSDGSFFLYSKLTVDKSRWQQGNVFSCSVMHEALHNHYTQKSLSLSPGK
Domain arrangement
N O Hinge H I Hinge 2 J K
VL CH3 Hinge VL CL Hinge 2 CH2 CH3
Mutations in first CH3 (Domain O)
T366K, 445K, 446S, 447C tripeptide insertion
Mutations in CH2 (Domain J)
L234A, L235A, P329K
Mutations in second CH3 (Domain K)
S354C, T366W
>T36 chain 4 [SEQ ID NO: 190]
EVQLVESGGGLVQPGGSLRLSCAASGFTFSDYDIHWVRQAPGKGLEWVGDITPYDG
TTNYADSVKGRFTISADTSKNTAYLQMNSLRAEDTAVYYCARLVGEIATGFDYWGQ
GTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPPKSC
Domain arrangement
L M
VH CH1
>T36 chain 5 [SEQ ID NO: 19]
EVQLVESGGGLVQPGGSLRLSCAASGFNIKDTYIHWVRQAPGKGLEWVARTYPTNG
YTRYADSVKGRFTISADTSKNTAYLQMNSLRAEDTAVYYCSRWGGDGFYAMDYW
GQGTLVTVSSPREPQVYTDPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSG
EC
Domain arrangement
P Q
VH CH3
Mutations in CH3 (Domain Q)
L351D, 445G, 446E, 447C tripeptide insertion

Domain Arrangement:

### 7. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### 8. EQUIVALENTS

While various specific embodiments have been illustrated and described, the above specification is not restrictive. It will be appreciated that various changes can be made without departing from the spirit and scope of the invention(s). Many variations will become apparent to those skilled in the art upon review of this specification.

What is claimed is:

1. A binding molecule comprising a first, second, third, fourth, and fifth polypeptide chain, wherein:
a. the first polypeptide chain comprises a domain A, a domain B, a domain D, and a domain E, wherein the domains are arranged, from N-terminus to C-terminus, in a A-B-D-E orientation, wherein domain A comprises a variable region domain amino acid sequence, domain B comprises a CH3 domain amino acid sequence, domain D comprises a constant region domain amino acid sequence, and domain E comprises a constant region domain amino acid sequence;
b. the second polypeptide chain comprises a domain F and a domain G, wherein the domains are arranged, from N-terminus to C-terminus, in a F-G orientation, and wherein domain F has a variable region domain amino acid sequence and domain G comprises a CH3 domain amino acid sequence;
c. the third polypeptide chain comprises a domain H, a domain I, a domain J, and a domain K, wherein the domains are arranged, from N-terminus to C-terminus, in a H-I-J-K orientation, and wherein domain H has a variable region domain amino acid sequence, domain I has a constant region amino acid sequence, and domains J and K have a constant region domain amino acid sequence;
d. the fourth polypeptide chain comprises a domain L and a domain M, wherein the domains are arranged, from N-terminus to C-terminus, in a L-M orientation, and wherein domain L has a variable region domain amino acid sequence, and domain M comprises a constant region amino acid sequence, or portion thereof;
e. the fifth polypeptide chain comprises a domain P and a domain Q, wherein the domains are arranged, from N-terminus to C-terminus, in a P-Q orientation, and wherein domain P has a variable region domain amino acid sequence and domain Q has a CH3 domain acid sequence;
f. the first polypeptide chain or the third polypeptide chain further comprises a domain N and a domain O, wherein domain N has a variable region domain amino acid sequence, wherein domain O has a CH3 domain amino acid sequence, wherein domains N and O are arranged, from N-terminus to C-terminus, in a N—O orientation, and wherein the C-terminus of domain O is attached, directly or indirectly, to the N-terminus of domain A of the first polypeptide chain or to the N-terminus of domain H of the third polypeptide chain;
g. domains A and F form a first antigen binding site (ABS), domains H and L form a second ABS, and domains N and P form a third ABS;
h. domains B and G form a first domain pair of associated constant region domains (“first domain pair”), domains I and M form a second domain pair of associated constant region domains (“second domain pair”), and domains Q and O form a third domain pair of associated constant region domains (“third domain pair”);
i. at least one of the first and third domain pairs is an IgA-CH3 domain pair and the second domain pair is a CH1/CL domain pair;
j. the first and the second polypeptides are associated through an interaction between the A and the F domains and an interaction between the B and the G domains, the third and the fourth polypeptides are associated through an interaction between the H and the L domains and an interaction between the I and the M domains, the first and the third polypeptides are associated through an interaction between the D and the J domains and an interaction between the E and the K domains, and either the first or third polypeptide chain is associated with the fifth polypeptide chain through an interaction between the N and the P domains and an interaction between the O and the Q domains to form the binding molecule.

2. The binding molecule of claim 1, wherein the first polypeptide chain further comprises domain N and domain O.

3. The binding molecule of claim 1, wherein the third polypeptide chain further comprises domain N and a domain O.

4. The binding molecule of any one of the preceding claims, wherein:
a. Domain A comprises a VL amino acid sequence, optionally wherein domain A is VL; and
b. Domain F comprises a VH amino acid sequence, optionally wherein domain F is VH.

5. The binding molecule of any one of the preceding claims, wherein domain I is CL and domain M is CH1.

6. The binding molecule of any one of the preceding claims, wherein:
a. Domain H comprises a VL amino acid sequence, optionally wherein domain His VL; and
b. Domain L comprises a VH amino acid sequence, optionally wherein domain L is VH.

7. The binding molecule of any one of the preceding claims, wherein:
a. domain N comprises a VL amino acid sequence, optionally wherein domain Nis VL; and
b. Domain P comprises a VH amino acid sequence, optionally wherein domain P is VH.

8. The binding molecule of any one of claims 1-7, wherein domains B and G are IgA-CH3 domains and the first domain pair is an IgA-CH3/IgA-CH3 domain pair.

9. The binding molecule of claim 8, wherein domains O and Q are IgA-CH3 domains and the third domain pair is an IgA-CH3/IgA-CH3 domain pair.

10. The binding molecule of claim 9, wherein
a. domain B and domain G each comprise an orthogonal amino acid substitution that provides a non-endogenous cysteine, the non-endogenous cysteines capable of forming a disulfide bridge, and domain O and Q comprise a first and second CH3 linker sequence, respectively; or
b. domain B and domain G comprise a first and second CH3 linker, respectively, and domain O and Q each comprise an orthogonal amino acid substitution that provides a non-endogenous cysteine, the non-endogenous cysteines capable of forming a disulfide bridge;
wherein the first and second CH3 linker sequence each comprise a cysteine capable of forming a disulfide bridge with the cysteine in the other CH3 linker sequence.

11. The binding molecule of claim 10, wherein the orthogonal amino acid substitutions providing non-endogenous cysteines are a P355C substitution in one IgA-CH3 domain and a H350C substitution in the other IgA-CH3 domain.

12. The binding molecule of claim 10 or 11, wherein one of the first and second CH3 linkers is AGC and the other CH3 linker is AGKGSC.

13. The binding molecule of claim 12, wherein either:
a. Domain B and domain G each comprise an orthogonal amino acid substitution that provides a non-endogenous cysteine, wherein domain B comprises an H350C mutation and domain G comprises a P355C mutation, and wherein domain O comprises a first CH3 linker which is AGC and domain Q comprises a second CH3 linker which is AGKGSC;
b. Domain B and domain G each comprise an orthogonal amino acid substitution that provides a non-endogenous cysteine, wherein domain B comprises an H350C mutation and domain G comprises a P355C mutation, and wherein domain O comprises a first CH3 linker which is AGKGSC and domain Q comprises a second CH3 linker which is AGC; or
c. Domain B comprises a first CH3 linker which is AGC, domain G comprises a second CH3 linker which is AGKGSC, domain O comprises an H350C mutation and domain Q comprises a P355C mutation.

14. The binding molecule of claim 8, wherein domains O and Q are IgG CH3 domains, optionally IgG1-CH3 domains.

15. The binding molecule of claim 14, wherein domain O and domain Q each comprise an orthogonal charge pair mutation.

16. The binding molecule of claim 15, wherein the orthogonal charge pair mutations comprise a T366K mutation in one of domains O and Q and a L351D mutation in the other domain.

17. The binding molecule of claim 16, wherein domain O comprises the T366K mutation and domain Q comprises the L351D mutation.

18. The binding molecule of any one of claims 14-17, wherein domain O and domain Q each comprise an amino acid modification that provides a non-endogenous cysteine, the non-endogenous cysteines capable of forming a disulfide bridge.

19. The binding molecule of claim 18, wherein the amino acid modifications are 447C modifications.

20. The binding molecule of any one of claims 14-19, wherein either:
a. Domain B comprises a first CH3 linker which is AGC, domain G comprises a second CH3 linker which is AGKGSC, domain O comprises a T366K mutation and 447C modification, and domain Q comprises a L351D mutation and 447C modification; or
b. Domain B comprises a H350C mutation, domain G comprises a P355C mutation, domain O comprises a T366K mutation and 447C modification, and domain Q comprises a L351D mutation and 447C modification.

21. The binding molecule of any one of claims 1-5, wherein
a. domains B and G are IgG-CH3 domains, optionally IgG1-CH3 domains, and the first domain pair is an IgG-CH3/IgG-CH3 domain pair; and
b. domains O and Q are IgA-CH3 domains and the third domain pair is an IgA-CH3/IgA-CH3 domain pair.

22. The binding molecule of claim 21, wherein domain B and domain G each comprise an amino acid modification that provides a non-endogenous cysteine, the non-endogenous cysteines capable of forming a disulfide bridge.

23. The binding molecule of claim 22, wherein the amino acid modifications are a 349C mutation in one of domains B and G and a 354C mutation in the other domain.

24. The binding molecule of claim 23, wherein domain B comprises a P343V mutation.

25. The binding molecule of claim 22, wherein the amino acid modifications are 447C modifications.

26. The binding molecule of any one of claims 21-25, wherein domain B and domain G each comprise an orthogonal charge pair mutation.

27. The binding molecule of claim 26, wherein the orthogonal charge pair mutations comprise a T366K mutation in one of domains B and G and a L351D mutation in the other domain.

28. The binding molecule of claim 27, wherein domain B comprises the T366K mutation and domain G comprises the L351D mutation.

29. The binding molecule of any one of claims 21-28, wherein domain O and domain Q each comprise an amino acid modification that provides a non-endogenous cysteine, the non-endogenous cysteines capable of forming a disulfide bridge, wherein the amino acid modifications of domains O and Q providing the non-endogenous cysteines are different from the amino acid modifications of domains B and G providing the non-endogenous cysteines.

30. The binding molecule of claim 29, wherein one of domains O and Q comprise an H350C mutation and the other domain comprises a P355C mutation, optionally wherein domain O comprises the H350C mutation and domain Q comprises the P355C mutation.

31. The binding molecule of claim 29, wherein domain O comprises a first CH3 linker and domain Q comprises a second CH3 linker, the first and second CH3 linkers each comprising the non-endogenous cysteines capable of forming the disulfide bridge.

32. The binding molecule of claim 31, wherein
a. domain B comprises a T366K mutation and 447C amino acid modification;
b. domain G comprises an L351D mutation and 447C amino acid modification;
c. the first CH3 linker is the amino acid sequence AGC; and
d. the second CH3 linker is the amino acid sequence AGKGSC.

33. The binding molecule of claim 24, wherein domain O comprises a first CH3 linker which is the amino acid sequence AGC and domain Q comprises a second CH3 linker which is the amino acid sequence AGKGSC.

34. The binding molecule of claim 3, wherein:
a. domain A comprises a VH amino acid sequence, optionally wherein domain A is VH; and
b. domain F comprises a VL amino acid sequence, optionally wherein domain F is VL.

35. The binding molecule of claim 34, wherein:
a. domain H comprises a VH amino acid sequence, optionally wherein domain H is VH; and
b. domain L comprises a VL amino acid sequence, optionally wherein domain L is VL.

36. The binding molecule of claim 34 or 35, wherein domain N comprises a VH amino acid sequence, optionally wherein domain N is VH.

37. The binding molecule of any one of claims 34-36, wherein domain I is CH1 and domain M is CL.

38. A binding molecule comprising a first, second, third, and fourth polypeptide chain, wherein:
a. the first polypeptide chain comprises a domain A, a domain B, a domain D, and a domain E, wherein the domains are arranged, from N-terminus to C-terminus, in a A-B-D-E orientation, wherein domain A comprises a VL amino acid sequence, domain B comprises an IgA-CH3 domain sequence, domain D comprises a constant region domain amino acid sequence, and domain E comprises a constant region domain amino acid sequence;
b. the second polypeptide chain comprises a domain F and a domain G, wherein the domains are arranged, from N-terminus to C-terminus, in a F-G orientation, and wherein domain F has a VH amino acid sequence and domain G comprises an IgA-CH3 domain sequence;
c. the third polypeptide chain comprises a domain H, a domain I, a domain J, and a domain K, wherein the domains are arranged, from N-terminus to C-terminus, in a H-I-J-K orientation, and wherein domain H has a variable region domain amino acid sequence, domain I has a constant region amino acid sequence, and domains J and K have a constant region domain amino acid sequence;
d. the fourth polypeptide chain comprises a domain L and a domain M, wherein the domains are arranged, from N-terminus to C-terminus, in a L-M orientation, and wherein domain L has a variable region domain amino acid sequence, and domain M comprises a constant region amino acid sequence, or portion thereof;
e. domains A and F form a first antigen binding site (ABS) and domains H and L form a second ABS; and
f. the first and the second polypeptides are associated through an interaction between the A and the F domains and an interaction between the B and the G domains, the third and the fourth polypeptides are associated through an interaction between the H and the L domains and an interaction between the I and the M domains, and the first and the third polypeptides are associated through an interaction between the D and the J domains and an interaction between the E and the K domains to form the binding molecule.

39. The binding molecule of claim 38, wherein domains B and G are IgA-CH3 domains.

40. The binding molecule of claim 38 or 39, wherein domains E and K comprise an IgG-CH3 amino acid sequence.

41. The binding molecule of claim 40, wherein domains E and K are IgG-CH3 amino acid domains.

42. The binding molecule of claim 40, wherein domains E and K comprise an IgG1-CH3 amino acid sequence.

43. The binding molecule of claim 42, wherein domains E and K are IgG1-CH3 domains.

44. The binding molecule of any one of claims 40-43, wherein the domains E and K further comprise knob-hole mutations.

45. The binding molecule of claim 44, wherein the knob-hole mutations comprise a T366W mutation in one domain selected from domains E and K and a 366S, 368A, and 407V mutations in the other domain selected from domains E and K.

46. The binding molecule of claim 45, wherein domain E comprises the T366W mutation and domain K comprises the 366S, 368A, and 407V mutations.

47. The binding molecule of claim 69, wherein domains A and H comprise a VL amino acid sequence, and wherein domains F and L comprise a VH amino acid sequence.

48. The binding molecule of any one of the preceding claims, wherein one of domains I and M is a CH1 domain and the other of domains I and M is a CL domain, and wherein domain I and domain M form a pair of associated CH1/CL domains (“CH1/CL domain pair”).

49. The binding molecule of claim 48, wherein the CH1 domain is an IgG CH1 domain and wherein the CL domain is an IgG CL domain.

50. The binding molecule of claim 49, wherein domain I is a CL domain and domain M is a CH1 domain.

51. The binding molecule of any one of the preceding claims, wherein domain B and domain D are attached via a first CH3 linker and wherein domain G comprises, at its C-terminus, a second CH3 linker, wherein the first CH3 linker and the second CH3 linker each comprise a cysteine capable of forming a disulfide bridge with the cysteine of the other CH3 linker.

52. The binding molecule of any one of claims 38-51, wherein domain B and domain G each comprise an engineered mutation, wherein the engineered mutation of domain B and the engineered mutation of domain G form a disulfide bond.

53. The binding molecule of claim 52, wherein either (i) the engineered mutation of domain B is a H350C mutation and the engineered mutation of domain G is a P355C mutation, or (ii) the engineered mutation of domain B is a P355C mutation and the engineered mutation of domain G is a H350C mutation.

54. The binding molecule of claim 53, wherein the engineered mutation of domain B is a H350C mutation and the engineered mutation of domain G is a P355C mutation.

55. The binding molecule of any one of the preceding claims, which is a bivalent molecule.

56. The binding molecule of any one of the preceding claims, wherein the first ABS and second ABS bind to the same epitope of the same antigen.

57. The binding molecule of any one of the preceding claims, wherein the first ABS and second ABS bind to different epitopes of the same antigen.

58. The binding molecule of any one of the preceding claims, wherein the first ABS binds to a first antigen and the second ABS binds to a second antigen which is different from the first antigen.

59. The binding molecule of any one of the preceding claims, wherein domain D and domain J each comprise a CH2 amino acid sequence.

60. The binding molecule of claim 59, wherein the CH2 amino acid sequence is an IgG CH2 amino acid sequence.

61. The binding molecule of claim 60, wherein domains D and J are IgG CH2 domains.

62. The binding molecule of claim 61, wherein domain D and domain J comprise one or more engineered mutations at positions L234, L235, and P329 of the CH2 domain.

63. The binding molecule of any one of the preceding claims, wherein domain E and domain K each comprise a CH3 amino acid sequence.

64. The binding molecule of claim 63, wherein the CH3 amino acid sequences of domains E and K are IgG CH3 amino acid sequences.

65. The binding molecule of claim 64, wherein domain E and domain K are IgG CH3 domains.

66. The binding molecule of any one of claims 48 and 1-62, wherein the CH1/CL domain pair comprises a first orthogonal modification which comprises an L128C mutation in the CH1 sequence and an F118C mutation in the CL sequence, and wherein the CH1/CL pair comprises a second orthogonal modification which is a charged-pair modification selected from Table 7.

67. The binding molecule of any one of claims 1-66, wherein domain A comprises a VL amino acid sequence and wherein domain F comprises a VH amino acid sequence.

68. The binding molecule of any one of the preceding claims, wherein domain H comprises a VH amino acid sequence and domain L comprises a VL amino acid sequence.

69. The binding molecule of any one of claims 38-67, wherein domain H comprises a VL amino acid sequence and domain L comprises a VH amino acid sequence.

70. The binding molecule of any one of the preceding claims, wherein domain N comprises a VL amino acid sequence and domain P comprises a VH amino acid sequence.

71. The binding molecule of any one of claims 51, 10, and 31, wherein the first CH3 linker is not identical to the second CH3 linker, optionally wherein the first CH3 linker is of a different length than the second CH3 linker.

72. The binding molecule of claim 71, wherein
a. the first CH3 linker sequence is the amino acid sequence AGC and the second CH3 linker sequence is the amino acid sequence AGKGSC,
b. the first CH3 linker sequence is the amino acid sequence AGKGC and the second CH3 linker sequence is the amino acid sequence AGC,
c. the first CH3 linker sequence is the amino acid sequence AGKGSC and the second CH3 linker sequence is the amino acid sequence AGC,
d. the first CH3 linker sequence is the amino acid sequence AGKC and the second CH3 linker sequence is the amino acid sequence AGC,
e. the first CH3 linker sequence is the amino acid sequence GEC and the second CH3 linker sequence is the amino acid sequence GEC,
f. the first CH3 linker sequence is the amino acid sequence AGC and the second CH3 linker sequence is the amino acid sequence AGKGSC,
g. the first CH3 linker sequence is the amino acid sequence AGKGC and the second CH3 linker sequence is the amino acid sequence AGC,
h. the first CH3 linker sequence is the amino acid sequence AGKGSC and the second CH3 linker sequence is the amino acid sequence AGC,
i. the first CH3 linker sequence is the amino acid sequence AGKC and the second CH3 linker sequence is the amino acid sequence AGC,
j. the first CH3 linker sequence is the amino acid sequence AGC and the second CH3 linker sequence is the amino acid sequence AGC,
k. the first CH3 linker sequence is the amino acid sequence AGC and the second CH3 linker sequence is the amino acid sequence AGKGC,
l. the first CH3 linker sequence is the amino acid sequence AGC and the second CH3 linker sequence is the amino acid sequence AGKC,
m. the first CH3 linker sequence is the amino acid sequence AGC and the second CH3 linker sequence is the amino acid sequence GEC,
n. the first CH3 linker sequence is the amino acid sequence AGC and the second CH3 linker sequence is the amino acid sequence PGKC,
o. the first CH3 linker sequence is the amino acid sequence AGKGC and the second CH3 linker sequence is the amino acid sequence AGKGC,
p. the first CH3 linker sequence is the amino acid sequence AGKGC and the second CH3 linker sequence is the amino acid sequence AGKGSC,
q. the first CH3 linker sequence is the amino acid sequence AGKGC and the second CH3 linker sequence is the amino acid sequence AGKC,
r. the first CH3 linker sequence is the amino acid sequence AGKGC and the second CH3 linker sequence is the amino acid sequence GEC,
s. the first CH3 linker sequence is the amino acid sequence AGKGC and the second CH3 linker sequence is the amino acid sequence PGKC,
t. the first CH3 linker sequence is the amino acid sequence AGKGSC and the second CH3 linker sequence is the amino acid sequence AGKGC,
u. the first CH3 linker sequence is the amino acid sequence AGKGSC and the second CH3 linker sequence is the amino acid sequence AGKGSC,
v. the first CH3 linker sequence is the amino acid sequence AGKGSC and the second CH3 linker sequence is the amino acid sequence AGKC,
w. the first CH3 linker sequence is the amino acid sequence AGKGSC and the second CH3 linker sequence is the amino acid sequence GEC,
x. the first CH3 linker sequence is the amino acid sequence AGKGSC and the second CH3 linker sequence is the amino acid sequence PGKC,
y. the first CH3 linker sequence is the amino acid sequence AGKC and the second CH3 linker sequence is the amino acid sequence AGKGC,
z. the first CH3 linker sequence is the amino acid sequence AGKC and the second CH3 linker sequence is the amino acid sequence AGKGSC,
aa. the first CH3 linker sequence is the amino acid sequence AGKC and the second CH3 linker sequence is the amino acid sequence AGKC,
bb. the first CH3 linker sequence is the amino acid sequence AGKC and the second CH3 linker sequence is the amino acid sequence GEC,
cc. the first CH3 linker sequence is the amino acid sequence AGKC and the second CH3 linker sequence is the amino acid sequence PGKC,
dd. the first CH3 linker sequence is the amino acid sequence GEC and the second CH3 linker sequence is the amino acid sequence AGC,
ee. the first CH3 linker sequence is the amino acid sequence GEC and the second CH3 linker sequence is the amino acid sequence AGKGC,
ff. the first CH3 linker sequence is the amino acid sequence GEC and the second CH3 linker sequence is the amino acid sequence AGKGSC,
gg. the first CH3 linker sequence is the amino acid sequence GEC and the second CH3 linker sequence is the amino acid sequence AGKC,
hh. the first CH3 linker sequence is the amino acid sequence GEC and the second CH3 linker sequence is the amino acid sequence GEC,
ii. the first CH3 linker sequence is the amino acid sequence GEC and the second CH3 linker sequence is the amino acid sequence PGKC,
jj. the first CH3 linker sequence is the amino acid sequence PGKC and the second CH3 linker sequence is the amino acid sequence AGC,
kk. the first CH3 linker sequence is the amino acid sequence PGKC and the second CH3 linker sequence is the amino acid sequence AGKGC,
ll. the first CH3 linker sequence is the amino acid sequence PGKC and the second CH3 linker sequence is the amino acid sequence AGKGSC,
mm. the first CH3 linker sequence is the amino acid sequence PGKC and the second CH3 linker sequence is the amino acid sequence AGKC,
nn. the first CH3 linker sequence is the amino acid sequence PGKC and the second CH3 linker sequence is the amino acid sequence GEC,
oo. and the first CH3 linker sequence is the amino acid sequence PGKC and the second CH3 linker sequence is the amino acid sequence PGKC.

73. The binding molecule of claim 72, wherein
a. the first CH3 linker sequence is the amino acid sequence AGC and the second CH3 linker sequence is the amino acid sequence AGKGSC,
b. the first CH3 linker sequence is the amino acid sequence AGKGC and the second CH3 linker sequence is the amino acid sequence AGC,
c. the first CH3 linker sequence is the amino acid sequence AGKGSC and the second CH3 linker sequence is the amino acid sequence AGC, and
d. the first CH3 linker sequence is the amino acid sequence AGKC and the second CH3 linker sequence is the amino acid sequence AGC.

74. The binding molecule of claim 73, wherein the first CH3 linker sequence is the amino acid sequence AGC and the second CH3 linker sequence is the amino acid sequence AGKGSC.

75. A binding molecule comprising a first, second, third, and fourth polypeptide chain, wherein: a) the first polypeptide chain comprises a domain A, a domain B, a domain D, and a domain E, wherein the domains are arranged, from N-terminus to C-terminus, in a A-B-D-E orientation, wherein domain A has a variable region domain amino acid sequence, and wherein domain B, domain D, and domain E have a constant region domain amino acid sequence; (b) the second polypeptide chain comprises a domain F and a domain G, wherein the domains are arranged, from N-terminus to C-terminus, in a F-G orientation, and wherein domain F has a variable region domain amino acid sequence and domain G has a constant region domain amino acid sequence; (c) the third polypeptide chain comprises a domain H, a domain I, a domain J, and a domain K, wherein the domains are arranged, from N-terminus to C-terminus, in a H-I-J-K orientation, and wherein domain H has a variable region domain amino acid sequence, domain I has a constant region amino acid sequence, and domains J and K have a constant region domain amino acid sequence; (d) the fourth polypeptide chain comprises a domain L and a domain M, wherein the domains are arranged, from N-terminus to C-terminus, in a L-M orientation, and wherein domain L has a variable region domain amino acid sequence, and domain M comprises a constant region amino acid sequence, or portion thereof; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the binding molecule; (h) domains D and J comprise CH2; (i) domains E and K comprise CH3; (j) domains D and J comprise one or more engineered mutations at positions L234, L235, and P329 of the CH2 domain.

76. The binding molecule of claim 75, wherein domains A and H comprise VL, domains B and G comprise CH3, domain I comprises CL or CH1, and domain M comprises CH1 or CL, optionally wherein domain I comprises CL and domain M comprises CH1.

77. The binding molecule of any one of claims 62, 75, and 76, wherein the one or more engineered mutations at positions L234, L235, and P329 of the CH2 domain are L234A, L235A, and P329K of the CH2 domain.

78. The binding molecule of any one of claims 62, 75, and 76, wherein the one or more engineered mutations at positions L234, L235, and P329 of the CH2 domain are L234A, L235A, and P329A of the CH2 domain.

79. The binding molecule of any one of claims 62, 75, and 76, wherein the one or more engineered mutations at positions L234, L235, and P329 of the CH2 domain are L234A, L235A, and P329G of the CH2 domain.

80. The binding molecule of any one of claims 62, 75, and 76, wherein the one or more engineered mutations at positions L234, L235, and P329 of the CH2 domain are L234G, L235G, and P329A of the CH2 domain.

81. The binding molecule of any one of claims 62, 75, and 76, wherein the one or more engineered mutations at positions L234, L235, and P329 of the CH2 domain are L234G, L235G, and P329G of the CH2 domain.

82. The binding molecule of any one of claims 62, 75, and 76, wherein the one or more engineered mutations at positions L234, L235, and P329 of the CH2 domain are L234G, L235G, and P329K of the CH2 domain.

83. A CD3 binding molecule, the binding molecule comprising a first antigen binding site specific for a CD3 antigen, wherein the first antigen binding site comprises:
a) a CDR1, a CDR2, and a CDR3 amino acid sequences of a specific light chain variable region (VL) from a specific CD3 antigen binding site, wherein the CDR1, CDR2, and CDR3 VL sequences are selected from Table 8; and
b) a CDR1, a CDR2, and a CDR3 amino acid sequences of a specific heavy chain variable region (VH) from the specific CD3 antigen binding site, wherein the CDR1, CDR2, and CDR3 VH sequences are selected from Table 8.

84. A humanized CD3 binding molecule, comprising:
a. A VH amino acid sequence from an SP34-89 antibody; and
b. A VL amino acid sequence from an SP34-89 antibody,
wherein the VH amino acid sequence comprises a VH mutation selected from the group consisting of N30S, G65D, and S68T.

85. The humanized CD3 binding molecule of claim 84, wherein the VL amino acid sequence is a wild-type SP34-89 VL sequence.

86. The humanized CD3 binding molecule of claim 84, wherein the VL amino acid sequence comprises a W57G mutation.

87. The humanized CD3 binding molecule of any of claims 84-86, wherein the VH mutation is N30S.

88. The humanized CD3 binding molecule of any of claims 84-86, wherein the VH mutation is G65D.

89. The humanized CD3 binding molecule of any of claims 84-86, wherein the VH mutation is S68T.

90. A binding molecule comprising a first, second, third, and fourth polypeptide chain, wherein: (a) the first polypeptide chain comprises a domain A, a domain B, a domain D, and a domain E, wherein the domains are arranged, from N-terminus to C-terminus, in a A-B-D-E orientation, wherein domain A has a variable region domain amino acid sequence, and wherein domain B, domain D, and domain E have a constant region domain amino acid sequence; (b) the second polypeptide chain comprises a domain F and a domain G, wherein the domains are arranged, from N-terminus to C-terminus, in a F-G orientation, and wherein domain F has a variable region domain amino acid sequence and domain G has a constant region domain amino acid sequence; (c) the third polypeptide chain comprises a domain H, a domain I, a domain J, and a domain K, wherein the domains are arranged, from N-terminus to C-terminus, in a H-I-J-K orientation, and wherein domain H has a variable region domain amino acid sequence, domain I has a constant region amino acid sequence, and domains J and K have a constant region domain amino acid sequence; (d) the fourth polypeptide chain comprises a domain L and a domain M, wherein the domains are arranged, from N-terminus to C-terminus, in a L-M orientation, and wherein domain L has a variable region domain amino acid sequence, and domain M comprises a constant region amino acid sequence, or portion thereof; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the binding molecule; (h) at least one of domains B and G or I and M form a CH1/CL domain pair; wherein the CH1/CL domain pair comprises a first orthogonal modification which comprises an L128C mutation in the CH1 sequence and an F118C mutation in the CL sequence, and wherein the CH1/CL pair comprises a second orthogonal modification which is a charged-pair modification selected from Table 7.

91. The binding molecule of claim 90, wherein the binding molecule is multispecific.

92. The binding molecule of claim 90 or 91, wherein domain A is a VL domain and domain F is a VH domain.

93. The binding molecule of any one of claims 90-92, wherein domain B and domain G are each immunoglobulin CH3 domains.

94. The binding molecule of any one of claims 90-93, wherein domain I is a CL domain and domain M is a CH1 domain.

95. The binding molecule of any one of claims 66, 90, and 91, wherein the second orthogonal modification comprises a G166D mutation in the CH1 sequence and a N138K mutation in the CL sequence.

96. The binding molecule of any one of claims 66, 90, and 91, wherein the second orthogonal modification comprises a G166K mutation in the CH1 sequence and a N138D mutation in the CL sequence.

97. The binding molecule of any one of the preceding claims, wherein the sequence that forms the junction between the A domain and the B domain is selected from IKRTPRP, IKRTTFRP, IKRTPREP and IKRTVREP.

98. The binding molecule of claim 97, wherein the sequence that forms the junction between the A domain and the B domain is selected from IKRTPRP and IKRTTFRP.

99. The binding molecule of any one of the preceding claims, wherein at least one CH3 amino acid sequence has a C-terminal tripeptide insertion connecting the CH3 amino acid sequence to a hinge amino acid sequence, wherein the tripeptide insertion is selected from the group consisting of PGK, KSC, and GEC.

100. The binding molecule of any one of the preceding claims, wherein the sequences are human sequences.

101. The binding molecule of any one of the preceding claims, wherein at least one CH3 amino acid sequence has one or more isoallotype mutations.

102. The binding molecule of claim 101, wherein the isoallotype mutations are D356E and L358M.

103. The binding molecule of any of the above claims, wherein the CL amino acid sequence is a Cₖₐₚₚₐ sequence.

104. An isolated polynucleotide or set of polynucleotides encoding one or more polypeptide chains of a binding molecule of any one of the preceding claims.

105. A vector or set of vectors comprising the isolated polynucleotide or set of polynucleotides of claim 104.

106. A pharmaceutical composition, comprising:
a. the binding molecule of any one of the preceding claims, and
b. a pharmaceutically acceptable carrier.

107. A method of treatment, comprising administering to a subject in need of treatment the pharmaceutical composition of claim 106.
